SEQUENCE LISTING (69 sequences)

SEQ ID NO: 1 - protein, 1997 aa, Homo sapiens
MALLIHLKTVSELRGRGDRIAKVTFRGQSFYSRVLENCEDVADFDETFRWPVASSIDRNEMLEIQVFNYSKVFSNKLIGTFRMVLQKVVEESHVEVTDTLIDDNNAIIKTSLCVEVRYQATDGTVGSWDDGDFLGDESLQEEEKDSQETDGLLPGSRPSSRPPGEKSFRRAGRSVFSAMKLGKNRSHKEEPQRPDEPAVLEMEDLDHLAIRLGDGLDPDSVSLASVTALTTNVSNKRSKPDIKMEPSAGRPMDYQVSITVIEARQLVGLNMDPVVCVEVGDDKKYTSMKESTNCPYYNEYFVFDFHVSPDVMFDKIIKISVIHSKNLLRSGTLVGSFKMDVGTVYSQPEHQFHHKWAILSDPDDISSGLKGYVKCDVAVVGKGDNIKTPHKANETDEDDIEGNLLLPEGVPPERQWARFYVKIYRAEGLPRMNTSLMANVKKAFIGENKDLVDPYVQVFFAGQKGKTSVQKSSYEPLWNEQVVFTDLFPPLCKRMKVQIRDSDKVNDVAIGTHFIDLRKISNDGDKGFLPTLGPAWVNMYGSTRNYTLLDEHQDLNEGLGEGVSFRARLLLGLAVEIVDTSNPELTSSTEVQVEQATPISESCAGKMEEFFLFGAFLEASMIDRRNGDKPITFEVTIGNYGNEVDGLSRPQRPRPRKEPGDEEEVDLIQNASDDEAGDAGDLASVSSTPPMRPQVTDRNYFHLPYLERKPCIYIKSWWPDQRRRLYNANIMDHIADKLEEGLNDIQEMIKTEKSYPERRLRGVLEELSCGCCRFLSLADKDQGHSSRTRLDRERLKSCMRELENMGQQARMLRAQVKRHTVRDKLRLCQNFLQKLRFLADEPQHSIPDIFIWMMSNNKRVAYARVPSKDLLFSIVEEETGKDCAKVKTLFLKLPGKRGFGSAGWTVQAKVELYLWLGLSKQRKEFLCGLPCGFQEVKAAQGLGLHAFPPVSLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGNATAGDLLAAFELLQIGPAGKADLPPINGPVDVDRGPIMPVPMGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPSWNTTVRLLRRCRVLCNGGSSSHSTGEVVVTMEPEVPIKKLETMVKLDATSEAVVKVDVAEEEKEKKKKKKGTAEEPEEEEPDESMLDWWSKYFASIDTMKEQLRQQEPSGIDLEEKEEVDNTEGLKGSMKGKEKARAAKEEKKKKTQSSGSGQGSEAPEKKKPKIDELKVYPKELESEFDNFEDWLHTFNLLRGKTGDDEDGSTEEERIVGRFKGSLCVYKVPLPEDVSREAGYDSTYGMFQGIPSNDPINVLVRVYVVRATDLHPADINGKADPYIAIRLGKTDIRDKENYISKQLNPVFGKSFDIEASFPMESMLTVAVYDWDLVGTDDLIGETKIDLENRFYSKHRATCGIAQTYSTHGYNIWRDPMKPSQILTRLCKDGKVDGPHFGPPGRVKVANRVFTGPSEIEDENGQRKPTDEHVALLALRHWEDIPRAGCRLVPEHVETRPLLNPDKPGIEQGRLELWVDMFPMDMPAPGTPLDISPRKPKKYELRVIIWNTDEVVLEDDDFFTGEKSSDIFVRGWLKGQQEDKQDTDVHYHSLTGEGNFNWRYLFPFDYLAAEEKIVISKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTSFIWFLNPLKSARYFLWHTYRWLLLKLLLLLLLLLLLALFLYSVPGYLVKKILGA

SEQ ID NO: 2 - protein, 1230 aa, Homo sapiens
MIKTEKSYPERRLRGVLEELSCGCCRFLSLADKDQGHSSRTRLDRERLKSCMRELENMGQQARMLRAQVKRHTVRDKLRLCQNFLQKLRFLADEPQHSIPDIFIWMMSNNKRVAYARVPSKDLLFSIVEEETGKDCAKVKTLFLKLPGKRGFGSAGWTVQAKVELYLWLGLSKQRKEFLCGLPCGFQEVKAAQGLGLHAFPPVSLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGNATAGDLLAAFELLQIGPAGKADLPPINGPVDVDRGPIMPVPMGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPSWNTTGEVVVTMEPEVPIKKLETMVKLDATSEAVVKVDVAEEEKEKKKKKKGTAEEPEEEEPDESMLDWWSKYFASIDTMKEQLRQQEPSGIDLEEKEEVDNTEGLKGSMKGKEKARAAKEEKKKKTQSSGSGQGSEAPEKKKPKIDELKVYPKELESEFDNFEDWLHTFNLLRGKTGDDEDGSTEEERIVGRFKGSLCVYKVPLPEDVSREAGYDSTYGMFQGIPSNDPINVLVRVYVVRATDLHPADINGKADPYIAIRLGKTDIRDKENYISKQLNPVFGKSFDIEASFPMESMLTVAVYDWDLVGTDDLIGETKIDLENRFYSKHRATCGIAQTYSTHGYNIWRDPMKPSQILTRLCKDGKVDGPHFGPPGRVKVANRVFTGPSEIEDENGQRKPTDEHVALLALRHWEDIPRAGCRLVPEHVETRPLLNPDKPGIEQGRLELWVDMFPMDMPAPGTPLDISPRKPKKYELRVIIWNTDEVVLEDDDFFTGEKSSDIFVRGWLKGQQEDKQDTDVHYHSLTGEGNFNWRYLFPFDYLAAEEKIVISKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTSFIWFLNPLKSARYFLWHTYRWLLLKLLLLLLLLLLLALFLYSVPGYLVKKILGA

SEQ ID NO: 3 - protein, 1230 aa, Homo sapiens
MIKTEKSYPERRLRGVLEELSCGCCRFLSLADKDQGHSSRTRLDRERLKSCMRELENMGQQARMLRAQVKRHTVRDKLRLCQNFLQKLRFLADEPQHSIPDIFIWMMSNNKRVAYARVPSKDLLFSIVEEETGKDCAKVKTLFLKLPGKRGFGSAGWTVQAKVELYLWLGLSKQRKEFLCGLPCGFQEVKAAQGLGLHAFPPVSLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGNATAGDLLAAFELLQIGPAGKADLPPINGPVDVDRGPIMPVPMGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPSWNTTGEVVVTMEPEVPIKKLETMVKLDATSEAVVKVDVAEEEKEKKKKKKGTAEEPEEEEPDESMLDWWSKYFASIDTMKEQLRQQEPSGIDLEEKEEVDNTEGLKGSMKGKEKARAAKEEKKKKTQSSGSGQGSEAPEKKKPKIDELKVYPKELESEFDNFEDWLHTFNLLRGKTGDDEDGSTEEERIVGRFKGSLCVYKVPLPEDVSREAGYDSTYGMFQGIPSNDPINVLVRVYVVRATDLHPADINGKADPYIAIRLGKTDIRDKENYISKQLNPVFGKSFDIEASFPMESMLTVAVYDWDLVGTDDLIGETKIDLENRFYSKHRATCGIAQTYSTHGYNIWRDPMKPSQILTRLCKDGKVDGPHFGPPGRVKVANRVFTGPSEIEDENGQRKPTDEHVALLALRHWEDIPRAGCRLVPEHVETRPLLNPDKPGIEQGRLELWVDMFPMDMPAPGTPLDISPRKPKKYELRVIIWNTDEVVLEDDDFFTGEKSSDIFVRGWLKGQQEDKQDTDVHYHSLTGEGNFNWRYLFPFDYLAAEEKIVISKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTAFVWFLNPLKSIKYLICTRYKWLIIKIVLALLGLLMLGLFLYSLPGYMVKKLLGA

SEQ ID NO: 4 - protein, 1307 aa, Homo sapiens
MMTDTQDGPSESSQIMRSLTPLINREEAFGEAGEAGLWPSITHTPDSQEEGLNDIQEMIKTEKSYPERRLRGVLEELSCGCCRFLSLADKDQGHSSRTRLDRERLKSCMRELENMGQQARMLRAQVKRHTVRDKLRLCQNFLQKLRFLADEPQHSIPDIFIWMMSNNKRVAYARVPSKDLLFSIVEEETGKDCAKVKTLFLKLPGKRGFGSAGWTVQAKVELYLWLGLSKQRKEFLCGLPCGFQEVKAAQGLGLHAFPPVSLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGNATAGDLLAAFELLQIGPAGKADLPPINGPVDVDRGPIMPVPMGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPSWNTTVRLLRRCRVLCNGGSSSHSTGEVVVTMEPEVPIKKLETMVKLDATSEAVVKVDVAEEEKEKKKKKKGTAEEPEEEEPDESMLDWWSKYFASIDTMKEQLRQQEPSGIDLEEKEEVDNTEGLKGSMKGKEKARAAKEEKKKKTQSSGSGQGSEAPEKKKPKIDELKVYPKELESEFDNFEDWLHTFNLLRGKTGDDEDGSTEEERIVGRFKGSLCVYKVPLPEDVSREAGYDSTYGMFQGIPSNDPINVLVRVYVVRATDLHPADINGKADPYIAIRLGKTDIRDKENYISKQLNPVFGKSFDIEASFPMESMLTVAVYDWDLVGTDDLIGETKIDLENRFYSKHRATCGIAQTYSTHGYNIWRDPMKPSQILTRLCKDGKVDGPHFGPPGRVKVANRVFTGPSEIEDENGQRKPTDEHVALLALRHWEDIPRAGCRLVPEHVETRPLLNPDKPGIEQGRLELWVDMFPMDMPAPGTPLDISPRKPKKYELRVIIWNTDEVVLEDDDFFTGEKSSDIFVRGWLKGQQEDKQDTDVHYHSLTGEGNFNWRYLFPFDYLAAEEKIVISKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTSFIWFLNPLKSARYFLWHTYRWLLLKLLLLLLLLLLLALFLYSVPGYLVKKILGA

SEQ ID NO: 5 - protein, 1997 aa, Homo sapiens
MALLIHLKTVSELRGRGDRIAKVTFRGQSFYSRVLENCEDVADFDETFRWPVASSIDRNEMLEIQVFNYSKVFSNKLIGTFRMVLQKVVEESHVEVTDTLIDDNNAIIKTSLCVEVRYQATDGTVGSWDDGDFLGDESLQEEEKDSQETDGLLPGSRPSSRPPGEKSFRRAGRSVFSAMKLGKNRSHKEEPQRPDEPAVLEMEDLDHLAIRLGDGLDPDSVSLASVTALTTNVSNKRSKPDIKMEPSAGRPMDYQVSITVIEARQLVGLNMDPVVCVEVGDDKKYTSMKESTNCPYYNEYFVFDFHVSPDVMFDKIIKISVIHSKNLLRSGTLVGSFKMDVGTVYSQPEHQFHHKWAILSDPDDISSGLKGYVKCDVAVVGKGDNIKTPHKANETDEDDIEGNLLLPEGVPPERQWARFYVKIYRAEGLPRMNTSLMANVKKAFIGENKDLVDPYVQVFFAGQKGKTSVQKSSYEPLWNEQVVFTDLFPPLCKRMKVQIRDSDKVNDVAIGTHFIDLRKISNDGDKGFLPTLGPAWVNMYGSTRNYTLLDEHQDLNEGLGEGVSFRARLLLGLAVEIVDTSNPELTSSTEVQVEQATPISESCAGKMEEFFLFGAFLEASMIDRRNGDKPITFEVTIGNYGNEVDGLSRPQRPRPRKEPGDEEEVDLIQNASDDEAGDAGDLASVSSTPPMRPQVTDRNYFHLPYLERKPCIYIKSWWPDQRRRLYNANIMDHIADKLEEGLNDIQEMIKTEKSYPERRLRGVLEELSCGCCRFLSLADKDQGHSSRTRLDRERLKSCMRELENMGQQARMLRAQVKRHTVRDKLRLCQNFLQKLRFLADEPQHSIPDIFIWMMSNNKRVAYARVPSKDLLFSIVEEETGKDCAKVKTLFLKLPGKRGFGSAGWTVQAKVELYLWLGLSKQRKEFLCGLPCGFQEVKAAQGLGLHAFPPVSLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGNATAGDLLAAFELLQIGPAGKADLPPINGPVDVDRGPIMPVPMGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPSWNTTVRLLRRCRVLCNGGSSSHSTGEVVVTMEPEVPIKKLETMVKLDATSEAVVKVDVAEEEKEKKKKKKGTAEEPEEEEPDESMLDWWSKYFASIDTMKEQLRQQEPSGIDLEEKEEVDNTEGLKGSMKGKEKARAAKEEKKKKTQSSGSGQGSEAPEKKKPKIDELKVYPKELESEFDNFEDWLHTFNLLRGKTGDDEDGSTEEERIVGRFKGSLCVYKVPLPEDVSREAGYDSTYGMFQGIPSNDPINVLVRVYVVRATDLHPADINGKADPYIAIRLGKTDIRDKENYISKQLNPVFGKSFDIEASFPMESMLTVAVYDWDLVGTDDLIGETKIDLENRFYSKHRATCGIAQTYSTHGYNIWRDPMKPSQILTRLCKDGKVDGPHFGPPGRVKVANRVFTGPSEIEDENGQRKPTDEHVALLALRHWEDIPRAGCRLVPEHVETRPLLNPDKPGIEQGRLELWVDMFPMDMPAPGTPLDISPRKPKKYELRVIIWNTDEVVLEDDDFFTGEKSSDIFVRGWLKGQQEDKQDTDVHYHSLTGEGNFNWRYLFPFDYLAAEEKIVISKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTAFVWFLNPLKSIKYLICTRYKWLIIKIVLALLGLLMLGLFLYSLPGYMVKKLLGA

SEQ ID NO: 6 - protein, 1997 aa, Mus musculus
MALIVHLKTVSELRGKGDRIAKVTFRGQSFYSRVLENCEGVADFDETFRWPVASSIDRNEVLEIQIFNYSKVFSNKLIGTFCMVLQKVVEENRVEVTDTLMDDSNAIIKTSLSMEVRYQATDGTVGPWDDGDFLGDESLQEEKDSQETDGLLPGSRPSTRISGEKSFRRAGRSVFSAMKLGKTRSHKEEPQRQDEPAVLEMEDLDHLAIQLGDGLDPDSVSLASVTALTSNVSNKRSKPDIKMEPSAGRPMDYQVSITVIEARQLVGLNMDPVVCVEVGDDKKYTSMKESTNCPYYNEYFVFDFHVSPDVMFDKIIKISVIHSKNLLRSGTLVGSFKMDVGTVYSQPEHQFHHKWAILSDPDDISAGLKGYVKCDVAVVGKGDNIKTPHKANETDEDDIEGNLLLPEGVPPERQWARFYVKIYRAEGLPRMNTSLMANVKKAFIGENKDLVDPYVQVFFAGQKGKTSVQKSSYEPLWNEQVVFTDLFPPLCKRMKVQIRDSDKVNDVAIGTHFIDLRKISNDGDKGFLPTLGPAWVNMYGSTRNYTLLDEHQDLNEGLGEGVSFRARLMLGLAVEILDTSNPELTSSTEVQVEQATPVSESCTGRMEEFFLFGAFLEASMIDRKNGDKPITFEVTIGNYGNEVDGMSRPLRPRPRKEPGDEEEVDLIQNSSDDEGDEAGDLASVSSTPPMRPQITDRNYFHLPYLERKPCIYIKSWWPDQRRRLYNANIMDHIADKLEEGLNDVQEMIKTEKSYPERRLRGVLEELSCGCHRFLSLSDKDQGRSSRTRLDRERLKSCMRELESMGQQAKSLRAQVKRHTVRDKLRSCQNFLQKLRFLADEPQHSIPDVFIWMMSNNKRIAYARVPSKDLLFSIVEEELGKDCAKVKTLFLKLPGKRGFGSAGWTVQAKLELYLWLGLSKQRKDFLCGLPCGFEEVKAAQGLGLHSFPPISLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGSATAGDLLAAFELLQIGPSGKADLPPINGPVDMDRGPIMPVPVGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPNWNTTVRLLRGCHRLRNGGPSSRPTGEVVVSMEPEEPVKKLETMVKLDATSDAVVKVDVAEDEKERKKKKKKGPSEEPEEEEPDESMLDWWSKYFASIDTMKEQLRQHETSGTDLEEKEEMESAEGLKGPMKSKEKSRAAKEEKKKKNQSPGPGQGSEAPEKKKAKIDELKVYPKELESEFDSFEDWLHTFNLLRGKTGDDEDGSTEEERIVGRFKGSLCVYKVPLPEDVSREAGYDPTYGMFQGIPSNDPINVLVRIYVVRATDLHPADINGKADPYIAIKLGKTDIRDKENYISKQLNPVFGKSFDIEASFPMESMLTVAVYDWDLVGTDDLIGETKIDLENRFYSKHRATCGIAQTYSIHGYNIWRDPMKPSQILTRLCKEGKVDGPHFGPHGRVRVANRVFTGPSEIEDENGQRKPTDEHVALSALRHWEDIPRVGCRLVPEHVETRPLLNPDKPGIEQGRLELWVDMFPMDMPAPGTPLDISPRKPKKYELRVIVWNTDEVVLEDDDFFTGEKSSDIFVRGWLKGQQEDKQDTDVHYHSLTGEGNFNWRYLFPFDYLAAEEKIVMSKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTSFIWFLNPLKSARYFLWHTYRWLLLKFLLLFLLLLLFALFLYSLPGYLAKKILGA

SEQ ID NO: 7 - protein, 1977 aa, Mus musculus
MALIVHLKTVSELRGKGDRIAKVTFRGQSFYSRVLENCEGVADFDETFRWPVASSIDRNEVLEIQIFNYSKVFSNKLIGTFCMVLQKVVEENRVEVTDTLMDDSNAIIKTSLSMEVRYQATDGTVGPWDDGDFLGDESLQEEKDSQETDGLLPGSRPSTRISGEKSFRRAGRSVFSAMKLGKTRSHKEEPQRQDEPAVLEMEDLDHLAIQLGDGLDPDSVSLASVTALTSNVSNKRSKPDIKMEPSAGRPMDYQVSITVIEARQLVGLNMDPVVCVEVGDDKKYTSMKESTNCPYYNEYFVFDFHVSPDVMFDKIIKISVIHSKNLLRSGTLVGSFKMDVGTVYSQPEHQFHHKWAILSDPDDISAGLKGYVKCDVAVVGKGDNIKTPHKANETDEDDIEGNLLLPEGVPPERQWARFYVKIYRAEGLPRMNTSLMANVKKAFIGENKDLVDPYVQVFFAGQKGKTSVQKSSYEPLWNEQVVFTDLFPPLCKRMKVQIRDSDKVNDVAIGTHFIDLRKISNDGDKGFLPTLGPAWVNMYGSTRNYTLLDEHQDLNEGLGEGVSFRARLMLGLAVEILDTSNPELTSSTEVQVEQATPVSESCTGRMEEFFLFGAFLEASMIDRKNGDKPITFEVTIGNYGNEVDGMSRPLRPRPRKEPGDEEEVDLIQNSSDDEGDEAGDLASVSSTPPMRPQITDRNYFHLPYLERKPCIYIKSWWPDQRRRLYNANIMDHIADKLEEGLNDVQEMIKTEKSYPERRLRGVLEELSCGCHRFLSLSDKDQGRSSRTRLDRERLKSCMRELESMGQQAKSLRAQVKRHTVRDKLRSCQNFLQKLRFLADEPQHSIPDVFIWMMSNNKRIAYARVPSKDLLFSIVEEELGKDCAKVKTLFLKLPGKRGFGSAGWTVQAKLELYLWLGLSKQRKDFLCGLPCGFEEVKAAQGLGLHSFPPISLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGSATAGDLLAAFELLQIGPSGKADLPPINGPVDMDRGPIMPVPVGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPNWNTTGEVVVSMEPEEPVKKLETMVKLDATSDAVVKVDVAEDEKERKKKKKKGPSEEPEEEEPDESMLDWWSKYFASIDTMKEQLRQHETSGTDLEEKEEMESAEGLKGPMKSKEKSRAAKEEKKKKNQSPGPGQGSEAPEKKKAKIDELKVYPKELESEFDSFEDWLHTFNLLRGKTGDDEDGSTEEERIVGRFKGSLCVYKVPLPEDVSREAGYDPTYGMFQGIPSNDPINVLVRIYVVRATDLHPADINGKADPYIAIKLGKTDIRDKENYISKQLNPVFGKSFDIEASFPMESMLTVAVYDWDLVGTDDLIGETKIDLENRFYSKHRATCGIAQTYSIHGYNIWRDPMKPSQILTRLCKEGKVDGPHFGPHGRVRVANRVFTGPSEIEDENGQRKPTDEHVALSALRHWEDIPRVGCRLVPEHVETRPLLNPDKPGIEQGRLELWVDMFPMDMPAPGTPLDISPRKPKKYELRVIVWNTDEVVLEDDDFFTGEKSSDIFVRGWLKGQQEDKQDTDVHYHSLTGEGNFNWRYLFPFDYLAAEEKIVMSKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTSFIWFLNPLKSARYFLWHTYRWLLLKFLLLFLLLLLFALFLYSLPGYLAKKILGA

SEQ ID NO: 8 - protein, 1992 aa, Mus musculus
MALIVHLKTVSELRGKGDRIAKVTFRGQSFYSRVLENCEGVADFDETFRWPVASSIDRNEVLEIQIFNYSKVFSNKLIGTFCMVLQKVVEENRVEVTDTLMDDSNAIIKTSLSMEVRYQATDGTVGPWDDGDFLGDESLQEEKDSQETDGLLPGSRPSTRISGEKSFRSKGREKTKGGRDGEHKAGRSVFSAMKLGKTRSHKEEPQRQDEPAVLEMEDLDHLAIQLGDGLDPDSVSLASVTALTSNVSNKRSKPDIKMEPSAGRPMDYQVSITVIEARQLVGLNMDPVVCVEVGDDKKYTSMKESTNCPYYNEYFVFDFHVSPDVMFDKIIKISVIHSKNLLRSGTLVGSFKMDVGTVYSQPEHQFHHKWAILSDPDDISAGLKGYVKCDVAVVGKGDNIKTPHKANETDEDDIEGNLLLPEGVPPERQWARFYVKIYRAEGLPRMNTSLMANVKKAFIGENKDLVDPYVQVFFAGQKGKTSVQKSSYEPLWNEQVVFTDLFPPLCKRMKVQIRDSDKVNDVAIGTHFIDLRKISNDGDKGFLPTLGPAWVNMYGSTRNYTLLDEHQDLNEGLGEGVSFRARLMLGLAVEILDTSNPELTSSTEVQVEQATPVSESCTGRMEEFFLFGAFLEASMIDRKNGDKPITFEVTIGNYGNEVDGMSRPLRPRPRKEPGDEEEVDLIQNSSDDEGDEAGDLASVSSTPPMRPQITDRNYFHLPYLERKPCIYIKSWWPDQRRRLYNANIMDHIADKLEEGLNDVQEMIKTEKSYPERRLRGVLEELSCGCHRFLSLSDKDQGRSSRTRLDRERLKSCMRELESMGQQAKSLRAQVKRHTVRDKLRSCQNFLQKLRFLADEPQHSIPDVFIWMMSNNKRIAYARVPSKDLLFSIVEEELGKDCAKVKTLFLKLPGKRGFGSAGWTVQAKLELYLWLGLSKQRKDFLCGLPCGFEEVKAAQGLGLHSFPPISLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGSATAGDLLAAFELLQIGPSGKADLPPINGPVDMDRGPIMPVPVGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPNWNTTGEVVVSMEPEEPVKKLETMVKLDATSDAVVKVDVAEDEKERKKKKKKGPSEEPEEEEPDESMLDWWSKYFASIDTMKEQLRQHETSGTDLEEKEEMESAEGLKGPMKSKEKSRAAKEEKKKKNQSPGPGQGSEAPEKKKAKIDELKVYPKELESEFDSFEDWLHTFNLLRGKTGDDEDGSTEEERIVGRFKGSLCVYKVPLPEDVSREAGYDPTYGMFQGIPSNDPINVLVRIYVVRATDLHPADINGKADPYIAIKLGKTDIRDKENYISKQLNPVFGKSFDIEASFPMESMLTVAVYDWDLVGTDDLIGETKIDLENRFYSKHRATCGIAQTYSIHGYNIWRDPMKPSQILTRLCKEGKVDGPHFGPHGRVRVANRVFTGPSEIEDENGQRKPTDEHVALSALRHWEDIPRVGCRLVPEHVETRPLLNPDKPGIEQGRLELWVDMFPMDMPAPGTPLDISPRKPKKYELRVIVWNTDEVVLEDDDFFTGEKSSDIFVRGWLKGQQEDKQDTDVHYHSLTGEGNFNWRYLFPFDYLAAEEKIVMSKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTAFVWFLNPLKSIKYLICTRYKWLIIKIVLALLGLLMLALFLYSLPGYMVKKLLGA

SEQ ID NO: 9 - protein, 1977 aa, Mus musculus
MALIVHLKTVSELRGKGDRIAKVTFRGQSFYSRVLENCEGVADFDETFRWPVASSIDRNEVLEIQIFNYSKVFSNKLIGTFCMVLQKVVEENRVEVTDTLMDDSNAIIKTSLSMEVRYQATDGTVGPWDDGDFLGDESLQEEKDSQETDGLLPGSRPSTRISGEKSFRRAGRSVFSAMKLGKTRSHKEEPQRQDEPAVLEMEDLDHLAIQLGDGLDPDSVSLASVTALTSNVSNKRSKPDIKMEPSAGRPMDYQVSITVIEARQLVGLNMDPVVCVEVGDDKKYTSMKESTNCPYYNEYFVFDFHVSPDVMFDKIIKISVIHSKNLLRSGTLVGSFKMDVGTVYSQPEHQFHHKWAILSDPDDISAGLKGYVKCDVAVVGKGDNIKTPHKANETDEDDIEGNLLLPEGVPPERQWARFYVKIYRAEGLPRMNTSLMANVKKAFIGENKDLVDPYVQVFFAGQKGKTSVQKSSYEPLWNEQVVFTDLFPPLCKRMKVQIRDSDKVNDVAIGTHFIDLRKISNDGDKGFLPTLGPAWVNMYGSTRNYTLLDEHQDLNEGLGEGVSFRARLMLGLAVEILDTSNPELTSSTEVQVEQATPVSESCTGRMEEFFLFGAFLEASMIDRKNGDKPITFEVTIGNYGNEVDGMSRPLRPRPRKEPGDEEEVDLIQNSSDDEGDEAGDLASVSSTPPMRPQITDRNYFHLPYLERKPCIYIKSWWPDQRRRLYNANIMDHIADKLEEGLNDVQEMIKTEKSYPERRLRGVLEELSCGCHRFLSLSDKDQGRSSRTRLDRERLKSCMRELESMGQQAKSLRAQVKRHTVRDKLRSCQNFLQKLRFLADEPQHSIPDVFIWMMSNNKRIAYARVPSKDLLFSIVEEELGKDCAKVKTLFLKLPGKRGFGSAGWTVQAKLELYLWLGLSKQRKDFLCGLPCGFEEVKAAQGLGLHSFPPISLVYTKKQAFQLRAHMYQARSLFAADSSGLSDPFARVFFINQSQCTEVLNETLCPTWDQMLVFDNLELYGEAHELRDDPPIIVIEIYDQDSMGKADFMGRTFAKPLVKMADEAYCPPRFPPQLEYYQIYRGSATAGDLLAAFELLQIGPSGKADLPPINGPVDMDRGPIMPVPVGIRPVLSKYRVEVLFWGLRDLKRVNLAQVDRPRVDIECAGKGVQSSLIHNYKKNPNFNTLVKWFEVDLPENELLHPPLNIRVVDCRAFGRYTLVGSHAVSSLRRFIYRPPDRSAPNWNTTGEVVVSMEPEEPVKKLETMVKLDATSDAVVKVDVAEDEKERKKKKKKGPSEEPEEEEPDESMLDWWSKYFASIDTMKEQLRQHETSGTDLEEKEEMESAEGLKGPMKSKEKSRAAKEEKKKKNQSPGPGQGSEAPEKKKAKIDELKVYPKELESEFDSFEDWLHTFNLLRGKTGDDEDGSTEEERIVGRFKGSLCVYKVPLPEDVSREAGYDPTYGMFQGIPSNDPINVLVRIYVVRATDLHPADINGKADPYIAIKLGKTDIRDKENYISKQLNPVFGKSFDIEASFPMESMLTVAVYDWDLVGTDDLIGETKIDLENRFYSKHRATCGIAQTYSIHGYNIWRDPMKPSQILTRLCKEGKVDGPHFGPHGRVRVANRVFTGPSEIEDENGQRKPTDEHVALSALRHWEDIPRVGCRLVPEHVETRPLLNPDKPGIEQGRLELWVDMFPMDMPAPGTPLDISPRKPKKYELRVIVWNTDEVVLEDDDFFTGEKSSDIFVRGWLKGQQEDKQDTDVHYHSLTGEGNFNWRYLFPFDYLAAEEKIVMSKKESMFSWDETEYKIPARLTLQIWDADHFSADDFLGAIELDLNRFPRGAKTAKQCTMEMATGEVDVPLVSIFKQKRVKGWWPLLARNENDEFELTGKVEAELHLLTAEEAEKNPVGLARNEPDPLEKPNRPDTAFVWFLNPLKSIKYLICTRYKWLIIKIVLALLGLLMLALFLYSLPGYMVKKLLGA

SEQ ID NO: 10 - DNA, 7171 bp, Homo sapiens
  source 1..7171: mol_type = genomic DNA
atcggaggggggtcgggaggaggaggaggaggcagcggcagagaagagagaggcgtgtgagccgtgctccaccggctagctccttcccgctgctcctgcctggcagtgccaggcagcccacaccagcatggccttgctcatccacctcaagacagtctcggagctgcggggcaggggcgaccggatcgccaaagtgactttccgagggcaatccttctactctcgggtcctggagaactgtgaggatgtggctgactttgatgagacatttcggtggccggtggccagcagcatcgacagaaatgagatgctggagattcaggttttcaactacagcaaagtcttcagcaacaagctcatcgggaccttccgcatggtgctgcagaaggtggtagaggagagccatgtggaggtgactgacacgctgattgatgacaacaatgctatcatcaagaccagcctgtgcgtggaggtccggtatcaggccactgacggcacagtgggctcctgggacgatggggacttcctgggagatgagtctcttcaagaggaagagaaggacagccaagagacggatggactgctcccaggctcccggcccagctcccggcccccaggagagaagagcttccggagagccgggaggagcgtgttctccgccatgaagctcggcaaaaaccggtctcacaaggaggagccccaaagaccagatgaaccggcggtgctggagatggaagaccttgaccatctggccattcggctaggagatggactggatcccgactcggtgtctctagcctcagtcacagctctcaccactaatgtctccaacaagcgatctaagccagacattaagatggagccaagtgctgggcggcccatggattaccaggtcagcatcacggtgatcgaggcccggcagctggtgggcttgaacatggaccctgtggtgtgcgtggaggtgggtgacgacaagaagtacacatccatgaaggagtccactaactgcccctattacaacgagtacttcgtcttcgacttccatgtctctccggatgtcatgtttgacaagatcatcaagatttcggtgattcactccaagaacctgctgcgcagtggcaccctggtgggctccttcaaaatggacgtgggaaccgtgtactcgcagccagagcaccagttccatcacaagtgggccatcctgtctgaccccgatgacatctcctcggggctgaagggctacgtgaagtgtgacgttgccgtggtgggcaaaggggacaacatcaagacgccccacaaggccaatgagaccgacgaagatgacattgaggggaacttgctgctccccgagggggtgccccccgaacgccagtgggcccggttctatgtgaaaatttaccgagcagaggggctgccccgtatgaacacaagcctcatggccaatgtaaagaaggctttcatcggtgaaaacaaggacctcgtggacccctacgtgcaagtcttctttgctggccagaagggcaagacttcagtgcagaagagcagctatgagcccctgtggaatgagcaggtcgtctttacagacctcttccccccactctgcaaacgcatgaaggtgcagatccgagactcggacaaggtcaacgacgtggccatcggcacccacttcattgacctgcgcaagatttctaatgacggagacaaaggcttcctgcccacactgggcccagcctgggtgaacatgtacggctccacacgtaactacacgctgctggatgagcatcaggacctgaacgagggcctgggggagggtgtgtccttccgggcccggctcctgctgggcctggctgtggagatcgtagacacctccaaccctgagctcaccagctccacagaggtgcaggtggagcaggccacgcccatctcggagagctgtgcaggtaaaatggaagaattctttctctttggagccttcctggaggcctcaatgatcgaccggagaaacggagacaagcccatcacctttgaggtcaccataggcaactatgggaacgaagttgatggcctgtcccggccccagcggcctcggccccggaaggagccgggggatgaggaagaagtagacctgattcagaacgcaagtgatgacgaggccggtgatgccggggacctggcctcagtctcctccactccaccaatgcggccccaggtcaccgacaggaactacttccatctgccctacctggagcgaaagccctgcatctacatcaagagctggtggccggaccagcgccgccgcctctacaatgccaacatcatggaccacattgccgacaagctggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggaaaacatggggcagcaggccaggatgctgcgggcccaggtgaagcggcacacggtgcgggacaagctgaggctgtgccagaacttcctgcagaagctgcgcttcctggcggacgagccccagcacagcattcccgacatcttcatctggatgatgagcaacaacaagcgtgtcgcctatgcccgtgtgccctccaaggacctgctcttctccatcgtggaggaggagactggcaaggactgcgccaaggtcaagacgctcttccttaagctgccagggaagcggggcttcggctcggcaggctggacagtgcaggccaaggtggagctgtacctgtggctgggcctcagcaaacagcgcaaggagttcctgtgcggcctgccctgtggcttccaggaggtcaaggcagcccagggcctgggcctgcatgccttcccacccgtcagcctggtctacaccaagaagcaggcgttccagctccgagcgcacatgtaccaggcccgcagcctctttgccgccgacagcagcggactctcagacccctttgcccgcgtcttcttcatcaatcagagtcagtgcacagaggtgctgaatgagaccctgtgtcccacctgggaccagatgctggtgttcgacaacctggagctctatggtgaagctcatgagctgagggacgatccgcccatcattgtcattgaaatctatgaccaggattccatgggcaaagctgacttcatgggccggaccttcgccaaacccctggtgaagatggcagacgaggcgtactgcccaccccgcttcccacctcagctcgagtactaccagatctaccgtggcaacgccacagctggagacctgctggcggccttcgagctgctgcagattggaccagcagggaaggctgacctgccccccatcaatggcccggtggacgtggaccgaggtcccatcatgcccgtgcccatgggcatccggcccgtgctcagcaagtaccgagtggaggtgctgttctggggcctacgggacctaaagcgggtgaacctggcccaggtggaccggccacgggtggacatcgagtgtgcagggaagggggtgcagtcgtccctgatccacaattataagaagaaccccaacttcaacaccctcgtcaagtggtttgaagtggacctcccagagaacgagctgctgcacccgcccttgaacatccgtgtggtggactgccgggccttcggtcgctacacactggtgggctcccatgccgtcagctccctgcgacgcttcatctaccggcccccagaccgctcggcccccagctggaacaccacggtcaggcttctccggcgctgccgtgtgctgtgcaatgggggctcctcctctcactccacaggggaggttgtggtgactatggagccagaggtacccatcaagaaactggagaccatggtgaagctggacgcgacttctgaagctgttgtcaaggtggatgtggctgaggaggagaaggagaagaagaagaagaagaagggcactgcggaggagccagaggaggaggagccagacgagagcatgctggactggtggtccaagtactttgcctccattgacaccatgaaggagcaacttcgacaacaagagccctctggaattgacttggaggagaaggaggaagtggacaataccgagggcctgaaggggtcaatgaagggcaaggagaaggcaagggctgccaaagaggagaagaagaagaaaactcagagctctggctctggccaggggtccgaggcccccgagaagaagaaacccaagattgatgagcttaaggtataccccaaagagctggagtccgagtttgataactttgaggactggctgcacactttcaacttgcttcggggcaagaccggggatgatgaggatggctccaccgaggaggagcgcattgtgggacgcttcaagggctccctctgcgtgtacaaagtgccactcccagaggacgtgtcccgggaagccggctacgactccacctacggcatgttccagggcatcccgagcaatgaccccatcaatgtgctggtccgagtctatgtggtccgggccacggacctgcaccctgctgacatcaacggcaaagctgacccctacatcgccatccggctaggcaagactgacatccgcgacaaggagaactacatctccaagcagctcaaccctgtctttgggaagtcctttgacatcgaggcctccttccccatggaatccatgctgacggtggctgtgtatgactgggacctggtgggcactgatgacctcattggggaaaccaagatcgacctggagaaccgcttctacagcaagcaccgcgccacctgcggcatcgcccagacctactccacacatggctacaatatctggcgggaccccatgaagcccagccagatcctgacccgcctctgcaaagacggcaaagtggacggcccccactttgggccccctgggagagtgaaggtggccaaccgcgtcttcactgggccctctgagattgaggacgagaacggtcagaggaagcccacagacgagcatgtggcgctgttggccctgaggcactgggaggacatcccccgcgcaggctgccgcctggtgccagagcatgtggagacgaggccgctgctcaaccccgacaagccgggcatcgagcagggccgcctggagctgtgggtggacatgttccccatggacatgccagcccctgggacgcctctggacatctcacctcggaagcccaagaagtacgagctgcgggtcatcatctggaacacagatgaggtggtcttggaggacgacgacttcttcacaggggagaagtccagtgacatcttcgtgagggggtggctgaagggccagcaggaggacaagcaggacacagacgtccactaccactccctcactggcgagggcaacttcaactggcgctacctgttccccttcgactacctggcggcggaggagaagatcgtcatctccaagaaggagtccatgttctcctgggacgagaccgagtacaagatccccgcgcggctcaccctgcagatctgggatgcggaccacttctccgctgacgacttcctgggggccatcgagctggacctgaaccggttcccgcggggcgcaaagacagccaagcagtgcaccatggagatggccaccggggaggtggacgtgcccctcgtgtccatcttcaagcaaaagcgcgtcaaaggctggtggcccctcctggcccgcaatgagaacgatgagtttgagctcacgggcaaggtggaggctgagctgcatttactgacagcagaggaggcagagaagaacccagtgggcctggcccgcaatgaacctgaccccctagagaaacccaaccggcccgacacgagcttcatctggttcctgaaccctctcaagtcggctcgctacttcttgtggcacacgtatcgctggctgctcctcaaactgttgctgctcctgctgctgctcctcctcctcgccctgttcctctactctgtgcctggctacctggtcaagaaaatcctcggggcctgagcccagtggcctcctggccggcccgacacggccttcgtctggttcctcaaccctctcaagtccatcaagtacctcatctgcacccggtacaagtggctcatcatcaagatcgtgctggcgctgttggggctgctcatgttggggctcttcctctacagcctccctggctacatggtcaaaaagctccttggggcatgaaggccgccagctcccgccagccgctccccagccctgccgcatttcctttcagtggcttggactctttcccatctcccctggggagcctgaggagcccagcgtccactcttcatgccttgggccgagcctgcctcctgcttgcgggggccgcctgtcctcactgccccaggctgcggcttgcccagtcccgcccctctgacccctgcctgtgggctggggagccttggatggggtggggacctggaatgggtctctcttgccccacctggctgaggcgccacccttcttcaggcccaggctccagaggaagactcctgaaaccctccccaggtcttccaagtacaggattgaagctttagtgaaattaaccaaggaccatgggtcagtgcccagggctttaaaaagaatgaacgagcaaaaggtatccccgccgtgacccctgcagatagcaccggtctttgatccgcagcaggggccagaccctgcccacaagtcccagcgcggctgcttctgccactgctgggctccacttggctcctctcacttcccagggggtcgcctgtcctgcctgtgggtttccatggcttcccagagctccctctgccccagccagcgcctccaggcccagctgaggagctgtgagaagcagcagaggggactccccatcccgggcacaccctgtcctcccacccctgcccccttgcccttccagccctttcagctgcagctgggagctggcccgtcaagtgctgcccctgcctgtgtctgggtttctgttggctgtttttcttttcttgagtggtgatttttctctaaataaaagaagtcaagcactgaaaaaaaaaaaaaaaa

SEQ ID NO: 11 - DNA, 4969 bp, Homo sapiens
  source 1..4969: mol_type = genomic DNA
ccgtgagttctgcccaggccctgtgagctcaccagagccacagactcacagcccagaggtggcttcttccttcaggaactgaagaacccccatgaacaccaacatctccaggttctgagaacagaacctgggaaattgatgacttcctcatgatgaccgatactcaggatggccctagcgagagctcccagatcatgaggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggaaaacatggggcagcaggccaggatgctgcgggcccaggtgaagcggcacacggtgcgggacaagctgaggctgtgccagaacttcctgcagaagctgcgcttcctggcggacgagccccagcacagcattcccgacatcttcatctggatgatgagcaacaacaagcgtgtcgcctatgcccgtgtgccctccaaggacctgctcttctccatcgtggaggaggagactggcaaggactgcgccaaggtcaagacgctcttccttaagctgccagggaagcggggcttcggctcggcaggctggacagtgcaggccaaggtggagctgtacctgtggctgggcctcagcaaacagcgcaaggagttcctgtgcggcctgccctgtggcttccaggaggtcaaggcagcccagggcctgggcctgcatgccttcccacccgtcagcctggtctacaccaagaagcaggcgttccagctccgagcgcacatgtaccaggcccgcagcctctttgccgccgacagcagcggactctcagacccctttgcccgcgtcttcttcatcaatcagagtcagtgcacagaggtgctgaatgagaccctgtgtcccacctgggaccagatgctggtgttcgacaacctggagctctatggtgaagctcatgagctgagggacgatccgcccatcattgtcattgaaatctatgaccaggattccatgggcaaagctgacttcatgggccggaccttcgccaaacccctggtgaagatggcagacgaggcgtactgcccaccccgcttcccacctcagctcgagtactaccagatctaccgtggcaacgccacagctggagacctgctggcggccttcgagctgctgcagattggaccagcagggaaggctgacctgccccccatcaatggcccggtggacgtggaccgaggtcccatcatgcccgtgcccatgggcatccggcccgtgctcagcaagtaccgagtggaggtgctgttctggggcctacgggacctaaagcgggtgaacctggcccaggtggaccggccacgggtggacatcgagtgtgcagggaagggggtgcagtcgtccctgatccacaattataagaagaaccccaacttcaacaccctcgtcaagtggtttgaagtggacctcccagagaacgagctgctgcacccgcccttgaacatccgtgtggtggactgccgggccttcggtcgctacacactggtgggctcccatgccgtcagctccctgcgacgcttcatctaccggcccccagaccgctcggcccccagctggaacaccacgggggaggttgtggtgactatggagccagaggtacccatcaagaaactggagaccatggtgaagctggacgcgacttctgaagctgttgtcaaggtggatgtggctgaggaggagaaggagaagaagaagaagaagaagggcactgcggaggagccagaggaggaggagccagacgagagcatgctggactggtggtccaagtactttgcctccattgacaccatgaaggagcaacttcgacaacaagagccctctggaattgacttggaggagaaggaggaagtggacaataccgagggcctgaaggggtcaatgaagggcaaggagaaggcaagggctgccaaagaggagaagaagaagaaaactcagagctctggctctggccaggggtccgaggcccccgagaagaagaaacccaagattgatgagcttaaggtataccccaaagagctggagtccgagtttgataactttgaggactggctgcacactttcaacttgcttcggggcaagaccggggatgatgaggatggctccaccgaggaggagcgcattgtgggacgcttcaagggctccctctgcgtgtacaaagtgccactcccagaggacgtgtcccgggaagccggctacgactccacctacggcatgttccagggcatcccgagcaatgaccccatcaatgtgctggtccgagtctatgtggtccgggccacggacctgcaccctgctgacatcaacggcaaagctgacccctacatcgccatccggctaggcaagactgacatccgcgacaaggagaactacatctccaagcagctcaaccctgtctttgggaagtcctttgacatcgaggcctccttccccatggaatccatgctgacggtggctgtgtatgactgggacctggtgggcactgatgacctcattggggaaaccaagatcgacctggagaaccgcttctacagcaagcaccgcgccacctgcggcatcgcccagacctactccacacatggctacaatatctggcgggaccccatgaagcccagccagatcctgacccgcctctgcaaagacggcaaagtggacggcccccactttgggccccctgggagagtgaaggtggccaaccgcgtcttcactgggccctctgagattgaggacgagaacggtcagaggaagcccacagacgagcatgtggcgctgttggccctgaggcactgggaggacatcccccgcgcaggctgccgcctggtgccagagcatgtggagacgaggccgctgctcaaccccgacaagccgggcatcgagcagggccgcctggagctgtgggtggacatgttccccatggacatgccagcccctgggacgcctctggacatctcacctcggaagcccaagaagtacgagctgcgggtcatcatctggaacacagatgaggtggtcttggaggacgacgacttcttcacaggggagaagtccagtgacatcttcgtgagggggtggctgaagggccagcaggaggacaagcaggacacagacgtccactaccactccctcactggcgagggcaacttcaactggcgctacctgttccccttcgactacctggcggcggaggagaagatcgtcatctccaagaaggagtccatgttctcctgggacgagaccgagtacaagatccccgcgcggctcaccctgcagatctgggatgcggaccacttctccgctgacgacttcctgggggccatcgagctggacctgaaccggttcccgcggggcgcaaagacagccaagcagtgcaccatggagatggccaccggggaggtggacgtgcccctcgtgtccatcttcaagcaaaagcgcgtcaaaggctggtggcccctcctggcccgcaatgagaacgatgagtttgagctcacgggcaaggtggaggctgagctgcatttactgacagcagaggaggcagagaagaacccagtgggcctggcccgcaatgaacctgaccccctagagaaacccaaccggcccgacacgagcttcatctggttcctgaaccctctcaagtcggctcgctacttcttgtggcacacgtatcgctggctgctcctcaaactgttgctgctcctgctgctgctcctcctcctcgccctgttcctctactctgtgcctggctacctggtcaagaaaatcctcggggcctgagcccagtggcctcctggccggcccgacacggccttcgtctggttcctcaaccctctcaagtccatcaagtacctcatctgcacccggtacaagtggctcatcatcaagatcgtgctggcgctgttggggctgctcatgttggggctcttcctctacagcctccctggctacatggtcaaaaagctccttggggcatgaaggccgccagctcccgccagccgctccccagccctgccgcatttcctttcagtggcttggactctttcccatctcccctggggagcctgaggagcccagcgtccactcttcatgccttgggccgagcctgcctcctgcttgcgggggccgcctgtcctcactgccccaggctgcggcttgcccagtcccgcccctctgacccctgcctgtgggctggggagccttggatggggtggggacctggaatgggtctctcttgccccacctggctgaggcgccacccttcttcaggcccaggctccagaggaagactcctgaaaccctccccaggtcttccaagtacaggattgaagctttagtgaaattaaccaaggaccatgggtcagtgcccagggctttaaaaagaatgaacgagcaaaaggtatccccgccgtgacccctgcagatagcaccggtctttgatccgcagcaggggccagaccctgcccacaagtcccagcgcggctgcttctgccactgctgggctccacttggctcctctcacttcccagggggtcgcctgtcctgcctgtgggtttccatggcttcccagagctccctctgccccagccagcgcctccaggcccagctgaggagctgtgagaagcagcagaggggactccccatcccgggcacaccctgtcctcccacccctgcccccttgcccttccagccctttcagctgcagctgggagctggcccgtcaagtgctgcccctgcctgtgtctgggtttctgttggctgtttttcttttcttgagtggtgatttttctctaaataaaagaagtcaagcactgaaaaaaaaaaaaaaaa

SEQ ID NO: 12 - DNA, 4771 bp, Homo sapiens
  source 1..4771: mol_type = genomic DNA
ccgtgagttctgcccaggccctgtgagctcaccagagccacagactcacagcccagaggtggcttcttccttcaggaactgaagaacccccatgaacaccaacatctccaggttctgagaacagaacctgggaaattgatgacttcctcatgatgaccgatactcaggatggccctagcgagagctcccagatcatgaggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggaaaacatggggcagcaggccaggatgctgcgggcccaggtgaagcggcacacggtgcgggacaagctgaggctgtgccagaacttcctgcagaagctgcgcttcctggcggacgagccccagcacagcattcccgacatcttcatctggatgatgagcaacaacaagcgtgtcgcctatgcccgtgtgccctccaaggacctgctcttctccatcgtggaggaggagactggcaaggactgcgccaaggtcaagacgctcttccttaagctgccagggaagcggggcttcggctcggcaggctggacagtgcaggccaaggtggagctgtacctgtggctgggcctcagcaaacagcgcaaggagttcctgtgcggcctgccctgtggcttccaggaggtcaaggcagcccagggcctgggcctgcatgccttcccacccgtcagcctggtctacaccaagaagcaggcgttccagctccgagcgcacatgtaccaggcccgcagcctctttgccgccgacagcagcggactctcagacccctttgcccgcgtcttcttcatcaatcagagtcagtgcacagaggtgctgaatgagaccctgtgtcccacctgggaccagatgctggtgttcgacaacctggagctctatggtgaagctcatgagctgagggacgatccgcccatcattgtcattgaaatctatgaccaggattccatgggcaaagctgacttcatgggccggaccttcgccaaacccctggtgaagatggcagacgaggcgtactgcccaccccgcttcccacctcagctcgagtactaccagatctaccgtggcaacgccacagctggagacctgctggcggccttcgagctgctgcagattggaccagcagggaaggctgacctgccccccatcaatggcccggtggacgtggaccgaggtcccatcatgcccgtgcccatgggcatccggcccgtgctcagcaagtaccgagtggaggtgctgttctggggcctacgggacctaaagcgggtgaacctggcccaggtggaccggccacgggtggacatcgagtgtgcagggaagggggtgcagtcgtccctgatccacaattataagaagaaccccaacttcaacaccctcgtcaagtggtttgaagtggacctcccagagaacgagctgctgcacccgcccttgaacatccgtgtggtggactgccgggccttcggtcgctacacactggtgggctcccatgccgtcagctccctgcgacgcttcatctaccggcccccagaccgctcggcccccagctggaacaccacgggggaggttgtggtgactatggagccagaggtacccatcaagaaactggagaccatggtgaagctggacgcgacttctgaagctgttgtcaaggtggatgtggctgaggaggagaaggagaagaagaagaagaagaagggcactgcggaggagccagaggaggaggagccagacgagagcatgctggactggtggtccaagtactttgcctccattgacaccatgaaggagcaacttcgacaacaagagccctctggaattgacttggaggagaaggaggaagtggacaataccgagggcctgaaggggtcaatgaagggcaaggagaaggcaagggctgccaaagaggagaagaagaagaaaactcagagctctggctctggccaggggtccgaggcccccgagaagaagaaacccaagattgatgagcttaaggtataccccaaagagctggagtccgagtttgataactttgaggactggctgcacactttcaacttgcttcggggcaagaccggggatgatgaggatggctccaccgaggaggagcgcattgtgggacgcttcaagggctccctctgcgtgtacaaagtgccactcccagaggacgtgtcccgggaagccggctacgactccacctacggcatgttccagggcatcccgagcaatgaccccatcaatgtgctggtccgagtctatgtggtccgggccacggacctgcaccctgctgacatcaacggcaaagctgacccctacatcgccatccggctaggcaagactgacatccgcgacaaggagaactacatctccaagcagctcaaccctgtctttgggaagtcctttgacatcgaggcctccttccccatggaatccatgctgacggtggctgtgtatgactgggacctggtgggcactgatgacctcattggggaaaccaagatcgacctggagaaccgcttctacagcaagcaccgcgccacctgcggcatcgcccagacctactccacacatggctacaatatctggcgggaccccatgaagcccagccagatcctgacccgcctctgcaaagacggcaaagtggacggcccccactttgggccccctgggagagtgaaggtggccaaccgcgtcttcactgggccctctgagattgaggacgagaacggtcagaggaagcccacagacgagcatgtggcgctgttggccctgaggcactgggaggacatcccccgcgcaggctgccgcctggtgccagagcatgtggagacgaggccgctgctcaaccccgacaagccgggcatcgagcagggccgcctggagctgtgggtggacatgttccccatggacatgccagcccctgggacgcctctggacatctcacctcggaagcccaagaagtacgagctgcgggtcatcatctggaacacagatgaggtggtcttggaggacgacgacttcttcacaggggagaagtccagtgacatcttcgtgagggggtggctgaagggccagcaggaggacaagcaggacacagacgtccactaccactccctcactggcgagggcaacttcaactggcgctacctgttccccttcgactacctggcggcggaggagaagatcgtcatctccaagaaggagtccatgttctcctgggacgagaccgagtacaagatccccgcgcggctcaccctgcagatctgggatgcggaccacttctccgctgacgacttcctgggggccatcgagctggacctgaaccggttcccgcggggcgcaaagacagccaagcagtgcaccatggagatggccaccggggaggtggacgtgcccctcgtgtccatcttcaagcaaaagcgcgtcaaaggctggtggcccctcctggcccgcaatgagaacgatgagtttgagctcacgggcaaggtggaggctgagctgcatttactgacagcagaggaggcagagaagaacccagtgggcctggcccgcaatgaacctgaccccctagagaaacccaaccggcccgacacggccttcgtctggttcctcaaccctctcaagtccatcaagtacctcatctgcacccggtacaagtggctcatcatcaagatcgtgctggcgctgttggggctgctcatgttggggctcttcctctacagcctccctggctacatggtcaaaaagctccttggggcatgaaggccgccagctcccgccagccgctccccagccctgccgcatttcctttcagtggcttggactctttcccatctcccctggggagcctgaggagcccagcgtccactcttcatgccttgggccgagcctgcctcctgcttgcgggggccgcctgtcctcactgccccaggctgcggcttgcccagtcccgcccctctgacccctgcctgtgggctggggagccttggatggggtggggacctggaatgggtctctcttgccccacctggctgaggcgccacccttcttcaggcccaggctccagaggaagactcctgaaaccctccccaggtcttccaagtacaggattgaagctttagtgaaattaaccaaggaccatgggtcagtgcccagggctttaaaaagaatgaacgagcaaaaggtatccccgccgtgacccctgcagatagcaccggtctttgatccgcagcaggggccagaccctgcccacaagtcccagcgcggctgcttctgccactgctgggctccacttggctcctctcacttcccagggggtcgcctgtcctgcctgtgggtttccatggcttcccagagctccctctgccccagccagcgcctccaggcccagctgaggagctgtgagaagcagcagaggggactccccatcccgggcacaccctgtcctcccacccctgcccccttgcccttccagccctttcagctgcagctgggagctggcccgtcaagtgctgcccctgcctgtgtctgggtttctgttggctgtttttcttttcttgagtggtgatttttctctaaataaaagaagtcaagcactgaaaaaaaaaaaaaaaa

SEQ ID NO: 13 - DNA, 5123 bp, Homo sapiens
  source 1..5123: mol_type = genomic DNA
ccgtgagttctgcccaggccctgtgagctcaccagagccacagactcacagcccagaggtggcttcttccttcaggaactgaagaacccccatgaacaccaacatctccaggttctgagaacagaacctgggaaattgatgacttcctcatgatgaccgatactcaggatggccctagcgagagctcccagatcatgaggtccctcactcccctgatcaacagggaggaggcatttggggaggctggggaggcggggctgtggcccagcatcacccacactcctgattcacaggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggaaaacatggggcagcaggccaggatgctgcgggcccaggtgaagcggcacacggtgcgggacaagctgaggctgtgccagaacttcctgcagaagctgcgcttcctggcggacgagccccagcacagcattcccgacatcttcatctggatgatgagcaacaacaagcgtgtcgcctatgcccgtgtgccctccaaggacctgctcttctccatcgtggaggaggagactggcaaggactgcgccaaggtcaagacgctcttccttaagctgccagggaagcggggcttcggctcggcaggctggacagtgcaggccaaggtggagctgtacctgtggctgggcctcagcaaacagcgcaaggagttcctgtgcggcctgccctgtggcttccaggaggtcaaggcagcccagggcctgggcctgcatgccttcccacccgtcagcctggtctacaccaagaagcaggcgttccagctccgagcgcacatgtaccaggcccgcagcctctttgccgccgacagcagcggactctcagacccctttgcccgcgtcttcttcatcaatcagagtcagtgcacagaggtgctgaatgagaccctgtgtcccacctgggaccagatgctggtgttcgacaacctggagctctatggtgaagctcatgagctgagggacgatccgcccatcattgtcattgaaatctatgaccaggattccatgggcaaagctgacttcatgggccggaccttcgccaaacccctggtgaagatggcagacgaggcgtactgcccaccccgcttcccacctcagctcgagtactaccagatctaccgtggcaacgccacagctggagacctgctggcggccttcgagctgctgcagattggaccagcagggaaggctgacctgccccccatcaatggcccggtggacgtggaccgaggtcccatcatgcccgtgcccatgggcatccggcccgtgctcagcaagtaccgagtggaggtgctgttctggggcctacgggacctaaagcgggtgaacctggcccaggtggaccggccacgggtggacatcgagtgtgcagggaagggggtgcagtcgtccctgatccacaattataagaagaaccccaacttcaacaccctcgtcaagtggtttgaagtggacctcccagagaacgagctgctgcacccgcccttgaacatccgtgtggtggactgccgggccttcggtcgctacacactggtgggctcccatgccgtcagctccctgcgacgcttcatctaccggcccccagaccgctcggcccccagctggaacaccacggtcaggcttctccggcgctgccgtgtgctgtgcaatgggggctcctcctctcactccacaggggaggttgtggtgactatggagccagaggtacccatcaagaaactggagaccatggtgaagctggacgcgacttctgaagctgttgtcaaggtggatgtggctgaggaggagaaggagaagaagaagaagaagaagggcactgcggaggagccagaggaggaggagccagacgagagcatgctggactggtggtccaagtactttgcctccattgacaccatgaaggagcaacttcgacaacaagagccctctggaattgacttggaggagaaggaggaagtggacaataccgagggcctgaaggggtcaatgaagggcaaggagaaggcaagggctgccaaagaggagaagaagaagaaaactcagagctctggctctggccaggggtccgaggcccccgagaagaagaaacccaagattgatgagcttaaggtataccccaaagagctggagtccgagtttgataactttgaggactggctgcacactttcaacttgcttcggggcaagaccggggatgatgaggatggctccaccgaggaggagcgcattgtgggacgcttcaagggctccctctgcgtgtacaaagtgccactcccagaggacgtgtcccgggaagccggctacgactccacctacggcatgttccagggcatcccgagcaatgaccccatcaatgtgctggtccgagtctatgtggtccgggccacggacctgcaccctgctgacatcaacggcaaagctgacccctacatcgccatccggctaggcaagactgacatccgcgacaaggagaactacatctccaagcagctcaaccctgtctttgggaagtcctttgacatcgaggcctccttccccatggaatccatgctgacggtggctgtgtatgactgggacctggtgggcactgatgacctcattggggaaaccaagatcgacctggagaaccgcttctacagcaagcaccgcgccacctgcggcatcgcccagacctactccacacatggctacaatatctggcgggaccccatgaagcccagccagatcctgacccgcctctgcaaagacggcaaagtggacggcccccactttgggccccctgggagagtgaaggtggccaaccgcgtcttcactgggccctctgagattgaggacgagaacggtcagaggaagcccacagacgagcatgtggcgctgttggccctgaggcactgggaggacatcccccgcgcaggctgccgcctggtgccagagcatgtggagacgaggccgctgctcaaccccgacaagccgggcatcgagcagggccgcctggagctgtgggtggacatgttccccatggacatgccagcccctgggacgcctctggacatctcacctcggaagcccaagaagtacgagctgcgggtcatcatctggaacacagatgaggtggtcttggaggacgacgacttcttcacaggggagaagtccagtgacatcttcgtgagggggtggctgaagggccagcaggaggacaagcaggacacagacgtccactaccactccctcactggcgagggcaacttcaactggcgctacctgttccccttcgactacctggcggcggaggagaagatcgtcatctccaagaaggagtccatgttctcctgggacgagaccgagtacaagatccccgcgcggctcaccctgcagatctgggatgcggaccacttctccgctgacgacttcctgggggccatcgagctggacctgaaccggttcccgcggggcgcaaagacagccaagcagtgcaccatggagatggccaccggggaggtggacgtgcccctcgtgtccatcttcaagcaaaagcgcgtcaaaggctggtggcccctcctggcccgcaatgagaacgatgagtttgagctcacgggcaaggtggaggctgagctgcatttactgacagcagaggaggcagagaagaacccagtgggcctggcccgcaatgaacctgaccccctagagaaacccaaccggcccgacacgagcttcatctggttcctgaaccctctcaagtcggctcgctacttcttgtggcacacgtatcgctggctgctcctcaaactgttgctgctcctgctgctgctcctcctcctcgccctgttcctctactctgtgcctggctacctggtcaagaaaatcctcggggcctgagcccagtggcctcctggccggcccgacacggccttcgtctggttcctcaaccctctcaagtccatcaagtacctcatctgcacccggtacaagtggctcatcatcaagatcgtgctggcgctgttggggctgctcatgttggggctcttcctctacagcctccctggctacatggtcaaaaagctccttggggcatgaaggccgccagctcccgccagccgctccccagccctgccgcatttcctttcagtggcttggactctttcccatctcccctggggagcctgaggagcccagcgtccactcttcatgccttgggccgagcctgcctcctgcttgcgggggccgcctgtcctcactgccccaggctgcggcttgcccagtcccgcccctctgacccctgcctgtgggctggggagccttggatggggtggggacctggaatgggtctctcttgccccacctggctgaggcgccacccttcttcaggcccaggctccagaggaagactcctgaaaccctccccaggtcttccaagtacaggattgaagctttagtgaaattaaccaaggaccatgggtcagtgcccagggctttaaaaagaatgaacgagcaaaaggtatccccgccgtgacccctgcagatagcaccggtctttgatccgcagcaggggccagaccctgcccacaagtcccagcgcggctgcttctgccactgctgggctccacttggctcctctcacttcccagggggtcgcctgtcctgcctgtgggtttccatggcttcccagagctccctctgccccagccagcgcctccaggcccagctgaggagctgtgagaagcagcagaggggactccccatcccgggcacaccctgtcctcccacccctgcccccttgcccttccagccctttcagctgcagctgggagctggcccgtcaagtgctgcccctgcctgtgtctgggtttctgttggctgtttttcttttcttgagtggtgatttttctctaaataaaagaagtcaagcactgaaaaaaaaaaaaaaaa

SEQ ID NO: 14 - DNA, 6973 bp, Homo sapiens
  source 1..6973: mol_type = genomic DNA
atcggaggggggtcgggaggaggaggaggaggcagcggcagagaagagagaggcgtgtgagccgtgctccaccggctagctccttcccgctgctcctgcctggcagtgccaggcagcccacaccagcatggccttgctcatccacctcaagacagtctcggagctgcggggcaggggcgaccggatcgccaaagtgactttccgagggcaatccttctactctcgggtcctggagaactgtgaggatgtggctgactttgatgagacatttcggtggccggtggccagcagcatcgacagaaatgagatgctggagattcaggttttcaactacagcaaagtcttcagcaacaagctcatcgggaccttccgcatggtgctgcagaaggtggtagaggagagccatgtggaggtgactgacacgctgattgatgacaacaatgctatcatcaagaccagcctgtgcgtggaggtccggtatcaggccactgacggcacagtgggctcctgggacgatggggacttcctgggagatgagtctcttcaagaggaagagaaggacagccaagagacggatggactgctcccaggctcccggcccagctcccggcccccaggagagaagagcttccggagagccgggaggagcgtgttctccgccatgaagctcggcaaaaaccggtctcacaaggaggagccccaaagaccagatgaaccggcggtgctggagatggaagaccttgaccatctggccattcggctaggagatggactggatcccgactcggtgtctctagcctcagtcacagctctcaccactaatgtctccaacaagcgatctaagccagacattaagatggagccaagtgctgggcggcccatggattaccaggtcagcatcacggtgatcgaggcccggcagctggtgggcttgaacatggaccctgtggtgtgcgtggaggtgggtgacgacaagaagtacacatccatgaaggagtccactaactgcccctattacaacgagtacttcgtcttcgacttccatgtctctccggatgtcatgtttgacaagatcatcaagatttcggtgattcactccaagaacctgctgcgcagtggcaccctggtgggctccttcaaaatggacgtgggaaccgtgtactcgcagccagagcaccagttccatcacaagtgggccatcctgtctgaccccgatgacatctcctcggggctgaagggctacgtgaagtgtgacgttgccgtggtgggcaaaggggacaacatcaagacgccccacaaggccaatgagaccgacgaagatgacattgaggggaacttgctgctccccgagggggtgccccccgaacgccagtgggcccggttctatgtgaaaatttaccgagcagaggggctgccccgtatgaacacaagcctcatggccaatgtaaagaaggctttcatcggtgaaaacaaggacctcgtggacccctacgtgcaagtcttctttgctggccagaagggcaagacttcagtgcagaagagcagctatgagcccctgtggaatgagcaggtcgtctttacagacctcttccccccactctgcaaacgcatgaaggtgcagatccgagactcggacaaggtcaacgacgtggccatcggcacccacttcattgacctgcgcaagatttctaatgacggagacaaaggcttcctgcccacactgggcccagcctgggtgaacatgtacggctccacacgtaactacacgctgctggatgagcatcaggacctgaacgagggcctgggggagggtgtgtccttccgggcccggctcctgctgggcctggctgtggagatcgtagacacctccaaccctgagctcaccagctccacagaggtgcaggtggagcaggccacgcccatctcggagagctgtgcaggtaaaatggaagaattctttctctttggagccttcctggaggcctcaatgatcgaccggagaaacggagacaagcccatcacctttgaggtcaccataggcaactatgggaacgaagttgatggcctgtcccggccccagcggcctcggccccggaaggagccgggggatgaggaagaagtagacctgattcagaacgcaagtgatgacgaggccggtgatgccggggacctggcctcagtctcctccactccaccaatgcggccccaggtcaccgacaggaactacttccatctgccctacctggagcgaaagccctgcatctacatcaagagctggtggccggaccagcgccgccgcctctacaatgccaacatcatggaccacattgccgacaagctggaagaaggcctgaacgacatacaggagatgatcaaaacggagaagtcctaccctgagcgtcgcctgcggggcgtcctggaggagctgagctgtggctgctgccgcttcctctccctcgctgacaaggaccagggccactcatcccgcaccaggcttgaccgggagcgcctcaagtcctgcatgagggagctggaaaacatggggcagcaggccaggatgctgcgggcccaggtgaagcggcacacggtgcgggacaagctgaggctgtgccagaacttcctgcagaagctgcgcttcctggcggacgagccccagcacagcattcccgacatcttcatctggatgatgagcaacaacaagcgtgtcgcctatgcccgtgtgccctccaaggacctgctcttctccatcgtggaggaggagactggcaaggactgcgccaaggtcaagacgctcttccttaagctgccagggaagcggggcttcggctcggcaggctggacagtgcaggccaaggtggagctgtacctgtggctgggcctcagcaaacagcgcaaggagttcctgtgcggcctgccctgtggcttccaggaggtcaaggcagcccagggcctgggcctgcatgccttcccacccgtcagcctggtctacaccaagaagcaggcgttccagctccgagcgcacatgtaccaggcccgcagcctctttgccgccgacagcagcggactctcagacccctttgcccgcgtcttcttcatcaatcagagtcagtgcacagaggtgctgaatgagaccctgtgtcccacctgggaccagatgctggtgttcgacaacctggagctctatggtgaagctcatgagctgagggacgatccgcccatcattgtcattgaaatctatgaccaggattccatgggcaaagctgacttcatgggccggaccttcgccaaacccctggtgaagatggcagacgaggcgtactgcccaccccgcttcccacctcagctcgagtactaccagatctaccgtggcaacgccacagctggagacctgctggcggccttcgagctgctgcagattggaccagcagggaaggctgacctgccccccatcaatggcccggtggacgtggaccgaggtcccatcatgcccgtgcccatgggcatccggcccgtgctcagcaagtaccgagtggaggtgctgttctggggcctacgggacctaaagcgggtgaacctggcccaggtggaccggccacgggtggacatcgagtgtgcagggaagggggtgcagtcgtccctgatccacaattataagaagaaccccaacttcaacaccctcgtcaagtggtttgaagtggacctcccagagaacgagctgctgcacccgcccttgaacatccgtgtggtggactgccgggccttcggtcgctacacactggtgggctcccatgccgtcagctccctgcgacgcttcatctaccggcccccagaccgctcggcccccagctggaacaccacggtcaggcttctccggcgctgccgtgtgctgtgcaatgggggctcctcctctcactccacaggggaggttgtggtgactatggagccagaggtacccatcaagaaactggagaccatggtgaagctggacgcgacttctgaagctgttgtcaaggtggatgtggctgaggaggagaaggagaagaagaagaagaagaagggcactgcggaggagccagaggaggaggagccagacgagagcatgctggactggtggtccaagtactttgcctccattgacaccatgaaggagcaacttcgacaacaagagccctctggaattgacttggaggagaaggaggaagtggacaataccgagggcctgaaggggtcaatgaagggcaaggagaaggcaagggctgccaaagaggagaagaagaagaaaactcagagctctggctctggccaggggtccgaggcccccgagaagaagaaacccaagattgatgagcttaaggtataccccaaagagctggagtccgagtttgataactttgaggactggctgcacactttcaacttgcttcggggcaagaccggggatgatgaggatggctccaccgaggaggagcgcattgtgggacgcttcaagggctccctctgcgtgtacaaagtgccactcccagaggacgtgtcccgggaagccggctacgactccacctacggcatgttccagggcatcccgagcaatgaccccatcaatgtgctggtccgagtctatgtggtccgggccacggacctgcaccctgctgacatcaacggcaaagctgacccctacatcgccatccggctaggcaagactgacatccgcgacaaggagaactacatctccaagcagctcaaccctgtctttgggaagtcctttgacatcgaggcctccttccccatggaatccatgctgacggtggctgtgtatgactgggacctggtgggcactgatgacctcattggggaaaccaagatcgacctggagaaccgcttctacagcaagcaccgcgccacctgcggcatcgcccagacctactccacacatggctacaatatctggcgggaccccatgaagcccagccagatcctgacccgcctctgcaaagacggcaaagtggacggcccccactttgggccccctgggagagtgaaggtggccaaccgcgtcttcactgggccctctgagattgaggacgagaacggtcagaggaagcccacagacgagcatgtggcgctgttggccctgaggcactgggaggacatcccccgcgcaggctgccgcctggtgccagagcatgtggagacgaggccgctgctcaaccccgacaagccgggcatcgagcagggccgcctggagctgtgggtggacatgttccccatggacatgccagcccctgggacgcctctggacatctcacctcggaagcccaagaagtacgagctgcgggtcatcatctggaacacagatgaggtggtcttggaggacgacgacttcttcacaggggagaagtccagtgacatcttcgtgagggggtggctgaagggccagcaggaggacaagcaggacacagacgtccactaccactccctcactggcgagggcaacttcaactggcgctacctgttccccttcgactacctggcggcggaggagaagatcgtcatctccaagaaggagtccatgttctcctgggacgagaccgagtacaagatccccgcgcggctcaccctgcagatctgggatgcggaccacttctccgctgacgacttcctgggggccatcgagctggacctgaaccggttcccgcggggcgcaaagacagccaagcagtgcaccatggagatggccaccggggaggtggacgtgcccctcgtgtccatcttcaagcaaaagcgcgtcaaaggctggtggcccctcctggcccgcaatgagaacgatgagtttgagctcacgggcaaggtggaggctgagctgcatttactgacagcagaggaggcagagaagaacccagtgggcctggcccgcaatgaacctgaccccctagagaaacccaaccggcccgacacggccttcgtctggttcctcaaccctctcaagtccatcaagtacctcatctgcacccggtacaagtggctcatcatcaagatcgtgctggcgctgttggggctgctcatgttggggctcttcctctacagcctccctggctacatggtcaaaaagctccttggggcatgaaggccgccagctcccgccagccgctccccagccctgccgcatttcctttcagtggcttggactctttcccatctcccctggggagcctgaggagcccagcgtccactcttcatgccttgggccgagcctgcctcctgcttgcgggggccgcctgtcctcactgccccaggctgcggcttgcccagtcccgcccctctgacccctgcctgtgggctggggagccttggatggggtggggacctggaatgggtctctcttgccccacctggctgaggcgccacccttcttcaggcccaggctccagaggaagactcctgaaaccctccccaggtcttccaagtacaggattgaagctttagtgaaattaaccaaggaccatgggtcagtgcccagggctttaaaaagaatgaacgagcaaaaggtatccccgccgtgacccctgcagatagcaccggtctttgatccgcagcaggggccagaccctgcccacaagtcccagcgcggctgcttctgccactgctgggctccacttggctcctctcacttcccagggggtcgcctgtcctgcctgtgggtttccatggcttcccagagctccctctgccccagccagcgcctccaggcccagctgaggagctgtgagaagcagcagaggggactccccatcccgggcacaccctgtcctcccacccctgcccccttgcccttccagccctttcagctgcagctgggagctggcccgtcaagtgctgcccctgcctgtgtctgggtttctgttggctgtttttcttttcttgagtggtgatttttctctaaataaaagaagtcaagcactgaaaaaaaaaaaaaaaa

SEQ ID NO: 15 - DNA, 7125 bp, Mus musculus
  source 1..7125: mol_type = genomic DNA
ttggttgccttggtctctgtgggcagcagcaggaggaggcggcagcagccagagaagagggaggcgtgtgagccacactccaccagcgagcttcttcccgctgctctggaactgcccaggctctccccaccagcatggccctgattgttcacctcaagactgtctcagagctccgaggcaaaggtgaccggattgccaaagtcactttccgagggcagtctttctactcccgggtcctggagaactgcgagggtgtggctgactttgatgagacgttccggtggccagtggccagcagcatcgaccggaatgaagtgttggagattcagattttcaactacagcaaagtcttcagcaacaagctgatagggaccttctgcatggtgctgcagaaagtggtggaggagaatcgggtagaggtgaccgacacgctgatggatgacagcaatgctatcatcaagaccagcctgagcatggaggtccggtatcaggccacagatggcactgtgggcccctgggatgatggagacttcctgggagatgaatccctccaggaggagaaggacagccaggagacagatgggctgctacctggttcccgacccagcacccggatatctggcgagaagagctttcgcagagcgggaaggagtgtgttctcggccatgaaactcggcaaaactcggtcccacaaagaggagccccaaagacaagatgagccagcagtgctggagatggaggacctggaccacctagccattcagctgggggatgggctggatcctgactccgtgtctctagcctcggtcaccgctctcaccagcaatgtctccaacaaacggtctaagccagatattaagatggagcccagtgctggaaggcccatggattaccaggtcagcatcacagtgattgaggctcggcagctggtgggcttgaacatggaccctgtggtgtgtgtggaggtgggtgatgacaagaaatacacgtcaatgaaggagtccacaaactgcccttactacaacgagtactttgtcttcgacttccatgtctctcctgatgtcatgtttgacaagatcatcaagatctcggttatccattctaagaacctgcttcggagcggcaccctggtgggttccttcaaaatggatgtggggactgtgtattcccagcctgaacaccagttccatcacaaatgggccatcctgtcagaccccgatgacatctctgctgggttgaagggttatgtaaagtgtgatgtcgctgtggtgggcaagggagacaacatcaagacaccccacaaggccaacgagacggatgaggacgacattgaagggaacttgctgctccccgagggcgtgccccccgaacggcagtgggcacggttctatgtgaaaatttaccgagcagagggactgccccggatgaacacaagcctcatggccaacgtgaagaaggcgttcatcggtgagaacaaggacctcgtcgacccctatgtgcaagtcttctttgctggacaaaagggcaaaacatcagtgcagaagagcagctatgagccgctatggaatgagcaggtcgtcttcacagacttgttccccccactctgcaaacgcatgaaggtgcagatccgggactctgacaaggtcaatgatgtggccatcggcacccacttcatcgacctgcgcaagatttccaacgatggagacaaaggcttcctgcctaccctcggtccagcctgggtgaacatgtacggctccacgcgcaactacacactgctggacgagcaccaggacttgaatgaaggcctgggggagggtgtgtccttccgggcccgcctcatgttgggactagctgtggagatcctggacacctccaacccagagctcaccagctccacggaggtgcaggtggagcaggccacgcctgtctcggagagctgcacagggagaatggaagaattttttctatttggagccttcttggaagcctcaatgattgaccggaaaaatggggacaagccaattacctttgaggtgaccataggaaactacggcaatgaagtcgatggtatgtcccggcccctgaggcctcggccccggaaagagcctggggatgaagaagaggtagacctgattcagaactccagtgacgatgaaggtgacgaagccggggacctggcctcggtgtcctccaccccacctatgcggccccagatcacggacaggaactatttccacctgccctacctggagcgcaagccctgcatctatatcaagagctggtggcctgaccagaggcggcgcctctacaatgccaacatcatggatcacattgctgacaagctggaagaaggcctgaatgatgtacaggagatgatcaaaacggagaagtcctacccggagcgccgcctgcggggtgtgctagaggaactcagctgtggctgccaccgcttcctctccctctcggacaaggaccagggccgctcgtcccgcaccaggctggatcgagagcgtcttaagtcctgtatgagggagttggagagcatgggacagcaggccaagagcctgagggctcaggtgaagcggcacactgttcgggacaagctgaggtcatgccagaactttctgcagaagctacgcttcctggcggatgagccccagcacagcattcctgatgtgttcatttggatgatgagcaacaacaaacgtatcgcctatgcccgcgtgccttccaaagacctgctcttctccatcgtggaggaggaactgggcaaggactgcgccaaagtcaagaccctcttcctgaagctgccagggaagaggggcttcggctcggcaggctggacagtacaggccaagctggagctctacctgtggctgggcctcagcaagcagcgaaaggacttcctgtgtggtctgccctgtggcttcgaggaggtcaaggcagcccaaggcctgggcctgcattcctttccgcccatcagcctagtctacaccaagaagcaagccttccagctccgagcacacatgtatcaggcccgaagcctctttgctgctgacagcagtgggctctctgatccctttgcccgtgtcttcttcatcaaccagagccaatgcactgaggttctaaacgagacactgtgtcccacctgggaccagatgctggtatttgacaacctggagctgtacggtgaagctcacgagttacgagatgatccccccatcattgtcattgaaatctacgaccaggacagcatgggcaaagccgacttcatgggccggaccttcgccaagcccctggtgaagatggcagatgaagcatactgcccacctcgcttcccgccgcagcttgagtactaccagatctaccgaggcagtgccactgccggagacctactggctgccttcgagctgctgcagattgggccatcagggaaggctgacctgccacccatcaatggcccagtggacatggacagagggcccatcatgcctgtgcccgtgggaatccggccagtgctcagcaagtaccgagtggaggtgctgttctggggcctgagggacctaaagagggtgaacctggcccaggtggaccgaccacgggtggacatcgagtgtgcaggaaagggggtacaatcctccctgattcacaattataagaagaaccccaacttcaacacgctggtcaagtggtttgaagtggacctcccggagaatgagctcctgcacccacccttgaacatccgagtggtagattgccgggcctttggacgatacaccctggtgggttcccacgcagtcagctcactgaggcgcttcatctaccgacctccagaccgctcagcccccaactggaacaccacagtcaggctgctccggggctgccacaggctgcgcaatgggggcccctcttctcgccccacaggggaggttgtagtaagcatggagcctgaggagccagttaagaagctggagaccatggtgaaactggatgcgacttctgatgctgtggtcaaggtggatgtggctgaagatgagaaggaaaggaagaagaagaaaaagaaaggcccgtcagaggagccagaggaggaagagcccgatgagagcatgctggattggtggtccaagtacttcgcctccatcgacacaatgaaggagcaacttcgacaacatgagacctctggaactgacttggaagagaaggaagagatggaaagcgctgagggcctgaagggaccaatgaagagcaaggagaagtccagagctgcaaaggaggagaaaaagaagaaaaaccagagccctggccctggccagggatcggaggctcctgagaagaagaaagccaagatcgatgagcttaaggtgtaccccaaggagctggaatcggagtttgacagctttgaggactggctgcacaccttcaacctgttgaggggcaagacgggagatgatgaggatggctccacagaggaggagcgcatagtaggccgattcaagggctccctctgtgtgtacaaagtgccactcccagaagatgtatctcgagaagctggctatgatcccacctatggaatgttccagggcatcccaagcaatgaccccatcaatgtgctggtccgaatctatgtggtccgggccacagacctgcacccggccgacatcaatggcaaagctgacccctatattgccatcaagttaggcaagaccgacatccgagacaaggagaactacatctccaagcagctcaaccctgtgtttgggaagtcctttgacattgaggcctccttccccatggagtccatgttgacagtggccgtgtacgactgggatctggtgggcactgatgacctcatcggagaaaccaagattgacctggaaaaccgcttctacagcaagcatcgcgccacctgcggcatcgcacagacctattccatacatggctacaatatctggagggaccccatgaagcccagccagatcctgacacgcctctgtaaagagggcaaagtggacggcccccactttggtccccatgggagagtgagggttgccaaccgtgtcttcacggggccttcagaaatagaggatgagaatggtcagaggaagcccacagatgagcacgtggcactgtctgctctgagacactgggaggacatcccccgggtgggctgccgccttgtgccggaacacgtggagaccaggccgctgctcaaccctgacaagccaggcattgagcagggccgcctggagctgtgggtggacatgttccccatggacatgccagcccctgggacacctctggatatatcccccaggaaacccaagaagtacgagctgcgggtcatcgtgtggaacacagacgaggtggtcctggaagacgatgatttcttcacgggagagaagtccagtgacatttttgtgagggggtggctgaagggccagcaggaggacaaacaggacacagatgtccactatcactccctcacgggggagggcaacttcaactggagatacctcttccccttcgactacctagcggccgaagagaagatcgttatgtccaaaaaggagtctatgttctcctgggatgagacggagtacaagatccctgcgcggctcaccctgcagatctgggacgctgaccacttctcggctgacgacttcctgggggctatcgagctggacctgaaccggttcccgaggggcgctaagacagccaagcagtgcaccatggagatggccaccggggaggtggacgtacccctggtttccatctttaaacagaaacgtgtcaaaggctggtggcccctcctggcccgcaatgagaatgatgagtttgagctcacaggcaaagtggaggcggagctacacctactcacggcagaggaggcagagaagaaccctgtgggcctggctcgcaatgaacctgatcccctagaaaaacccaatcggccggacacaagcttcatctggttcttgaaccctctcaagtctgcccgctacttcctgtggcatacctaccgctggctactcctcaaattcctgctgctcttcctcctgctgctgctcttcgccctgtttctctactctctgcctggctacctggccaagaagatccttggggcctgagccctgcagtcgcctaggcctgccggcctgacacggcattcgtctggttcctgaacccactcaaatctatcaagtacctcatctgcacccggtacaagtggctgatcatcaagatcgtgctggcgctgctggggctgctcatgctggccctcttcctttacagcctcccaggctacatggtcaagaagctcctaggggcctgaagtgtgccccaccccagcccgctccagcatccctccaggggctgctgcgtattttgccttccctcacctggactctctcccaactccctgaggagccctcccacgcctgccagccttgagcaagacacctgcttgctggacttcatccccaccccacacccaaactgttgcttgcctgatcttgtcccaggcctgcctggggtttggggcacagttggcctccaaaaccagataccctcttgtctaaagtaccaggttcctctgcccaaccccaagagtggtagtggcccaaccctccctgtgctttccaaatcttgtcttaaggcaccagtgaaattaaccaagaaacgcggagcaatgcccaaggctctgatgagtaggaacacgtggaaagcaccaggaatgccagcagaggcgaggcggcacacctctctgcagagcatccaggccgagcggcgggcagcggccagctgcttctgcgcatgctctcctcttggctctgcttctttctcacagtcacagtcacttcacagcttagccttgggcttcccatcacttccaggggtgcctctgccttggccagtgtgtgtcagctagtacacaagctccaagtgtgaatcaggtgtactggccgtcctgaagactgactgccctgtccttcctgccgacagccacacccgagtgtacacttaaagcggtgcccttctgcctctgtgggcctgctggctgctgttcctttcttgagtgtgattttttttttctctccctcaataaaataaatcaaactctgagac

SEQ ID NO: 16 - DNA, 7065 bp, Mus musculus
  source 1..7065: mol_type = genomic DNA
ttggttgccttggtctctgtgggcagcagcaggaggaggcggcagcagccagagaagagggaggcgtgtgagccacactccaccagcgagcttcttcccgctgctctggaactgcccaggctctccccaccagcatggccctgattgttcacctcaagactgtctcagagctccgaggcaaaggtgaccggattgccaaagtcactttccgagggcagtctttctactcccgggtcctggagaactgcgagggtgtggctgactttgatgagacgttccggtggccagtggccagcagcatcgaccggaatgaagtgttggagattcagattttcaactacagcaaagtcttcagcaacaagctgatagggaccttctgcatggtgctgcagaaagtggtggaggagaatcgggtagaggtgaccgacacgctgatggatgacagcaatgctatcatcaagaccagcctgagcatggaggtccggtatcaggccacagatggcactgtgggcccctgggatgatggagacttcctgggagatgaatccctccaggaggagaaggacagccaggagacagatgggctgctacctggttcccgacccagcacccggatatctggcgagaagagctttcgcagagcgggaaggagtgtgttctcggccatgaaactcggcaaaactcggtcccacaaagaggagccccaaagacaagatgagccagcagtgctggagatggaggacctggaccacctagccattcagctgggggatgggctggatcctgactccgtgtctctagcctcggtcaccgctctcaccagcaatgtctccaacaaacggtctaagccagatattaagatggagcccagtgctggaaggcccatggattaccaggtcagcatcacagtgattgaggctcggcagctggtgggcttgaacatggaccctgtggtgtgtgtggaggtgggtgatgacaagaaatacacgtcaatgaaggagtccacaaactgcccttactacaacgagtactttgtcttcgacttccatgtctctcctgatgtcatgtttgacaagatcatcaagatctcggttatccattctaagaacctgcttcggagcggcaccctggtgggttccttcaaaatggatgtggggactgtgtattcccagcctgaacaccagttccatcacaaatgggccatcctgtcagaccccgatgacatctctgctgggttgaagggttatgtaaagtgtgatgtcgctgtggtgggcaagggagacaacatcaagacaccccacaaggccaacgagacggatgaggacgacattgaagggaacttgctgctccccgagggcgtgccccccgaacggcagtgggcacggttctatgtgaaaatttaccgagcagagggactgccccggatgaacacaagcctcatggccaacgtgaagaaggcgttcatcggtgagaacaaggacctcgtcgacccctatgtgcaagtcttctttgctggacaaaagggcaaaacatcagtgcagaagagcagctatgagccgctatggaatgagcaggtcgtcttcacagacttgttccccccactctgcaaacgcatgaaggtgcagatccgggactctgacaaggtcaatgatgtggccatcggcacccacttcatcgacctgcgcaagatttccaacgatggagacaaaggcttcctgcctaccctcggtccagcctgggtgaacatgtacggctccacgcgcaactacacactgctggacgagcaccaggacttgaatgaaggcctgggggagggtgtgtccttccgggcccgcctcatgttgggactagctgtggagatcctggacacctccaacccagagctcaccagctccacggaggtgcaggtggagcaggccacgcctgtctcggagagctgcacagggagaatggaagaattttttctatttggagccttcttggaagcctcaatgattgaccggaaaaatggggacaagccaattacctttgaggtgaccataggaaactacggcaatgaagtcgatggtatgtcccggcccctgaggcctcggccccggaaagagcctggggatgaagaagaggtagacctgattcagaactccagtgacgatgaaggtgacgaagccggggacctggcctcggtgtcctccaccccacctatgcggccccagatcacggacaggaactatttccacctgccctacctggagcgcaagccctgcatctatatcaagagctggtggcctgaccagaggcggcgcctctacaatgccaacatcatggatcacattgctgacaagctggaagaaggcctgaatgatgtacaggagatgatcaaaacggagaagtcctacccggagcgccgcctgcggggtgtgctagaggaactcagctgtggctgccaccgcttcctctccctctcggacaaggaccagggccgctcgtcccgcaccaggctggatcgagagcgtcttaagtcctgtatgagggagttggagagcatgggacagcaggccaagagcctgagggctcaggtgaagcggcacactgttcgggacaagctgaggtcatgccagaactttctgcagaagctacgcttcctggcggatgagccccagcacagcattcctgatgtgttcatttggatgatgagcaacaacaaacgtatcgcctatgcccgcgtgccttccaaagacctgctcttctccatcgtggaggaggaactgggcaaggactgcgccaaagtcaagaccctcttcctgaagctgccagggaagaggggcttcggctcggcaggctggacagtacaggccaagctggagctctacctgtggctgggcctcagcaagcagcgaaaggacttcctgtgtggtctgccctgtggcttcgaggaggtcaaggcagcccaaggcctgggcctgcattcctttccgcccatcagcctagtctacaccaagaagcaagccttccagctccgagcacacatgtatcaggcccgaagcctctttgctgctgacagcagtgggctctctgatccctttgcccgtgtcttcttcatcaaccagagccaatgcactgaggttctaaacgagacactgtgtcccacctgggaccagatgctggtatttgacaacctggagctgtacggtgaagctcacgagttacgagatgatccccccatcattgtcattgaaatctacgaccaggacagcatgggcaaagccgacttcatgggccggaccttcgccaagcccctggtgaagatggcagatgaagcatactgcccacctcgcttcccgccgcagcttgagtactaccagatctaccgaggcagtgccactgccggagacctactggctgccttcgagctgctgcagattgggccatcagggaaggctgacctgccacccatcaatggcccagtggacatggacagagggcccatcatgcctgtgcccgtgggaatccggccagtgctcagcaagtaccgagtggaggtgctgttctggggcctgagggacctaaagagggtgaacctggcccaggtggaccgaccacgggtggacatcgagtgtgcaggaaagggggtacaatcctccctgattcacaattataagaagaaccccaacttcaacacgctggtcaagtggtttgaagtggacctcccggagaatgagctcctgcacccacccttgaacatccgagtggtagattgccgggcctttggacgatacaccctggtgggttcccacgcagtcagctcactgaggcgcttcatctaccgacctccagaccgctcagcccccaactggaacaccacaggggaggttgtagtaagcatggagcctgaggagccagttaagaagctggagaccatggtgaaactggatgcgacttctgatgctgtggtcaaggtggatgtggctgaagatgagaaggaaaggaagaagaagaaaaagaaaggcccgtcagaggagccagaggaggaagagcccgatgagagcatgctggattggtggtccaagtacttcgcctccatcgacacaatgaaggagcaacttcgacaacatgagacctctggaactgacttggaagagaaggaagagatggaaagcgctgagggcctgaagggaccaatgaagagcaaggagaagtccagagctgcaaaggaggagaaaaagaagaaaaaccagagccctggccctggccagggatcggaggctcctgagaagaagaaagccaagatcgatgagcttaaggtgtaccccaaggagctggaatcggagtttgacagctttgaggactggctgcacaccttcaacctgttgaggggcaagacgggagatgatgaggatggctccacagaggaggagcgcatagtaggccgattcaagggctccctctgtgtgtacaaagtgccactcccagaagatgtatctcgagaagctggctatgatcccacctatggaatgttccagggcatcccaagcaatgaccccatcaatgtgctggtccgaatctatgtggtccgggccacagacctgcacccggccgacatcaatggcaaagctgacccctatattgccatcaagttaggcaagaccgacatccgagacaaggagaactacatctccaagcagctcaaccctgtgtttgggaagtcctttgacattgaggcctccttccccatggagtccatgttgacagtggccgtgtacgactgggatctggtgggcactgatgacctcatcggagaaaccaagattgacctggaaaaccgcttctacagcaagcatcgcgccacctgcggcatcgcacagacctattccatacatggctacaatatctggagggaccccatgaagcccagccagatcctgacacgcctctgtaaagagggcaaagtggacggcccccactttggtccccatgggagagtgagggttgccaaccgtgtcttcacggggccttcagaaatagaggatgagaatggtcagaggaagcccacagatgagcacgtggcactgtctgctctgagacactgggaggacatcccccgggtgggctgccgccttgtgccggaacacgtggagaccaggccgctgctcaaccctgacaagccaggcattgagcagggccgcctggagctgtgggtggacatgttccccatggacatgccagcccctgggacacctctggatatatcccccaggaaacccaagaagtacgagctgcgggtcatcgtgtggaacacagacgaggtggtcctggaagacgatgatttcttcacgggagagaagtccagtgacatttttgtgagggggtggctgaagggccagcaggaggacaaacaggacacagatgtccactatcactccctcacgggggagggcaacttcaactggagatacctcttccccttcgactacctagcggccgaagagaagatcgttatgtccaaaaaggagtctatgttctcctgggatgagacggagtacaagatccctgcgcggctcaccctgcagatctgggacgctgaccacttctcggctgacgacttcctgggggctatcgagctggacctgaaccggttcccgaggggcgctaagacagccaagcagtgcaccatggagatggccaccggggaggtggacgtacccctggtttccatctttaaacagaaacgtgtcaaaggctggtggcccctcctggcccgcaatgagaatgatgagtttgagctcacaggcaaagtggaggcggagctacacctactcacggcagaggaggcagagaagaaccctgtgggcctggctcgcaatgaacctgatcccctagaaaaacccaatcggccggacacaagcttcatctggttcttgaaccctctcaagtctgcccgctacttcctgtggcatacctaccgctggctactcctcaaattcctgctgctcttcctcctgctgctgctcttcgccctgtttctctactctctgcctggctacctggccaagaagatccttggggcctgagccctgcagtcgcctaggcctgccggcctgacacggcattcgtctggttcctgaacccactcaaatctatcaagtacctcatctgcacccggtacaagtggctgatcatcaagatcgtgctggcgctgctggggctgctcatgctggccctcttcctttacagcctcccaggctacatggtcaagaagctcctaggggcctgaagtgtgccccaccccagcccgctccagcatccctccaggggctgctgcgtattttgccttccctcacctggactctctcccaactccctgaggagccctcccacgcctgccagccttgagcaagacacctgcttgctggacttcatccccaccccacacccaaactgttgcttgcctgatcttgtcccaggcctgcctggggtttggggcacagttggcctccaaaaccagataccctcttgtctaaagtaccaggttcctctgcccaaccccaagagtggtagtggcccaaccctccctgtgctttccaaatcttgtcttaaggcaccagtgaaattaaccaagaaacgcggagcaatgcccaaggctctgatgagtaggaacacgtggaaagcaccaggaatgccagcagaggcgaggcggcacacctctctgcagagcatccaggccgagcggcgggcagcggccagctgcttctgcgcatgctctcctcttggctctgcttctttctcacagtcacagtcacttcacagcttagccttgggcttcccatcacttccaggggtgcctctgccttggccagtgtgtgtcagctagtacacaagctccaagtgtgaatcaggtgtactggccgtcctgaagactgactgccctgtccttcctgccgacagccacacccgagtgtacacttaaagcggtgcccttctgcctctgtgggcctgctggctgctgttcctttcttgagtgtgattttttttttctctccctcaataaaataaatcaaactctgagac

SEQ ID NO: 17 - DNA, 6907 bp, Mus musculus
  source 1..6907: mol_type = genomic DNA
ttggttgccttggtctctgtgggcagcagcaggaggaggcggcagcagccagagaagagggaggcgtgtgagccacactccaccagcgagcttcttcccgctgctctggaactgcccaggctctccccaccagcatggccctgattgttcacctcaagactgtctcagagctccgaggcaaaggtgaccggattgccaaagtcactttccgagggcagtctttctactcccgggtcctggagaactgcgagggtgtggctgactttgatgagacgttccggtggccagtggccagcagcatcgaccggaatgaagtgttggagattcagattttcaactacagcaaagtcttcagcaacaagctgatagggaccttctgcatggtgctgcagaaagtggtggaggagaatcgggtagaggtgaccgacacgctgatggatgacagcaatgctatcatcaagaccagcctgagcatggaggtccggtatcaggccacagatggcactgtgggcccctgggatgatggagacttcctgggagatgaatccctccaggaggagaaggacagccaggagacagatgggctgctacctggttcccgacccagcacccggatatctggcgagaagagctttcgcagcaaaggcagagagaagaccaagggaggcagagatggcgagcacaaagcgggaaggagtgtgttctcggccatgaaactcggcaaaactcggtcccacaaagaggagccccaaagacaagatgagccagcagtgctggagatggaggacctggaccacctagccattcagctgggggatgggctggatcctgactccgtgtctctagcctcggtcaccgctctcaccagcaatgtctccaacaaacggtctaagccagatattaagatggagcccagtgctggaaggcccatggattaccaggtcagcatcacagtgattgaggctcggcagctggtgggcttgaacatggaccctgtggtgtgtgtggaggtgggtgatgacaagaaatacacgtcaatgaaggagtccacaaactgcccttactacaacgagtactttgtcttcgacttccatgtctctcctgatgtcatgtttgacaagatcatcaagatctcggttatccattctaagaacctgcttcggagcggcaccctggtgggttccttcaaaatggatgtggggactgtgtattcccagcctgaacaccagttccatcacaaatgggccatcctgtcagaccccgatgacatctctgctgggttgaagggttatgtaaagtgtgatgtcgctgtggtgggcaagggagacaacatcaagacaccccacaaggccaacgagacggatgaggacgacattgaagggaacttgctgctccccgagggcgtgccccccgaacggcagtgggcacggttctatgtgaaaatttaccgagcagagggactgccccggatgaacacaagcctcatggccaacgtgaagaaggcgttcatcggtgagaacaaggacctcgtcgacccctatgtgcaagtcttctttgctggacaaaagggcaaaacatcagtgcagaagagcagctatgagccgctatggaatgagcaggtcgtcttcacagacttgttccccccactctgcaaacgcatgaaggtgcagatccgggactctgacaaggtcaatgatgtggccatcggcacccacttcatcgacctgcgcaagatttccaacgatggagacaaaggcttcctgcctaccctcggtccagcctgggtgaacatgtacggctccacgcgcaactacacactgctggacgagcaccaggacttgaatgaaggcctgggggagggtgtgtccttccgggcccgcctcatgttgggactagctgtggagatcctggacacctccaacccagagctcaccagctccacggaggtgcaggtggagcaggccacgcctgtctcggagagctgcacagggagaatggaagaattttttctatttggagccttcttggaagcctcaatgattgaccggaaaaatggggacaagccaattacctttgaggtgaccataggaaactacggcaatgaagtcgatggtatgtcccggcccctgaggcctcggccccggaaagagcctggggatgaagaagaggtagacctgattcagaactccagtgacgatgaaggtgacgaagccggggacctggcctcggtgtcctccaccccacctatgcggccccagatcacggacaggaactatttccacctgccctacctggagcgcaagccctgcatctatatcaagagctggtggcctgaccagaggcggcgcctctacaatgccaacatcatggatcacattgctgacaagctggaagaaggcctgaatgatgtacaggagatgatcaaaacggagaagtcctacccggagcgccgcctgcggggtgtgctagaggaactcagctgtggctgccaccgcttcctctccctctcggacaaggaccagggccgctcgtcccgcaccaggctggatcgagagcgtcttaagtcctgtatgagggagttggagagcatgggacagcaggccaagagcctgagggctcaggtgaagcggcacactgttcgggacaagctgaggtcatgccagaactttctgcagaagctacgcttcctggcggatgagccccagcacagcattcctgatgtgttcatttggatgatgagcaacaacaaacgtatcgcctatgcccgcgtgccttccaaagacctgctcttctccatcgtggaggaggaactgggcaaggactgcgccaaagtcaagaccctcttcctgaagctgccagggaagaggggcttcggctcggcaggctggacagtacaggccaagctggagctctacctgtggctgggcctcagcaagcagcgaaaggacttcctgtgtggtctgccctgtggcttcgaggaggtcaaggcagcccaaggcctgggcctgcattcctttccgcccatcagcctagtctacaccaagaagcaagccttccagctccgagcacacatgtatcaggcccgaagcctctttgctgctgacagcagtgggctctctgatccctttgcccgtgtcttcttcatcaaccagagccaatgcactgaggttctaaacgagacactgtgtcccacctgggaccagatgctggtatttgacaacctggagctgtacggtgaagctcacgagttacgagatgatccccccatcattgtcattgaaatctacgaccaggacagcatgggcaaagccgacttcatgggccggaccttcgccaagcccctggtgaagatggcagatgaagcatactgcccacctcgcttcccgccgcagcttgagtactaccagatctaccgaggcagtgccactgccggagacctactggctgccttcgagctgctgcagattgggccatcagggaaggctgacctgccacccatcaatggcccagtggacatggacagagggcccatcatgcctgtgcccgtgggaatccggccagtgctcagcaagtaccgagtggaggtgctgttctggggcctgagggacctaaagagggtgaacctggcccaggtggaccgaccacgggtggacatcgagtgtgcaggaaagggggtacaatcctccctgattcacaattataagaagaaccccaacttcaacacgctggtcaagtggtttgaagtggacctcccggagaatgagctcctgcacccacccttgaacatccgagtggtagattgccgggcctttggacgatacaccctggtgggttcccacgcagtcagctcactgaggcgcttcatctaccgacctccagaccgctcagcccccaactggaacaccacaggggaggttgtagtaagcatggagcctgaggagccagttaagaagctggagaccatggtgaaactggatgcgacttctgatgctgtggtcaaggtggatgtggctgaagatgagaaggaaaggaagaagaagaaaaagaaaggcccgtcagaggagccagaggaggaagagcccgatgagagcatgctggattggtggtccaagtacttcgcctccatcgacacaatgaaggagcaacttcgacaacatgagacctctggaactgacttggaagagaaggaagagatggaaagcgctgagggcctgaagggaccaatgaagagcaaggagaagtccagagctgcaaaggaggagaaaaagaagaaaaaccagagccctggccctggccagggatcggaggctcctgagaagaagaaagccaagatcgatgagcttaaggtgtaccccaaggagctggaatcggagtttgacagctttgaggactggctgcacaccttcaacctgttgaggggcaagacgggagatgatgaggatggctccacagaggaggagcgcatagtaggccgattcaagggctccctctgtgtgtacaaagtgccactcccagaagatgtatctcgagaagctggctatgatcccacctatggaatgttccagggcatcccaagcaatgaccccatcaatgtgctggtccgaatctatgtggtccgggccacagacctgcacccggccgacatcaatggcaaagctgacccctatattgccatcaagttaggcaagaccgacatccgagacaaggagaactacatctccaagcagctcaaccctgtgtttgggaagtcctttgacattgaggcctccttccccatggagtccatgttgacagtggccgtgtacgactgggatctggtgggcactgatgacctcatcggagaaaccaagattgacctggaaaaccgcttctacagcaagcatcgcgccacctgcggcatcgcacagacctattccatacatggctacaatatctggagggaccccatgaagcccagccagatcctgacacgcctctgtaaagagggcaaagtggacggcccccactttggtccccatgggagagtgagggttgccaaccgtgtcttcacggggccttcagaaatagaggatgagaatggtcagaggaagcccacagatgagcacgtggcactgtctgctctgagacactgggaggacatcccccgggtgggctgccgccttgtgccggaacacgtggagaccaggccgctgctcaaccctgacaagccaggcattgagcagggccgcctggagctgtgggtggacatgttccccatggacatgccagcccctgggacacctctggatatatcccccaggaaacccaagaagtacgagctgcgggtcatcgtgtggaacacagacgaggtggtcctggaagacgatgatttcttcacgggagagaagtccagtgacatttttgtgagggggtggctgaagggccagcaggaggacaaacaggacacagatgtccactatcactccctcacgggggagggcaacttcaactggagatacctcttccccttcgactacctagcggccgaagagaagatcgttatgtccaaaaaggagtctatgttctcctgggatgagacggagtacaagatccctgcgcggctcaccctgcagatctgggacgctgaccacttctcggctgacgacttcctgggggctatcgagctggacctgaaccggttcccgaggggcgctaagacagccaagcagtgcaccatggagatggccaccggggaggtggacgtacccctggtttccatctttaaacagaaacgtgtcaaaggctggtggcccctcctggcccgcaatgagaatgatgagtttgagctcacaggcaaagtggaggcggagctacacctactcacggcagaggaggcagagaagaaccctgtgggcctggctcgcaatgaacctgatcccctagaaaaacccaaccggcctgacacggcattcgtctggttcctgaacccactcaaatctatcaagtacctcatctgcacccggtacaagtggctgatcatcaagatcgtgctggcgctgctggggctgctcatgctggccctcttcctttacagcctcccaggctacatggtcaagaagctcctaggggcctgaagtgtgccccaccccagcccgctccagcatccctccaggggctgctgcgtattttgccttccctcacctggactctctcccaactccctgaggagccctcccacgcctgccagccttgagcaagacacctgcttgctggacttcatccccaccccacacccaaactgttgcttgcctgatcttgtcccaggcctgcctggggtttggggcacagttggcctccaaaaccagataccctcttgtctaaagtaccaggttcctctgcccaaccccaagagtggtagtggcccaaccctccctgtgctttccaaatcttgtcttaaggcaccagtgaaattaaccaagaaacgcggagcaatgcccaaggctctgatgagtaggaacacgtggaaagcaccaggaatgccagcagaggcgaggcggcacacctctctgcagagcatccaggccgagcggcgggcagcggccagctgcttctgcgcatgctctcctcttggctctgcttctttctcacagtcacagtcacttcacagcttagccttgggcttcccatcacttccaggggtgcctctgccttggccagtgtgtgtcagctagtacacaagctccaagtgtgaatcaggtgtactggccgtcctgaagactgactgccctgtccttcctgccgacagccacacccgagtgtacacttaaagcggtgcccttctgcctctgtgggcctgctggctgctgttcctttcttgagtgtgattttttttttctctccctcaataaaataaatcaaactctgagac

SEQ ID NO: 18 - DNA, 6862 bp, Mus musculus
  source 1..6862: mol_type = genomic DNA
ttggttgccttggtctctgtgggcagcagcaggaggaggcggcagcagccagagaagagggaggcgtgtgagccacactccaccagcgagcttcttcccgctgctctggaactgcccaggctctccccaccagcatggccctgattgttcacctcaagactgtctcagagctccgaggcaaaggtgaccggattgccaaagtcactttccgagggcagtctttctactcccgggtcctggagaactgcgagggtgtggctgactttgatgagacgttccggtggccagtggccagcagcatcgaccggaatgaagtgttggagattcagattttcaactacagcaaagtcttcagcaacaagctgatagggaccttctgcatggtgctgcagaaagtggtggaggagaatcgggtagaggtgaccgacacgctgatggatgacagcaatgctatcatcaagaccagcctgagcatggaggtccggtatcaggccacagatggcactgtgggcccctgggatgatggagacttcctgggagatgaatccctccaggaggagaaggacagccaggagacagatgggctgctacctggttcccgacccagcacccggatatctggcgagaagagctttcgcagagcgggaaggagtgtgttctcggccatgaaactcggcaaaactcggtcccacaaagaggagccccaaagacaagatgagccagcagtgctggagatggaggacctggaccacctagccattcagctgggggatgggctggatcctgactccgtgtctctagcctcggtcaccgctctcaccagcaatgtctccaacaaacggtctaagccagatattaagatggagcccagtgctggaaggcccatggattaccaggtcagcatcacagtgattgaggctcggcagctggtgggcttgaacatggaccctgtggtgtgtgtggaggtgggtgatgacaagaaatacacgtcaatgaaggagtccacaaactgcccttactacaacgagtactttgtcttcgacttccatgtctctcctgatgtcatgtttgacaagatcatcaagatctcggttatccattctaagaacctgcttcggagcggcaccctggtgggttccttcaaaatggatgtggggactgtgtattcccagcctgaacaccagttccatcacaaatgggccatcctgtcagaccccgatgacatctctgctgggttgaagggttatgtaaagtgtgatgtcgctgtggtgggcaagggagacaacatcaagacaccccacaaggccaacgagacggatgaggacgacattgaagggaacttgctgctccccgagggcgtgccccccgaacggcagtgggcacggttctatgtgaaaatttaccgagcagagggactgccccggatgaacacaagcctcatggccaacgtgaagaaggcgttcatcggtgagaacaaggacctcgtcgacccctatgtgcaagtcttctttgctggacaaaagggcaaaacatcagtgcagaagagcagctatgagccgctatggaatgagcaggtcgtcttcacagacttgttccccccactctgcaaacgcatgaaggtgcagatccgggactctgacaaggtcaatgatgtggccatcggcacccacttcatcgacctgcgcaagatttccaacgatggagacaaaggcttcctgcctaccctcggtccagcctgggtgaacatgtacggctccacgcgcaactacacactgctggacgagcaccaggacttgaatgaaggcctgggggagggtgtgtccttccgggcccgcctcatgttgggactagctgtggagatcctggacacctccaacccagagctcaccagctccacggaggtgcaggtggagcaggccacgcctgtctcggagagctgcacagggagaatggaagaattttttctatttggagccttcttggaagcctcaatgattgaccggaaaaatggggacaagccaattacctttgaggtgaccataggaaactacggcaatgaagtcgatggtatgtcccggcccctgaggcctcggccccggaaagagcctggggatgaagaagaggtagacctgattcagaactccagtgacgatgaaggtgacgaagccggggacctggcctcggtgtcctccaccccacctatgcggccccagatcacggacaggaactatttccacctgccctacctggagcgcaagccctgcatctatatcaagagctggtggcctgaccagaggcggcgcctctacaatgccaacatcatggatcacattgctgacaagctggaagaaggcctgaatgatgtacaggagatgatcaaaacggagaagtcctacccggagcgccgcctgcggggtgtgctagaggaactcagctgtggctgccaccgcttcctctccctctcggacaaggaccagggccgctcgtcccgcaccaggctggatcgagagcgtcttaagtcctgtatgagggagttggagagcatgggacagcaggccaagagcctgagggctcaggtgaagcggcacactgttcgggacaagctgaggtcatgccagaactttctgcagaagctacgcttcctggcggatgagccccagcacagcattcctgatgtgttcatttggatgatgagcaacaacaaacgtatcgcctatgcccgcgtgccttccaaagacctgctcttctccatcgtggaggaggaactgggcaaggactgcgccaaagtcaagaccctcttcctgaagctgccagggaagaggggcttcggctcggcaggctggacagtacaggccaagctggagctctacctgtggctgggcctcagcaagcagcgaaaggacttcctgtgtggtctgccctgtggcttcgaggaggtcaaggcagcccaaggcctgggcctgcattcctttccgcccatcagcctagtctacaccaagaagcaagccttccagctccgagcacacatgtatcaggcccgaagcctctttgctgctgacagcagtgggctctctgatccctttgcccgtgtcttcttcatcaaccagagccaatgcactgaggttctaaacgagacactgtgtcccacctgggaccagatgctggtatttgacaacctggagctgtacggtgaagctcacgagttacgagatgatccccccatcattgtcattgaaatctacgaccaggacagcatgggcaaagccgacttcatgggccggaccttcgccaagcccctggtgaagatggcagatgaagcatactgcccacctcgcttcccgccgcagcttgagtactaccagatctaccgaggcagtgccactgccggagacctactggctgccttcgagctgctgcagattgggccatcagggaaggctgacctgccacccatcaatggcccagtggacatggacagagggcccatcatgcctgtgcccgtgggaatccggccagtgctcagcaagtaccgagtggaggtgctgttctggggcctgagggacctaaagagggtgaacctggcccaggtggaccgaccacgggtggacatcgagtgtgcaggaaagggggtacaatcctccctgattcacaattataagaagaaccccaacttcaacacgctggtcaagtggtttgaagtggacctcccggagaatgagctcctgcacccacccttgaacatccgagtggtagattgccgggcctttggacgatacaccctggtgggttcccacgcagtcagctcactgaggcgcttcatctaccgacctccagaccgctcagcccccaactggaacaccacaggggaggttgtagtaagcatggagcctgaggagccagttaagaagctggagaccatggtgaaactggatgcgacttctgatgctgtggtcaaggtggatgtggctgaagatgagaaggaaaggaagaagaagaaaaagaaaggcccgtcagaggagccagaggaggaagagcccgatgagagcatgctggattggtggtccaagtacttcgcctccatcgacacaatgaaggagcaacttcgacaacatgagacctctggaactgacttggaagagaaggaagagatggaaagcgctgagggcctgaagggaccaatgaagagcaaggagaagtccagagctgcaaaggaggagaaaaagaagaaaaaccagagccctggccctggccagggatcggaggctcctgagaagaagaaagccaagatcgatgagcttaaggtgtaccccaaggagctggaatcggagtttgacagctttgaggactggctgcacaccttcaacctgttgaggggcaagacgggagatgatgaggatggctccacagaggaggagcgcatagtaggccgattcaagggctccctctgtgtgtacaaagtgccactcccagaagatgtatctcgagaagctggctatgatcccacctatggaatgttccagggcatcccaagcaatgaccccatcaatgtgctggtccgaatctatgtggtccgggccacagacctgcacccggccgacatcaatggcaaagctgacccctatattgccatcaagttaggcaagaccgacatccgagacaaggagaactacatctccaagcagctcaaccctgtgtttgggaagtcctttgacattgaggcctccttccccatggagtccatgttgacagtggccgtgtacgactgggatctggtgggcactgatgacctcatcggagaaaccaagattgacctggaaaaccgcttctacagcaagcatcgcgccacctgcggcatcgcacagacctattccatacatggctacaatatctggagggaccccatgaagcccagccagatcctgacacgcctctgtaaagagggcaaagtggacggcccccactttggtccccatgggagagtgagggttgccaaccgtgtcttcacggggccttcagaaatagaggatgagaatggtcagaggaagcccacagatgagcacgtggcactgtctgctctgagacactgggaggacatcccccgggtgggctgccgccttgtgccggaacacgtggagaccaggccgctgctcaaccctgacaagccaggcattgagcagggccgcctggagctgtgggtggacatgttccccatggacatgccagcccctgggacacctctggatatatcccccaggaaacccaagaagtacgagctgcgggtcatcgtgtggaacacagacgaggtggtcctggaagacgatgatttcttcacgggagagaagtccagtgacatttttgtgagggggtggctgaagggccagcaggaggacaaacaggacacagatgtccactatcactccctcacgggggagggcaacttcaactggagatacctcttccccttcgactacctagcggccgaagagaagatcgttatgtccaaaaaggagtctatgttctcctgggatgagacggagtacaagatccctgcgcggctcaccctgcagatctgggacgctgaccacttctcggctgacgacttcctgggggctatcgagctggacctgaaccggttcccgaggggcgctaagacagccaagcagtgcaccatggagatggccaccggggaggtggacgtacccctggtttccatctttaaacagaaacgtgtcaaaggctggtggcccctcctggcccgcaatgagaatgatgagtttgagctcacaggcaaagtggaggcggagctacacctactcacggcagaggaggcagagaagaaccctgtgggcctggctcgcaatgaacctgatcccctagaaaaacccaaccggcctgacacggcattcgtctggttcctgaacccactcaaatctatcaagtacctcatctgcacccggtacaagtggctgatcatcaagatcgtgctggcgctgctggggctgctcatgctggccctcttcctttacagcctcccaggctacatggtcaagaagctcctaggggcctgaagtgtgccccaccccagcccgctccagcatccctccaggggctgctgcgtattttgccttccctcacctggactctctcccaactccctgaggagccctcccacgcctgccagccttgagcaagacacctgcttgctggacttcatccccaccccacacccaaactgttgcttgcctgatcttgtcccaggcctgcctggggtttggggcacagttggcctccaaaaccagataccctcttgtctaaagtaccaggttcctctgcccaaccccaagagtggtagtggcccaaccctccctgtgctttccaaatcttgtcttaaggcaccagtgaaattaaccaagaaacgcggagcaatgcccaaggctctgatgagtaggaacacgtggaaagcaccaggaatgccagcagaggcgaggcggcacacctctctgcagagcatccaggccgagcggcgggcagcggccagctgcttctgcgcatgctctcctcttggctctgcttctttctcacagtcacagtcacttcacagcttagccttgggcttcccatcacttccaggggtgcctctgccttggccagtgtgtgtcagctagtacacaagctccaagtgtgaatcaggtgtactggccgtcctgaagactgactgccctgtccttcctgccgacagccacacccgagtgtacacttaaagcggtgcccttctgcctctgtgggcctgctggctgctgttcctttcttgagtgtgattttttttttctctccctcaataaaataaatcaaactctgagac

SEQ ID NO: 19 - DNA, 77 bp, synthetic construct
  source 1..77: mol_type = other DNA
gggattttgccgatttcggcctattggttaaaaaatgagctgatttaacaaaaatttaacgcgaattttaacaaaat

SEQ ID NO: 20 - DNA, 82 bp, synthetic construct
  source 1..82: mol_type = other DNA
gtaagtatcaaggttacaagacaggtttaaggagaccaatagaaactgggcttgtcgagacagagaagactcttgcgtttct

SEQ ID NO: 21 - DNA, 51 bp, synthetic construct
  source 1..51: mol_type = other DNA
gataggcacctattggtcttactgacatccactttgcctttctctccacag

SEQ ID NO: 22 - DNA, 48 bp, synthetic construct
  source 1..48: mol_type = other DNA
gcctgcaagaactggttcagcagcctgagccacttcgtgatccacctg

SEQ ID NO: 23 - DNA, 548 bp, synthetic construct
  source 1..548: mol_type = other DNA
gatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcga

SEQ ID NO: 24 - DNA, 454 bp, Mus musculus
  source 1..454: mol_type = genomic DNA
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagagctgaaccttagccttgccttcctgggtacccttctgagcctcactgtcttctgtgagatgggcaaagtgcgggtgtgactccttggcaacggtgttacaccagggcaggtaaagttgtagttatttgtggggtacaccaggactgttaaaggtgtaactat

SEQ ID NO: 25 - DNA, 1157 bp, Mus musculus
  source 1..1157: mol_type = genomic DNA
ggtctcacccagcattttcacttctaataagttcaaatgtgatacggcacctttctaaaaattagttttcagggaaatagggttcaaaactggtagtggtagggtccattctcacgacccccaggcctgctaaccctgaccaagctacctattacttaccctcctctttctcctcctcctctttctccttctcctgcttcccctcttccttctccctcccttcctctccctcctccccctccttggctgtgatcagatccagagcctgaatgagcctcctgaccccacacccccactagcatgggcctgcaagtgcccagaagtccctcctgcctcctaaactgcccagccgatccattagctcttccttcttcccagtgaaagaagcaggcacagcctgtccctcccgttctacagaaaggaagctacagcacagggagggccaaaggccttcctgggactagacagttgatcaacagcaggactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccacc

SEQ ID NO: 26 - DNA, 76 bp, Mus musculus
  source 1..76: mol_type = genomic DNA
cccatgtcagctgcttgtgctttccagagacaaaacaggaataatagatgtcattaaatatacattgggccccagg

SEQ ID NO: 27 - DNA, 95 bp, Mus musculus
  source 1..95: mol_type = genomic DNA
agcctgagcctcctttccatctctgtggaggcagacataggacccccaacaaacagcatgcaggttgggagccagccacaggacccaggtaaggg

SEQ ID NO: 28 - DNA, 171 bp, synthetic construct
  source 1..171: mol_type = other DNA
cccatgtcagctgcttgtgctttccagagacaaaacaggaataatagatgtcattaaatatacattgggccccaggagcctgagcctcctttccatctctgtggaggcagacataggacccccaacaaacagcatgcaggttgggagccagccacaggacccaggtaaggg

SEQ ID NO: 29 - DNA, 171 bp, synthetic construct
  source 1..171: mol_type = other DNA
agcctgagcctcctttccatctctgtggaggcagacataggacccccaacaaacagcatgcaggttgggagccagccacaggacccaggtaagggcccatgtcagctgcttgtgctttccagagacaaaacaggaataatagatgtcattaaatatacattgggccccagg

SEQ ID NO: 30 - DNA, 184 bp, Mus musculus
  source 1..184: mol_type = genomic DNA
cccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggg

SEQ ID NO: 31 - DNA, 82 bp, Mus musculus
  source 1..82: mol_type = genomic DNA
tgaggtgggagctgggctctccctgatgtattattcagctccctggagttggccagctcctgttacactggccacagccctg

SEQ ID NO: 32 - DNA, 106 bp, Mus musculus
  source 1..106: mol_type = genomic DNA
cacaggcctcccagtctaccctcactagaaaacaacacccaggcactttccaccacctctcaaaggtgaaacccaaggctggtctagagaatgaattatggatcct

SEQ ID NO: 33 - DNA, 188 bp, synthetic construct
  source 1..188: mol_type = other DNA
tgaggtgggagctgggctctccctgatgtattattcagctccctggagttggccagctcctgttacactggccacagccctgcacaggcctcccagtctaccctcactagaaaacaacacccaggcactttccaccacctctcaaaggtgaaacccaaggctggtctagagaatgaattatggatcct

SEQ ID NO: 34 - DNA, 188 bp, synthetic construct
  source 1..188: mol_type = other DNA
cacaggcctcccagtctaccctcactagaaaacaacacccaggcactttccaccacctctcaaaggtgaaacccaaggctggtctagagaatgaattatggatccttgaggtgggagctgggctctccctgatgtattattcagctccctggagttggccagctcctgttacactggccacagccctg

SEQ ID NO: 35 - DNA, 430 bp, Mus musculus
  source 1..430: mol_type = genomic DNA
tgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcct

SEQ ID NO: 36 - DNA, 1611 bp, synthetic construct
  source 1..1611: mol_type = other DNA
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagagctgaaccttagccttgccttcctgggtacccttctgagcctcactgtcttctgtgagatgggcaaagtgcgggtgtgactccttggcaacggtgttacaccagggcaggtaaagttgtagttatttgtggggtacaccaggactgttaaaggtgtaactatggtctcacccagcattttcacttctaataagttcaaatgtgatacggcacctttctaaaaattagttttcagggaaatagggttcaaaactggtagtggtagggtccattctcacgacccccaggcctgctaaccctgaccaagctacctattacttaccctcctctttctcctcctcctctttctccttctcctgcttcccctcttccttctccctcccttcctctccctcctccccctccttggctgtgatcagatccagagcctgaatgagcctcctgaccccacacccccactagcatgggcctgcaagtgcccagaagtccctcctgcctcctaaactgcccagccgatccattagctcttccttcttcccagtgaaagaagcaggcacagcctgtccctcccgttctacagaaaggaagctacagcacagggagggccaaaggccttcctgggactagacagttgatcaacagcaggactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccacc

SEQ ID NO: 37 - DNA, 1611 bp, synthetic construct
  source 1..1611: mol_type = other DNA
ggtctcacccagcattttcacttctaataagttcaaatgtgatacggcacctttctaaaaattagttttcagggaaatagggttcaaaactggtagtggtagggtccattctcacgacccccaggcctgctaaccctgaccaagctacctattacttaccctcctctttctcctcctcctctttctccttctcctgcttcccctcttccttctccctcccttcctctccctcctccccctccttggctgtgatcagatccagagcctgaatgagcctcctgaccccacacccccactagcatgggcctgcaagtgcccagaagtccctcctgcctcctaaactgcccagccgatccattagctcttccttcttcccagtgaaagaagcaggcacagcctgtccctcccgttctacagaaaggaagctacagcacagggagggccaaaggccttcctgggactagacagttgatcaacagcaggactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccaccctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagagctgaaccttagccttgccttcctgggtacccttctgagcctcactgtcttctgtgagatgggcaaagtgcgggtgtgactccttggcaacggtgttacaccagggcaggtaaagttgtagttatttgtggggtacaccaggactgttaaaggtgtaactat

SEQ ID NO: 38 - DNA, 965 bp, synthetic construct
  source 1..965: mol_type = other DNA
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccacc

SEQ ID NO: 39 - DNA, 520 bp, synthetic construct
  source 1..520: mol_type = other DNA
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctttttatgaggtgggagctgggctctccctgatgtattattcagctccctggagttggccagctcctgttacactggccacagccctgggcatccgctgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactg

SEQ ID NO: 40 - DNA, 235 bp, Homo sapiens
  source 1..235: mol_type = genomic DNA
gtatgccttttgagatggatgcagcaggttctgtgaggctgccaggaggggtagagttcccgggggcctcgggccccgctggagtgtggagcaggcccatgctcagctctccaggctgttcgtggctcccctgtcagctgctcactcctttccagagacaaaacaggaataatagacatcattaaatatacatagggccccaggcggtcggcgtggtgggctgggcctcccttcc

SEQ ID NO: 41 - DNA, 688 bp, Homo sapiens
  source 1..688: mol_type = genomic DNA
tgccctgccttctgagccggcagcctggctccccaccccatgtattattcagctcctgagagccagccagctcctgttacactgaccgcagcccagcacctgctctgcccattcccctcctcccttgcctaggacctagagggttcaaagttctcctccaagatgacttggtgggctttggccatcccaccctaggccccacttctggcccagtgcaggtgtgctggtgatttagggcaggtggcattccatctctgtggctcaatgtcttcctctgtgaagccgaagtgacccaagggctcccttcatggggttgagccagctgtggcccagggagggcctaaccaggatgagcactgatgttgccatgacgactccgaggccagaatgtctcccccagcacaggcctcataggcaggcttccccatcctggtaaacaacacccacacactttctactactgctctagggtgaaacccaaggcgctctagaggagatgaattatggatccgccctcccggaatcctggctcggccctccccacgccacccagggccagtcgggtctgctcacagcccgaggaggccgcgtgtccagccgcgggcaagagacagagcaggtccctgtgtctccaagtccctgagcccgtgacaccggccccaggccctgtagagagcaggcagccacc

SEQ ID NO: 42 - DNA, 77 bp, Homo sapiens
  source 1..77: mol_type = genomic DNA
cccctgtcagctgctcactcctttccagagacaaaacaggaataatagacatcattaaatatacatagggccccagg

SEQ ID NO: 43 - DNA, 82 bp, Homo sapiens
  source 1..82: mol_type = genomic DNA
tgagccggcagcctggctccccaccccatgtattattcagctcctgagagccagccagctcctgttacactgaccgcagccc

SEQ ID NO: 44 - DNA, 111 bp, Homo sapiens
  source 1..111: mol_type = genomic DNA
cacaggcctcataggcaggcttccccatcctggtaaacaacacccacacactttctactactgctctagggtgaaacccaaggcgctctagaggagatgaattatggatcc

SEQ ID NO: 45 - DNA, 193 bp, synthetic construct
  source 1..193: mol_type = other DNA
tgagccggcagcctggctccccaccccatgtattattcagctcctgagagccagccagctcctgttacactgaccgcagccccacaggcctcataggcaggcttccccatcctggtaaacaacacccacacactttctactactgctctagggtgaaacccaaggcgctctagaggagatgaattatggatcc

SEQ ID NO: 46 - DNA, 193 bp, synthetic construct
  source 1..193: mol_type = other DNA
cacaggcctcataggcaggcttccccatcctggtaaacaacacccacacactttctactactgctctagggtgaaacccaaggcgctctagaggagatgaattatggatcctgagccggcagcctggctccccaccccatgtattattcagctcctgagagccagccagctcctgttacactgaccgcagccc

SEQ ID NO: 47 - DNA, 499 bp, Homo sapiens
  source 1..499: mol_type = genomic DNA
tgagccggcagcctggctccccaccccatgtattattcagctcctgagagccagccagctcctgttacactgaccgcagcccagcacctgctctgcccattcccctcctcccttgcctaggacctagagggttcaaagttctcctccaagatgacttggtgggctttggccatcccaccctaggccccacttctggcccagtgcaggtgtgctggtgatttagggcaggtggcattccatctctgtggctcaatgtcttcctctgtgaagccgaagtgacccaagggctcccttcatggggttgagccagctgtggcccagggagggcctaaccaggatgagcactgatgttgccatgacgactccgaggccagaatgtctcccccagcacaggcctcataggcaggcttccccatcctggtaaacaacacccacacactttctactactgctctagggtgaaacccaaggcgctctagaggagatgaattatggatcc

SEQ ID NO: 48 - DNA, 1262 bp, synthetic construct
  source 1..1262: mol_type = other DNA
gtatgccttttgagatggatgcagcaggttctgtgaggctgccaggaggggtagagttcccgggggcctcgggccccgctggagtgtggagcaggcccatgctcagctctccaggctgttcgtggctcccctgtcagctgctcactcctttccagagacaaaacaggaataatagacatcattaaatatacatagggccccaggcggtcggcgtggtgggctgggcctcccttccccataacactgagctgctctgctgggccaaccgtgctcctgggccagccagaggacccccatgaggcggcatgcaggcggggagcaggccacagaacgcaggtaaggagaccttagcctagagtccttggggtctgtcactggccaccctcgcatcccaggctgcaggaaactgaggcccagagaggacaaggactttcctggacccacacagccagtcagtgacagagcctagggtctgagccaggcctgacccaacctccatttctgcctctctacccctgcccccgccccaacacacacacacacacaagtggagttccactgaaacgcccctccttgccctgccttctgagccggcagcctggctccccaccccatgtattattcagctcctgagagccagccagctcctgttacactgaccgcagcccagcacctgctctgcccattcccctcctcccttgcctaggacctagagggttcaaagttctcctccaagatgacttggtgggctttggccatcccaccctaggccccacttctggcccagtgcaggtgtgctggtgatttagggcaggtggcattccatctctgtggctcaatgtcttcctctgtgaagccgaagtgacccaagggctcccttcatggggttgagccagctgtggcccagggagggcctaaccaggatgagcactgatgttgccatgacgactccgaggccagaatgtctcccccagcacaggcctcataggcaggcttccccatcctggtaaacaacacccacacactttctactactgctctagggtgaaacccaaggcgctctagaggagatgaattatggatccgccctcccggaatcctggctcggccctccccacgccacccagggccagtcgggtctgctcacagcccgaggaggccgcgtgtccagccgcgggcaagagacagagcaggtccctgtgtctccaagtccctgagcccgtgacaccggccccaggccctgtagagagcaggcagccacc

SEQ ID NO: 49 - DNA, 649 bp, synthetic construct
  source 1..649: mol_type = other DNA
gcaggcccatgctcagctctccaggctgttcgtggctcccctgtcagctgctcactcctttccagagacaaaacaggaataatagacatcattaaatatacatagggccccaggcggtcggcgtggtgggctgggcctcccttccccataacactgagctgctctgctgggccaaccgtgctcctgggccagccagaggacccccatgaggcggcatgcaggcggggagcaggccacagaacgcaggtaaggagaccttgccttctgagccggcagcctggctccccaccccatgtattattcagctcctgagagccagccagctcctgttacactgaccgcagcccagcacctgctctgcccattcccctcctcccttgcctaggacctagagggttcaaagttctcctccaagatgacttggtgggctttggccatcgggcctaaccaggatgagcactgatgttgccatgacgactccgaggccagaatgtctcccccagcacaggcctcataggcaggcttccccatcctggtaaacaacacccacacactttctactactgctctagggtgaaacccaaggcgctctagaggagatgaattatggatccgccctcccggaatcctggctcggccctccccacgc

SEQ ID NO: 50 - DNA, 240 bp, Mus musculus
  source 1..240: mol_type = genomic DNA
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctt

SEQ ID NO: 51 - DNA, 95 bp, Mus musculus
  source 1..95: mol_type = genomic DNA
tttatgaggtgggagctgggctctccctgatgtattattcagctccctggagttggccagctcctgttacactggccacagccctgggcatccgc

SEQ ID NO: 52 - DNA, 185 bp, Mus musculus
  source 1..185: mol_type = genomic DNA
tgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactg

SEQ ID NO: 53 - DNA, 335 bp, synthetic construct
  source 1..335: mol_type = other DNA
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctttttatgaggtgggagctgggctctccctgatgtattattcagctccctggagttggccagctcctgttacactggccacagccctgggcatccgc

SEQ ID NO: 54 - DNA, 425 bp, synthetic construct
  source 1..425: mol_type = other DNA
ctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactg

SEQ ID NO: 55 - DNA, 280 bp, synthetic construct
  source 1..280: mol_type = other DNA
tttatgaggtgggagctgggctctccctgatgtattattcagctccctggagttggccagctcctgttacactggccacagccctgggcatccgctgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactg

SEQ ID NO: 56 - DNA, 520 bp, synthetic construct
  source 1..520: mol_type = other DNA
tttatgaggtgggagctgggctctccctgatgtattattcagctccctggagttggccagctcctgttacactggccacagccctgggcatccgcctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactg

SEQ ID NO: 57 - DNA, 520 bp, synthetic construct
  source 1..520: mol_type = other DNA
tgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctttttatgaggtgggagctgggctctccctgatgtattattcagctccctggagttggccagctcctgttacactggccacagccctgggcatccgc

SEQ ID NO: 58 - DNA, 520 bp, synthetic construct
  source 1..520: mol_type = other DNA
tttatgaggtgggagctgggctctccctgatgtattattcagctccctggagttggccagctcctgttacactggccacagccctgggcatccgctgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgctgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctt

SEQ ID NO: 59 - DNA, 964 bp, synthetic construct
  source 1..964: mol_type = other DNA
tgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtccttaagcttctgccactggctccggcattgcagagagaagagaaggggcggcagactggagagctgggctccatttttgttccttggtgccctgcccctccccatgacctgcagagacattcagcctgccaggctttatgaggtgggagctgggctctccctgatgtattattcagctccctggagttggccagctcctgttacactggccacagccctgggcatccgcttctcacttctagtttcccctccaaggtaatgtggtgggtcatgatcattctatcctggcttcagggacctgactccactttggggccattcgaggggtctagggtagatgatgtccccctgtggggattaatgtcctgctctgtaaaactgagctagctgagatccaggagggcttggccagagacagcaagttgttgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactgaggagagactgtaggttcagctacaagcaaaaagacctagctggtctccaagcagtgtctccaagtccctgaacctgtgacacctgccccaggcatcatcaggcacagagggccacc

SEQ ID NO: 60 - DNA, 519 bp, synthetic construct
  source 1..519: mol_type = other DNA
tgcagctcagcctactacttgctttccaggctgttcctagttcccatgtcagctgcttgtgctttccagagacaaaacaggaataatagatgtcattaaatatacattgggccccaggcggtcaatgtggcagcctgagcctcctttccatctctgtggaggcagacataggacccccaacaaacagcatgcaggttgggagccagccacaggacccaggtaaggggccctgggtcctttttatgaggtgggagctgggctctccctgatgtattattcagctccctggagttggccagctcctgttacactggccacagccctgggcatccgctgccatggtgactttaaagccaggttgctgccccagcacaggcctcccagtctaccctcactagaaaacaacacccaggcactttccaccacctctcaaaggtgaaacccaaggctggtctagagaatgaattatggatcctcgctgtccgtgccacccagctagtcccagcggctcagacactg

SEQ ID NO: 61 - DNA, 425 bp, synthetic construct
  source 1..425: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaa

SEQ ID NO: 62 - DNA, 865 bp, Homo sapiens
  source 1..865: mol_type = genomic DNA
ccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccgtataggaggaccgtgtaggccttcctgtcccgggccttgccagcggccagcccgatgaaggagctccctcgcagggggtagcctccgaaggagaagacgtgggagtggtcggcagtgacgaggctcagcgtgtcctcctcgctggtgagctggcccgccctctcaatggcgtcgtcgaacatgatcgtctcagtcagtgcccggtaagccctgctttcatgatgaccatggtcgatgcgaccaccctccacgaagaggaagaagccgcgggggtgtctgctcagcaggcgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacgtaccgggcaccctggcgttcgccgagccattcctgcaccagattcttcccgtccagcctggtcccaccttggctgtagtcatctgggtactcagggtctggggttcccatgcgaaacatgtactttcggcctcca

SEQ ID NO: 63 - DNA, 437 bp, Homo sapiens
  source 1..437: mol_type = genomic DNA
ccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccgtataggaggaccgtgtaggccttcctgtcccgggccttgccagcggccagcccgatgaaggagctccctcgcagggggtagcctccgaaggagaagacgtgggagtggtcggcagtgacgaggctcagcgtgtcctcctcgctggtga

SEQ ID NO: 64 - DNA, 428 bp, Homo sapiens
  source 1..428: mol_type = genomic DNA
gctggcccgccctctcaatggcgtcgtcgaacatgatcgtctcagtcagtgcccggtaagccctgctttcatgatgaccatggtcgatgcgaccaccctccacgaagaggaagaagccgcgggggtgtctgctcagcaggcgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacgtaccgggcaccctggcgttcgccgagccattcctgcaccagattcttcccgtccagcctggtcccaccttggctgtagtcatctgggtactcagggtctggggttcccatgcgaaacatgtactttcggcctcca

SEQ ID NO: 65 - DNA, 287 bp, Homo sapiens
  source 1..287: mol_type = genomic DNA
ccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttc

SEQ ID NO: 66 - DNA, 290 bp, Homo sapiens
  source 1..290: mol_type = genomic DNA
cgtataggaggaccgtgtaggccttcctgtcccgggccttgccagcggccagcccgatgaaggagctccctcgcagggggtagcctccgaaggagaagacgtgggagtggtcggcagtgacgaggctcagcgtgtcctcctcgctggtgagctggcccgccctctcaatggcgtcgtcgaacatgatcgtctcagtcagtgcccggtaagccctgctttcatgatgaccatggtcgatgcgaccaccctccacgaagaggaagaagccgcgggggtgtctgctcagcagg

SEQ ID NO: 67 - DNA, 288 bp, Homo sapiens
  source 1..288: mol_type = genomic DNA
cgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacgtaccgggcaccctggcgttcgccgagccattcctgcaccagattcttcccgtccagcctggtcccaccttggctgtagtcatctgggtactcagggtctggggttcccatgcgaaacatgtactttcggcctcca

SEQ ID NO: 68 - DNA, 84 bp, synthetic construct
  source 1..84: mol_type = other DNA
gtaagtatcaaggttacaagacaggtttaaggagaccaatagaaactgggcttgtcgagacagagaagactcttgcgtttctga

SEQ ID NO: 69 - DNA, 49 bp, synthetic construct
  source 1..49: mol_type = other DNA
taggcacctattggtcttactgacatccactttgcctttctctccacag

(19) United States
(12) Patent Application Publication
(10) Pub. No.: US 2026/0035716 A1
(43) Pub. Date: Feb. 5, 2026

(54) COMPOSITIONS AND METHODS FOR TREATING SENSORINEURAL HEARING LOSS USING OTOFERLIN DUAL VECTOR SYSTEMS

(71) Applicants: Decibel Therapeutics, Inc., Boston, MA (US); Regeneron Pharmaceuticals, Inc., Tarrytown, NY (US)

(72) Inventors: Joseph BURNS, Newton, MA (US); Kathryn ELLIS, Arlington, MA (US); Adam PALERMO, Somerville, MA (US); Martin SCHWANDER, Auburndale, MA (US); Jonathon WHITTON, Cambridge, MA (US); Leah SABIN, Goldens Bridge, NY (US); Christos KYRATSOUS, Irvington, NY (US); Meghan DRUMMOND SAMUELSON, Katonah, NY (US)

(21) Appl. No.: 19/303,522

(22) Filed: Aug. 19, 2025

Related U.S. Application Data
(63) Continuation of application No. 17/395,711, filed on Aug. 6, 2021, now Pat. No. 12,410,442.
Application No. 17/395,711 is a continuation of application No. PCT/US2020/017257, filed on Feb. 7, 2020.
(60) Provisional application No. 62/802,890, filed on Feb. 8, 2019.
(60) Provisional application No. 62/928,279, filed on Oct. 30, 2019.
(60) Provisional application No. 62/965,770, filed on Jan. 24, 2020.

(51) Int. Cl. C12N 15/86; A61K 48/00; C07K 14/705

(52) U.S. Cl. CPC C12N 15/86; A61K 48/005; C07K 14/705; C12N 2800/40; C12N 2830/008; C12N 2840/44

(57) ABSTRACT

The disclosure features compositions and methods for the treatment of sensorineural hearing loss and auditory neuropathy, particularly forms of the disease that are associated with mutations in otoferlin (OTOF), by way of OTOF gene therapy. The disclosure provides a variety of compositions that include a first nucleic acid vector that contains a polynucleotide encoding an N-terminal portion of an OTOF protein and a second nucleic acid vector that contains a polynucleotide encoding a C-terminal portion of an OTOF protein. These vectors can be used to increase the expression of OTOF in a subject, such as a human subject suffering from sensorineural hearing loss.

### SEQUENCE LISTING

The instant application contains a Sequence Listing which has been submitted electronically in XML format and is hereby incorporated by reference in its entirety. Said XML copy, created Aug. 12, 2025, is named 51471-003006_Sequence_Listing_8_12_25.xml and is 153,354 bytes in size.

### FIELD OF THE INVENTION

Described herein are compositions and methods for the treatment of sensorineural hearing loss and auditory neuropathy, particularly forms of the disease that are associated with mutations in otoferlin (OTOF), by way of OTOF gene therapy. The disclosure provides dual vector systems that include a first nucleic acid vector that contains a polynucleotide encoding an N-terminal portion of an OTOF protein and a second nucleic acid vector that contains a polynucleotide encoding a C-terminal portion of an OTOF protein. These vectors can be used to increase the expression of or provide wild-type OTOF to a subject, such as a human subject suffering from sensorineural hearing loss.

### BACKGROUND

Sensorineural hearing loss is a type of hearing loss caused by defects in the cells of the inner ear or the neural pathways that project from the inner ear to the brain. Although sensorineural hearing loss is often acquired, and can be caused by noise, infections, head trauma, ototoxic drugs, or aging, there are also congenital forms of sensorineural hearing loss associated with autosomal recessive mutations. One such form of autosomal recessive sensorineural hearing loss is associated with mutation of the otoferlin (OTOF) gene, which is implicated in prelingual nonsyndromic hearing loss. In recent years, efforts to treat hearing loss have increasingly focused on gene therapy as a possible solution; however, OTOF is too large to allow for treatment using standard gene therapy approaches. There is a need for new therapeutics to treat OTOF-related sensorineural hearing loss.

### SUMMARY OF THE INVENTION

The present invention provides compositions and methods for treating sensorineural hearing loss or auditory neuropathy in a subject, such as a human subject. The compositions and methods of the disclosure pertain to dual vector systems for the delivery of a polynucleotide encoding an otoferlin (OTOF) protein to a subject having or at risk of developing sensorineural hearing loss or auditory neuropathy (e.g., a subject with a mutation in OTOF). For example, using the compositions and methods described herein, a first nucleic acid vector and a second nucleic acid vector that each encode a portion of a functional OTOF protein may be delivered to a subject by way of viral gene therapy. The compositions and methods described herein may also be used to increase expression of a WT OTOF protein in a cochlear hair cell (e.g., an inner hair cell).

In a first aspect, the invention provides a composition comprising: a first nucleic acid vector containing a promoter selected from the group consisting of a myosin 15 (Myo15) promoter, a vesicular glutamate transporter 3 (VGLUT3) promoter, and a fibroblast growth factor 8 (FGF8) promoter operably linked to a first coding polynucleotide that encodes an N-terminal portion of an otoferlin (OTOF) protein; and a second nucleic acid vector containing a second coding polynucleotide that encodes a C-terminal portion of an OTOF protein and a polyadenylation (poly(A)) sequence positioned at the 3′ end of the second coding polynucleotide; wherein the first coding polynucleotide partially overlaps with the second coding polynucleotide, wherein neither the first nor second nucleic acid vector encodes a full-length OTOF protein, and, when introduced into a mammalian cell, the two first and second nucleic acid vectors undergo homologous recombination to form a recombined nucleic acid that encodes a full-length OTOF protein.

In another aspect, the invention provides a dual vector system comprising: a first nucleic acid vector containing a promoter selected from the group consisting of a myosin 15 (Myo15) promoter, a vesicular glutamate transporter 3 (VGLUT3) promoter, and a fibroblast growth factor 8 (FGF8) promoter operably linked to a first coding polynucleotide that encodes an N-terminal portion of an otoferlin (OTOF) protein; and a second nucleic acid vector containing a second coding polynucleotide that encodes a C-terminal portion of an OTOF protein and a polyadenylation (poly(A)) sequence positioned at the 3′ end of the second coding polynucleotide; wherein the first coding polynucleotide partially overlaps with the second coding polynucleotide, wherein neither the first nor second nucleic acid vector encodes a full-length OTOF protein, and, when introduced into a mammalian cell, the two first and second nucleic acid vectors undergo homologous recombination to form a recombined nucleic acid that encodes a full-length OTOF protein.

In another aspect, the invention provides a composition comprising: a first nucleic acid vector containing a promoter selected from the group consisting of a CAG promoter, a cytomegalovirus (CMV) promoter, a truncated CMV-chicken β-actin promoter (smCBA promoter), a Myo15 promoter, a Myosin 7A (Myo7A) promoter, a Myosin 6 (Myo6) promoter, a POU Class 4 Homeobox 3 (POU4F3) promoter, an OTOF promoter, an FGF8 promoter, and a VGLUT3 promoter operably linked to a first coding polynucleotide that encodes an N-terminal portion of a human OTOF protein; and a second nucleic acid vector containing a second coding polynucleotide that encodes a C-terminal portion of a human OTOF protein and a polyadenylation (poly(A)) sequence positioned at the 3′ end of the second coding polynucleotide; wherein the first coding polynucleotide partially overlaps with the second coding polynucleotide, wherein neither the first nor second nucleic acid vector encodes a full-length OTOF protein, and, when introduced into a mammalian cell, the two first and second nucleic acid vectors undergo homologous recombination to form a recombined nucleic acid that encodes a full-length OTOF protein.

In another aspect, the invention provides a dual vector system comprising: a first nucleic acid vector containing a promoter selected from the group consisting of a CAG promoter, a cytomegalovirus (CMV) promoter, a smCBA promoter, a Myo15 promoter, a Myosin 7A (Myo7A) promoter, a Myosin 6 (Myo6) promoter, a POU Class 4 Homeobox 3 (POU4F3) promoter, an OTOF promoter, an FGF8 promoter, and a VGLUT3 promoter operably linked to a first coding polynucleotide that encodes an N-terminal portion of a human OTOF protein; and a second nucleic acid vector containing a second coding polynucleotide that encodes a C-terminal portion of a human OTOF protein and a polyadenylation (poly(A)) sequence positioned at the 3′ end of the second coding polynucleotide; wherein the first coding polynucleotide partially overlaps with the second coding polynucleotide, wherein neither the first nor second nucleic acid vector encodes a full-length OTOF protein, and, when introduced into a mammalian cell, the two first and second nucleic acid vectors undergo homologous recombination to form a recombined nucleic acid that encodes a full-length OTOF protein.

In some embodiments of any of the foregoing aspects, the first and second nucleic acid vectors are adeno-associated virus (AAV) vectors. In some embodiments of any of the foregoing aspects, the serotype of the AAV vectors is selected from the group consisting of AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, and PHP.S.

In another aspect, the invention provides a composition comprising: a first nucleic acid vector containing a promoter selected from the group consisting of a CAG promoter, a CMV promoter, a smCBA promoter, a Myo15 promoter, a Myo7A promoter, a Myo6 promoter, a POU4F3 promoter, an OTOF promoter, an FGF8 promoter, and a VGLUT3 promoter operably linked to a first coding polynucleotide that encodes an N-terminal portion of an OTOF protein; and a second nucleic acid vector containing a second coding polynucleotide that encodes a C-terminal portion of an OTOF protein and a polyadenylation (poly(A)) sequence positioned at the 3′ end of the second coding polynucleotide; wherein the first coding polynucleotide partially overlaps with the second coding polynucleotide, wherein neither the first nor second nucleic acid vector encodes a full-length OTOF protein, and, when introduced into a mammalian cell, the two first and second nucleic acid vectors undergo homologous recombination to form a recombined nucleic acid that encodes a full-length OTOF protein, and wherein the first and second nucleic acid vectors are AAV vectors having a serotype selected from the group consisting of AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, and PHP.S.

In another aspect, the invention provides a dual vector system comprising: a first nucleic acid vector containing a promoter selected from the group consisting of a CAG promoter, a CMV promoter, a smCBA promoter, a Myo15 promoter, a Myo7A promoter, a Myo6 promoter, a POU4F3 promoter, an OTOF promoter, an FGF8 promoter, and a VGLUT3 promoter operably linked to a first coding polynucleotide that encodes an N-terminal portion of an OTOF protein; and a second nucleic acid vector containing a second coding polynucleotide that encodes a C-terminal portion of an OTOF protein and a polyadenylation (poly(A)) sequence positioned at the 3′ end of the second coding polynucleotide; wherein the first coding polynucleotide partially overlaps with the second coding polynucleotide, wherein neither the first nor second nucleic acid vector encodes a full-length OTOF protein, and, when introduced into a mammalian cell, the two first and second nucleic acid vectors undergo homologous recombination to form a recombined nucleic acid that encodes a full-length OTOF protein, and wherein the first and second nucleic acid vectors are AAV vectors having a serotype selected from the group consisting of AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, and PHP.S.

In another aspect, the invention provides a composition comprising: a first nucleic acid vector containing a promoter operably linked to a first coding polynucleotide that encodes an N-terminal portion of a human OTOF protein; and a second nucleic acid vector containing a second coding polynucleotide that encodes a C-terminal portion of a human OTOF protein and a polyadenylation (poly(A)) sequence positioned at the 3′ end of the second coding polynucleotide; wherein the first coding polynucleotide partially overlaps with the second coding polynucleotide, wherein neither the first nor second nucleic acid vector encodes a full-length OTOF protein, and, when introduced into a mammalian cell, the two first and second nucleic acid vectors undergo homologous recombination to form a recombined nucleic acid that encodes a full-length OTOF protein, and wherein the first and second nucleic acid vectors are AAV vectors having a serotype selected from the group consisting of AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, and PHP.S.

In another aspect, the invention provides a dual vector system comprising: a first nucleic acid vector containing a promoter operably linked to a first coding polynucleotide that encodes an N-terminal portion of a human OTOF protein; and a second nucleic acid vector containing a second coding polynucleotide that encodes a C-terminal portion of a human OTOF protein and a polyadenylation (poly(A)) sequence positioned at the 3′ end of the second coding polynucleotide; wherein the first coding polynucleotide partially overlaps with the second coding polynucleotide, wherein neither the first nor second nucleic acid vector encodes a full-length OTOF protein, and, when introduced into a mammalian cell, the two first and second nucleic acid vectors undergo homologous recombination to form a recombined nucleic acid that encodes a full-length OTOF protein, and wherein the first and second nucleic acid vectors are AAV vectors having a serotype selected from the group consisting of AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, and PHP.S.

In some embodiments of any of the foregoing aspects, the first and second coding polynucleotides that encode the OTOF protein (e.g., the human OTOF protein) do not contain introns.

In some embodiments of any of the foregoing aspects, the OTOF protein is a mammalian OTOF protein.

In some embodiments of any of the foregoing aspects, the OTOF protein is a murine OTOF protein. In some embodiments of any of the foregoing aspects, the murine OTOF protein has at least 85% sequence identity (e.g., 85%, 86%, 87%, 88%, 89%, 90%, 91%, 92%, 93%, 94%, 95%, 96%, 97%, 98%, 99%, or 100% identity) to the sequence of SEQ ID NO: 6, SEQ ID NO: 7, SEQ ID NO: 8, or SEQ ID NO: 9. In some embodiments of any of the foregoing aspects, the OTOF protein comprises or consists of the sequence of SEQ ID NO: 6.

In some embodiments of any of the foregoing aspects, the OTOF protein is a human OTOF protein. In some embodiments of any of the foregoing aspects, the human OTOF protein has at least 85% sequence identity (e.g., 85%, 86%, 87%, 88%, 89%, 90%, 91%, 92%, 93%, 94%, 95%, 96%, 97%, 98%, 99%, or 100% identity) to the sequence of SEQ ID NO: 1, SEQ ID NO: 2, SEQ ID NO:3, SEQ ID NO: 4, or SEQ ID NO: 5. In some embodiments of any of the foregoing aspects, the OTOF protein comprises or consists of the sequence of SEQ ID NO: 1. In some embodiments of any of the foregoing aspects, the OTOF protein comprises or consists of the sequence of SEQ ID NO: 2. In some embodiments of any of the foregoing aspects, the OTOF protein comprises or consists of the sequence of SEQ ID NO: 3. In some embodiments of any of the foregoing aspects, the OTOF protein comprises or consists of the sequence of SEQ ID NO: 4. In some embodiments of any of the foregoing aspects, the OTOF protein comprises or consists of the sequence of SEQ ID NO: 5.

In some embodiments of any of the foregoing aspects, the serotype of the AAV vectors is AAV1. In some embodiments of any of the foregoing aspects, the serotype of the AAV vectors is AAV9. In some embodiments of any of the foregoing aspects, the serotype of the AAV vectors is AAV6. In some embodiments of any of the foregoing aspects, the serotype of the AAV vectors is Anc80. In some embodiments of any of the foregoing aspects, the serotype of the AAV vectors is Anc80L65. In some embodiments of any of the foregoing aspects, the serotype of the AAV vectors is DJ/9. In some embodiments of any of the foregoing aspects, the serotype of the AAV vectors is 7m8. In some embodiments of any of the foregoing aspects, the serotype of the AAV vectors is AAV2. In some embodiments of any of the foregoing aspects, the serotype of the AAV vectors is AAV2quad(Y-F). In some embodiments of any of the foregoing aspects, the serotype of the AAV vectors is PHP.B. In some embodiments of any of the foregoing aspects, the serotype of the AAV vectors is AAV8. In some embodiments of any of the foregoing aspects, the first and second nucleic acid vectors have the same serotype (e.g., both the first and second nucleic acid vector are AAV vectors having an AAV1 serotype or an AAV9 serotype). In some embodiments of any of the foregoing aspects, the first and second nucleic acid vectors have different serotypes (e.g., the first nucleic acid vector is an AAV having an AAV1 serotype, and the second nucleic acid vector is an AAV having an AAV9 serotype).

In some embodiments of any of the foregoing aspects, each of the first and second coding polynucleotides encode about half of the OTOF protein sequence.

In some embodiments of any of the foregoing aspects, wherein the first coding polynucleotide overlaps with the second coding polynucleotide by at least 1 kilobase (kb).

In some embodiments of any of the foregoing aspects, the region of overlap between the first and second coding polynucleotides is centered at an OTOF exon boundary. In some embodiments of any of the foregoing aspects, the first coding polynucleotide encodes an N-terminal portion of the OTOF protein containing the OTOF N-terminus to 500 kb 3′ of the exon boundary at the center of the overlap region; and the second coding polynucleotide encodes a C-terminal portion of the OTOF protein containing 500 kb 5′ of the exon boundary at the center of the overlap region to the OTOF C-terminus.

In some embodiments of any of the foregoing aspects, the exon boundary at the center of the overlap region is not within a portion of the first coding polynucleotide or second coding polynucleotide that encodes a C2 domain.

In some embodiments of any of the foregoing aspects, the promoter is a Myo15 promoter.

In some embodiments of any of the foregoing aspects, the promoter is a long promoter (e.g., a promoter that is longer than 1 kb, e.g., 1.1 kb, 1.25 kb, 1.5 kb, 1.75 kb, 2 kb, 2.5 kb, 3 kb or longer). In some embodiments, the long promoter is a Myo15 promoter that is longer than 1 kb (e.g., a Myo15 promoter comprising or consisting of a sequence with at least 85% sequence identity (e.g., 85%, 90%, 95%, 96%, 97%, 98%, 99%, or more, sequence identity) to the sequence of SEQ ID NO: 36).

In some embodiments of any of the foregoing aspects, the exon boundary is selected such that the first coding polynucleotide encodes the entire C2C domain and the second coding polynucleotide encodes the entire C2D domain. In some embodiments of any of the foregoing aspects, the first coding polynucleotide contains exons 1-21 of a polynucleotide encoding the OTOF protein and 500 kb 3′ of the exon 21/22 boundary; and the second coding polynucleotide contains 500 kb 5′ of the exon 21/22 boundary and exons 22-48 of a polynucleotide encoding the OTOF protein. In some embodiments of any of the foregoing aspects, the first nucleic acid vector and the second nucleic acid vector do not contain OTOF untranslated regions (UTRs).

In some embodiments of any of the foregoing aspects, the promoter is a short promoter (e.g., a promoter that is 1 kb or shorter, e.g., approximately 1 kb, 950 bp, 900 bp, 850 bp, 800 bp, 750 bp, 700 bp, 650 bp, 600 bp, 550 bp 500 bp, 450 bp, 400 bp, 350 bp, 300 bp or shorter). In some embodiments, the short promoter is a CAG promoter. In some embodiments, the short promoter is a CMV promoter. In some embodiments, the short promoter is a smCBA promoter. In some embodiments, the short promoter is a Myo15 promoter that is 1 kb or shorter (e.g., a Myo15 promoter having a sequence with at least 85% sequence identity (e.g., 85%, 90%, 95%, 96%, 97%, 98%, 99%, or more, sequence identity) to any one of SEQ ID NOs: 38, 39, or 49-60).

In some embodiments of any of the foregoing aspects, the exon boundary is within a portion of the first coding polynucleotide and the second coding polynucleotide that encodes the C2D domain. In some embodiments of any of the foregoing aspects, the first coding polynucleotide contains exons 1-24 of a polynucleotide encoding the OTOF protein and 500 kb 3′ of the exon 24/25 boundary; and the second coding polynucleotide contains 500 kb 5′ of the exon 24/25 boundary and exons 25-48 of a polynucleotide encoding the OTOF protein. In some embodiments of any of the foregoing aspects, the first and second nucleic acid vectors include OTOF UTRs (e.g., full-length 3′ and 5′ UTRs).

In some embodiments of any of the foregoing aspects, the exon boundary is selected such that the first coding polynucleotide encodes the entire C2D domain and the second coding polynucleotide encodes the entire C2E domain. In some embodiments of any of the foregoing aspects, the first coding polynucleotide contains exons 1-28 of a polynucleotide encoding the OTOF protein and 500 kb 3′ of the exon 28/29 boundary; and the second coding polynucleotide contains 500 kb 5′ of the exon 28/29 boundary and exons 29-48 of a polynucleotide encoding the OTOF protein. In some embodiments of any of the foregoing aspects, the second nucleic acid vector contains a full-length OTOF 3′ UTR.

In another aspect, the invention provides a composition comprising: a first nucleic acid vector containing a promoter selected from the group consisting of a Myo15 promoter, a VGLUT3 promoter, and an FGF8 promoter operably linked to a first coding polynucleotide that encodes an N-terminal portion of an OTOF protein, and a splice donor signal sequence positioned at the 3′ end of the first coding polynucleotide; and a second nucleic acid vector containing a splice acceptor signal sequence, a second coding polynucleotide that encodes a C-terminal portion of an OTOF protein positioned at the 3′ end of the splice acceptor signal sequence, and a poly(A) sequence at the 3′ end of the second coding polynucleotide; wherein the first coding polynucleotide and the second coding polynucleotide do not overlap, and wherein neither the first nor second nucleic acid vector encodes a full-length OTOF protein.

In another aspect, the invention provides a dual vector system comprising: a first nucleic acid vector containing a promoter selected from the group consisting of a Myo15 promoter, a VGLUT3 promoter, and an FGF8 promoter operably linked to a first coding polynucleotide that encodes an N-terminal portion of an OTOF protein, and a splice donor signal sequence positioned at the 3′ end of the first coding polynucleotide; and a second nucleic acid vector containing a splice acceptor signal sequence, a second coding polynucleotide that encodes a C-terminal portion of an OTOF protein positioned at the 3′ end of the splice acceptor signal sequence, and a poly(A) sequence at the 3′ end of the second coding polynucleotide; wherein the first coding polynucleotide and the second coding polynucleotide do not overlap, and wherein neither the first nor second nucleic acid vector encodes a full-length OTOF protein.

In another aspect, the invention provides a composition comprising: a first nucleic acid vector containing a promoter selected from the group consisting of a Myo15 promoter, a VGLUT3 promoter, and an FGF8 promoter operably linked to a first coding polynucleotide that encodes an N-terminal portion of an OTOF protein, a splice donor signal sequence positioned at the 3′ end of the first coding polynucleotide, and a recombinogenic region positioned 3′ of the splice donor signal sequence; and a second nucleic acid vector containing a second recombinogenic region, a splice acceptor signal sequence positioned 3′ of the second recombinogenic region, a second coding polynucleotide that encodes a C-terminal portion of an OTOF protein positioned at the 3′ end of the splice acceptor signal sequence, and a poly(A) sequence positioned at the 3′ end of the second coding polynucleotide; wherein the first coding polynucleotide and the second coding polynucleotide do not overlap, and wherein neither the first nor second nucleic acid vector encodes a full-length OTOF protein.

In another aspect, the invention provides a dual vector system comprising: a first nucleic acid vector containing a promoter selected from the group consisting of a Myo15 promoter, a VGLUT3 promoter, and an FGF8 promoter operably linked to a first coding polynucleotide that encodes an N-terminal portion of an OTOF protein, a splice donor signal sequence positioned at the 3′ end of the first coding polynucleotide, and a recombinogenic region positioned 3′ of the splice donor signal sequence; and a second nucleic acid vector containing a second recombinogenic region, a splice acceptor signal sequence positioned 3′ of the second recombinogenic region, a second coding polynucleotide that encodes a C-terminal portion of an OTOF protein positioned at the 3′ end of the splice acceptor signal sequence, and a poly(A) sequence positioned at the 3′ end of the second coding polynucleotide; wherein the first coding polynucleotide and the second coding polynucleotide do not overlap, and wherein neither the first nor second nucleic acid vector encodes a full-length OTOF protein.

In another aspect, the invention provides a composition comprising: a first nucleic acid vector containing a promoter selected from the group consisting of a CAG promoter, a CMV promoter, a smCBA promoter, a Myo15 promoter, a Myo7A promoter, a Myo6 promoter, a POU4F3 promoter, an OTOF promoter, an FGF8 promoter, and a VGLUT3 promoter operably linked to a first coding polynucleotide that encodes an N-terminal portion of a human OTOF protein, and a splice donor signal sequence positioned at the 3′ end of the first coding polynucleotide; and a second nucleic acid vector containing a splice acceptor signal sequence, a second coding polynucleotide that encodes a C-terminal portion of a human OTOF protein positioned at the 3′ end of the splice acceptor signal sequence, and a poly(A) sequence at the 3′ end of the second coding polynucleotide; wherein the first coding polynucleotide and the second coding polynucleotide do not overlap, and wherein neither the first nor second nucleic acid vector encodes a full-length OTOF protein.

In another aspect, the invention provides a dual vector system comprising: a first nucleic acid vector containing a promoter selected from the group consisting of a CAG promoter, a CMV promoter, a smCBA promoter, a Myo15 promoter, a Myo7A promoter, a Myo6 promoter, a POU4F3 promoter, an OTOF promoter, an FGF8 promoter, and a VGLUT3 promoter operably linked to a first coding polynucleotide that encodes an N-terminal portion of a human OTOF protein, and a splice donor signal sequence positioned at the 3′ end of the first coding polynucleotide; and a second nucleic acid vector containing a splice acceptor signal sequence, a second coding polynucleotide that encodes a C-terminal portion of a human OTOF protein positioned at the 3′ end of the splice acceptor signal sequence, and a poly(A) sequence at the 3′ end of the second coding polynucleotide; wherein the first coding polynucleotide and the second coding polynucleotide do not overlap, and wherein neither the first nor second nucleic acid vector encodes a full-length OTOF protein.

In some embodiments of any of the foregoing aspects, the first and second nucleic acid vectors are adeno-associated virus (AAV) vectors. In some embodiments of any of the foregoing aspects, the serotype of the AAV vectors is selected from the group consisting of AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, and PHP.S.

In another aspect, the invention provides a composition comprising: a first nucleic acid vector containing a promoter selected from the group consisting of a CAG promoter, a CMV promoter, a smCBA promoter, a Myo15 promoter, a Myo7A promoter, a Myo6 promoter, a POU4F3 promoter, an OTOF promoter, an FGF8 promoter, and a VGLUT3 promoter operably linked to a first coding polynucleotide that encodes an N-terminal portion of an OTOF protein, and a splice donor signal sequence positioned at the 3′ end of the first coding polynucleotide; and a second nucleic acid vector containing a splice acceptor signal sequence, a second coding polynucleotide that encodes a C-terminal portion of an OTOF protein positioned at the 3′ end of the splice acceptor signal sequence, and a poly(A) sequence at the 3′ end of the second coding polynucleotide; wherein the first coding polynucleotide and the second coding polynucleotide do not overlap, wherein neither the first nor second nucleic acid vector encodes a full-length OTOF protein, and wherein the first and second nucleic acid vectors are AAV vectors having a serotype selected from the group consisting of AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, and PHP.S.

In another aspect, the invention provides a dual vector system comprising: a first nucleic acid vector containing a promoter selected from the group consisting of a CAG promoter, a CMV promoter, a smCBA promoter, a Myo15 promoter, a Myo7A promoter, a Myo6 promoter, a POU4F3 promoter, an OTOF promoter, an FGF8 promoter, and a VGLUT3 promoter operably linked to a first coding polynucleotide that encodes an N-terminal portion of an OTOF protein, and a splice donor signal sequence positioned at the 3′ end of the first coding polynucleotide; and a second nucleic acid vector containing a splice acceptor signal sequence, a second coding polynucleotide that encodes a C-terminal portion of an OTOF protein positioned at the 3′ end of the splice acceptor signal sequence, and a poly(A) sequence at the 3′ end of the second coding polynucleotide; wherein the first coding polynucleotide and the second coding polynucleotide do not overlap, wherein neither the first nor second nucleic acid vector encodes a full-length OTOF protein, and wherein the first and second nucleic acid vectors are AAV vectors having a serotype selected from the group consisting of AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, and PHP.S.

In another aspect, the invention provides a composition comprising: a first nucleic acid vector containing a promoter operably linked to a first coding polynucleotide that encodes an N-terminal portion of a human OTOF protein, and a splice donor signal sequence positioned at the 3′ end of the first coding polynucleotide; and a second nucleic acid vector containing a splice acceptor signal sequence, a second coding polynucleotide that encodes a C-terminal portion of a human OTOF protein positioned at the 3′ end of the splice acceptor signal sequence, and a poly(A) sequence at the 3′ end of the second coding polynucleotide; wherein the first coding polynucleotide and the second coding polynucleotide do not overlap, wherein neither the first nor second nucleic acid vector encodes a full-length OTOF protein, and wherein the first and second nucleic acid vectors are AAV vectors having a serotype selected from the group consisting of AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, and PHP.S.

In another aspect, the invention provides a dual vector system comprising: a first nucleic acid vector containing a promoter operably linked to a first coding polynucleotide that encodes an N-terminal portion of a human OTOF protein, and a splice donor signal sequence positioned at the 3′ end of the first coding polynucleotide; and a second nucleic acid vector containing a splice acceptor signal sequence, a second coding polynucleotide that encodes a C-terminal portion of a human OTOF protein positioned at the 3′ end of the splice acceptor signal sequence, and a poly(A) sequence at the 3′ end of the second coding polynucleotide; wherein the first coding polynucleotide and the second coding polynucleotide do not overlap, wherein neither the first nor second nucleic acid vector encodes a full-length OTOF protein, and wherein the first and second nucleic acid vectors are AAV vectors having a serotype selected from the group consisting of AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, and PHP.S.

In another aspect, the invention provides a composition comprising: a first nucleic acid vector containing a promoter selected from the group consisting of a CAG promoter, a CMV promoter, a smCBA promoter, a Myo15 promoter, a Myo7A promoter, a Myo6 promoter, a POU4F3 promoter, an OTOF promoter, an FGF8 promoter, and a VGLUT3 promoter operably linked to a first coding polynucleotide that encodes an N-terminal portion of a human OTOF protein, a splice donor signal sequence positioned at the 3′ end of the first coding polynucleotide, and a recombinogenic region positioned 3′ of the splice donor signal sequence; and a second nucleic acid vector containing a second recombinogenic region, a splice acceptor signal sequence positioned 3′ of the second recombinogenic region, a second coding polynucleotide that encodes a C-terminal portion of a human OTOF protein positioned at the 3′ end of the splice acceptor signal sequence, and a poly(A) sequence positioned at the 3′ end of the second coding polynucleotide; wherein the first coding polynucleotide and the second coding polynucleotide do not overlap, and wherein neither the first nor second nucleic acid vector encodes a full-length OTOF protein.

In another aspect, the invention provides a dual vector system comprising: a first nucleic acid vector containing a promoter selected from the group consisting of a CAG promoter, a CMV promoter, a smCBA promoter, a Myo15 promoter, a Myo7A promoter, a Myo6 promoter, a POU4F3 promoter, an OTOF promoter, an FGF8 promoter, and a VGLUT3 promoter operably linked to a first coding polynucleotide that encodes an N-terminal portion of a human OTOF protein, a splice donor signal sequence positioned at the 3′ end of the first coding polynucleotide, and a recombinogenic region positioned 3′ of the splice donor signal sequence; and a second nucleic acid vector containing a second recombinogenic region, a splice acceptor signal sequence positioned 3′ of the second recombinogenic region, a second coding polynucleotide that encodes a C-terminal portion of a human OTOF protein positioned at the 3′ end of the splice acceptor signal sequence, and a poly(A) sequence positioned at the 3′ end of the second coding polynucleotide; wherein the first coding polynucleotide and the second coding polynucleotide do not overlap, and wherein neither the first nor second nucleic acid vector encodes a full-length OTOF protein.

In some embodiments of any of the foregoing aspects, the first and second nucleic acid vectors are AAV vectors. In some embodiments of any of the foregoing aspects, the serotype of the AAV vectors is selected from the group consisting of AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, and PHP.S.

In another aspect, the invention provides a composition comprising: a first nucleic acid vector containing a promoter selected from the group consisting of a CAG promoter, a CMV promoter, a smCBA promoter, a Myo15 promoter, a Myo7A promoter, a Myo6 promoter, a POU4F3 promoter, an OTOF promoter, an FGF8 promoter, and a VGLUT3 promoter operably linked to a first coding polynucleotide that encodes an N-terminal portion of an OTOF protein, a splice donor signal sequence positioned at the 3′ end of the first coding polynucleotide, and a recombinogenic region positioned 3′ of the splice donor signal sequence; and a second nucleic acid vector containing a second recombinogenic region, a splice acceptor signal sequence positioned 3′ of the second recombinogenic region, a second coding polynucleotide that encodes a C-terminal portion of an OTOF protein positioned at the 3′ end of the splice acceptor signal sequence, and a poly(A) sequence positioned at the 3′ end of the second coding polynucleotide; wherein the first coding polynucleotide and the second coding polynucleotide do not overlap, wherein neither the first nor second nucleic acid vector encodes a full-length OTOF protein, and wherein the first and second nucleic acid vectors are AAV vectors having a serotype selected from the group consisting of AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, and PHP.S.

In another aspect, the invention provides a dual vector system comprising: a first nucleic acid vector containing a promoter selected from the group consisting of a CAG promoter, a CMV promoter, a smCBA promoter, a Myo15 promoter, a Myo7A promoter, a Myo6 promoter, a POU4F3 promoter, an OTOF promoter, an FGF8 promoter, and a VGLUT3 promoter operably linked to a first coding polynucleotide that encodes an N-terminal portion of an OTOF protein, a splice donor signal sequence positioned at the 3′ end of the first coding polynucleotide, and a recombinogenic region positioned 3′ of the splice donor signal sequence; and a second nucleic acid vector containing a second recombinogenic region, a splice acceptor signal sequence positioned 3′ of the second recombinogenic region, a second coding polynucleotide that encodes a C-terminal portion of an OTOF protein positioned at the 3′ end of the splice acceptor signal sequence, and a poly(A) sequence positioned at the 3′ end of the second coding polynucleotide; wherein the first coding polynucleotide and the second coding polynucleotide do not overlap, wherein neither the first nor second nucleic acid vector encodes a full-length OTOF protein, and wherein the first and second nucleic acid vectors are AAV vectors having a serotype selected from the group consisting of AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, and PHP.S.

In another aspect, the invention provides a composition comprising: a first nucleic acid vector containing a promoter operably linked to a first coding polynucleotide that encodes an N-terminal portion of a human OTOF protein, a splice donor signal sequence positioned at the 3′ end of the first coding polynucleotide, and a recombinogenic region positioned 3′ of the splice donor signal sequence; and a second nucleic acid vector containing a second recombinogenic region, a splice acceptor signal sequence positioned 3′ of the second recombinogenic region, a second coding polynucleotide that encodes a C-terminal portion of a human OTOF protein positioned at the 3′ end of the splice acceptor signal sequence, and a poly(A) sequence positioned at the 3′ end of the second coding polynucleotide; wherein the first coding polynucleotide and the second coding polynucleotide do not overlap, wherein neither the first nor second nucleic acid vector encodes a full-length OTOF protein, and wherein the first and second nucleic acid vectors are AAV vectors having a serotype selected from the group consisting of AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, and PHP.S.

In another aspect, the invention provides a dual vector system comprising: a first nucleic acid vector containing a promoter operably linked to a first coding polynucleotide that encodes an N-terminal portion of a human OTOF protein, a splice donor signal sequence positioned at the 3′ end of the first coding polynucleotide, and a recombinogenic region positioned 3′ of the splice donor signal sequence; and a second nucleic acid vector containing a second recombinogenic region, a splice acceptor signal sequence positioned 3′ of the second recombinogenic region, a second coding polynucleotide that encodes a C-terminal portion of a human OTOF protein positioned at the 3′ end of the splice acceptor signal sequence, and a poly(A) sequence positioned at the 3′ end of the second coding polynucleotide; wherein the first coding polynucleotide and the second coding polynucleotide do not overlap, wherein neither the first nor second nucleic acid vector encodes a full-length OTOF protein, and wherein the first and second nucleic acid vectors are AAV vectors having a serotype selected from the group consisting of AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, and PHP.S.

In some embodiments of any of the foregoing aspects, the first and second coding polynucleotides that encode the OTOF protein (e.g., the human OTOF protein) do not contain introns.

In some embodiments of any of the foregoing aspects, the OTOF protein is a mammalian OTOF protein.

In some embodiments of any of the foregoing aspects, the OTOF protein is a murine OTOF protein. In some embodiments of any of the foregoing aspects, the murine OTOF protein has at least 85% sequence identity (e.g., 85%, 86%, 87%, 88%, 89%, 90%, 91%, 92%, 93%, 94%, 95%, 96%, 97%, 98%, 99%, or 100% identity) to the sequence of SEQ ID NO: 6, SEQ ID NO: 7, SEQ ID NO: 8, or SEQ ID NO: 9. In some embodiments of any of the foregoing aspects, the murine OTOF protein comprises or consists of the sequence of SEQ ID NO: 6.

In some embodiments of any of the foregoing aspects, the OTOF protein is a human OTOF protein. In some embodiments of any of the foregoing aspects, the human OTOF protein has at least 85% sequence identity (e.g., 85%, 86%, 87%, 88%, 89%, 90%, 91%, 92%, 93%, 94%, 95%, 96%, 97%, 98%, 99%, or 100% identity) to the sequence of SEQ ID NO: 1, SEQ ID NO: 2, SEQ ID NO:3, SEQ ID NO: 4, or SEQ ID NO: 5. In some embodiments of any of the foregoing aspects, the human OTOF protein comprises or consists of the sequence of SEQ ID NO: 1. In some embodiments of any of the foregoing aspects, the human OTOF protein comprises or consists of the sequence of SEQ ID NO: 2. In some embodiments of any of the foregoing aspects, the human OTOF protein comprises or consists of the sequence of SEQ ID NO: 3. In some embodiments of any of the foregoing aspects, the human OTOF protein comprises or consists of the sequence of SEQ ID NO: 4. In some embodiments of any of the foregoing aspects, the human OTOF protein comprises or consists of the sequence of SEQ ID NO: 5.

In some embodiments of any of the foregoing aspects, the serotype of the AAV vectors is AAV1. In some embodiments of any of the foregoing aspects, the serotype of the AAV vectors is AAV9. In some embodiments of any of the foregoing aspects, the serotype of the AAV vectors is AAV6. In some embodiments of any of the foregoing aspects, the serotype of the AAV vectors is Anc80. In some embodiments of any of the foregoing aspects, the serotype of the AAV vectors is Anc80L65. In some embodiments of any of the foregoing aspects, the serotype of the AAV vectors is DJ/9. In some embodiments of any of the foregoing aspects, the serotype of the AAV vectors is 7m8. In some embodiments of any of the foregoing aspects, the serotype of the AAV vectors is AAV2. In some embodiments of any of the foregoing aspects, the serotype of the AAV vectors is AAV2quad(Y-F). In some embodiments of any of the foregoing aspects, the serotype of the AAV vectors is PHP.B. In some embodiments of any of the foregoing aspects, the serotype of the AAV vectors is AAV8. In some embodiments of any of the foregoing aspects, the first and second nucleic acid vectors have the same serotype (e.g., both the first and second nucleic acid vector are AAV vectors having an AAV1 serotype or an AAV9 serotype). In some embodiments of any of the foregoing aspects, the first and second nucleic acid vectors have different serotypes (e.g., the first nucleic acid vector is an AAV having an AAV1 serotype, and the second nucleic acid vector is an AAV having an AAV9 serotype).

In some embodiments, of any of the foregoing aspects, the first and second recombinogenic regions are the same. In some embodiments, of any of the foregoing aspects, the recombinogenic region is an AP gene fragment or an F1 phage AK gene. In some embodiments of any of the foregoing aspects, the recombinogenic region is an F1 phage AK gene. In some embodiments of any of the foregoing aspects, the F1 phage AK gene comprises or consists of the sequence of SEQ ID NO: 19. In some embodiments of any of the foregoing aspects, the recombinogenic region is an AP gene fragment. In some embodiments of any of the foregoing aspects, the AP gene fragment comprises or consists of the sequence of any one of SEQ ID NOs: 62-67.

In some embodiments of any of the foregoing aspects, the first nucleic acid vector further includes a degradation signal sequence positioned 3′ of the recombinogenic region; and the second nucleic acid vector further includes a degradation signal sequence positioned between the recombinogenic region and the splice acceptor signal sequence. In some embodiments of any of the foregoing aspects, the degradation signal sequence comprises or consists of the sequence of SEQ ID NO: 22.

In some embodiments of any of the foregoing aspects, each of the first and second coding polynucleotides encode about half of the OTOF protein sequence.

In some embodiments of any of the foregoing aspects, the division between the first and second coding polynucleotides is at an OTOF exon boundary.

In some embodiments of any of the foregoing aspects, the OTOF exon boundary is not within a portion of the first coding polynucleotide or second coding polynucleotide that encodes a C2 domain.

In some embodiments of any of the foregoing aspects, the promoter is a short promoter (e.g., a promoter that is 1 kb or shorter, e.g., approximately 1 kb, 950 bp, 900 bp, 850 bp, 800 bp, 750 bp, 700 bp, 650 bp, 600 bp, 550 bp 500 bp, 450 bp, 400 bp, 350 bp, 300 bp or shorter). In some embodiments, the short promoter is a CAG promoter. In some embodiments, the short promoter is a CMV promoter. In some embodiments, the short promoter is a smCBA promoter. In some embodiments, the short promoter is a Myo15 promoter that is 1 kb or shorter (e.g., a Myo15 promoter having a sequence with at least 85% sequence identity (e.g., 85%, 90%, 95%, 96%, 97%, 98%, 99%, or more, sequence identity) to any one of SEQ ID NOs: 38, 39, or 49-60).

In some embodiments of any of the foregoing aspects, the exon boundary is selected such that the first coding polynucleotide encodes the entire C2D domain and the second coding polynucleotide encodes the entire C2E domain. In some embodiments of any of the foregoing aspects, the first coding polynucleotide encodes exons 1-26 of a polynucleotide encoding the OTOF protein and the second coding polynucleotide encodes exons 27-48 of a polynucleotide encoding the OTOF protein. In some embodiments of any of the foregoing aspects, the first coding polynucleotide encodes exons 1-28 of a polynucleotide encoding the OTOF protein and the second coding polynucleotide encodes exons 29-48 of a polynucleotide encoding the OTOF protein. In some embodiments of any of the foregoing aspects, the first and second nucleic acid vectors contain OTOF UTRs (e.g., full length 3′ and 5′ UTRs).

In some embodiments of any of the foregoing aspects, the promoter is a Myo15 promoter.

In some embodiments of any of the foregoing aspects, the promoter is a long promoter (e.g., a promoter that is longer than 1 kb, e.g., 1.1 kb, 1.25 kb, 1.5 kb, 1.75 kb, 2 kb, 2.5 kb, 3 kb or longer). In some embodiments, the long promoter is a Myo15 promoter that is longer than 1 kb (e.g., a Myo15 promoter comprising or consisting of a sequence with at least 85% sequence identity (e.g., 85%, 90%, 95%, 96%, 97%, 98%, 99%, or more, sequence identity) to the sequence of SEQ ID NO: 36).

In some embodiments of any of the foregoing aspects, the OTOF exon boundary is not within a portion of the first coding polynucleotide or second coding polynucleotide that encodes a C2 domain.

In some embodiments of any of the foregoing aspects, the exon boundary is selected such that the first coding polynucleotide encodes the entire C2C domain and the second coding polynucleotide encodes the entire C2D domain. In some embodiments of any of the foregoing aspects, the first coding polynucleotide encodes exons 1-19 of a polynucleotide encoding the OTOF protein and the second coding polynucleotide encodes exons 20-48 of a polynucleotide encoding the OTOF protein. In some embodiments of any of the foregoing aspects, the first coding polynucleotide encodes exons 1-20 of a polynucleotide encoding the OTOF protein and the second coding polynucleotide encodes exons 21-48 of a polynucleotide encoding the OTOF protein. In some embodiments of any of the foregoing aspects, the first nucleic acid vector and the second nucleic acid vector do not contain OTOF UTRs.

In some embodiments of any of the foregoing aspects, the exon boundary is within a portion of the first coding polynucleotide and the second coding polynucleotide that encodes the C2D domain. In some embodiments of any of the foregoing aspects, the first coding polynucleotide encodes exons 1-25 of a polynucleotide encoding the OTOF protein and the second coding polynucleotide encodes exons 26-48 of a polynucleotide encoding the OTOF protein. In some embodiments of any of the foregoing aspects, the first coding polynucleotide encodes exons 1-24 of a polynucleotide encoding the OTOF protein and the second coding polynucleotide encodes exons 25-48 of a polynucleotide encoding the OTOF protein. In some embodiments of any of the foregoing aspects, the second nucleic acid vector contains a full-length OTOF 3′ UTR.

In some embodiments of any of the foregoing aspects, the splice donor sequence comprises or consists of the sequence of SEQ ID NO: 20. In some embodiments of any of the foregoing aspects, the splice donor sequence comprises or consists of the sequence of SEQ ID NO: 68.

In some embodiments of any of the foregoing aspects, the splice acceptor sequence comprises or consists of the sequence of SEQ ID NO: 21. In some embodiments of any of the foregoing aspects, the splice acceptor sequence comprises or consists of the sequence of SEQ ID NO: 69.

In some embodiments, the first and second nucleic acid vectors contain inverted terminal repeats (ITRs). In some embodiments of any of the foregoing aspects, the ITRs are AAV2 ITRs.

In some embodiments of any of the foregoing aspects, the poly(A) sequence is a bovine growth hormone (bGH) poly(A) signal sequence.

In some embodiments of any of the foregoing aspects, the second nucleic acid vector contains a Woodchuck Hepatitis Virus Posttranscriptional Regulatory Element (WPRE). In some embodiments of any of the foregoing aspects, the WPRE comprises or consists of the sequence of SEQ ID NO: 23. In some embodiments of any of the foregoing aspects, the WPRE comprises or consists of the sequence of SEQ ID NO: 61.

In some embodiments of any of the foregoing aspects, the composition contains a pharmaceutically acceptable excipient.

In another aspect, the invention provides a kit containing a composition of the invention.

In another aspect, the invention provides a method of increasing OTOF expression in a subject in need thereof by administering to the subject a therapeutically effective amount of a composition of the invention.

In another aspect, the invention provides a method of treating a subject having or at risk of developing sensorineural hearing loss by administering to the subject a therapeutically effective amount of a composition of the invention.

In another aspect, the invention provides a method of treating a subject having or at risk of developing auditory neuropathy by administering to the subject a therapeutically effective amount of a composition of the invention.

In another aspect, the invention provides a method of increasing OTOF expression in a subject in need thereof by administering to the subject a therapeutically effective amount of a pair of nucleic acid vectors listed in Table 4.

In another aspect, the invention provides a method of treating a subject having or at risk of developing sensorineural hearing loss by administering to the subject a therapeutically effective amount of a pair of nucleic acid vectors listed in Table 4.

In another aspect, the invention provides a method of treating a subject having or at risk of developing auditory neuropathy by administering to the subject a therapeutically effective amount of a pair of nucleic acid vectors listed in Table 4.

In some embodiments of any of the foregoing aspects, the subject has a mutation in OTOF.

In some embodiments of any of the foregoing aspects, the subject has been identified as having a mutation in OTOF.

In some embodiments of any of the foregoing aspects, the method further includes the step of identifying the subject as having a mutation in OTOF prior to administering the composition or the pair of nucleic acid vectors.

In some embodiments of any of the foregoing aspects, the method further includes the step of evaluating the hearing of the subject prior to administering the composition or the pair of nucleic acid vectors.

In some embodiments of any of the foregoing aspects, the composition or the pair of nucleic acid vectors is administered locally to the ear. In some embodiments of any of the foregoing aspects, the nucleic acid vectors are administered concurrently. In some embodiments of any of the foregoing aspects, the nucleic acid vectors are administered sequentially.

In some embodiments of any of the foregoing aspects, the method increases OTOF expression in a cochlear hair cell. In some embodiments of any of the foregoing aspects, the cochlear hair cell is an inner hair cell.

In some embodiments of any of the foregoing aspects, the subject is a mammal. In some embodiments of any of the foregoing aspects, the subject is a human.

In some embodiments of any of the foregoing aspects, the method further includes the step of evaluating the hearing of the subject after administering the composition or the pair of nucleic acid vectors. In some embodiments of any of the foregoing aspects, the method further includes the step of evaluating OTOF expression after administering the composition or the pair of nucleic acid vectors.

In some embodiments of any of the foregoing aspects, the composition or the pair of nucleic acid vectors increases OTOF expression in a cell (e.g., a cochlear hair cell), improves hearing (e.g., as assessed by standard tests, such as audiometry, auditory brainstem response (ABR), electrocochleography (ECOG), and otoacoustic emissions), prevents or reduces hearing loss, delays the development of hearing loss, slows the progression of hearing loss, improves speech discrimination, or improves hair cell function.

In some embodiments of any of the foregoing aspects, the composition or the pair of nucleic acid vectors is administered in an amount sufficient to increase OTOF expression in a cochlear hair cell, prevent or reduce hearing loss, delay the development of hearing loss, slow the progression of hearing loss, improve hearing (e.g., as assessed by standard tests, such as audiometry, ABR, ECOG, and otoacoustic emissions), improve speech discrimination, or improve hair cell function.

In another aspect, the invention provides a method of increasing OTOF expression in a cell by introducing a composition of the invention into the cell.

In another aspect, the invention provides a method of increasing OTOF expression in a cell by introducing a pair of nucleic acid vectors listed in Table 4 into the cell.

In some embodiments of any of the foregoing aspects, the cell is a cochlear hair cell. In some embodiments of any of the foregoing aspects, the cell is an inner hair cell.

In some embodiments of any of the foregoing aspects, the cell is a mammalian cell. In some embodiments of any of the foregoing aspects, the cell is a human cell.

In some embodiments of any of the foregoing aspects, the OTOF protein encoded by the pair of nucleic acid vectors has at least 85% sequence identity (e.g., 85%, 86%, 87%, 88%, 89%, 90%, 91%, 92%, 93%, 94%, 95%, 96%, 97%, 98%, 99%, or 100% identity) to the sequence of SEQ ID NO: 1, SEQ ID NO: 2, SEQ ID NO:3, SEQ ID NO: 4, or SEQ ID NO: 5. In some embodiments of any of the foregoing aspects, the OTOF protein encoded by the pair of nucleic acid vectors comprises or consists of the sequence of SEQ ID NO: 1. In some embodiments of any of the foregoing aspects, the OTOF protein encoded by the pair of nucleic acid vectors comprises or consists of the sequence of SEQ ID NO: 2. In some embodiments of any of the foregoing aspects, the OTOF protein encoded by the pair of nucleic acid vectors comprises or consists of the sequence of SEQ ID NO: 3. In some embodiments of any of the foregoing aspects, the OTOF protein encoded by the pair of nucleic acid vectors comprises or consists of the sequence of SEQ ID NO: 4. In some embodiments of any of the foregoing aspects, the OTOF protein encoded by the pair of nucleic acid vectors comprises or consists of the sequence of SEQ ID NO: 5.

In some embodiments of any of the foregoing aspects, the nucleic acid vectors are AAV vectors. In some embodiments of any of the foregoing aspects, the serotype of the AAV vectors is selected from the group consisting of AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, or 7m8. In some embodiments of any of the foregoing aspects, the serotype of the AAV vectors is AAV1. In some embodiments of any of the foregoing aspects, the serotype of the AAV vectors is AAV9. In some embodiments of any of the foregoing aspects, the serotype of the AAV vectors is AAV6. In some embodiments of any of the foregoing aspects, the serotype of the AAV vectors is Anc80. In some embodiments of any of the foregoing aspects, the serotype of the AAV vectors is Anc80L65. In some embodiments of any of the foregoing aspects, the serotype of the AAV vectors is DJ/9. In some embodiments of any of the foregoing aspects, the serotype of the AAV vectors is 7m8. In some embodiments of any of the foregoing aspects, the serotype of the AAV vectors is AAV2. In some embodiments of any of the foregoing aspects, the serotype of the AAV vectors is AAV2quad(Y-F). In some embodiments of any of the foregoing aspects, the serotype of the AAV vectors is PHP.B. In some embodiments of any of the foregoing aspects, the serotype of the AAV vectors is AAV8. In some embodiments of any of the foregoing aspects, the first and second nucleic acid vectors have the same serotype (e.g., both the first and second nucleic acid vector are AAV vectors having an AAV1 serotype or an AAV9 serotype). In some embodiments of any of the foregoing aspects, the first and second nucleic acid vectors have different serotypes (e.g., the first nucleic acid vector is an AAV having an AAV1 serotype, and the second nucleic acid vector is an AAV having an AAV9 serotype).

In some embodiments of any of the foregoing aspects, the vectors contain AAV2 ITRs.

In some embodiments of any of the foregoing aspects, the second nucleic acid vector in the pair of nucleic acid vectors contains a WPRE. In some embodiments of any of the foregoing aspects, the WPRE comprises or consists of the sequence of SEQ ID NO: 23. In some embodiments of any of the foregoing aspects, the WPRE comprises or consists of the sequence of SEQ ID NO: 61.

In some embodiments of any of the foregoing aspects, the nucleic acid vectors are overlapping dual vectors.

In some embodiments of any of the foregoing aspects, the nucleic acid vectors are trans-splicing dual vectors.

In some embodiments of any of the foregoing aspects, the nucleic acid vectors are dual hybrid vectors.

In some embodiments of any of the foregoing aspects, the recombinogenic region in the dual hybrid vectors is an AP gene fragment or an F1 phage AK gene. In some embodiments of any of the foregoing aspects, the F1 phage AK gene comprises or consists of the sequence of SEQ ID NO: 19. In some embodiments of any of the foregoing aspects, the AP gene fragment comprises or consists of the sequence of any one of SEQ ID NOs: 62-67. In some embodiments of any of the foregoing aspects, the first nucleic acid vector in the pair of nucleic acid vectors further contains a degradation signal sequence positioned 3′ of the recombinogenic region; and the second nucleic acid vector in the pair of nucleic acid vectors further contains a degradation signal sequence positioned between the recombinogenic region and the splice acceptor sequence. In some embodiments of any of the foregoing aspects, the degradation signal sequence comprises or consists of the sequence of SEQ ID NO: 22.

In some embodiments of any of the foregoing aspects, the splice donor sequence in the first nucleic acid vector comprises or consists of the sequence of SEQ ID NO: 20. In some embodiments of any of the foregoing aspects, the splice donor sequence comprises or consists of the sequence of SEQ ID NO: 68.

In some embodiments of any of the foregoing aspects, the splice acceptor sequence in the second nucleic acid vector comprises or consists of the sequence of SEQ ID NO: 21. In some embodiments of any of the foregoing aspects, the splice acceptor sequence comprises or consists of the sequence of SEQ ID NO: 69.

In some embodiments of any of the foregoing aspects, the Myo15 promoter comprises or consists of a first region having at least 85% sequence identity (e.g., 85%, 90%, 95%, 96%, 97%, 98%, 99%, or more, sequence identity) to SEQ ID NO: 24 or a functional portion or derivative thereof including the sequence of SEQ ID NO: 26 and/or SEQ ID NO: 27, operably linked to a second region having at least 85% sequence identity (e.g., 85%, 90%, 95%, 96%, 97%, 98%, 99%, or more, sequence identity) to SEQ ID NO: 25 or a functional portion or derivative thereof including the sequence of SEQ ID NO: 31 and/or SEQ ID NO: 32, optionally containing a linker including one to one hundred nucleotides (e.g., 1-5, 1-10, 1-15, 1-20, 1-25, 1-30, 1-35, 1-40, 1-45, 1-50, 1-60, 1-70, 1-80, 1-90, 10-20, 10-30, 10-40, 10-50, 10-60, 10-70, 10-80, 10-90, 10-100, 20-30, 20-40, 20-50, 20-60, 20-70, 20-80, 20-90, or 20-100 nucleotides) between the first region and the second region. In some embodiments, the first region comprises or consists of the sequence of SEQ ID NO: 24. In some embodiments, the second region comprises or consists of the sequence of SEQ ID NO: 25.

In some embodiments of any of the foregoing aspects, the Myo15 promoter comprises or consists of the sequence of SEQ ID NO: 36. In some embodiments of any of the foregoing aspects, the Myo15 promoter comprises or consists of the sequence of SEQ ID NO: 38. In some embodiments of any of the foregoing aspects, the Myo15 promoter comprises or consists of the sequence of SEQ ID NO: 39. In some embodiments of any of the foregoing aspects, the Myo15 promoter comprises or consists of the sequence of SEQ ID NO: 53. In some embodiments of any of the foregoing aspects, the Myo15 promoter comprises or consists of the sequence of SEQ ID NO: 54. In some embodiments of any of the foregoing aspects, the Myo15 promoter comprises or consists of the sequence of SEQ ID NO: 59. In some embodiments of any of the foregoing aspects, the Myo15 promoter comprises or consists of the sequence of SEQ ID NO: 60.

In some embodiments of any of the foregoing aspects, the Myo15 promoter comprises or consists of a first region having at least 85% sequence identity (e.g., 85%, 90%, 95%, 96%, 97%, 98%, 99%, or more, sequence identity) to SEQ ID NO: 25 or a functional portion or derivative thereof including the sequence of SEQ ID NO: 31 and/or SEQ ID NO: 32, operably linked to a second region having at least 85% sequence identity (e.g., 85%, 90%, 95%, 96%, 97%, 98%, 99%, or more, sequence identity) to SEQ ID NO: 24 or a functional portion or derivative thereof including the sequence of SEQ ID NO: 26 and/or SEQ ID NO: 27, optionally containing a linker including one to one hundred nucleotides (e.g., 1-5, 1-10, 1-15, 1-20, 1-25, 1-30, 1-35, 1-40, 1-45, 1-50, 1-60, 1-70, 1-80, 1-90, 10-20, 10-30, 10-40, 10-50, 10-60, 10-70, 10-80, 10-90, 10-100, 20-30, 20-40, 20-50, 20-60, 20-70, 20-80, 20-90, or 20-100 nucleotides) between the first region and the second region. In some embodiments, the first region comprises or consists of the sequence of SEQ ID NO: 25. In some embodiments, the second region comprises or consists of the sequence of SEQ ID NO: 24.

In some embodiments of any of the foregoing aspects, the Myo15 promoter comprises or consists of the sequence of SEQ ID NO: 37. In some embodiments of any of the foregoing aspects, the Myo15 promoter comprises or consists of the sequence of SEQ ID NO: 58.

In some embodiments of any of the foregoing aspects, the Myo15 promoter comprises or consists of a region having at least 85% sequence identity (e.g., 85%, 90%, 95%, 96%, 97%, 98%, 99%, or more, sequence identity) to SEQ ID NO: 24 or a functional portion or derivative thereof including the sequence of SEQ ID NO: 26 and/or SEQ ID NO: 27. In some embodiments, the region comprises or consists of the sequence of SEQ ID NO: 24.

In some embodiments of any of the foregoing aspects, the Myo15 promoter comprises or consists of a region having at least 85% sequence identity (e.g., 85%, 90%, 95%, 96%, 97%, 98%, 99%, or more, sequence identity) to SEQ ID NO: 25 or a functional portion or derivative thereof including the sequence of SEQ ID NO: 31 and/or SEQ ID NO: 32. In some embodiments, the region comprises or consists of the sequence of SEQ ID NO: 25.

In some embodiments of any of the foregoing aspects, the functional portion of SEQ ID NO: 24 contains the sequence of SEQ ID NO: 26. In some embodiments of any of the foregoing aspects, the functional portion of SEQ ID NO: 24 contains the sequence of SEQ ID NO: 27. In some embodiments of any of the foregoing aspects, the functional portion of SEQ ID NO: 24 contains the sequence of SEQ ID NO: 26 and the sequence of SEQ ID NO: 27. In some embodiments of any of the foregoing aspects, the functional portion of SEQ ID NO: 24 contains the sequence of SEQ ID NO: 28. In some embodiments of any of the foregoing aspects, the functional portion of SEQ ID NO: 24 contains the sequence of SEQ ID NO: 29. In some embodiments of any of the foregoing aspects, the functional portion of SEQ ID NO: 24 contains the sequence of SEQ ID NO: 30. In some embodiments of any of the foregoing aspects, the functional portion of SEQ ID NO: 24 contains the sequence of SEQ ID NO: 50.

In some embodiments of any of the foregoing aspects, the functional portion of SEQ ID NO: 25 contains the sequence of SEQ ID NO: 31. In some embodiments of any of the foregoing aspects, the functional portion of SEQ ID NO: 25 contains the sequence of SEQ ID NO: 32. In some embodiments of any of the foregoing aspects, the functional portion of SEQ ID NO: 25 contains the sequence of SEQ ID NO: 51. In some embodiments of any of the foregoing aspects, the functional portion of SEQ ID NO: 25 contains the sequence of SEQ ID NO: 51. In some embodiments of any of the foregoing aspects, the functional portion of SEQ ID NO: 25 contains the sequence of SEQ ID NO: 31 and the sequence of SEQ ID NO: 32. In some embodiments of any of the foregoing aspects, the functional portion of SEQ ID NO: 25 contains the sequence of SEQ ID NO: 33. In some embodiments of any of the foregoing aspects, the functional portion of SEQ ID NO: 25 contains the sequence of SEQ ID NO: 34. In some embodiments of any of the foregoing aspects, the functional portion of SEQ ID NO: 25 contains the sequence of SEQ ID NO: 35. In some embodiments of any of the foregoing aspects, the functional portion of SEQ ID NO: 25 contains the sequence of SEQ ID NO: 55.

In some embodiments of any of the foregoing aspects, the Myo15 promoter has at least 85% sequence identity (e.g., 85%, 90%, 95%, 96%, 97%, 98%, 99%, or more, sequence identity) to the nucleic acid sequence of any one of SEQ ID NOs: 50-58. In some embodiments, the Myo15 promoter comprises or consists of the sequence of SEQ ID NO: 50. In some embodiments, the Myo15 promoter comprises or consists of the sequence of SEQ ID NO: 51. In some embodiments, the Myo15 promoter comprises or consists of the sequence of SEQ ID NO: 52. In some embodiments, the Myo15 promoter comprises or consists of the sequence of SEQ ID NO: 53. In some embodiments, the Myo15 promoter comprises or consists of the sequence of SEQ ID NO: 54. In some embodiments, the Myo15 promoter comprises or consists of the sequence of SEQ ID NO: 55. In some embodiments, the Myo15 promoter comprises or consists of the sequence of SEQ ID NO: 56. In some embodiments, the Myo15 promoter comprises or consists of the sequence of SEQ ID NO: 57. In some embodiments, the Myo15 promoter comprises or consists of the sequence of SEQ ID NO: 58.

In some embodiments of any of the foregoing aspects, the Myo15 promoter comprises or consists of a first region having at least 85% sequence identity (e.g., 85%, 90%, 95%, 96%, 97%, 98%, 99%, or more, sequence identity) to SEQ ID NO: 40 or a functional portion or derivative thereof including the sequence of SEQ ID NO: 42, joined (e.g., operably linked) to a second region having at least 85% sequence identity (e.g., 85%, 90%, 95%, 96%, 97%, 98%, 99%, or more, sequence identity) to SEQ ID NO: 41 or a functional portion or derivative thereof including the sequence of SEQ ID NO: 43 and/or SEQ ID NO: 44, optionally containing a linker including one to four hundred nucleotides (e.g., 1-5, 1-10, 1-15, 1-20, 1-25, 1-30, 1-35, 1-40, 1-45, 1-50, 1-60, 1-70, 1-80, 1-90, 1-100, 1-125, 1-150, 1-175, 1-200, 1-225, 1-250, 1-275, 1-300, 1-325, 1-350, 1-375, 1-400, 10-20, 10-30, 10-40, 10-50, 10-60, 10-70, 10-80, 10-90, 10-100, 20-30, 20-40, 20-50, 20-60, 20-70, 20-80, 20-90, 20-100, 30-100, 40-100, 50-100, 50-150, 50-200, 50-250, 50-300, 50-350, 50-400, 100-150, 100-200, 100-250, 100-300, 100-350, 100-400, 150-200, 150-250, 150-300, 150-350, 150-400, 200-250, 200-300, 200-350, 200-400, 250-300, 250-350, 250-400, 300-400, or 350-400 nucleotides) between the first region and the second region. In some embodiments, the first region comprises or consists of the sequence of SEQ ID NO: 40. In some embodiments, the second region comprises or consists of the sequence of SEQ ID NO: 41.

In some embodiments of any of the foregoing aspects, the Myo15 promoter comprises or consists of the sequence of SEQ ID NO: 48.

In some embodiments of any of the foregoing aspects, the Myo15 promoter comprises or consists of the sequence of SEQ ID NO: 49.

In some embodiments of any of the foregoing aspects, the Myo15 promoter comprises or consists of a first region having at least 85% sequence identity (e.g., 85%, 90%, 95%, 96%, 97%, 98%, 99%, or more, sequence identity) to SEQ ID NO: 41 or a functional portion or derivative thereof including the sequence of SEQ ID NO: 43 and/or SEQ ID NO: 44, joined (e.g., operably linked) to a second region having at least 85% sequence identity (e.g., 85%, 90%, 95%, 96%, 97%, 98%, 99%, or more, sequence identity) to SEQ ID NO: 40 or a functional portion or derivative thereof including the sequence of SEQ ID NO: 42, optionally containing a linker including one to four hundred nucleotides (e.g., 1-5, 1-10, 1-15, 1-20, 1-25, 1-30, 1-35, 1-40, 1-45, 1-50, 1-60, 1-70, 1-80, 1-90, 1-100, 1-125, 1-150, 1-175, 1-200, 1-225, 1-250, 1-275, 1-300, 1-325, 1-350, 1-375, 1-400, 10-20, 10-30, 10-40, 10-50, 10-60, 10-70, 10-80, 10-90, 10-100, 20-30, 20-40, 20-50, 20-60, 20-70, 20-80, 20-90, 20-100, 30-100, 40-100, 50-100, 50-150, 50-200, 50-250, 50-300, 50-350, 50-400, 100-150, 100-200, 100-250, 100-300, 100-350, 100-400, 150-200, 150-250, 150-300, 150-350, 150-400, 200-250, 200-300, 200-350, 200-400, 250-300, 250-350, 250-400, 300-400, or 350-400 nucleotides) between the first region and the second region. In some embodiments, the first region comprises or consists of the sequence of SEQ ID NO: 41. In some embodiments, the second region comprises or consists of the sequence of SEQ ID NO: 40.

In some embodiments of any of the foregoing aspects, the Myo15 promoter comprises or consists of a region having at least 85% sequence identity (e.g., 85%, 90%, 95%, 96%, 97%, 98%, 99%, or more, sequence identity) to SEQ ID NO: 40 or a functional portion or derivative thereof including the sequence of SEQ ID NO: 42. In some embodiments, the region comprises or consists of the sequence of SEQ ID NO: 40.

In some embodiments of any of the foregoing aspects, the Myo15 promoter comprises or consists of a region having at least 85% sequence identity (e.g., 85%, 90%, 95%, 96%, 97%, 98%, 99%, or more, sequence identity) to SEQ ID NO: 41 or a functional portion or derivative thereof including the sequence of SEQ ID NO: 43 and/or SEQ ID NO: 44. In some embodiments, the region comprises or consists of the sequence of SEQ ID NO: 41.

In some embodiments of any of the foregoing aspects, the functional portion of SEQ ID NO: 40 contains the sequence of SEQ ID NO: 42.

In some embodiments of any of the foregoing aspects, the functional portion of SEQ ID NO: 41 contains the sequence of SEQ ID NO: 43. In some embodiments of any of the foregoing aspects, the functional portion of SEQ ID NO: 41 contains the sequence of SEQ ID NO: 44. In some embodiments of any of the foregoing aspects, the functional portion of SEQ ID NO: 41 contains the sequence of SEQ ID NO: 43 and the sequence of SEQ ID NO: 44. In some embodiments of any of the foregoing aspects, the functional portion of SEQ ID NO: 41 contains the sequence of SEQ ID NO: 45. In some embodiments of any of the foregoing aspects, the functional portion of SEQ ID NO: 41 contains the sequence of SEQ ID NO: 46. In some embodiments of any of the foregoing aspects, the functional portion of SEQ ID NO: 41 contains the sequence of SEQ ID NO: 47.

In some embodiments of any of the foregoing aspects, the Myo15 promoter induces transgene expression when operably linked to a transgene and introduced into a hair cell.

### Definitions

As used herein, the term “about” refers to a value that is within 10% above or below the value being described.

As used herein, “administration” refers to providing or giving a subject a therapeutic agent (e.g., a composition containing a first nucleic acid vector containing a polynucleotide that encodes an N-terminal portion of an otoferlin protein and a second nucleic acid vector containing a polynucleotide that encodes a C-terminal portion of an otoferlin protein), by any effective route. Exemplary routes of administration are described herein below.

As used herein, the term “cell type” refers to a group of cells sharing a phenotype that is statistically separable based on gene expression data. For instance, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, the term “cochlear hair cell” refers to group of specialized cells in the inner ear that are involved in sensing sound. There are two types of cochlear hair cells: inner hair cells and outer hair cells. Damage to cochlear hair cells and genetic mutations that disrupt cochlear hair cell function are implicated in hearing loss and deafness.

As used herein, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume. These properties are summarized for each of the twenty naturally-occurring amino acids in table 1 below.

TABLE 1
Representative physicochemical properties
of naturally-occurring amino acids

| | | | | Electrostatic | |
|---|---|---|---|---|---|
| | 3 | 1 | Side- | character at | |
| | Letter | Letter | chain | physiological pH | Steric |
| Amino Acid | Code | Code | Polarity | (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral and | large |
| | | | | cationic forms in | |
| | | | | equilibrium | |
| | | | | at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non-polar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small,
100-150 is intermediate,
150-200 is large, and
>200 is bulky

From this table it is appreciated that the conservative amino acid families include (i) G, A, V, L and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the term “degradation signal sequence” refers to a sequence (e.g., a nucleotide sequence that can be translated into an amino acid sequence) that mediates the degradation of a polypeptide in which it is contained. Degradation signal sequences can be included in the nucleic acid vectors of the invention to reduce or prevent the expression of portions of otoferlin proteins that have not undergone recombination and/or splicing. An exemplary degradation signal sequence for use in the invention is GCCTGCAAGAACTGGTTCAGCAGCCTGAGCCACTTCGTGATCCACCTG (SEQ ID NO: 22).

As used herein, the terms “effective amount,” “therapeutically effective amount,” and a “sufficient amount” of a composition, vector construct, or viral vector described herein refer to a quantity sufficient to, when administered to the subject in need thereof, including a mammal, for example a human, effect beneficial or desired results, including clinical results, and, as such, an “effective amount” or synonym thereto depends upon the context in which it is being applied. For example, in the context of treating sensorineural hearing loss, it is an amount of the composition, vector construct, or viral vector sufficient to achieve a treatment response as compared to the response obtained without administration of the composition, vector construct, or viral vector. The amount of a given composition described herein that will correspond to such an amount will vary depending upon various factors, such as the given agent, the pharmaceutical formulation, the route of administration, the type of disease or disorder, the identity of the subject (e.g. age, sex, weight) or host being treated, and the like, but can nevertheless be routinely determined by one skilled in the art. Also, as used herein, a “therapeutically effective amount” of a composition, vector construct, or viral vector of the present disclosure is an amount which results in a beneficial or desired result in a subject as compared to a control. Note that when a combination of active ingredients is administered, the effective amount of the combination may or may not include amounts of each ingredient that would have been effective if administered individually. As defined herein, a therapeutically effective amount of a composition, vector construct, viral vector or cell of the present disclosure may be readily determined by one of ordinary skill by routine methods known in the art. Dosage regime may be adjusted to provide the optimum therapeutic response.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell, e.g., a human cochlear hair cell).

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell, e.g., a human cochlear hair cell). Exogenous materials include those that are provided from an external source to an organism or to cultured matter extracted there from.

As used herein, the term “hair cell-specific expression” refers to production of an RNA transcript or polypeptide primarily within hair cells (e.g., cochlear hair cells) as compared to other cell types of the inner ear (e.g., spiral ganglion neurons, glia, or other inner ear cell types). Hair cell-specific expression of a transgene can be confirmed by comparing transgene expression (e.g., RNA or protein expression) between various cell types of the inner ear (e.g., hair cells vs. non-hair cells) using any standard technique (e.g., quantitative RT PCR, immunohistochemistry, Western Blot analysis, or measurement of the fluorescence of a reporter (e.g., GFP) operably linked to a promoter). A hair cell-specific promoter induces expression (e.g., RNA or protein expression) of a transgene to which it is operably linked that is at least 50% greater (e.g., 50%, 75%, 100%, 125%, 150%, 175%, 200% greater or more) in hair cells (e.g., cochlear hair cells) compared to at least 3 (e.g., 3, 4, 5, 6, 7, 8, 9, 10, or more) of the following inner ear cell types: Border cells, inner phalangeal cells, inner pillar cells, outer pillar cells, first row Deiter cells, second row Deiter cells, third row Deiter cells, Hensen's cells, Claudius cells, inner sulcus cells, outer sulcus cells, spiral prominence cells, root cells, interdental cells, basal cells of the stria vascularis, intermediate cells of the stria vascularis, marginal cells of the stria vascularis, spiral ganglion neurons, Schwann cells.

As used herein, the terms “increasing” and “decreasing” refer to modulating resulting in, respectively, greater or lesser amounts, of function, expression, or activity of a metric relative to a reference. For example, subsequent to administration of a composition in a method described herein, the amount of a marker of a metric (e.g., OTOF expression) as described herein may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the marker prior to administration. Generally, the metric is measured subsequent to administration at a time that the administration has had the recited effect, e.g., at least one week, one month, 3 months, or 6 months, after a treatment regimen has begun.

As used herein, the term “intron” refers to a region within the coding region of a gene, the nucleotide sequence of which is not translated into the amino acid sequence of the corresponding protein. The term intron also refers to the corresponding region of the RNA transcribed from a gene. Introns are transcribed into pre-mRNA, but are removed during processing, and are not included in the mature mRNA.

As used herein, “locally” or “local administration” means administration at a particular site of the body intended for a local effect and not a systemic effect. Examples of local administration are epicutaneous, inhalational, intra-articular, intrathecal, intravaginal, intravitreal, intrauterine, intra-lesional administration, lymph node administration, intratumoral administration, administration to the inner ear, and administration to a mucous membrane of the subject, wherein the administration is intended to have a local and not a systemic effect.

As used herein, the term “operably linked” refers to a first molecule that can be joined to a second molecule, wherein the molecules are so arranged that the first molecule affects the function of the second molecule. The term “operably linked” includes the juxtaposition of two or more components (e.g., a promoter and another sequence element) such that both components function normally and allow for the possibility that at least one of the components can mediate a function that is exerted upon at least one of the other components. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. In additional embodiments, two portions of a transcription regulatory element are operably linked to one another if they are joined such that the transcription-activating functionality of one portion is not adversely affected by the presence of the other portion. Two transcription regulatory elements may be operably linked to one another by way of a linker nucleic acid (e.g., an intervening non-coding nucleic acid) or may be operably linked to one another with no intervening nucleotides present.

As used herein, the terms “otoferlin” and “OTOF” refer to the gene associated with nonsyndromic recessive deafness DNFB9. The terms “otoferlin” and “OTOF” also refer to variants of wild-type OTOF protein and nucleic acids encoding the same, such as variant proteins having at least 85% sequence identity (e.g., 85%, 86%, 87%, 88%, 89%, 90%, 91%, 92%, 93%, 94%, 95%, 96%, 97%, 98%, 99%, or 99.9% identity, or more) to the amino acid sequence of a wild-type OTOF protein (e.g., any one of SEQ ID NOs: 1-5) or polynucleotides having at least 85% sequence identity (e.g., 85%, 86%, 87%, 88%, 89%, 90%, 91%, 92%, 93%, 94%, 95%, 96%, 97%, 98%, 99%, or 99.9% identity, or more) to the nucleic acid sequence of a wild-type OTOF gene, provided that the OTOF analog encoded retains the therapeutic function of wild-type OTOF. As used herein, OTOF may refer to the protein localized to inner hair cells or to the gene encoding this protein, depending upon the context, as will be appreciated by one of skill in the art.

As used herein, the term “plasmid” refers to a to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, the terms “nucleic acid” and “polynucleotide,” used interchangeably herein, refer to a polymeric form of nucleosides in any length. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules containing nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e., the succession of letters used as abbreviations for bases) that biochemically characterizes a specific nucleic acid. A polynucleotide sequence presented herein is presented in a 5′ to 3′ direction unless otherwise indicated.

As used herein, the terms “complementarity” or “complementary” of nucleic acids means that a nucleotide sequence in one strand of nucleic acid, due to orientation of its nucleobase groups, forms hydrogen bonds with another sequence on an opposing nucleic acid strand. The complementary bases in DNA are typically A with T and C with G. In RNA, they are typically C with G and U with A. Complementarity can be perfect or substantial/sufficient. Perfect complementarity between two nucleic acids means that the two nucleic acids can form a duplex in which every base in the duplex is bonded to a complementary base by Watson-Crick pairing. “Substantial” or “sufficient” complementary means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations to predict the Tm (melting temperature) of hybridized strands, or by empirical determination of Tm by using routine methods. Tm includes the temperature at which a population of hybridization complexes formed between two nucleic acid strands are 50% denatured (i.e., a population of double-stranded nucleic acid molecules becomes half dissociated into single strands). At a temperature below the Tm, formation of a hybridization complex is favored, whereas at a temperature above the Tm, melting or separation of the strands in the hybridization complex is favored. Tm may be estimated for a nucleic acid having a known G+C content in an aqueous 1 M NaCl solution by using, e.g., Tm=81.5+0.41(% G+C), although other known Tm computations take into account nucleic acid structural characteristics.

As used herein, the term “promoter” refers to a recognition site on DNA that is bound by an RNA polymerase. The polymerase drives transcription of the transgene. Exemplary promoters suitable for use with the compositions and methods described herein include ubiquitous promoters (e.g., the CAG promoter, cytomegalovirus (CMV) promoter, and a truncated form of the chimeric CMV-chicken β-actin promoter (CBA), in which the hybrid chicken β-actin/rabbit β-globin intron is greatly shortened to produce a smaller version of the promoter called smCBA), cochlear hair cell-specific promoters (e.g., the Myosin 15 (Myo15) promoter, the Myosin 7A (Myo7A) promoter, the Myosin 6 (Myo6) promoter, the POU Class 4 Homeobox 3 (POU4F3) promoter), and inner hair cell-specific promoters (e.g., the Fibroblast growth factor 8 (FGF8) promoter, the vesicular glutamate transporter 3 (VGLUT3) promoter, and the OTOF promoter).

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

$$100 multiplied by ( the fraction X / Y )$$

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

The term “derivative” as used herein refers to a nucleic acid, peptide, or protein or a variant or analog thereof comprising one or more mutations and/or chemical modifications as compared to a corresponding full-length wild-type nucleic acid, peptide, or protein. Non-limiting examples of chemical modifications involving nucleic acids include, for example, modifications to the base moiety, sugar moiety, phosphate moiety, phosphate-sugar backbone, or a combination thereof.

As used herein, the term “pharmaceutical composition” refers to a mixture containing a therapeutic agent, optionally in combination with one or more pharmaceutically acceptable excipients, diluents, and/or carriers, to be administered to a subject, such as a mammal, e.g., a human, in order to prevent, treat or control a particular disease or condition affecting or that may affect the subject.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio. Preferably, the term “pharmaceutically acceptable” means approved by a regulatory agency of the Federal or a state government or listed in the U.S. Pharmacopeia or other generally recognized pharmacopeia for use in mammals, and more particularly in humans.

As used herein, the term “recombinogenic region” refers to a region of homology that mediates recombination between two different sequences.

As used herein, the term “regulatory sequence” includes promoters, enhancers and other expression control elements (e.g., polyadenylation signals) that control the transcription or translation of the polynucleotides that encode OTOF. Such regulatory sequences are described, for example, in Goeddel, Gene Expression Technology: Methods in Enzymology 185 (Academic Press, San Diego, CA, 1990); incorporated herein by reference.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from a subject.

As used herein, the term “transfection” refers to any of a wide variety of techniques commonly used for the introduction of exogenous DNA into a prokaryotic or eukaryotic host cell, e.g., electroporation, lipofection, calcium-phosphate precipitation, DEAE-dextran transfection, Nucleofection, squeeze-poration, sonoporation, optical transfection, Magnetofection, impalefection and the like.

As used herein, the terms “subject” and “patient” refer to an animal (e.g., a mammal, such as a human), veterinary animals (e.g., cats, dogs, cows, horses, sheep, pigs, etc.) and experimental animal models of diseases (e.g., mice, rats). A subject to be treated according to the methods described herein may be one who has been diagnosed with hearing loss (e.g., hearing loss associated with a mutation in OTOF), or one at risk of developing these conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the terms “transduction” and “transduce” refer to a method of introducing a vector construct or a part thereof into a cell. Wherein the vector construct is contained in a viral vector such as for example an AAV vector, transduction refers to viral infection of the cell and subsequent transfer and integration of the vector construct or part thereof into the cell genome.

As used herein, “treatment” and “treating” of a state, disorder or condition can include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

As used herein, the term “vector” includes a nucleic acid vector, e.g., a DNA vector, such as a plasmid, an RNA vector, virus or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO94/11026; incorporated herein by reference as it pertains to vectors suitable for the expression of a gene of interest. Expression vectors suitable for use with the compositions and methods described herein contain a polynucleotide sequence as well as, e.g., additional sequence elements used for the expression of proteins and/or the integration of these polynucleotide sequences into the genome of a mammalian cell. Certain vectors that can be used for the expression of OTOF as described herein include vectors that contain regulatory sequences, such as promoter and enhancer regions, which direct gene transcription. Other useful vectors for expression of OTOF contain polynucleotide sequences that enhance the rate of translation of these genes or improve the stability or nuclear export of the mRNA that results from gene transcription. These sequence elements include, e.g., 5′ and 3′ untranslated regions and a polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the term “wild-type” refers to a genotype with the highest frequency for a particular gene in a given organism.

### BRIEF DESCRIPTION OF THE DRAWINGS

FIGS. 1A and 1B are maps of the 5′ and 3′ vectors in an overlapping dual vector system. The 5′ vector contains AAV2 ITRs, and a CAG promoter operably linked to exons 1-24 of a polynucleotide encoding the human OTOF protein of SEQ ID NO: 1 and the 500 kb immediately 3′ of the exon 24/25 boundary (FIG. 1A). The 3′ vector contains AAV2 ITRs, the 500 kb immediately 5′ of the exon 24/25 boundary and exons 25-48 of a polynucleotide encoding the human OTOF protein of SEQ ID NO: 1, and a WPRE sequence (SEQ ID NO: 23) (FIG. 11B).

FIGS. 2A and 2B are maps of the 5′ and 3′ vectors in an overlapping dual vector system. The 5′ vector contains AAV2 ITRs, and a CAG promoter operably linked to exons 1-28 of a polynucleotide encoding the human OTOF protein of SEQ ID NO: 1 and the 500 kb immediately 3′ of the exon 28/29 boundary (FIG. 2A). The 3′ vector contains AAV2 ITRs, the 500 kb immediately 5′ of the exon 28/29 boundary and exons 29-48 of a polynucleotide encoding the human OTOF protein of SEQ ID NO: 1, the full-length human OTOF 3′ UTR, and a WPRE sequence (SEQ ID NO: 23) (FIG. 2B).

FIGS. 3A and 3B are maps of the 5′ and 3′ vectors in an overlapping dual vector system. The 5′ vector contains AAV2 ITRs, and a Myo15 promoter (SEQ ID NO: 36) operably linked to exons 1-21 of a polynucleotide encoding the human OTOF protein of SEQ ID NO: 1 and the 500 kb immediately 3′ of the exon 21/22 boundary (FIG. 3A). The 3′ vector contains AAV2 ITRs, the 500 kb immediately 5′ of the exon 21/22 boundary and exons 22-48 of a polynucleotide encoding the human OTOF protein of SEQ ID NO: 1, and a WPRE sequence (SEQ ID NO: 23) (FIG. 3B).

FIGS. 4A and 4B are maps of the 5′ and 3′ vectors in an overlapping dual vector system. The 5′ vector contains AAV2 ITRs, and a CAG promoter operably linked to exons 1-24 of a polynucleotide encoding the mouse OTOF protein of SEQ ID NO: 6 and the 500 kb immediately 3′ of the exon 24/25 boundary (FIG. 4A). The 3′ vector contains AAV2 ITRs, the 500 kb immediately 5′ of the exon 24/25 boundary and exons 25-48 of a polynucleotide encoding the mouse OTOF protein of SEQ ID NO: 6, and a WPRE sequence (SEQ ID NO: 23) (FIG. 4B).

FIGS. 5A and 5B are maps of the 5′ and 3′ vectors in an overlapping dual vector system. The 5′ vector contains AAV2 ITRs, a CMV enhancer, and a CMV promoter operably linked to exons 1-24 of a polynucleotide encoding the mouse OTOF protein of SEQ ID NO: 6 and the 500 kb immediately 3′ of the exon 24/25 boundary (FIG. 5A). The 3′ vector contains AAV2 ITRs, the 500 kb immediately 5′ of the exon 24/25 boundary and exons 25-48 of a polynucleotide encoding the mouse OTOF protein of SEQ ID NO: 6, and a WPRE sequence (SEQ ID NO: 23) (FIG. 5B).

FIGS. 6A and 6B are maps of the 5′ and 3′ vectors in an overlapping dual vector system. The 5′ vector contains AAV2 ITRs, and a CAG promoter operably linked to exons 1-28 of a polynucleotide encoding the mouse OTOF protein of SEQ ID NO: 6 and the 500 kb immediately 3′ of the exon 28/29 boundary (FIG. 6A). The 3′ vector contains AAV2 ITRs, the 500 kb immediately 5′ of the exon 28/29 boundary and exons 29-48 of a polynucleotide encoding the mouse OTOF protein of SEQ ID NO: 6, the full-length mouse OTOF 3′ UTR, and a WPRE sequence (SEQ ID NO: 23) (FIG. 6B).

FIGS. 7A and 7B are maps of the 5′ and 3′ vectors in an overlapping dual vector system. The 5′ vector contains AAV2 ITRs, and a Myo15 promoter (SEQ ID NO: 36) operably linked to exons 1-21 of a polynucleotide encoding the mouse OTOF protein of SEQ ID NO: 6 and the 500 kb immediately 3′ of the exon 21/22 boundary (FIG. 7A). The 3′ vector contains AAV2 ITRs, the 500 kb immediately 5′ of the exon 21/22 boundary and exons 22-48 of a polynucleotide encoding the mouse OTOF protein of SEQ ID NO: 6, and a WPRE sequence (SEQ ID NO: 23) (FIG. 7B).

FIGS. 8A and 8B are maps of the 5′ and 3′ vectors in a trans-splicing dual vector system. The 5′ vector contains AAV2 ITRs, a CAG promoter operably linked to the full-length human OTOF 5′ UTR and exons 1-26 of a polynucleotide encoding the human OTOF protein of SEQ ID NO: 1, and a splice donor sequence (SD) (FIG. 8A). The 3′ vector contains AAV2 ITRs, a splice acceptor sequence (SA), exons 27-48 of a polynucleotide encoding the human OTOF protein of SEQ ID NO: 1, the full-length human OTOF 3′ UTR, and a WPRE sequence (SEQ ID NO: 23) (FIG. 8B).

FIGS. 9A and 9B are maps of the 5′ and 3′ vectors in a trans-splicing dual vector system. The 5′ vector contains AAV2 ITRs, a Myo15 promoter (SEQ ID NO: 36) operably linked to exons 1-19 of a polynucleotide encoding the human OTOF protein of SEQ ID NO: 1, and a splice donor sequence (SD) (FIG. 9A). The 3′ vector contains AAV2 ITRs, a splice acceptor sequence (SA), exons 20-48 of a polynucleotide encoding the human OTOF protein of SEQ ID NO: 1, and a WPRE sequence (SEQ ID NO: 23) (FIG. 9B).

FIGS. 10A and 10B are maps of the 5′ and 3′ vectors in a trans-splicing dual vector system. The 5′ vector contains AAV2 ITRs, a Myo15 promoter (SEQ ID NO: 36) operably linked to exons 1-25 of a polynucleotide encoding the human OTOF protein of SEQ ID NO: 1, and a splice donor sequence (SD) (FIG. 10A). The 3′ vector contains AAV2 ITRs, a splice acceptor sequence (SA), exons 26-48 of a polynucleotide encoding the human OTOF protein of SEQ ID NO: 1, the full-length human OTOF 3′ UTR, and a WPRE sequence (SEQ ID NO: 23) (FIG. 101B).

FIGS. 11A and 11B are maps of the 5′ and 3′ vectors in a trans-splicing dual vector system. The 5′ vector contains AAV2 ITRs, a CAG promoter operably linked to the full-length mouse OTOF 5′ UTR and exons 1-28 of a polynucleotide encoding the mouse OTOF protein of SEQ ID NO: 6, and a splice donor sequence (SD) (FIG. 11A). The 3′ vector contains AAV2 ITRs, a splice acceptor sequence (SA), exons 29-48 of a polynucleotide encoding the mouse OTOF protein of SEQ ID NO: 6, the full-length mouse OTOF 3′ UTR, and a WPRE sequence (SEQ ID NO: 23) (FIG. 11B).

FIGS. 12A and 12B are maps of the 5′ and 3′ vectors in a trans-splicing dual vector system. The 5′ vector contains AAV2 ITRs, a Myo15 promoter (SEQ ID NO: 36) operably linked to exons 1-20 of a polynucleotide encoding the mouse OTOF protein of SEQ ID NO: 6, and a splice donor sequence (SD) (FIG. 12A). The 3′ vector contains AAV2 ITRs, a splice acceptor sequence (SA), exons 21-48 of a polynucleotide encoding the mouse OTOF protein of SEQ ID NO: 6, and a WPRE sequence (SEQ ID NO: 23) (FIG. 12B).

FIGS. 13A and 13B are maps of the 5′ and 3′ vectors in a trans-splicing dual vector system. The 5′ vector contains AAV2 ITRs, a Myo15 promoter (SEQ ID NO: 36) operably linked to exons 1-24 of a polynucleotide encoding the mouse OTOF protein of SEQ ID NO: 6, and a splice donor sequence (SD) (FIG. 13A). The 3′ vector contains AAV2 ITRs, a splice acceptor sequence (SA), exons 25-48 of a polynucleotide encoding the mouse OTOF protein of SEQ ID NO: 6, the full-length mouse OTOF 3′ UTR, and a WPRE sequence (SEQ ID NO: 23) (FIG. 13B).

FIGS. 14A and 14B are maps of the 5′ and 3′ vectors in a dual hybrid vector system. The 5′ vector contains AAV2 ITRs, a CAG promoter operably linked to the full-length human OTOF 5′ UTR and exons 1-26 of a polynucleotide encoding the human OTOF protein of SEQ ID NO: 1, a splice donor sequence (SD), a recombinogenic region (AK), and a degradation signal sequence (CL1) (FIG. 14A). The 3′ vector contains AAV2 ITRs, a recombinogenic region (AK), a degradation signal sequence (CL1), a splice acceptor sequence (SA), exons 27-48 of a polynucleotide encoding the human OTOF protein of SEQ ID NO: 1, the full-length human OTOF 3′ UTR, and a WPRE sequence (SEQ ID NO: 23) (FIG. 14B).

FIGS. 15A and 15B are maps of the 5′ and 3′ vectors in a dual hybrid vector system. The 5′ vector contains AAV2 ITRs, a Myo15 promoter (SEQ ID NO: 36) operably linked to exons 1-19 of a polynucleotide encoding the human OTOF protein of SEQ ID NO: 1, a splice donor sequence (SD), a recombinogenic region (AK), and a degradation signal sequence (CL1) (FIG. 15A). The 3′ vector contains AAV2 ITRs, a recombinogenic region (AK), a degradation signal sequence (CL1), a splice acceptor sequence (SA), exons 20-48 of a polynucleotide encoding the human OTOF protein of SEQ ID NO: 1, and a WPRE sequence (SEQ ID NO: 23) (FIG. 15B).

FIGS. 16A and 16B are maps of the 5′ and 3′ vectors in a dual hybrid vector system. The 5′ vector contains AAV2 ITRs, a Myo15 promoter (SEQ ID NO: 36) operably linked to exons 1-25 of a polynucleotide encoding the human OTOF protein of SEQ ID NO: 1, a splice donor sequence (SD), a recombinogenic region (AK), and a degradation signal sequence (CL1) (FIG. 16A). The 3′ vector contains AAV2 ITRs, a recombinogenic region (AK), a degradation signal sequence (CL1), a splice acceptor sequence (SA), exons 26-48 of a polynucleotide encoding the human OTOF protein of SEQ ID NO: 1, the full-length human OTOF 3′ UTR, and a WPRE sequence (SEQ ID NO: 23) (FIG. 16B).

FIGS. 17A and 17B are maps of the 5′ and 3′ vectors in a dual hybrid vector system. The 5′ vector contains AAV2 ITRs, a CAG promoter operably linked to the full-length mouse OTOF 5′ UTR and exons 1-28 of a polynucleotide encoding the mouse OTOF protein of SEQ ID NO: 6, a splice donor sequence (SD), a recombinogenic region (AK), and a degradation signal sequence (CL1) (FIG. 17A). The 3′ vector contains AAV2 ITRs, a recombinogenic region (AK), a degradation signal sequence (CL1), a splice acceptor sequence (SA), exons 29-48 of a polynucleotide encoding the mouse OTOF protein of SEQ ID NO: 6, the full-length mouse OTOF 3′ UTR, and a WPRE sequence (SEQ ID NO: 23) (FIG. 17B).

FIGS. 18A and 18B are maps of the 5′ and 3′ vectors in a dual hybrid vector system. The 5′ vector contains AAV2 ITRs, a Myo15 promoter (SEQ ID NO: 36) operably linked to exons 1-20 of a polynucleotide encoding the mouse OTOF protein of SEQ ID NO: 6, a splice donor sequence (SD), a recombinogenic region (AK), and a degradation signal sequence (CL1) (FIG. 18A). The 3′ vector contains AAV2 ITRs, a recombinogenic region (AK), a degradation signal sequence (CL1), a splice acceptor sequence (SA), exons 21-48 of a polynucleotide encoding the mouse OTOF protein of SEQ ID NO: 6, and a WPRE sequence (SEQ ID NO: 23) (FIG. 18B).

FIGS. 19A and 19B are maps of the 5′ and 3′ vectors in a dual hybrid vector system. The 5′ vector contains AAV2 ITRs, a Myo15 promoter (SEQ ID NO: 36) operably linked to exons 1-24 of a polynucleotide encoding the mouse OTOF protein of SEQ ID NO: 6, a splice donor sequence (SD), a recombinogenic region (AK), and a degradation signal sequence (CL1) (FIG. 19A). The 3′ vector contains AAV2 ITRs, a recombinogenic region (AK), a degradation signal sequence (CL1), a splice acceptor sequence (SA), exons 25-48 of a polynucleotide encoding the mouse OTOF protein of SEQ ID NO: 6, the full-length mouse OTOF 3′ UTR, and a WPRE sequence (SEQ ID NO: 23) (FIG. 19B).

FIGS. 20A and 20B are images demonstrating that polynucleotides encoded by dual hybrid vectors (FIG. 20A) and overlapping vectors (FIG. 20B) undergo recombination in cell culture (top). Beneath each image is a schematic depicting the type of recombination that occurs in each dual vector system (bottom). Inner ear-derived House Ear Institute-Organ of Corti 1 (HEI-OC1) cells were transfected with plasmids containing either the 5′ half of OTOF alone, the 3′ half of OTOF alone, or both the 5′ and 3′ halves together. For the dual hybrid vector experiment, the 5′ vector contained a Myo15 promoter (SEQ ID NO: 36) operably linked to exons 1-19 of a polynucleotide encoding the human OTOF protein of SEQ ID NO: 1, a splice donor sequence (SD), a recombinogenic region (AK), and a degradation signal sequence (CL1); and the 3′ vector contained a recombinogenic region (AK), a degradation signal sequence (CL1), a splice acceptor sequence (SA), exons 20-48 of a polynucleotide encoding the human OTOF protein of SEQ ID NO: 1, and a WPRE sequence (SEQ ID NO: 23). For the overlapping dual vector experiment, the 5′ vector contained a Myo15 promoter (SEQ ID NO: 36) operably linked to exons 1-21 of a polynucleotide encoding the human OTOF protein of SEQ ID NO: 1 and the 500 kb immediately 3′ of the exon 21/22 boundary; and the 3′ vector contained the 500 kb immediately 5′ of the exon 21/22 boundary and exons 22-48 of a polynucleotide encoding the human OTOF protein of SEQ ID NO: 1, and a WPRE sequence (SEQ ID NO: 23). Full-length otoferlin was transfected as a positive control and untransfected cells were used a negative control. Genomic DNA was extracted from cells using a standard column from a gDNA isolation kit. PCR primers were designed to anneal outside of the region of splice sites or overlap and PCR was performed using standard molecular biology techniques. Amplicons were visualized using gel electrophoresis. The white box in the gel image indicates the lane in which 5′ and 3′ halves were transfected together and where a roughly 1 kb amplicon is seen, indicating recombination.

FIGS. 21A-21C are images demonstrating that polynucleotides encoded by dual hybrid vectors (FIG. 21A), trans-splicing vectors (FIG. 21B), and overlapping vectors (FIG. 21C) undergo recombination in cell culture and produce OTOF protein (top). Beneath each image is a schematic depicting the type of recombination that occurs in each dual vector system (bottom). Inner ear derived HEI-OC1 cells were transfected with plasmids containing either the 5′ half of otoferlin alone, the 3′ half of otoferlin alone, or both 5′ and 3′ halves together. For the dual hybrid vector experiment, the 5′ vector contained a CAG promoter operably linked to the full-length human OTOF 5′ UTR and exons 1-26 of a polynucleotide encoding the human OTOF protein of SEQ ID NO: 1, a splice donor sequence (SD), a recombinogenic region (AK), and a degradation signal sequence (CL1); and the 3′ vector contained a recombinogenic region (AK), a degradation signal sequence (CL1), a splice acceptor sequence (SA), exons 27-48 of a polynucleotide encoding the human OTOF protein of SEQ ID NO: 1, the full-length human OTOF 3′ UTR, and a WPRE sequence (SEQ ID NO: 23). For the trans-splicing dual vector experiment, the 5′ vector contained a Myo15 promoter (SEQ ID NO: 36) operably linked to exons 1-24 of a polynucleotide encoding the mouse OTOF protein of SEQ ID NO: 6, and a splice donor sequence (SD); and the 3′ vector contained a splice acceptor sequence (SA), exons 25-48 of a polynucleotide encoding the mouse OTOF protein of SEQ ID NO: 6, the full-length mouse OTOF 3′ UTR, and a WPRE sequence (SEQ ID NO: 23). For the overlapping dual vector experiment, the 5′ vector contained a CMV promoter operably linked to exons 1-24 of a polynucleotide encoding the mouse OTOF protein of SEQ ID NO: 6 and the 500 kb immediately 3′ of the exon 24/25 boundary; and the 3′ vector contained the 500 kb immediately 5′ of the exon 24/25 boundary and exons 25-48 of a polynucleotide encoding the mouse OTOF protein of SEQ ID NO: 6, and a WPRE sequence (SEQ ID NO: 23). Cells were fixed in 4% PFA 48 hours post transfection and processed for immunohistochemistry. An antibody specific to OTOF protein labels some cells in the 5′ alone transfected cells, no cells in the 3′ alone transfected cells, and more cells than both previous conditions combined in the 5′+3′ transfected cells.

FIGS. 22A-22C are a series of graphs showing electrophysiological signatures of hearing function in mice treated with viral vectors expressing OTOF via dual hybrid vector systems. Homozygous OTOF-Q828X mice (a mouse model of human OTOF mutation p.Gln828Ter) were treated with an AAV1-Myo15 (SEQ ID NO: 38)-hOTOF (isoform 5, SEQ ID NO: 5) dual hybrid vector system by injection through the round window membrane and auditory brainstem response (ABR) thresholds were used to assess hearing function (FIG. 22A). Untreated animals (untreated Otof HOM) showed no detectable recovery in hearing function, whereas treated animals exhibited robust recovery, which was consistent from four weeks post-treatment (Otof HOM at 4 weeks after treatment) to eight weeks post-treatment (Otof HOM at 8-11 weeks after treatment). ABR thresholds from heterozygous mice (Otof HET) were also tested.

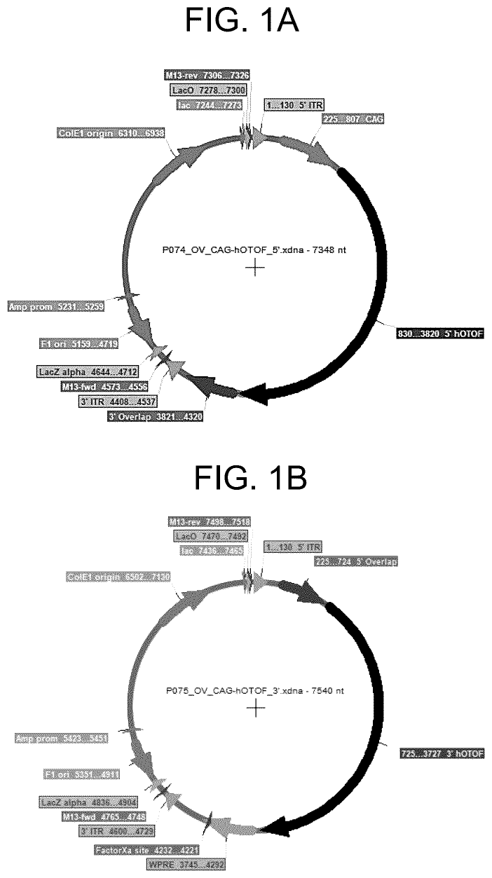

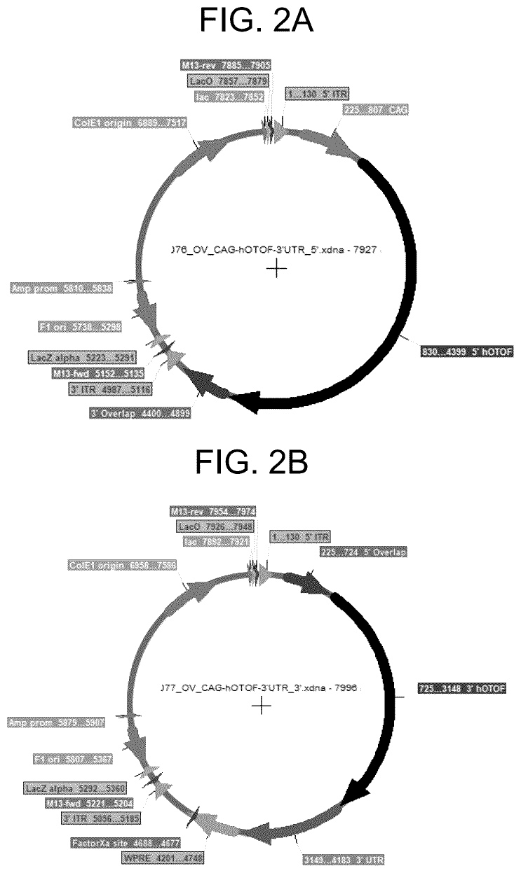

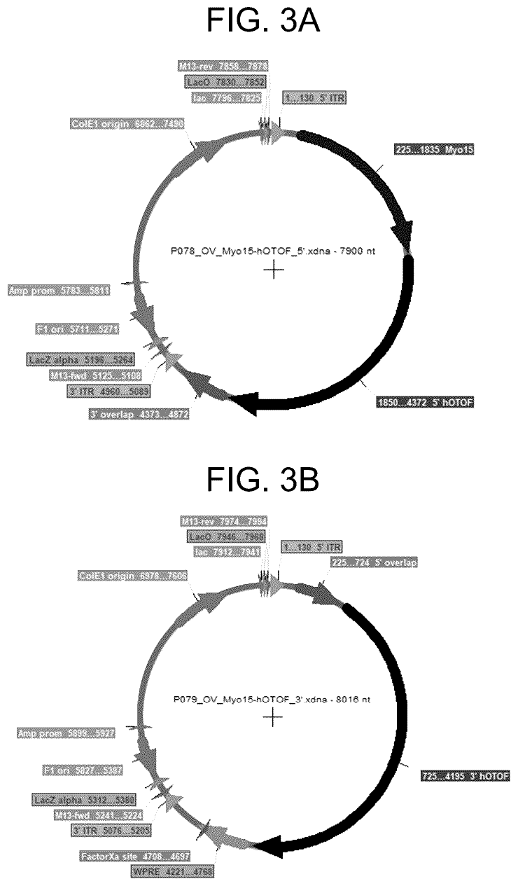

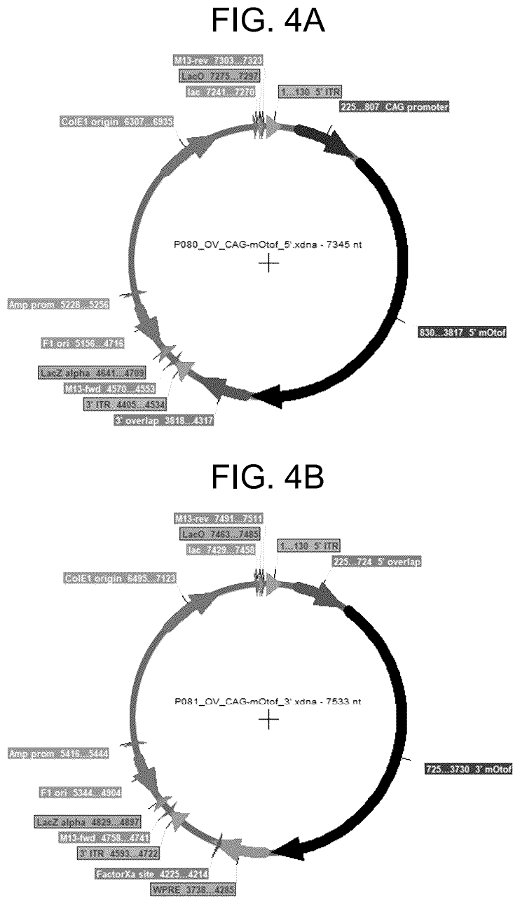

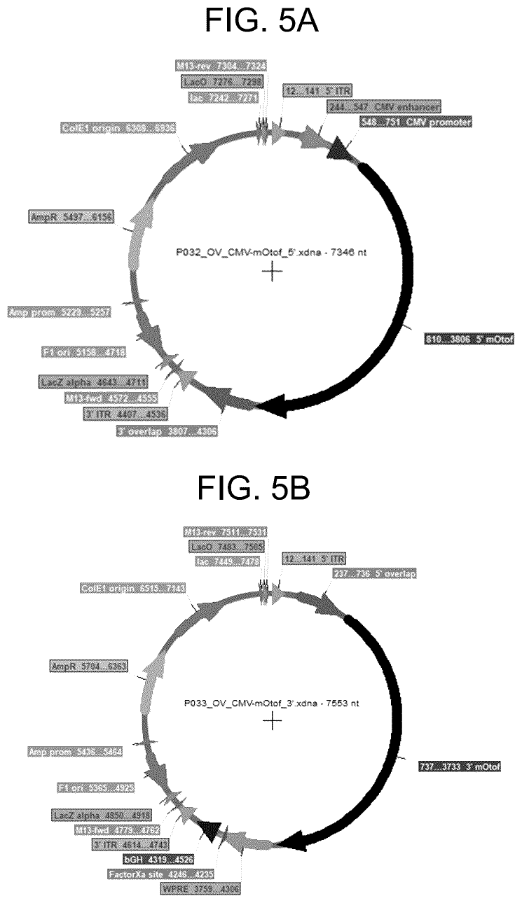

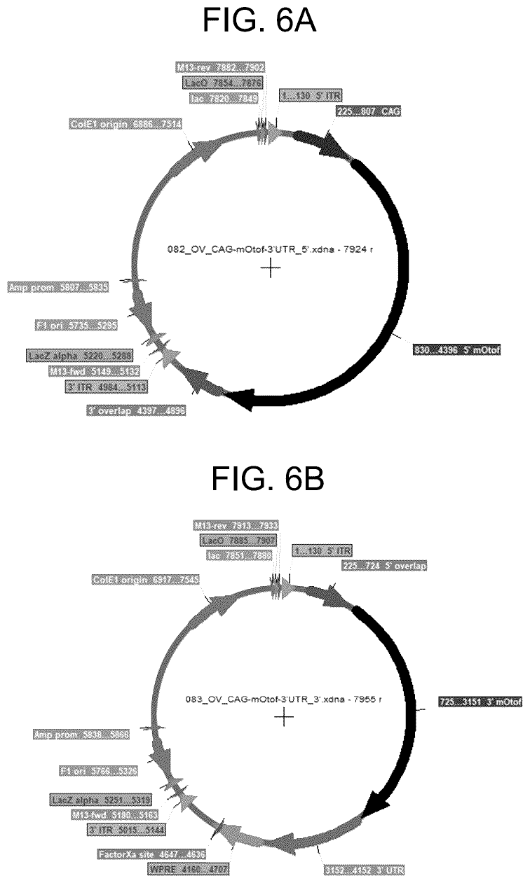

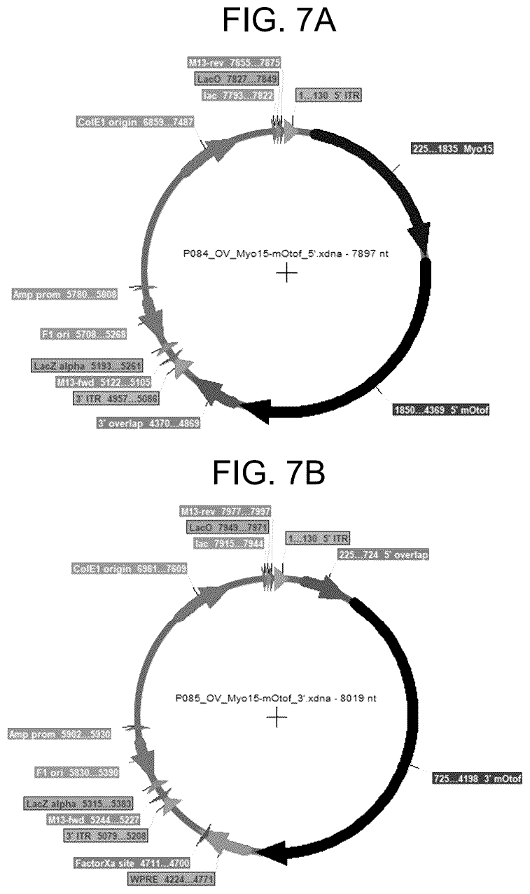

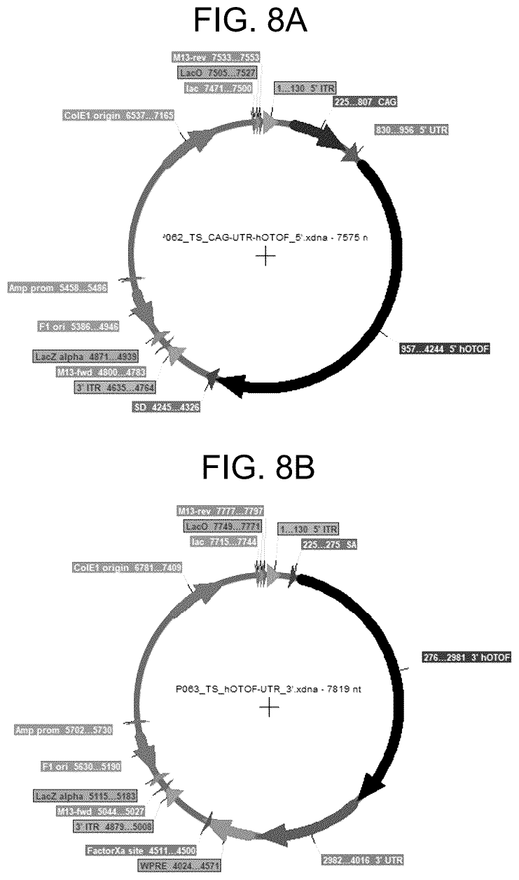

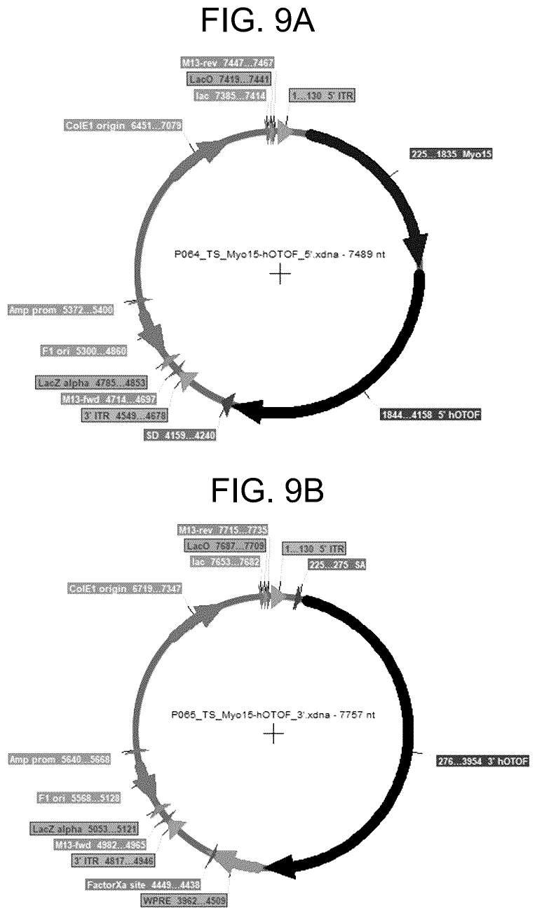

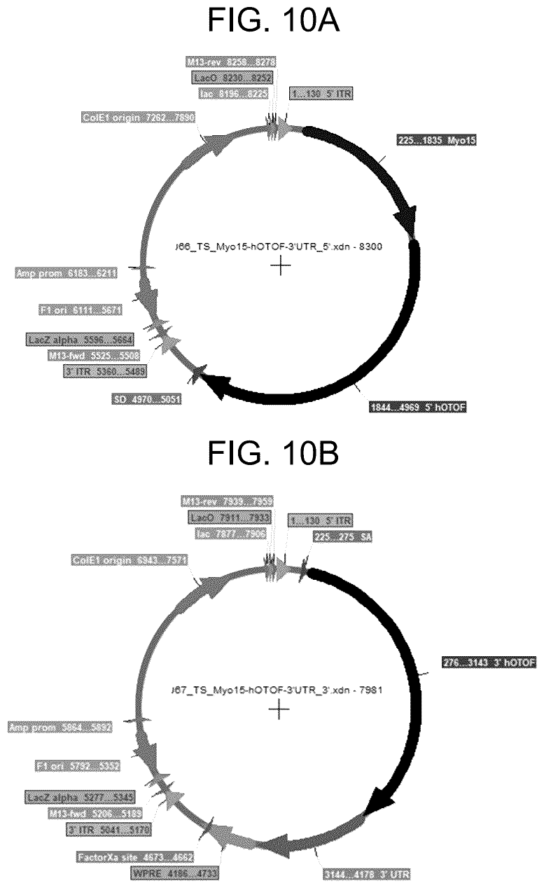

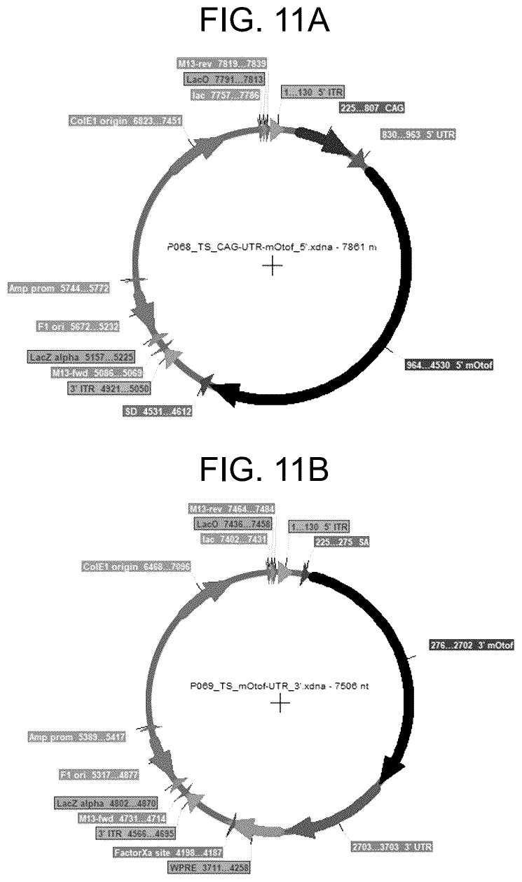

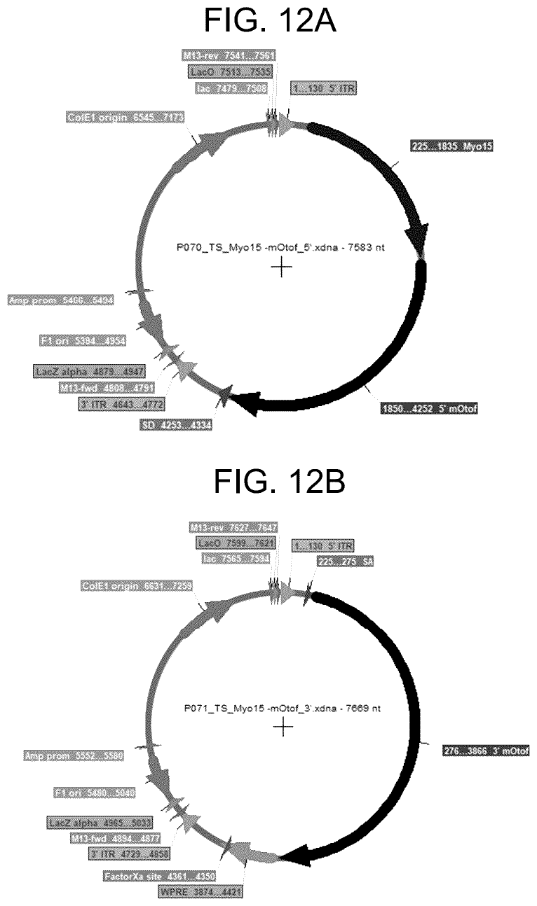

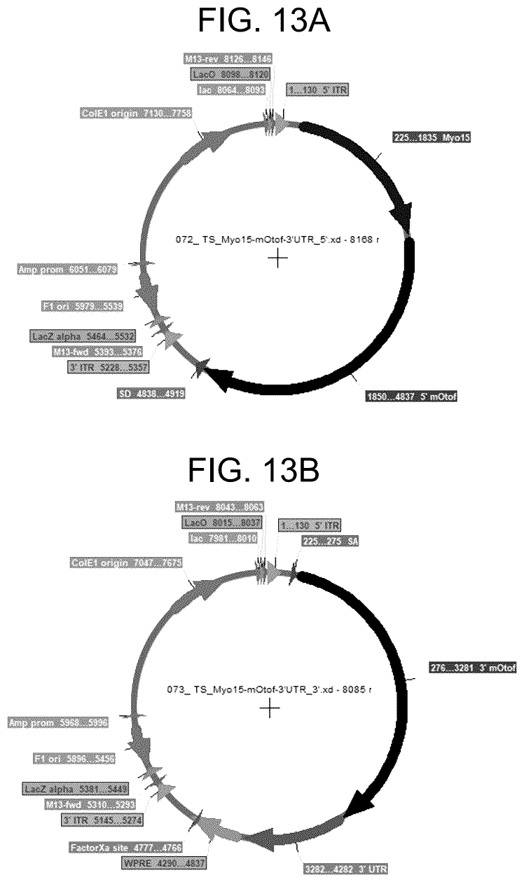

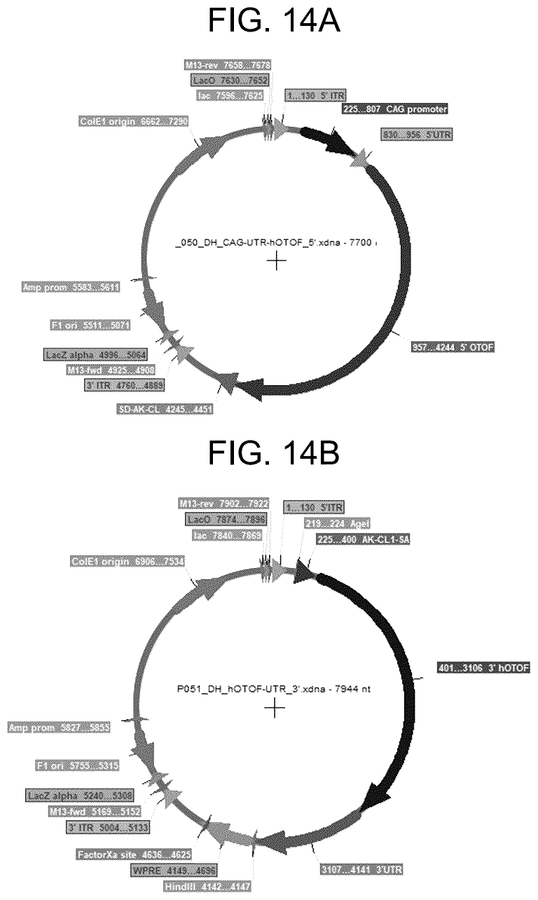

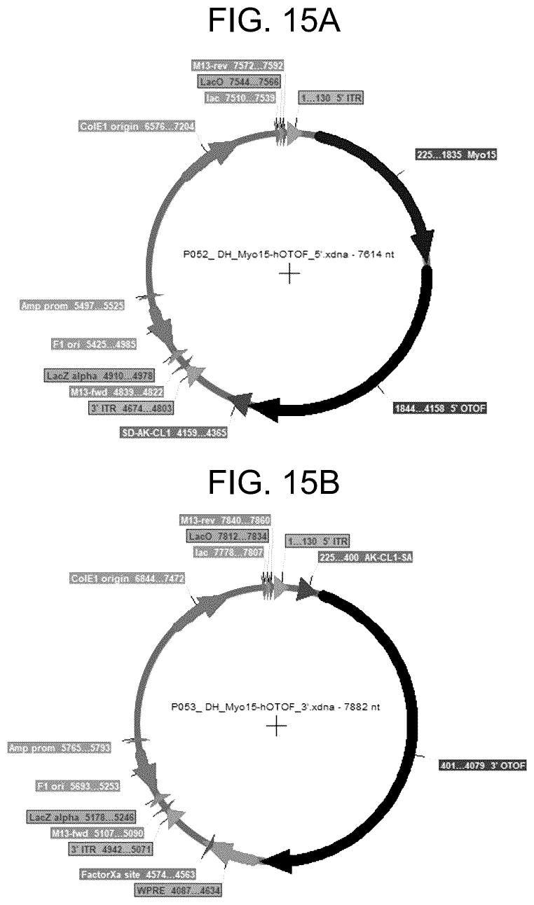

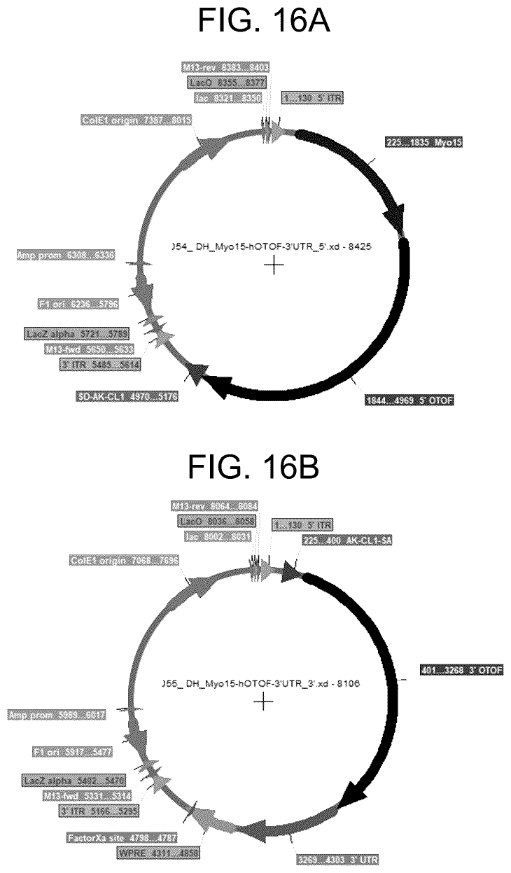

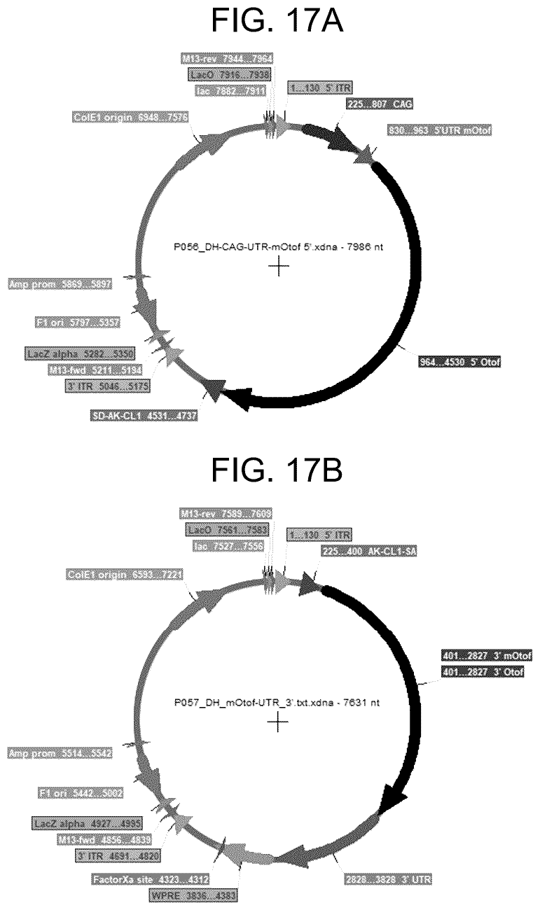

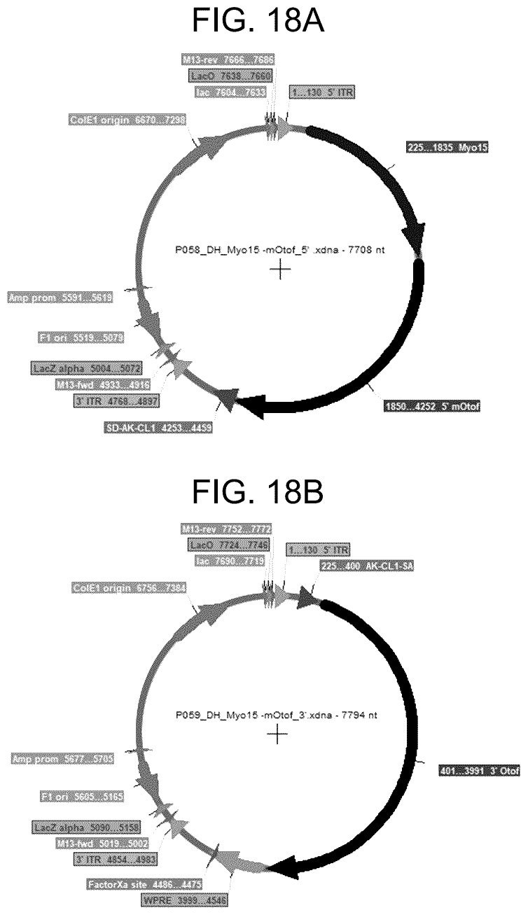

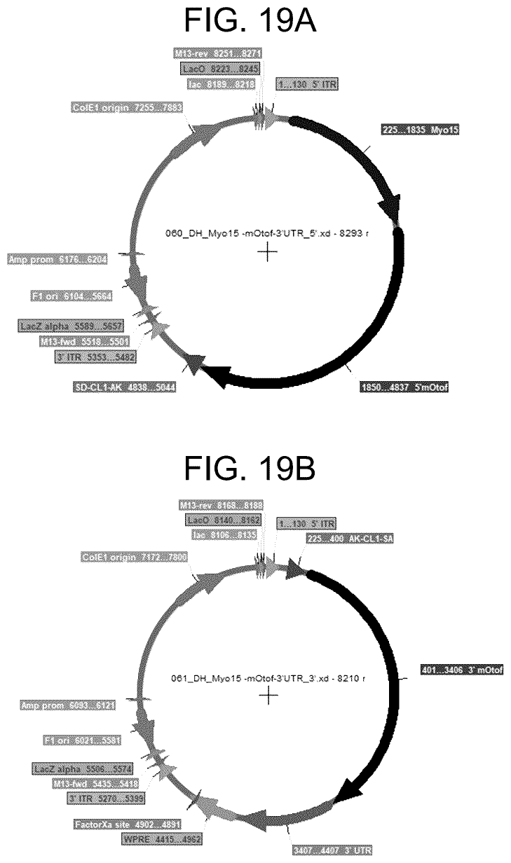

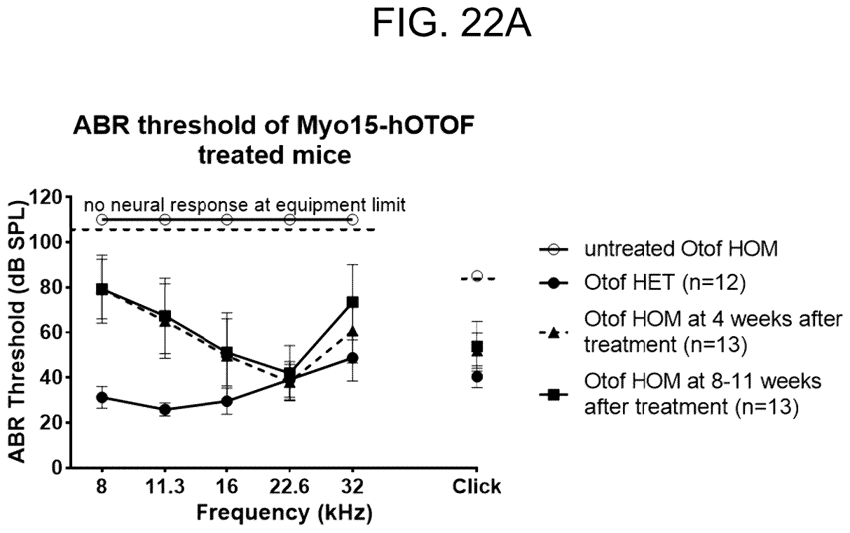

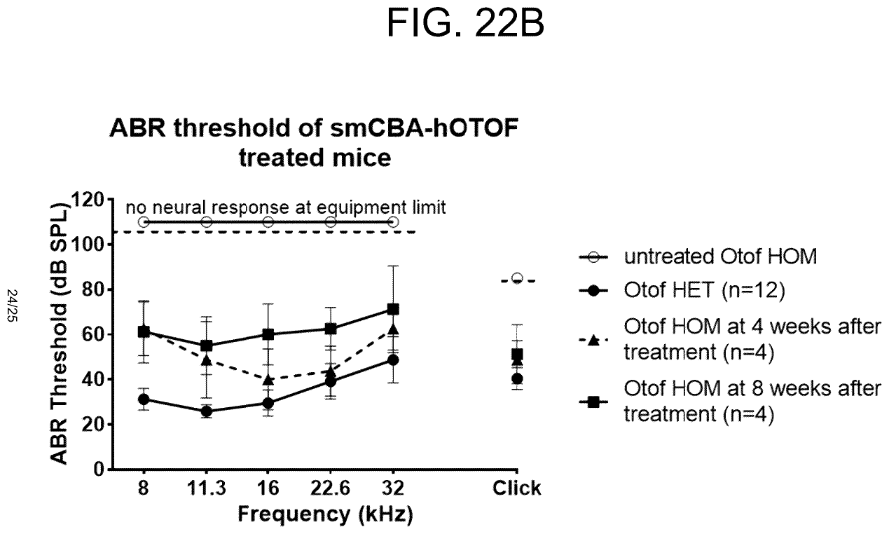

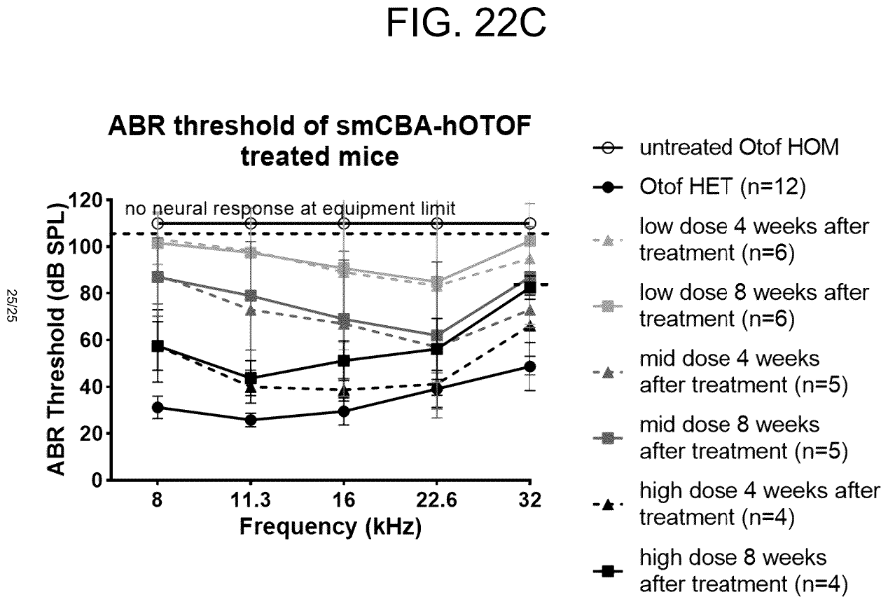

In another set of experiments, Homozygous OTOF-Q828X mice were treated with an AAV1-truncated chimeric CMV-chicken β-actin (smCBA)-hOTOF (isoform 5, SEQ ID NO: 5) dual hybrid vector system by injection through the round window membrane and ABR thresholds were used to assess hearing function as described above (FIG. 22B). Untreated animals showed no detectable recovery in hearing function, while treated animals exhibited a robust recovery at four weeks post-treatment (Otof HOM at 4 weeks after treatment). When the same mice were evaluated at eight weeks post-treatment (Otof HOM at 8 weeks after treatment), ABR thresholds increased, suggesting less durable recovery with the smCBA promoter. ABR thresholds from heterozygous mice were also tested.

In yet another set of experiments, homozygous OTOF-Q828X mice were treated with an AAV1-smCBA-hOTOF (isoform 5, SEQ ID NO: 5) dual hybrid vector system by injection through the round window membrane at low, medium (mid), and high doses and ABR thresholds were used to assess hearing function at four weeks and eight weeks post-treatment (FIG. 22C). A dose-dependent recovery in ABR thresholds was observed at both timepoints. When comparing the eight weeks versus the four weeks timepoints, recovery was steady for the low and mid doses, but decreased for the high dose animals. ABR thresholds for HET animals were also tested.

### DETAILED DESCRIPTION

Described herein are compositions and methods for the treatment of sensorineural hearing loss or auditory neuropathy in a subject (such as a mammalian subject, for instance, a human) by administering a first nucleic acid vector containing a promoter and a polynucleotide encoding an N-terminal portion of an otoferlin (OTOF) protein (e.g., a wild-type (WT) OTOF protein) and a second nucleic acid vector containing a polynucleotide encoding a C-terminal portion of an OTOF protein and a polyadenylation (poly(A)) sequence. When introduced into a mammalian cell, such as a cochlear hair cell, the polynucleotides encoded by the two nucleic acid vectors can combine to form a nucleic acid molecule that encodes the full-length OTOF protein. The compositions and methods described herein can, therefore, be used to induce or increase expression of WT OTOF in cochlear hair cells of a subject who has an OTOF deficiency (e.g., low OTOF expression or an OTOF mutation that impairs OTOF expression or function).

### Otoferlin

OTOF is a 230 kDa membrane protein that contains at least six C2 domains implicated in calcium, phospholipid, and protein binding. It is encoded by a gene that contains 48 exons, and the full-length protein is made up of 1,997 amino acids. OTOF is located at ribbon synapses in inner hair cells, where it is believed to function as a calcium sensor in synaptic vesicle fusion, triggering the fusion of neurotransmitter-containing vesicles with the plasma membrane. It has also been implicated in vesicle replenishment and clathrin-mediated endocytosis, and has been shown to interact with Myosin VI, Rab8b, SNARE proteins, calcium channel Cav1.3, Ergic2, and AP-2. The mechanism by which OTOF mediates exocytosis and the physiological significance of its interactions with its binding partners remain to be determined.

### Otoferlin-Associated Hearing Loss

OTOF was first identified by a study investigating the genetics of a non-syndromic form of deafness, autosomal recessive deafness-9 (DFNB9). Mutations in OTOF have since been found to cause sensorineural hearing loss in patients throughout the world, with many patients carrying OTOF mutations having auditory neuropathy, a disorder in which the inner ear detects sound, but is unable to properly transmit sound from the ear to the brain. These patients have an abnormal auditory brainstem response (ABR) and impaired speech discrimination with initially normal otoacoustic emissions. Patients carrying homozygous or compound heterozygous mutations often develop hearing loss in early childhood, and the severity of hearing impairment has been found to vary with the location and type of mutation in OTOF.

The compositions and methods described herein can be used to treat sensorineural hearing loss or auditory neuropathy by administering a first nucleic acid vector containing a polynucleotide encoding an N-terminal portion of an OTOF protein and a second nucleic acid vector containing a polynucleotide encoding a C-terminal portion of an OTOF protein. The full-length OTOF coding sequence is too large to include in the type of vector that is commonly used for gene therapy (e.g., an adeno-associated virus (AAV) vector, which is thought to have a packaging limit of 5 kb). The compositions and methods described herein overcome this problem by dividing the OTOF coding sequence between two different nucleic acid vectors that can recombine in a cell to reconstitute the full-length OTOF sequence. These compositions and methods can be used to treat subjects having one or more mutations in the OTOF gene, e.g., an OTOF mutation that reduces OTOF expression, reduces OTOF function, or is associated with hearing loss. When the first and second nucleic acid vectors are administered in a composition, the polynucleotides encoding the N-terminal and C-terminal portions of OTOF can combine within a cell (e.g., a human cell, e.g., a cochlear hair cell) to form a single nucleic acid molecule that contains the full-length OTOF coding sequence (e.g., through homologous recombination and/or splicing).

The nucleic acid vectors used in the compositions and methods described herein include nucleic acid sequences that encode wild-type OTOF, or a variant thereof, such as a nucleic acid sequences that, when combined, encode a protein having at least 85% sequence identity (e.g., 85%, 90%, 95%, 96%, 97%, 98%, 99%, or more, sequence identity) to the amino acid sequence of wild-type human or mouse OTOF. The polynucleotides used in the nucleic acid vectors described herein encode an N-terminal portion and a C-terminal portion of an OTOF amino acid sequence in Table 2 below (e.g., two portions that, when combined, encode a full-length OTOF amino acid sequence listed in Table 2, e.g., any one of SEQ ID NOs: 1-5).

According to the methods described herein, a subject can be administered a composition containing a first nucleic acid vector and a second nucleic acid vector that encode an N-terminal and C-terminal portion, respectively, of a nucleic acid sequence encoding the amino acid sequence of any one of SEQ ID NOs: 1-5, or a nucleic acid sequence encoding an amino acid sequence having at least 85% sequence identity (e.g., 85%, 90%, 95%, 96%, 97%, 98%, 99%, or more, sequence identity) to the amino acid sequence of any one of SEQ ID NOs: 1-5, or a nucleic acid sequence encoding an amino acid sequence that contains one or more conservative amino acid substitutions relative to any one of SEQ ID NOs: 1-5 (e.g., 1, 2, 3, 4, 5, 6, 7, 8, 9, or 10 or more conservative amino acid substitutions), provided that the OTOF analog encoded retains the therapeutic function of wild-type OTOF (e.g., the ability to regulate exocytosis at ribbon synapses).

TABLE 2
OTOF Sequences

| SEQ ID | | |
|---|---|---|
| NO. | Sequence Name | Sequence |
| 1 | OTOF-201 protein | MALLIHLKTVSELRGRGDRIAKVTFRGQSFYSRVLENCEDVADFDE |
| | (NP_919224.1), | TFRWPVASSIDRNEMLEIQVFNYSKVFSNKLIGTFRMVLQKVVEES |
| | human otoferlin | HVEVTDTLIDDNNAIIKTSLCVEVRYQATDGTVGSWDDGDFLGDES |
| | isoform a, 1997 aa | LQEEEKDSQETDGLLPGSRPSSRPPGEKSFRRAGRSVFSAMKLGK |
| | | NRSHKEEPQRPDEPAVLEMEDLDHLAIRLGDGLDPDSVSLASVTAL |
| | | TTNVSNKRSKPDIKMEPSAGRPMDYQVSITVIEARQLVGLNMDPVV |
| | | CVEVGDDKKYTSMKESTNCPYYNEYFVFDFHVSPDVMFDKIIKISVI |
| | | HSKNLLRSGTLVGSFKMDVGTVYSQPEHQFHHKWAILSDPDDISS |
| | | GLKGYVKCDVAVVGKGDNIKTPHKANETDEDDIEGNLLLPEGVPPE |
| | | RQWARFYVKIYRAEGLPRMNTSLMANVKKAFIGENKDLVDPYVQV |
| | | FFAGQKGKTSVQKSSYEPLWNEQVVFTDLFPPLCKRMKVQIRDSD |
| | | KVNDVAIGTHFIDLRKISNDGDKGFLPTLGPAWVNMYGSTRNYTLL |
| | | DEHQDLNEGLGEGVSFRARLLLGLAVEIVDTSNPELTSSTEVQVEQ |
| | | ATPISESCAGKMEEFFLFGAFLEASMIDRRNGDKPITFEVTIGNYGN |
| | | EVDGLSRPQRPRPRKEPGDEEEVDLIQNASDDEAGDAGDLASVSS |
| | | TPPMRPQVTDRNYFHLPYLERKPCIYIKSWWPDQRRRLYNANIMD |
| | | HIADKLEEGLNDIQEMIKTEKSYPERRLRGVLEELSCGCCRFLSLAD |
| | | KDQGHSSRTRLDRERLKSCMRELENMGQQARMLRAQVKRHTVRD |
| | | KLRLCQNFLQKLRFLADEPQHSIPDIFIWMMSNNKRVAYARVPSKD |
| | | LLFSIVEEETGKDCAKVKTLFLKLPGKRGFGSAGWTVQAKVELYLW |
| | | LGLSKQRKEFLCGLPCGFQEVKAAQGLGLHAFPPVSLVYTKKQAF |
| | | QLRAHMYQARSLFAADSSGLSDPFARVFFINQSQCTEVLNETLCPT |
| | | WDQMLVFDNLELYGEAHELRDDPPIIVIEIYDQDSMGKADFMGRTF |
| | | AKPLVKMADEAYCPPRFPPQLEYYQIYRGNATAGDLLAAFELLQIG |
| | | PAGKADLPPINGPVDVDRGPIMPVPMGIRPVLSKYRVEVLFWGLRD |
| | | LKRVNLAQVDRPRVDIECAGKGVQSSLIHNYKKNPNFNTLVKWFEV |
| | | DLPENELLHPPLNIRVVDCRAFGRYTLVGSHAVSSLRRFIYRPPDRS |
| | | APSWNTTVRLLRRCRVLCNGGSSSHSTGEVVVTMEPEVPIKKLET |
| | | MVKLDATSEAVVKVDVAEEEKEKKKKKKGTAEEPEEEEPDESMLD |
| | | WWSKYFASIDTMKEQLRQQEPSGIDLEEKEEVDNTEGLKGSMKGK |
| | | EKARAAKEEKKKKTQSSGSGQGSEAPEKKKPKIDELKVYPKELESE |
| | | FDNFEDWLHTFNLLRGKTGDDEDGSTEEERIVGRFKGSLCVYKVPL |
| | | PEDVSREAGYDSTYGMFQGIPSNDPINVLVRVYVVRATDLHPADIN |
| | | GKADPYIAIRLGKTDIRDKENYISKQLNPVFGKSFDIEASFPMESMLT |
| | | VAVYDWDLVGTDDLIGETKIDLENRFYSKHRATCGIAQTYSTHGYNI |
| | | WRDPMKPSQILTRLCKDGKVDGPHFGPPGRVKVANRVFTGPSEIE |
| | | DENGQRKPTDEHVALLALRHWEDIPRAGCRLVPEHVETRPLLNPD |
| | | KPGIEQGRLELWVDMFPMDMPAPGTPLDISPRKPKKYELRVIIWNT |
| | | DEVVLEDDDFFTGEKSSDIFVRGWLKGQQEDKQDTDVHYHSLTGE |
| | | GNFNWRYLFPFDYLAAEEKIVISKKESMFSWDETEYKIPARLTLQIW |
| | | DADHFSADDFLGAIELDLNRFPRGAKTAKQCTMEMATGEVDVPLV |
| | | SIFKQKRVKGWWPLLARNENDEFELTGKVEAELHLLTAEEAEKNPV |
| | | GLARNEPDPLEKPNRPDTSFIWFLNPLKSARYFLWHTYRWLLLKLL |
| | | LLLLLLLLLALFLYSVPGYLVKKILGA |
| 2 | OTOF-202 protein | MIKTEKSYPERRLRGVLEELSCGCCRFLSLADKDQGHSSRTRLDRE |
| | (NP_004793.2), | RLKSCMRELENMGQQARMLRAQVKRHTVRDKLRLCQNFLQKLRF |
| | human otoferlin | LADEPQHSIPDIFIWMMSNNKRVAYARVPSKDLLFSIVEEETGKDCA |
| | isoform b, 1230 aa | KVKTLFLKLPGKRGFGSAGWTVQAKVELYLWLGLSKQRKEFLCGL |
| | | PCGFQEVKAAQGLGLHAFPPVSLVYTKKQAFQLRAHMYQARSLFA |
| | | ADSSGLSDPFARVFFINQSQCTEVLNETLCPTWDQMLVFDNLELYG |
| | | EAHELRDDPPIIVIEIYDQDSMGKADFMGRTFAKPLVKMADEAYCPP |
| | | RFPPQLEYYQIYRGNATAGDLLAAFELLQIGPAGKADLPPINGPVDV |
| | | DRGPIMPVPMGIRPVLSKYRVEVLFWGLRDLKRVNLAQVDRPRVDI |
| | | ECAGKGVQSSLIHNYKKNPNFNTLVKWFEVDLPENELLHPPLNIRV |
| | | VDCRAFGRYTLVGSHAVSSLRRFIYRPPDRSAPSWNTTGEVVVTM |
| | | EPEVPIKKLETMVKLDATSEAVVKVDVAEEEKEKKKKKKGTAEEPE |
| | | EEEPDESMLDWWSKYFASIDTMKEQLRQQEPSGIDLEEKEEVDNT |
| | | EGLKGSMKGKEKARAAKEEKKKKTQSSGSGQGSEAPEKKKPKIDE |
| | | LKVYPKELESEFDNFEDWLHTFNLLRGKTGDDEDGSTEEERIVGRF |
| | | KGSLCVYKVPLPEDVSREAGYDSTYGMFQGIPSNDPINVLVRVYVV |
| | | RATDLHPADINGKADPYIAIRLGKTDIRDKENYISKQLNPVFGKSFDI |
| | | EASFPMESMLTVAVYDWDLVGTDDLIGETKIDLENRFYSKHRATCG |
| | | IAQTYSTHGYNIWRDPMKPSQILTRLCKDGKVDGPHFGPPGRVKVA |
| | | NRVFTGPSEIEDENGQRKPTDEHVALLALRHWEDIPRAGCRLVPEH |
| | | VETRPLLNPDKPGIEQGRLELWVDMFPMDMPAPGTPLDISPRKPKK |
| | | YELRVIIWNTDEVVLEDDDFFTGEKSSDIFVRGWLKGQQEDKQDTD |
| | | VHYHSLTGEGNFNWRYLFPFDYLAAEEKIVISKKESMFSWDETEYKI |
| | | PARLTLQIWDADHFSADDFLGAIELDLNRFPRGAKTAKQCTMEMAT |
| | | GEVDVPLVSIFKQKRVKGWWPLLARNENDEFELTGKVEAELHLLTA |
| | | EEAEKNPVGLARNEPDPLEKPNRPDTSFIWFLNPLKSARYFLWHTY |
| | | RWLLLKLLLLLLLLLLLALFLYSVPGYLVKKILGA |
| 3 | OTOF-203 protein | MIKTEKSYPERRLRGVLEELSCGCCRFLSLADKDQGHSSRTRLDRE |
| | (NP_919304.1), | RLKSCMRELENMGQQARMLRAQVKRHTVRDKLRLCQNFLQKLRF |
| | human otoferlin | LADEPQHSIPDIFIWMMSNNKRVAYARVPSKDLLFSIVEEETGKDCA |
| | isoform d, 1230 aa | KVKTLFLKLPGKRGFGSAGWTVQAKVELYLWLGLSKQRKEFLCGL |
| | | PCGFQEVKAAQGLGLHAFPPVSLVYTKKQAFQLRAHMYQARSLFA |
| | | ADSSGLSDPFARVFFINQSQCTEVLNETLCPTWDQMLVFDNLELYG |
| | | EAHELRDDPPIIVIEIYDQDSMGKADFMGRTFAKPLVKMADEAYCPP |
| | | RFPPQLEYYQIYRGNATAGDLLAAFELLQIGPAGKADLPPINGPVDV |
| | | DRGPIMPVPMGIRPVLSKYRVEVLFWGLRDLKRVNLAQVDRPRVDI |
| | | ECAGKGVQSSLIHNYKKNPNFNTLVKWFEVDLPENELLHPPLNIRV |
| | | VDCRAFGRYTLVGSHAVSSLRRFIYRPPDRSAPSWNTTGEVVVTM |
| | | EPEVPIKKLETMVKLDATSEAVVKVDVAEEEKEKKKKKKGTAEEPE |
| | | EEEPDESMLDWWSKYFASIDTMKEQLRQQEPSGIDLEEKEEVDNT |
| | | EGLKGSMKGKEKARAAKEEKKKKTQSSGSGQGSEAPEKKKPKIDE |
| | | LKVYPKELESEFDNFEDWLHTFNLLRGKTGDDEDGSTEEERIVGRF |
| | | KGSLCVYKVPLPEDVSREAGYDSTYGMFQGIPSNDPINVLVRVYVV |
| | | RATDLHPADINGKADPYIAIRLGKTDIRDKENYISKQLNPVFGKSFDI |
| | | EASFPMESMLTVAVYDWDLVGTDDLIGETKIDLENRFYSKHRATCG |
| | | IAQTYSTHGYNIWRDPMKPSQILTRLCKDGKVDGPHFGPPGRVKVA |
| | | NRVFTGPSEIEDENGQRKPTDEHVALLALRHWEDIPRAGCRLVPEH |
| | | VETRPLLNPDKPGIEQGRLELWVDMFPMDMPAPGTPLDISPRKPKK |
| | | YELRVIIWNTDEVVLEDDDFFTGEKSSDIFVRGWLKGQQEDKQDTD |
| | | VHYHSLTGEGNFNWRYLFPFDYLAAEEKIVISKKESMFSWDETEYKI |
| | | PARLTLQIWDADHFSADDFLGAIELDLNRFPRGAKTAKQCTMEMAT |
| | | GEVDVPLVSIFKQKRVKGWWPLLARNENDEFELTGKVEAELHLLTA |
| | | EEAEKNPVGLARNEPDPLEKPNRPDTAFVWFLNPLKSIKYLICTRYK |
| | | WLIIKIVLALLGLLMLGLFLYSLPGYMVKKLLGA |
| 4 | OTOF-208 protein | MMTDTQDGPSESSQIMRSLTPLINREEAFGEAGEAGLWPSITHTPD |
| | (NP_919303.1), | SQEEGLNDIQEMIKTEKSYPERRLRGVLEELSCGCCRFLSLADKDQ |
| | human otoferlin | GHSSRTRLDRERLKSCMRELENMGQQARMLRAQVKRHTVRDKLR |
| | isoform c, 1307 aa | LCQNFLQKLRFLADEPQHSIPDIFIWMMSNNKRVAYARVPSKDLLFS |
| | | IVEEETGKDCAKVKTLFLKLPGKRGFGSAGWTVQAKVELYLWLGLS |
| | | KQRKEFLCGLPCGFQEVKAAQGLGLHAFPPVSLVYTKKQAFQLRA |
| | | HMYQARSLFAADSSGLSDPFARVFFINQSQCTEVLNETLCPTWDQ |
| | | MLVFDNLELYGEAHELRDDPPIIVIEIYDQDSMGKADFMGRTFAKPL |
| | | VKMADEAYCPPRFPPQLEYYQIYRGNATAGDLLAAFELLQIGPAGK |
| | | ADLPPINGPVDVDRGPIMPVPMGIRPVLSKYRVEVLFWGLRDLKRV |
| | | NLAQVDRPRVDIECAGKGVQSSLIHNYKKNPNFNTLVKWFEVDLPE |
| | | NELLHPPLNIRVVDCRAFGRYTLVGSHAVSSLRRFIYRPPDRSAPS |
| | | WNTTVRLLRRCRVLCNGGSSSHSTGEVVVTMEPEVPIKKLETMVK |
| | | LDATSEAVVKVDVAEEEKEKKKKKKGTAEEPEEEEPDESMLDWWS |
| | | KYFASIDTMKEQLRQQEPSGIDLEEKEEVDNTEGLKGSMKGKEKAR |
| | | AAKEEKKKKTQSSGSGQGSEAPEKKKPKIDELKVYPKELESEFDNF |
| | | EDWLHTFNLLRGKTGDDEDGSTEEERIVGRFKGSLCVYKVPLPED |
| | | VSREAGYDSTYGMFQGIPSNDPINVLVRVYVVRATDLHPADINGKA |
| | | DPYIAIRLGKTDIRDKENYISKQLNPVFGKSFDIEASFPMESMLTVAV |
| | | YDWDLVGTDDLIGETKIDLENRFYSKHRATCGIAQTYSTHGYNIWR |
| | | DPMKPSQILTRLCKDGKVDGPHFGPPGRVKVANRVFTGPSEIEDE |
| | | NGQRKPTDEHVALLALRHWEDIPRAGCRLVPEHVETRPLLNPDKP |
| | | GIEQGRLELWVDMFPMDMPAPGTPLDISPRKPKKYELRVIIWNTDE |
| | | VVLEDDDFFTGEKSSDIFVRGWLKGQQEDKQDTDVHYHSLTGEGN |
| | | FNWRYLFPFDYLAAEEKIVISKKESMFSWDETEYKIPARLTLQIWDA |
| | | DHFSADDFLGAIELDLNRFPRGAKTAKQCTMEMATGEVDVPLVSIF |
| | | KQKRVKGWWPLLARNENDEFELTGKVEAELHLLTAEEAEKNPVGL |
| | | ARNEPDPLEKPNRPDTSFIWFLNPLKSARYFLWHTYRWLLLKLLLLL |
| | | LLLLLLALFLYSVPGYLVKKILGA |
| 5 | OTOF-205 protein | MALLIHLKTVSELRGRGDRIAKVTFRGQSFYSRVLENCEDVADFDE |
| | (NP_001274418.1), | TFRWPVASSIDRNEMLEIQVFNYSKVFSNKLIGTFRMVLQKVVEES |
| | human otoferlin | HVEVTDTLIDDNNAIIKTSLCVEVRYQATDGTVGSWDDGDFLGDES |
| | isoform e, 1997 aa | LQEEEKDSQETDGLLPGSRPSSRPPGEKSFRRAGRSVFSAMKLGK |
| | | NRSHKEEPQRPDEPAVLEMEDLDHLAIRLGDGLDPDSVSLASVTAL |
| | | TTNVSNKRSKPDIKMEPSAGRPMDYQVSITVIEARQLVGLNMDPVV |
| | | CVEVGDDKKYTSMKESTNCPYYNEYFVFDFHVSPDVMFDKIIKISVI |
| | | HSKNLLRSGTLVGSFKMDVGTVYSQPEHQFHHKWAILSDPDDISS |
| | | GLKGYVKCDVAVVGKGDNIKTPHKANETDEDDIEGNLLLPEGVPPE |
| | | RQWARFYVKIYRAEGLPRMNTSLMANVKKAFIGENKDLVDPYVQV |
| | | FFAGQKGKTSVQKSSYEPLWNEQVVFTDLFPPLCKRMKVQIRDSD |
| | | KVNDVAIGTHFIDLRKISNDGDKGFLPTLGPAWVNMYGSTRNYTLL |
| | | DEHQDLNEGLGEGVSFRARLLLGLAVEIVDTSNPELTSSTEVQVEQ |
| | | ATPISESCAGKMEEFFLFGAFLEASMIDRRNGDKPITFEVTIGNYGN |
| | | EVDGLSRPQRPRPRKEPGDEEEVDLIQNASDDEAGDAGDLASVSS |
| | | TPPMRPQVTDRNYFHLPYLERKPCIYIKSWWPDQRRRLYNANIMD |
| | | HIADKLEEGLNDIQEMIKTEKSYPERRLRGVLEELSCGCCRFLSLAD |
| | | KDQGHSSRTRLDRERLKSCMRELENMGQQARMLRAQVKRHTVRD |
| | | KLRLCQNFLQKLRFLADEPQHSIPDIFIWMMSNNKRVAYARVPSKD |
| | | LLFSIVEEETGKDCAKVKTLFLKLPGKRGFGSAGWTVQAKVELYLW |
| | | LGLSKQRKEFLCGLPCGFQEVKAAQGLGLHAFPPVSLVYTKKQAF |
| | | QLRAHMYQARSLFAADSSGLSDPFARVFFINQSQCTEVLNETLCPT |
| | | WDQMLVFDNLELYGEAHELRDDPPIIVIEIYDQDSMGKADFMGRTF |
| | | AKPLVKMADEAYCPPRFPPQLEYYQIYRGNATAGDLLAAFELLQIG |
| | | PAGKADLPPINGPVDVDRGPIMPVPMGIRPVLSKYRVEVLFWGLRD |
| | | LKRVNLAQVDRPRVDIECAGKGVQSSLIHNYKKNPNFNTLVKWFEV |
| | | DLPENELLHPPLNIRVVDCRAFGRYTLVGSHAVSSLRRFIYRPPDRS |
| | | APSWNTTVRLLRRCRVLCNGGSSSHSTGEVVVTMEPEVPIKKLET |
| | | MVKLDATSEAVVKVDVAEEEKEKKKKKKGTAEEPEEEEPDESMLD |
| | | WWSKYFASIDTMKEQLRQQEPSGIDLEEKEEVDNTEGLKGSMKGK |
| | | EKARAAKEEKKKKTQSSGSGQGSEAPEKKKPKIDELKVYPKELESE |
| | | FDNFEDWLHTFNLLRGKTGDDEDGSTEEERIVGRFKGSLCVYKVPL |
| | | PEDVSREAGYDSTYGMFQGIPSNDPINVLVRVYVVRATDLHPADIN |
| | | GKADPYIAIRLGKTDIRDKENYISKQLNPVFGKSFDIEASFPMESMLT |
| | | VAVYDWDLVGTDDLIGETKIDLENRFYSKHRATCGIAQTYSTHGYNI |
| | | WRDPMKPSQILTRLCKDGKVDGPHFGPPGRVKVANRVFTGPSEIE |
| | | DENGQRKPTDEHVALLALRHWEDIPRAGCRLVPEHVETRPLLNPD |
| | | KPGIEQGRLELWVDMFPMDMPAPGTPLDISPRKPKKYELRVIIWNT |
| | | DEVVLEDDDFFTGEKSSDIFVRGWLKGQQEDKQDTDVHYHSLTGE |
| | | GNFNWRYLFPFDYLAAEEKIVISKKESMFSWDETEYKIPARLTLQIW |
| | | DADHFSADDFLGAIELDLNRFPRGAKTAKQCTMEMATGEVDVPLV |
| | | SIFKQKRVKGWWPLLARNENDEFELTGKVEAELHLLTAEEAEKNPV |
| | | GLARNEPDPLEKPNRPDTAFVWFLNPLKSIKYLICTRYKWLIIKIVLAL |
| | | LGLLMLGLFLYSLPGYMVKKLLGA |
| 6 | mOTOF-201_1 | MALIVHLKTVSELRGKGDRIAKVTFRGQSFYSRVLENCEGVADFDE |
| | protein | TFRWPVASSIDRNEVLEIQIFNYSKVFSNKLIGTFCMVLQKVVEENR |
| | (NP_114081.2), | VEVTDTLMDDSNAIIKTSLSMEVRYQATDGTVGPWDDGDFLGDESL |
| | mouse otoferlin | QEEKDSQETDGLLPGSRPSTRISGEKSFRRAGRSVFSAMKLGKTR |
| | isoform 2, 1997 aa | SHKEEPQRQDEPAVLEMEDLDHLAIQLGDGLDPDSVSLASVTALTS |
| | | NVSNKRSKPDIKMEPSAGRPMDYQVSITVIEARQLVGLNMDPVVCV |
| | | EVGDDKKYTSMKESTNCPYYNEYFVFDFHVSPDVMFDKIIKISVIHS |
| | | KNLLRSGTLVGSFKMDVGTVYSQPEHQFHHKWAILSDPDDISAGLK |
| | | GYVKCDVAVVGKGDNIKTPHKANETDEDDIEGNLLLPEGVPPERQ |
| | | WARFYVKIYRAEGLPRMNTSLMANVKKAFIGENKDLVDPYVQVFFA |
| | | GQKGKTSVQKSSYEPLWNEQVVFTDLFPPLCKRMKVQIRDSDKVN |
| | | DVAIGTHFIDLRKISNDGDKGFLPTLGPAWVNMYGSTRNYTLLDEH |
| | | QDLNEGLGEGVSFRARLMLGLAVEILDTSNPELTSSTEVQVEQATP |
| | | VSESCTGRMEEFFLFGAFLEASMIDRKNGDKPITFEVTIGNYGNEV |
| | | DGMSRPLRPRPRKEPGDEEEVDLIQNSSDDEGDEAGDLASVSSTP |
| | | PMRPQITDRNYFHLPYLERKPCIYIKSWWPDQRRRLYNANIMDHIA |
| | | DKLEEGLNDVQEMIKTEKSYPERRLRGVLEELSCGCHRFLSLSDKD |
| | | QGRSSRTRLDRERLKSCMRELESMGQQAKSLRAQVKRHTVRDKL |
| | | RSCQNFLQKLRFLADEPQHSIPDVFIWMMSNNKRIAYARVPSKDLL |
| | | FSIVEEELGKDCAKVKTLFLKLPGKRGFGSAGWTVQAKLELYLWLG |
| | | LSKQRKDFLCGLPCGFEEVKAAQGLGLHSFPPISLVYTKKQAFQLR |
| | | AHMYQARSLFAADSSGLSDPFARVFFINQSQCTEVLNETLCPTWD |
| | | QMLVFDNLELYGEAHELRDDPPIIVIEIYDQDSMGKADFMGRTFAKP |
| | | LVKMADEAYCPPRFPPQLEYYQIYRGSATAGDLLAAFELLQIGPSG |
| | | KADLPPINGPVDMDRGPIMPVPVGIRPVLSKYRVEVLFWGLRDLKR |
| | | VNLAQVDRPRVDIECAGKGVQSSLIHNYKKNPNFNTLVKWFEVDLP |
| | | ENELLHPPLNIRVVDCRAFGRYTLVGSHAVSSLRRFIYRPPDRSAP |
| | | NWNTTVRLLRGCHRLRNGGPSSRPTGEVVVSMEPEEPVKKLETM |
| | | VKLDATSDAVVKVDVAEDEKERKKKKKKGPSEEPEEEEPDESMLD |
| | | WWSKYFASIDTMKEQLRQHETSGTDLEEKEEMESAEGLKGPMKS |
| | | KEKSRAAKEEKKKKNQSPGPGQGSEAPEKKKAKIDELKVYPKELES |
| | | EFDSFEDWLHTFNLLRGKTGDDEDGSTEEERIVGRFKGSLCVYKV |
| | | PLPEDVSREAGYDPTYGMFQGIPSNDPINVLVRIYVVRATDLHPADI |
| | | NGKADPYIAIKLGKTDIRDKENYISKQLNPVFGKSFDIEASFPMESML |
| | | TVAVYDWDLVGTDDLIGETKIDLENRFYSKHRATCGIAQTYSIHGYN |
| | | IWRDPMKPSQILTRLCKEGKVDGPHFGPHGRVRVANRVFTGPSEIE |
| | | DENGQRKPTDEHVALSALRHWEDIPRVGCRLVPEHVETRPLLNPD |
| | | KPGIEQGRLELWVDMFPMDMPAPGTPLDISPRKPKKYELRVIVWNT |
| | | DEVVLEDDDFFTGEKSSDIFVRGWLKGQQEDKQDTDVHYHSLTGE |
| | | GNFNWRYLFPFDYLAAEEKIVMSKKESMFSWDETEYKIPARLTLQI |
| | | WDADHFSADDFLGAIELDLNRFPRGAKTAKQCTMEMATGEVDVPL |
| | | VSIFKQKRVKGWWPLLARNENDEFELTGKVEAELHLLTAEEAEKNP |
| | | VGLARNEPDPLEKPNRPDTSFIWFLNPLKSARYFLWHTYRWLLLKF |
| | | LLLFLLLLLFALFLYSLPGYLAKKILGA |
| 7 | mOTOF-201_2 | MALIVHLKTVSELRGKGDRIAKVTFRGQSFYSRVLENCEGVADFDE |
| | protein | TFRWPVASSIDRNEVLEIQIFNYSKVFSNKLIGTFCMVLQKVVEENR |
| | (NP_001273350.1), | VEVTDTLMDDSNAIIKTSLSMEVRYQATDGTVGPWDDGDFLGDESL |
| | mouse otoferlin | QEEKDSQETDGLLPGSRPSTRISGEKSFRRAGRSVFSAMKLGKTR |
| | isoform 3, 1977 aa | SHKEEPQRQDEPAVLEMEDLDHLAIQLGDGLDPDSVSLASVTALTS |
| | | NVSNKRSKPDIKMEPSAGRPMDYQVSITVIEARQLVGLNMDPVVCV |
| | | EVGDDKKYTSMKESTNCPYYNEYFVFDFHVSPDVMFDKIIKISVIHS |
| | | KNLLRSGTLVGSFKMDVGTVYSQPEHQFHHKWAILSDPDDISAGLK |
| | | GYVKCDVAVVGKGDNIKTPHKANETDEDDIEGNLLLPEGVPPERQ |
| | | WARFYVKIYRAEGLPRMNTSLMANVKKAFIGENKDLVDPYVQVFFA |
| | | GQKGKTSVQKSSYEPLWNEQVVFTDLFPPLCKRMKVQIRDSDKVN |
| | | DVAIGTHFIDLRKISNDGDKGFLPTLGPAWVNMYGSTRNYTLLDEH |
| | | QDLNEGLGEGVSFRARLMLGLAVEILDTSNPELTSSTEVQVEQATP |
| | | VSESCTGRMEEFFLFGAFLEASMIDRKNGDKPITFEVTIGNYGNEV |
| | | DGMSRPLRPRPRKEPGDEEEVDLIQNSSDDEGDEAGDLASVSSTP |
| | | PMRPQITDRNYFHLPYLERKPCIYIKSWWPDQRRRLYNANIMDHIA |
| | | DKLEEGLNDVQEMIKTEKSYPERRLRGVLEELSCGCHRFLSLSDKD |
| | | QGRSSRTRLDRERLKSCMRELESMGQQAKSLRAQVKRHTVRDKL |
| | | RSCQNFLQKLRFLADEPQHSIPDVFIWMMSNNKRIAYARVPSKDLL |
| | | FSIVEEELGKDCAKVKTLFLKLPGKRGFGSAGWTVQAKLELYLWLG |
| | | LSKQRKDFLCGLPCGFEEVKAAQGLGLHSFPPISLVYTKKQAFQLR |
| | | AHMYQARSLFAADSSGLSDPFARVFFINQSQCTEVLNETLCPTWD |
| | | QMLVFDNLELYGEAHELRDDPPIIVIEIYDQDSMGKADFMGRTFAKP |
| | | LVKMADEAYCPPRFPPQLEYYQIYRGSATAGDLLAAFELLQIGPSG |
| | | KADLPPINGPVDMDRGPIMPVPVGIRPVLSKYRVEVLFWGLRDLKR |
| | | VNLAQVDRPRVDIECAGKGVQSSLIHNYKKNPNFNTLVKWFEVDLP |
| | | ENELLHPPLNIRVVDCRAFGRYTLVGSHAVSSLRRFIYRPPDRSAP |
| | | NWNTTGEVVVSMEPEEPVKKLETMVKLDATSDAVVKVDVAEDEKE |
| | | RKKKKKKGPSEEPEEEEPDESMLDWWSKYFASIDTMKEQLRQHET |
| | | SGTDLEEKEEMESAEGLKGPMKSKEKSRAAKEEKKKKNQSPGPG |
| | | QGSEAPEKKKAKIDELKVYPKELESEFDSFEDWLHTFNLLRGKTGD |
| | | DEDGSTEEERIVGRFKGSLCVYKVPLPEDVSREAGYDPTYGMFQGI |
| | | PSNDPINVLVRIYVVRATDLHPADINGKADPYIAIKLGKTDIRDKENYI |
| | | SKQLNPVFGKSFDIEASFPMESMLTVAVYDWDLVGTDDLIGETKIDL |
| | | ENRFYSKHRATCGIAQTYSIHGYNIWRDPMKPSQILTRLCKEGKVD |
| | | GPHFGPHGRVRVANRVFTGPSEIEDENGQRKPTDEHVALSALRHW |
| | | EDIPRVGCRLVPEHVETRPLLNPDKPGIEQGRLELWVDMFPMDMP |
| | | APGTPLDISPRKPKKYELRVIVWNTDEVVLEDDDFFTGEKSSDIFVR |
| | | GWLKGQQEDKQDTDVHYHSLTGEGNFNWRYLFPFDYLAAEEKIV |
| | | MSKKESMFSWDETEYKIPARLTLQIWDADHFSADDFLGAIELDLNR |
| | | FPRGAKTAKQCTMEMATGEVDVPLVSIFKQKRVKGWWPLLARNEN |
| | | DEFELTGKVEAELHLLTAEEAEKNPVGLARNEPDPLEKPNRPDTSFI |
| | | WFLNPLKSARYFLWHTYRWLLLKFLLLFLLLLLFALFLYSLPGYLAKK |
| | | ILGA |
| 8 | mOTOF-202_1 | MALIVHLKTVSELRGKGDRIAKVTFRGQSFYSRVLENCEGVADFDE |
| | protein | TFRWPVASSIDRNEVLEIQIFNYSKVFSNKLIGTFCMVLQKVVEENR |
| | (NP_001093865.1), | VEVTDTLMDDSNAIIKTSLSMEVRYQATDGTVGPWDDGDFLGDESL |
| | mouse otoferlin | QEEKDSQETDGLLPGSRPSTRISGEKSFRSKGREKTKGGRDGEHK |
| | isoform 1, 1992 aa | AGRSVFSAMKLGKTRSHKEEPQRQDEPAVLEMEDLDHLAIQLGDG |
| | | LDPDSVSLASVTALTSNVSNKRSKPDIKMEPSAGRPMDYQVSITVIE |
| | | ARQLVGLNMDPVVCVEVGDDKKYTSMKESTNCPYYNEYFVFDFHV |
| | | SPDVMFDKIIKISVIHSKNLLRSGTLVGSFKMDVGTVYSQPEHQFHH |
| | | KWAILSDPDDISAGLKGYVKCDVAVVGKGDNIKTPHKANETDEDDIE |
| | | GNLLLPEGVPPERQWARFYVKIYRAEGLPRMNTSLMANVKKAFIGE |
| | | NKDLVDPYVQVFFAGQKGKTSVQKSSYEPLWNEQVVFTDLFPPLC |
| | | KRMKVQIRDSDKVNDVAIGTHFIDLRKISNDGDKGFLPTLGPAWVN |
| | | MYGSTRNYTLLDEHQDLNEGLGEGVSFRARLMLGLAVEILDTSNPE |
| | | LTSSTEVQVEQATPVSESCTGRMEEFFLFGAFLEASMIDRKNGDKP |
| | | ITFEVTIGNYGNEVDGMSRPLRPRPRKEPGDEEEVDLIQNSSDDEG |
| | | DEAGDLASVSSTPPMRPQITDRNYFHLPYLERKPCIYIKSWWPDQR |
| | | RRLYNANIMDHIADKLEEGLNDVQEMIKTEKSYPERRLRGVLEELSC |
| | | GCHRFLSLSDKDQGRSSRTRLDRERLKSCMRELESMGQQAKSLR |
| | | AQVKRHTVRDKLRSCQNFLQKLRFLADEPQHSIPDVFIWMMSNNK |
| | | RIAYARVPSKDLLFSIVEEELGKDCAKVKTLFLKLPGKRGFGSAGWT |
| | | VQAKLELYLWLGLSKQRKDFLCGLPCGFEEVKAAQGLGLHSFPPIS |
| | | LVYTKKQAFQLRAHMYQARSLFAADSSGLSDPFARVFFINQSQCTE |
| | | VLNETLCPTWDQMLVFDNLELYGEAHELRDDPPIIVIEIYDQDSMGK |
| | | ADFMGRTFAKPLVKMADEAYCPPRFPPQLEYYQIYRGSATAGDLLA |
| | | AFELLQIGPSGKADLPPINGPVDMDRGPIMPVPVGIRPVLSKYRVEV |
| | | LFWGLRDLKRVNLAQVDRPRVDIECAGKGVQSSLIHNYKKNPNFNT |
| | | LVKWFEVDLPENELLHPPLNIRVVDCRAFGRYTLVGSHAVSSLRRFI |
| | | YRPPDRSAPNWNTTGEVVVSMEPEEPVKKLETMVKLDATSDAVVK |
| | | VDVAEDEKERKKKKKKGPSEEPEEEEPDESMLDWWSKYFASIDTM |
| | | KEQLRQHETSGTDLEEKEEMESAEGLKGPMKSKEKSRAAKEEKKK |
| | | KNQSPGPGQGSEAPEKKKAKIDELKVYPKELESEFDSFEDWLHTF |
| | | NLLRGKTGDDEDGSTEEERIVGRFKGSLCVYKVPLPEDVSREAGY |
| | | DPTYGMFQGIPSNDPINVLVRIYVVRATDLHPADINGKADPYIAIKLG |
| | | KTDIRDKENYISKQLNPVFGKSFDIEASFPMESMLTVAVYDWDLVG |
| | | TDDLIGETKIDLENRFYSKHRATCGIAQTYSIHGYNIWRDPMKPSQIL |
| | | TRLCKEGKVDGPHFGPHGRVRVANRVFTGPSEIEDENGQRKPTDE |
| | | HVALSALRHWEDIPRVGCRLVPEHVETRPLLNPDKPGIEQGRLELW |
| | | VDMFPMDMPAPGTPLDISPRKPKKYELRVIVWNTDEVVLEDDDFFT |
| | | GEKSSDIFVRGWLKGQQEDKQDTDVHYHSLTGEGNFNWRYLFPF |
| | | DYLAAEEKIVMSKKESMFSWDETEYKIPARLTLQIWDADHFSADDF |
| | | LGAIELDLNRFPRGAKTAKQCTMEMATGEVDVPLVSIFKQKRVKGW |
| | | WPLLARNENDEFELTGKVEAELHLLTAEEAEKNPVGLARNEPDPLE |
| | | KPNRPDTAFVWFLNPLKSIKYLICTRYKWLIIKIVLALLGLLMLALFLY |
| | | SLPGYMVKKLLGA |
| 9 | mOTOF-202_2 | MALIVHLKTVSELRGKGDRIAKVTFRGQSFYSRVLENCEGVADFDE |
| | protein | TFRWPVASSIDRNEVLEIQIFNYSKVFSNKLIGTFCMVLQKVVEENR |
| | (NP_001300696.1), | VEVTDTLMDDSNAIIKTSLSMEVRYQATDGTVGPWDDGDFLGDESL |
| | mouse otoferlin | QEEKDSQETDGLLPGSRPSTRISGEKSFRRAGRSVFSAMKLGKTR |
| | isoform 4, 1977 aa | SHKEEPQRQDEPAVLEMEDLDHLAIQLGDGLDPDSVSLASVTALTS |
| | | NVSNKRSKPDIKMEPSAGRPMDYQVSITVIEARQLVGLNMDPVVCV |
| | | EVGDDKKYTSMKESTNCPYYNEYFVFDFHVSPDVMFDKIIKISVIHS |
| | | KNLLRSGTLVGSFKMDVGTVYSQPEHQFHHKWAILSDPDDISAGLK |
| | | GYVKCDVAVVGKGDNIKTPHKANETDEDDIEGNLLLPEGVPPERQ |
| | | WARFYVKIYRAEGLPRMNTSLMANVKKAFIGENKDLVDPYVQVFFA |
| | | GQKGKTSVQKSSYEPLWNEQVVFTDLFPPLCKRMKVQIRDSDKVN |
| | | DVAIGTHFIDLRKISNDGDKGFLPTLGPAWVNMYGSTRNYTLLDEH |
| | | QDLNEGLGEGVSFRARLMLGLAVEILDTSNPELTSSTEVQVEQATP |
| | | VSESCTGRMEEFFLFGAFLEASMIDRKNGDKPITFEVTIGNYGNEV |
| | | DGMSRPLRPRPRKEPGDEEEVDLIQNSSDDEGDEAGDLASVSSTP |
| | | PMRPQITDRNYFHLPYLERKPCIYIKSWWPDQRRRLYNANIMDHIA |
| | | DKLEEGLNDVQEMIKTEKSYPERRLRGVLEELSCGCHRFLSLSDKD |
| | | QGRSSRTRLDRERLKSCMRELESMGQQAKSLRAQVKRHTVRDKL |
| | | RSCQNFLQKLRFLADEPQHSIPDVFIWMMSNNKRIAYARVPSKDLL |
| | | FSIVEEELGKDCAKVKTLFLKLPGKRGFGSAGWTVQAKLELYLWLG |
| | | LSKQRKDFLCGLPCGFEEVKAAQGLGLHSFPPISLVYTKKQAFQLR |
| | | AHMYQARSLFAADSSGLSDPFARVFFINQSQCTEVLNETLCPTWD |
| | | QMLVFDNLELYGEAHELRDDPPIIVIEIYDQDSMGKADFMGRTFAKP |
| | | LVKMADEAYCPPRFPPQLEYYQIYRGSATAGDLLAAFELLQIGPSG |
| | | KADLPPINGPVDMDRGPIMPVPVGIRPVLSKYRVEVLFWGLRDLKR |
| | | VNLAQVDRPRVDIECAGKGVQSSLIHNYKKNPNFNTLVKWFEVDLP |
| | | ENELLHPPLNIRVVDCRAFGRYTLVGSHAVSSLRRFIYRPPDRSAP |
| | | NWNTTGEVVVSMEPEEPVKKLETMVKLDATSDAVVKVDVAEDEKE |
| | | RKKKKKKGPSEEPEEEEPDESMLDWWSKYFASIDTMKEQLRQHET |
| | | SGTDLEEKEEMESAEGLKGPMKSKEKSRAAKEEKKKKNQSPGPG |
| | | QGSEAPEKKKAKIDELKVYPKELESEFDSFEDWLHTFNLLRGKTGD |
| | | DEDGSTEEERIVGRFKGSLCVYKVPLPEDVSREAGYDPTYGMFQGI |
| | | PSNDPINVLVRIYVVRATDLHPADINGKADPYIAIKLGKTDIRDKENYI |
| | | SKQLNPVFGKSFDIEASFPMESMLTVAVYDWDLVGTDDLIGETKIDL |
| | | ENRFYSKHRATCGIAQTYSIHGYNIWRDPMKPSQILTRLCKEGKVD |
| | | GPHFGPHGRVRVANRVFTGPSEIEDENGQRKPTDEHVALSALRHW |
| | | EDIPRVGCRLVPEHVETRPLLNPDKPGIEQGRLELWVDMFPMDMP |
| | | APGTPLDISPRKPKKYELRVIVWNTDEVVLEDDDFFTGEKSSDIFVR |
| | | GWLKGQQEDKQDTDVHYHSLTGEGNFNWRYLFPFDYLAAEEKIV |
| | | MSKKESMFSWDETEYKIPARLTLQIWDADHFSADDFLGAIELDLNR |
| | | FPRGAKTAKQCTMEMATGEVDVPLVSIFKQKRVKGWWPLLARNEN |
| | | DEFELTGKVEAELHLLTAEEAEKNPVGLARNEPDPLEKPNRPDTAF |
| | | VWFLNPLKSIKYLICTRYKWLIIKIVLALLGLLMLALFLYSLPGYMVKK |
| | | LLGA |
| 10 | OTOF-201 transcript | ATCGGAGGGGGGTCGGGAGGAGGAGGAGGAGGCAGCGGCAG |
| | (NM_194248.1), | AGAAGAGAGAGGCGTGTGAGCCGTGCTCCACCGGCTAGCTCCT |
| | human otoferlin | TCCCGCTGCTCCTGCCTGGCAGTGCCAGGCAGCCCACACCAGC |
| | transcript variant 1, | ATGGCCTTGCTCATCCACCTCAAGACAGTCTCGGAGCTGCGGG |
| | 7156 bp, encodes | GCAGGGGCGACCGGATCGCCAAAGTGACTTTCCGAGGGCAATC |
| | the protein of SEQ | CTTCTACTCTCGGGTCCTGGAGAACTGTGAGGATGTGGCTGACT |
| | ID NO: 1 | TTGATGAGACATTTCGGTGGCCGGTGGCCAGCAGCATCGACAG |
| | | AAATGAGATGCTGGAGATTCAGGTTTTCAACTACAGCAAAGTCTT |
| | | CAGCAACAAGCTCATCGGGACCTTCCGCATGGTGCTGCAGAAG |
| | | GTGGTAGAGGAGAGCCATGTGGAGGTGACTGACACGCTGATTG |
| | | ATGACAACAATGCTATCATCAAGACCAGCCTGTGCGTGGAGGTC |
| | | CGGTATCAGGCCACTGACGGCACAGTGGGCTCCTGGGACGATG |
| | | GGGACTTCCTGGGAGATGAGTCTCTTCAAGAGGAAGAGAAGGA |
| | | CAGCCAAGAGACGGATGGACTGCTCCCAGGCTCCCGGCCCAGC |
| | | TCCCGGCCCCCAGGAGAGAAGAGCTTCCGGAGAGCCGGGAGG |
| | | AGCGTGTTCTCCGCCATGAAGCTCGGCAAAAACCGGTCTCACAA |
| | | GGAGGAGCCCCAAAGACCAGATGAACCGGCGGTGCTGGAGAT |
| | | GGAAGACCTTGACCATCTGGCCATTCGGCTAGGAGATGGACTG |
| | | GATCCCGACTCGGTGTCTCTAGCCTCAGTCACAGCTCTCACCAC |
| | | TAATGTCTCCAACAAGCGATCTAAGCCAGACATTAAGATGGAGC |
| | | CAAGTGCTGGGCGGCCCATGGATTACCAGGTCAGCATCACGGT |
| | | GATCGAGGCCCGGCAGCTGGTGGGCTTGAACATGGACCCTGTG |
| | | GTGTGCGTGGAGGTGGGTGACGACAAGAAGTACACATCCATGA |
| | | AGGAGTCCACTAACTGCCCCTATTACAACGAGTACTTCGTCTTC |
| | | GACTTCCATGTCTCTCCGGATGTCATGTTTGACAAGATCATCAAG |
| | | ATTTCGGTGATTCACTCCAAGAACCTGCTGCGCAGTGGCACCCT |
| | | GGTGGGCTCCTTCAAAATGGACGTGGGAACCGTGTACTCGCAG |
| | | CCAGAGCACCAGTTCCATCACAAGTGGGCCATCCTGTCTGACCC |
| | | CGATGACATCTCCTCGGGGCTGAAGGGCTACGTGAAGTGTGAC |
| | | GTTGCCGTGGTGGGCAAAGGGGACAACATCAAGACGCCCCACA |
| | | AGGCCAATGAGACCGACGAAGATGACATTGAGGGGAACTTGCT |
| | | GCTCCCCGAGGGGGTGCCCCCCGAACGCCAGTGGGCCCGGTT |
| | | CTATGTGAAAATTTACCGAGCAGAGGGGCTGCCCCGTATGAACA |
| | | CAAGCCTCATGGCCAATGTAAAGAAGGCTTTCATCGGTGAAAAC |
| | | AAGGACCTCGTGGACCCCTACGTGCAAGTCTTCTTTGCTGGCCA |
| | | GAAGGGCAAGACTTCAGTGCAGAAGAGCAGCTATGAGCCCCTG |
| | | TGGAATGAGCAGGTCGTCTTTACAGACCTCTTCCCCCCACTCTG |
| | | CAAACGCATGAAGGTGCAGATCCGAGACTCGGACAAGGTCAAC |
| | | GACGTGGCCATCGGCACCCACTTCATTGACCTGCGCAAGATTTC |
| | | TAATGACGGAGACAAAGGCTTCCTGCCCACACTGGGCCCAGCC |
| | | TGGGTGAACATGTACGGCTCCACACGTAACTACACGCTGCTGGA |
| | | TGAGCATCAGGACCTGAACGAGGGCCTGGGGGAGGGTGTGTC |
| | | CTTCCGGGCCCGGCTCCTGCTGGGCCTGGCTGTGGAGATCGTA |
| | | GACACCTCCAACCCTGAGCTCACCAGCTCCACAGAGGTGCAGG |
| | | TGGAGCAGGCCACGCCCATCTCGGAGAGCTGTGCAGGTAAAAT |
| | | GGAAGAATTCTTTCTCTTTGGAGCCTTCCTGGAGGCCTCAATGA |
| | | TCGACCGGAGAAACGGAGACAAGCCCATCACCTTTGAGGTCAC |
| | | CATAGGCAACTATGGGAACGAAGTTGATGGCCTGTCCCGGCCC |
| | | CAGCGGCCTCGGCCCCGGAAGGAGCCGGGGGATGAGGAAGAA |
| | | GTAGACCTGATTCAGAACGCAAGTGATGACGAGGCCGGTGATG |
| | | CCGGGGACCTGGCCTCAGTCTCCTCCACTCCACCAATGCGGCC |
| | | CCAGGTCACCGACAGGAACTACTTCCATCTGCCCTACCTGGAGC |
| | | GAAAGCCCTGCATCTACATCAAGAGCTGGTGGCCGGACCAGCG |
| | | CCGCCGCCTCTACAATGCCAACATCATGGACCACATTGCCGACA |
| | | AGCTGGAAGAAGGCCTGAACGACATACAGGAGATGATCAAAAC |
| | | GGAGAAGTCCTACCCTGAGCGTCGCCTGCGGGGCGTCCTGGA |
| | | GGAGCTGAGCTGTGGCTGCTGCCGCTTCCTCTCCCTCGCTGAC |
| | | AAGGACCAGGGCCACTCATCCCGCACCAGGCTTGACCGGGAGC |
| | | GCCTCAAGTCCTGCATGAGGGAGCTGGAAAACATGGGGCAGCA |
| | | GGCCAGGATGCTGCGGGCCCAGGTGAAGCGGCACACGGTGCG |
| | | GGACAAGCTGAGGCTGTGCCAGAACTTCCTGCAGAAGCTGCGC |
| | | TTCCTGGCGGACGAGCCCCAGCACAGCATTCCCGACATCTTCAT |
| | | CTGGATGATGAGCAACAACAAGCGTGTCGCCTATGCCCGTGTG |
| | | CCCTCCAAGGACCTGCTCTTCTCCATCGTGGAGGAGGAGACTG |
| | | GCAAGGACTGCGCCAAGGTCAAGACGCTCTTCCTTAAGCTGCC |
| | | AGGGAAGCGGGGCTTCGGCTCGGCAGGCTGGACAGTGCAGGC |
| | | CAAGGTGGAGCTGTACCTGTGGCTGGGCCTCAGCAAACAGCGC |
| | | AAGGAGTTCCTGTGCGGCCTGCCCTGTGGCTTCCAGGAGGTCA |
| | | AGGCAGCCCAGGGCCTGGGCCTGCATGCCTTCCCACCCGTCAG |
| | | CCTGGTCTACACCAAGAAGCAGGCGTTCCAGCTCCGAGCGCAC |
| | | ATGTACCAGGCCCGCAGCCTCTTTGCCGCCGACAGCAGCGGAC |
| | | TCTCAGACCCCTTTGCCCGCGTCTTCTTCATCAATCAGAGTCAG |
| | | TGCACAGAGGTGCTGAATGAGACCCTGTGTCCCACCTGGGACC |
| | | AGATGCTGGTGTTCGACAACCTGGAGCTCTATGGTGAAGCTCAT |
| | | GAGCTGAGGGACGATCCGCCCATCATTGTCATTGAAATCTATGA |
| | | CCAGGATTCCATGGGCAAAGCTGACTTCATGGGCCGGACCTTC |
| | | GCCAAACCCCTGGTGAAGATGGCAGACGAGGCGTACTGCCCAC |
| | | CCCGCTTCCCACCTCAGCTCGAGTACTACCAGATCTACCGTGGC |
| | | AACGCCACAGCTGGAGACCTGCTGGCGGCCTTCGAGCTGCTGC |
| | | AGATTGGACCAGCAGGGAAGGCTGACCTGCCCCCCATCAATGG |
| | | CCCGGTGGACGTGGACCGAGGTCCCATCATGCCCGTGCCCATG |
| | | GGCATCCGGCCCGTGCTCAGCAAGTACCGAGTGGAGGTGCTGT |
| | | TCTGGGGCCTACGGGACCTAAAGCGGGTGAACCTGGCCCAGGT |
| | | GGACCGGCCACGGGTGGACATCGAGTGTGCAGGGAAGGGGGT |
| | | GCAGTCGTCCCTGATCCACAATTATAAGAAGAACCCCAACTTCA |
| | | ACACCCTCGTCAAGTGGTTTGAAGTGGACCTCCCAGAGAACGA |
| | | GCTGCTGCACCCGCCCTTGAACATCCGTGTGGTGGACTGCCGG |
| | | GCCTTCGGTCGCTACACACTGGTGGGCTCCCATGCCGTCAGCT |
| | | CCCTGCGACGCTTCATCTACCGGCCCCCAGACCGCTCGGCCCC |
| | | CAGCTGGAACACCACGGTCAGGCTTCTCCGGCGCTGCCGTGTG |
| | | CTGTGCAATGGGGGCTCCTCCTCTCACTCCACAGGGGAGGTTG |
| | | TGGTGACTATGGAGCCAGAGGTACCCATCAAGAAACTGGAGAC |
| | | CATGGTGAAGCTGGACGCGACTTCTGAAGCTGTTGTCAAGGTG |
| | | GATGTGGCTGAGGAGGAGAAGGAGAAGAAGAAGAAGAAGAAGG |
| | | GCACTGCGGAGGAGCCAGAGGAGGAGGAGCCAGACGAGAGCA |
| | | TGCTGGACTGGTGGTCCAAGTACTTTGCCTCCATTGACACCATG |
| | | AAGGAGCAACTTCGACAACAAGAGCCCTCTGGAATTGACTTGGA |
| | | GGAGAAGGAGGAAGTGGACAATACCGAGGGCCTGAAGGGGTC |
| | | AATGAAGGGCAAGGAGAAGGCAAGGGCTGCCAAAGAGGAGAAG |
| | | AAGAAGAAAACTCAGAGCTCTGGCTCTGGCCAGGGGTCCGAGG |
| | | CCCCCGAGAAGAAGAAACCCAAGATTGATGAGCTTAAGGTATAC |
| | | CCCAAAGAGCTGGAGTCCGAGTTTGATAACTTTGAGGACTGGCT |
| | | GCACACTTTCAACTTGCTTCGGGGCAAGACCGGGGATGATGAG |
| | | GATGGCTCCACCGAGGAGGAGCGCATTGTGGGACGCTTCAAGG |
| | | GCTCCCTCTGCGTGTACAAAGTGCCACTCCCAGAGGACGTGTC |
| | | CCGGGAAGCCGGCTACGACTCCACCTACGGCATGTTCCAGGGC |
| | | ATCCCGAGCAATGACCCCATCAATGTGCTGGTCCGAGTCTATGT |
| | | GGTCCGGGCCACGGACCTGCACCCTGCTGACATCAACGGCAAA |
| | | GCTGACCCCTACATCGCCATCCGGCTAGGCAAGACTGACATCC |
| | | GCGACAAGGAGAACTACATCTCCAAGCAGCTCAACCCTGTCTTT |
| | | GGGAAGTCCTTTGACATCGAGGCCTCCTTCCCCATGGAATCCAT |
| | | GCTGACGGTGGCTGTGTATGACTGGGACCTGGTGGGCACTGAT |
| | | GACCTCATTGGGGAAACCAAGATCGACCTGGAGAACCGCTTCTA |
| | | CAGCAAGCACCGCGCCACCTGCGGCATCGCCCAGACCTACTCC |
| | | ACACATGGCTACAATATCTGGCGGGACCCCATGAAGCCCAGCC |
| | | AGATCCTGACCCGCCTCTGCAAAGACGGCAAAGTGGACGGCCC |
| | | CCACTTTGGGCCCCCTGGGAGAGTGAAGGTGGCCAACCGCGTC |
| | | TTCACTGGGCCCTCTGAGATTGAGGACGAGAACGGTCAGAGGA |
| | | AGCCCACAGACGAGCATGTGGCGCTGTTGGCCCTGAGGCACTG |
| | | GGAGGACATCCCCCGCGCAGGCTGCCGCCTGGTGCCAGAGCA |
| | | TGTGGAGACGAGGCCGCTGCTCAACCCCGACAAGCCGGGCATC |
| | | GAGCAGGGCCGCCTGGAGCTGTGGGTGGACATGTTCCCCATGG |
| | | ACATGCCAGCCCCTGGGACGCCTCTGGACATCTCACCTCGGAA |
| | | GCCCAAGAAGTACGAGCTGCGGGTCATCATCTGGAACACAGAT |
| | | GAGGTGGTCTTGGAGGACGACGACTTCTTCACAGGGGAGAAGT |
| | | CCAGTGACATCTTCGTGAGGGGGTGGCTGAAGGGCCAGCAGGA |
| | | GGACAAGCAGGACACAGACGTCCACTACCACTCCCTCACTGGC |
| | | GAGGGCAACTTCAACTGGCGCTACCTGTTCCCCTTCGACTACCT |
| | | GGCGGCGGAGGAGAAGATCGTCATCTCCAAGAAGGAGTCCATG |
| | | TTCTCCTGGGACGAGACCGAGTACAAGATCCCCGCGCGGCTCA |
| | | CCCTGCAGATCTGGGATGCGGACCACTTCTCCGCTGACGACTT |
| | | CCTGGGGGCCATCGAGCTGGACCTGAACCGGTTCCCGCGGGG |
| | | CGCAAAGACAGCCAAGCAGTGCACCATGGAGATGGCCACCGGG |
| | | GAGGTGGACGTGCCCCTCGTGTCCATCTTCAAGCAAAAGCGCG |
| | | TCAAAGGCTGGTGGCCCCTCCTGGCCCGCAATGAGAACGATGA |
| | | GTTTGAGCTCACGGGCAAGGTGGAGGCTGAGCTGCATTTACTG |
| | | ACAGCAGAGGAGGCAGAGAAGAACCCAGTGGGCCTGGCCCGC |
| | | AATGAACCTGACCCCCTAGAGAAACCCAACCGGCCCGACACGA |
| | | GCTTCATCTGGTTCCTGAACCCTCTCAAGTCGGCTCGCTACTTC |
| | | TTGTGGCACACGTATCGCTGGCTGCTCCTCAAACTGTTGCTGCT |
| | | CCTGCTGCTGCTCCTCCTCCTCGCCCTGTTCCTCTACTCTGTGC |
| | | CTGGCTACCTGGTCAAGAAAATCCTCGGGGCCTGAGCCCAGTG |
| | | GCCTCCTGGCCGGCCCGACACGGCCTTCGTCTGGTTCCTCAAC |
| | | CCTCTCAAGTCCATCAAGTACCTCATCTGCACCCGGTACAAGTG |
| | | GCTCATCATCAAGATCGTGCTGGCGCTGTTGGGGCTGCTCATGT |
| | | TGGGGCTCTTCCTCTACAGCCTCCCTGGCTACATGGTCAAAAAG |
| | | CTCCTTGGGGCATGAAGGCCGCCAGCTCCCGCCAGCCGCTCCC |
| | | CAGCCCTGCCGCATTTCCTTTCAGTGGCTTGGACTCTTTCCCAT |
| | | CTCCCCTGGGGAGCCTGAGGAGCCCAGCGTCCACTCTTCATGC |
| | | CTTGGGCCGAGCCTGCCTCCTGCTTGCGGGGGCCGCCTGTCCT |
| | | CACTGCCCCAGGCTGCGGCTTGCCCAGTCCCGCCCCTCTGACC |
| | | CCTGCCTGTGGGCTGGGGAGCCTTGGATGGGGTGGGGACCTG |
| | | GAATGGGTCTCTCTTGCCCCACCTGGCTGAGGCGCCACCCTTC |
| | | TTCAGGCCCAGGCTCCAGAGGAAGACTCCTGAAACCCTCCCCA |
| | | GGTCTTCCAAGTACAGGATTGAAGCTTTAGTGAAATTAACCAAG |
| | | GACCATGGGTCAGTGCCCAGGGCTTTAAAAAGAATGAACGAGC |
| | | AAAAGGTATCCCCGCCGTGACCCCTGCAGATAGCACCGGTCTTT |
| | | GATCCGCAGCAGGGGCCAGACCCTGCCCACAAGTCCCAGCGC |
| | | GGCTGCTTCTGCCACTGCTGGGCTCCACTTGGCTCCTCTCACTT |
| | | CCCAGGGGGTCGCCTGTCCTGCCTGTGGGTTTCCATGGCTTCC |
| | | CAGAGCTCCCTCTGCCCCAGCCAGCGCCTCCAGGCCCAGCTGA |
| | | GGAGCTGTGAGAAGCAGCAGAGGGGACTCCCCATCCCGGGCA |
| | | CACCCTGTCCTCCCACCCCTGCCCCCTTGCCCTTCCAGCCCTTT |
| | | CAGCTGCAGCTGGGAGCTGGCCCGTCAAGTGCTGCCCCTGCCT |
| | | GTGTCTGGGTTTCTGTTGGCTGTTTTTCTTTTCTTGAGTGGTGAT |
| | | TTTTCTCTAAATAAAAGAAGTCAAGCACTGAAAAAAAAAAAAAAA |
| | | A |
| 11 | OTOF-202 transcript | CCGTGAGTTCTGCCCAGGCCCTGTGAGCTCACCAGAGCCACAG |
| | (NM_004802.3), | ACTCACAGCCCAGAGGTGGCTTCTTCCTTCAGGAACTGAAGAAC |
| | human otoferlin | CCCCATGAACACCAACATCTCCAGGTTCTGAGAACAGAACCTGG |
| | transcript variant 2, | GAAATTGATGACTTCCTCATGATGACCGATACTCAGGATGGCCC |
| | 4954 bp, encodes | TAGCGAGAGCTCCCAGATCATGAGGAAGAAGGCCTGAACGACA |
| | the protein of SEQ | TACAGGAGATGATCAAAACGGAGAAGTCCTACCCTGAGCGTCG |
| | ID NO: 2 | CCTGCGGGGCGTCCTGGAGGAGCTGAGCTGTGGCTGCTGCCG |
| | | CTTCCTCTCCCTCGCTGACAAGGACCAGGGCCACTCATCCCGC |
| | | ACCAGGCTTGACCGGGAGCGCCTCAAGTCCTGCATGAGGGAGC |
| | | TGGAAAACATGGGGCAGCAGGCCAGGATGCTGCGGGCCCAGG |
| | | TGAAGCGGCACACGGTGCGGGACAAGCTGAGGCTGTGCCAGAA |
| | | CTTCCTGCAGAAGCTGCGCTTCCTGGCGGACGAGCCCCAGCAC |
| | | AGCATTCCCGACATCTTCATCTGGATGATGAGCAACAACAAGCG |
| | | TGTCGCCTATGCCCGTGTGCCCTCCAAGGACCTGCTCTTCTCCA |
| | | TCGTGGAGGAGGAGACTGGCAAGGACTGCGCCAAGGTCAAGAC |
| | | GCTCTTCCTTAAGCTGCCAGGGAAGCGGGGCTTCGGCTCGGCA |
| | | GGCTGGACAGTGCAGGCCAAGGTGGAGCTGTACCTGTGGCTGG |
| | | GCCTCAGCAAACAGCGCAAGGAGTTCCTGTGCGGCCTGCCCTG |
| | | TGGCTTCCAGGAGGTCAAGGCAGCCCAGGGCCTGGGCCTGCAT |
| | | GCCTTCCCACCCGTCAGCCTGGTCTACACCAAGAAGCAGGCGT |
| | | TCCAGCTCCGAGCGCACATGTACCAGGCCCGCAGCCTCTTTGC |
| | | CGCCGACAGCAGCGGACTCTCAGACCCCTTTGCCCGCGTCTTC |
| | | TTCATCAATCAGAGTCAGTGCACAGAGGTGCTGAATGAGACCCT |
| | | GTGTCCCACCTGGGACCAGATGCTGGTGTTCGACAACCTGGAG |
| | | CTCTATGGTGAAGCTCATGAGCTGAGGGACGATCCGCCCATCAT |
| | | TGTCATTGAAATCTATGACCAGGATTCCATGGGCAAAGCTGACT |
| | | TCATGGGCCGGACCTTCGCCAAACCCCTGGTGAAGATGGCAGA |
| | | CGAGGCGTACTGCCCACCCCGCTTCCCACCTCAGCTCGAGTAC |
| | | TACCAGATCTACCGTGGCAACGCCACAGCTGGAGACCTGCTGG |
| | | CGGCCTTCGAGCTGCTGCAGATTGGACCAGCAGGGAAGGCTGA |
| | | CCTGCCCCCCATCAATGGCCCGGTGGACGTGGACCGAGGTCCC |
| | | ATCATGCCCGTGCCCATGGGCATCCGGCCCGTGCTCAGCAAGT |
| | | ACCGAGTGGAGGTGCTGTTCTGGGGCCTACGGGACCTAAAGCG |
| | | GGTGAACCTGGCCCAGGTGGACCGGCCACGGGTGGACATCGA |
| | | GTGTGCAGGGAAGGGGGTGCAGTCGTCCCTGATCCACAATTAT |
| | | AAGAAGAACCCCAACTTCAACACCCTCGTCAAGTGGTTTGAAGT |
| | | GGACCTCCCAGAGAACGAGCTGCTGCACCCGCCCTTGAACATC |
| | | CGTGTGGTGGACTGCCGGGCCTTCGGTCGCTACACACTGGTGG |
| | | GCTCCCATGCCGTCAGCTCCCTGCGACGCTTCATCTACCGGCC |
| | | CCCAGACCGCTCGGCCCCCAGCTGGAACACCACGGGGGAGGT |
| | | TGTGGTGACTATGGAGCCAGAGGTACCCATCAAGAAACTGGAG |
| | | ACCATGGTGAAGCTGGACGCGACTTCTGAAGCTGTTGTCAAGGT |
| | | GGATGTGGCTGAGGAGGAGAAGGAGAAGAAGAAGAAGAAGAAG |
| | | GGCACTGCGGAGGAGCCAGAGGAGGAGGAGCCAGACGAGAGC |
| | | ATGCTGGACTGGTGGTCCAAGTACTTTGCCTCCATTGACACCAT |
| | | GAAGGAGCAACTTCGACAACAAGAGCCCTCTGGAATTGACTTGG |
| | | AGGAGAAGGAGGAAGTGGACAATACCGAGGGCCTGAAGGGGT |
| | | CAATGAAGGGCAAGGAGAAGGCAAGGGCTGCCAAAGAGGAGAA |
| | | GAAGAAGAAAACTCAGAGCTCTGGCTCTGGCCAGGGGTCCGAG |
| | | GCCCCCGAGAAGAAGAAACCCAAGATTGATGAGCTTAAGGTATA |
| | | CCCCAAAGAGCTGGAGTCCGAGTTTGATAACTTTGAGGACTGGC |
| | | TGCACACTTTCAACTTGCTTCGGGGCAAGACCGGGGATGATGA |
| | | GGATGGCTCCACCGAGGAGGAGCGCATTGTGGGACGCTTCAAG |
| | | GGCTCCCTCTGCGTGTACAAAGTGCCACTCCCAGAGGACGTGT |
| | | CCCGGGAAGCCGGCTACGACTCCACCTACGGCATGTTCCAGGG |
| | | CATCCCGAGCAATGACCCCATCAATGTGCTGGTCCGAGTCTATG |
| | | TGGTCCGGGCCACGGACCTGCACCCTGCTGACATCAACGGCAA |
| | | AGCTGACCCCTACATCGCCATCCGGCTAGGCAAGACTGACATC |
| | | CGCGACAAGGAGAACTACATCTCCAAGCAGCTCAACCCTGTCTT |
| | | TGGGAAGTCCTTTGACATCGAGGCCTCCTTCCCCATGGAATCCA |
| | | TGCTGACGGTGGCTGTGTATGACTGGGACCTGGTGGGCACTGA |
| | | TGACCTCATTGGGGAAACCAAGATCGACCTGGAGAACCGCTTCT |
| | | ACAGCAAGCACCGCGCCACCTGCGGCATCGCCCAGACCTACTC |
| | | CACACATGGCTACAATATCTGGCGGGACCCCATGAAGCCCAGC |
| | | CAGATCCTGACCCGCCTCTGCAAAGACGGCAAAGTGGACGGCC |
| | | CCCACTTTGGGCCCCCTGGGAGAGTGAAGGTGGCCAACCGCGT |
| | | CTTCACTGGGCCCTCTGAGATTGAGGACGAGAACGGTCAGAGG |
| | | AAGCCCACAGACGAGCATGTGGCGCTGTTGGCCCTGAGGCACT |
| | | GGGAGGACATCCCCCGCGCAGGCTGCCGCCTGGTGCCAGAGC |
| | | ATGTGGAGACGAGGCCGCTGCTCAACCCCGACAAGCCGGGCAT |
| | | CGAGCAGGGCCGCCTGGAGCTGTGGGTGGACATGTTCCCCATG |
| | | GACATGCCAGCCCCTGGGACGCCTCTGGACATCTCACCTCGGA |
| | | AGCCCAAGAAGTACGAGCTGCGGGTCATCATCTGGAACACAGA |
| | | TGAGGTGGTCTTGGAGGACGACGACTTCTTCACAGGGGAGAAG |
| | | TCCAGTGACATCTTCGTGAGGGGGTGGCTGAAGGGCCAGCAGG |
| | | AGGACAAGCAGGACACAGACGTCCACTACCACTCCCTCACTGG |
| | | CGAGGGCAACTTCAACTGGCGCTACCTGTTCCCCTTCGACTACC |
| | | TGGCGGCGGAGGAGAAGATCGTCATCTCCAAGAAGGAGTCCAT |
| | | GTTCTCCTGGGACGAGACCGAGTACAAGATCCCCGCGCGGCTC |
| | | ACCCTGCAGATCTGGGATGCGGACCACTTCTCCGCTGACGACTT |
| | | CCTGGGGGCCATCGAGCTGGACCTGAACCGGTTCCCGCGGGG |
| | | CGCAAAGACAGCCAAGCAGTGCACCATGGAGATGGCCACCGGG |
| | | GAGGTGGACGTGCCCCTCGTGTCCATCTTCAAGCAAAAGCGCG |
| | | TCAAAGGCTGGTGGCCCCTCCTGGCCCGCAATGAGAACGATGA |
| | | GTTTGAGCTCACGGGCAAGGTGGAGGCTGAGCTGCATTTACTG |
| | | ACAGCAGAGGAGGCAGAGAAGAACCCAGTGGGCCTGGCCCGC |
| | | AATGAACCTGACCCCCTAGAGAAACCCAACCGGCCCGACACGA |
| | | GCTTCATCTGGTTCCTGAACCCTCTCAAGTCGGCTCGCTACTTC |
| | | TTGTGGCACACGTATCGCTGGCTGCTCCTCAAACTGTTGCTGCT |
| | | CCTGCTGCTGCTCCTCCTCCTCGCCCTGTTCCTCTACTCTGTGC |
| | | CTGGCTACCTGGTCAAGAAAATCCTCGGGGCCTGAGCCCAGTG |
| | | GCCTCCTGGCCGGCCCGACACGGCCTTCGTCTGGTTCCTCAAC |
| | | CCTCTCAAGTCCATCAAGTACCTCATCTGCACCCGGTACAAGTG |
| | | GCTCATCATCAAGATCGTGCTGGCGCTGTTGGGGCTGCTCATGT |
| | | TGGGGCTCTTCCTCTACAGCCTCCCTGGCTACATGGTCAAAAAG |
| | | CTCCTTGGGGCATGAAGGCCGCCAGCTCCCGCCAGCCGCTCCC |
| | | CAGCCCTGCCGCATTTCCTTTCAGTGGCTTGGACTCTTTCCCAT |
| | | CTCCCCTGGGGAGCCTGAGGAGCCCAGCGTCCACTCTTCATGC |
| | | CTTGGGCCGAGCCTGCCTCCTGCTTGCGGGGGCCGCCTGTCCT |
| | | CACTGCCCCAGGCTGCGGCTTGCCCAGTCCCGCCCCTCTGACC |
| | | CCTGCCTGTGGGCTGGGGAGCCTTGGATGGGGTGGGGACCTG |
| | | GAATGGGTCTCTCTTGCCCCACCTGGCTGAGGCGCCACCCTTC |
| | | TTCAGGCCCAGGCTCCAGAGGAAGACTCCTGAAACCCTCCCCA |
| | | GGTCTTCCAAGTACAGGATTGAAGCTTTAGTGAAATTAACCAAG |
| | | GACCATGGGTCAGTGCCCAGGGCTTTAAAAAGAATGAACGAGC |
| | | AAAAGGTATCCCCGCCGTGACCCCTGCAGATAGCACCGGTCTTT |
| | | GATCCGCAGCAGGGGCCAGACCCTGCCCACAAGTCCCAGCGC |
| | | GGCTGCTTCTGCCACTGCTGGGCTCCACTTGGCTCCTCTCACTT |
| | | CCCAGGGGGTCGCCTGTCCTGCCTGTGGGTTTCCATGGCTTCC |
| | | CAGAGCTCCCTCTGCCCCAGCCAGCGCCTCCAGGCCCAGCTGA |
| | | GGAGCTGTGAGAAGCAGCAGAGGGGACTCCCCATCCCGGGCA |
| | | CACCCTGTCCTCCCACCCCTGCCCCCTTGCCCTTCCAGCCCTTT |
| | | CAGCTGCAGCTGGGAGCTGGCCCGTCAAGTGCTGCCCCTGCCT |
| | | GTGTCTGGGTTTCTGTTGGCTGTTTTTCTTTTCTTGAGTGGTGAT |
| | | TTTTCTCTAAATAAAAGAAGTCAAGCACTGAAAAAAAAAAAAAAA |
| | | A |
| 12 | OTOF-203 transcript | CCGTGAGTTCTGCCCAGGCCCTGTGAGCTCACCAGAGCCACAG |
| | (NM_194323.2), | ACTCACAGCCCAGAGGTGGCTTCTTCCTTCAGGAACTGAAGAAC |
| | human otoferlin | CCCCATGAACACCAACATCTCCAGGTTCTGAGAACAGAACCTGG |
| | transcript variant 4, | GAAATTGATGACTTCCTCATGATGACCGATACTCAGGATGGCCC |
| | 4756 bp, encodes | TAGCGAGAGCTCCCAGATCATGAGGAAGAAGGCCTGAACGACA |
| | the protein of SEQ | TACAGGAGATGATCAAAACGGAGAAGTCCTACCCTGAGCGTCG |
| | ID NO: 3 | CCTGCGGGGCGTCCTGGAGGAGCTGAGCTGTGGCTGCTGCCG |
| | | CTTCCTCTCCCTCGCTGACAAGGACCAGGGCCACTCATCCCGC |
| | | ACCAGGCTTGACCGGGAGCGCCTCAAGTCCTGCATGAGGGAGC |
| | | TGGAAAACATGGGGCAGCAGGCCAGGATGCTGCGGGCCCAGG |
| | | TGAAGCGGCACACGGTGCGGGACAAGCTGAGGCTGTGCCAGAA |
| | | CTTCCTGCAGAAGCTGCGCTTCCTGGCGGACGAGCCCCAGCAC |
| | | AGCATTCCCGACATCTTCATCTGGATGATGAGCAACAACAAGCG |
| | | TGTCGCCTATGCCCGTGTGCCCTCCAAGGACCTGCTCTTCTCCA |
| | | TCGTGGAGGAGGAGACTGGCAAGGACTGCGCCAAGGTCAAGAC |
| | | GCTCTTCCTTAAGCTGCCAGGGAAGCGGGGCTTCGGCTCGGCA |
| | | GGCTGGACAGTGCAGGCCAAGGTGGAGCTGTACCTGTGGCTGG |
| | | GCCTCAGCAAACAGCGCAAGGAGTTCCTGTGCGGCCTGCCCTG |
| | | TGGCTTCCAGGAGGTCAAGGCAGCCCAGGGCCTGGGCCTGCAT |
| | | GCCTTCCCACCCGTCAGCCTGGTCTACACCAAGAAGCAGGCGT |
| | | TCCAGCTCCGAGCGCACATGTACCAGGCCCGCAGCCTCTTTGC |
| | | CGCCGACAGCAGCGGACTCTCAGACCCCTTTGCCCGCGTCTTC |
| | | TTCATCAATCAGAGTCAGTGCACAGAGGTGCTGAATGAGACCCT |
| | | GTGTCCCACCTGGGACCAGATGCTGGTGTTCGACAACCTGGAG |
| | | CTCTATGGTGAAGCTCATGAGCTGAGGGACGATCCGCCCATCAT |
| | | TGTCATTGAAATCTATGACCAGGATTCCATGGGCAAAGCTGACT |
| | | TCATGGGCCGGACCTTCGCCAAACCCCTGGTGAAGATGGCAGA |
| | | CGAGGCGTACTGCCCACCCCGCTTCCCACCTCAGCTCGAGTAC |
| | | TACCAGATCTACCGTGGCAACGCCACAGCTGGAGACCTGCTGG |
| | | CGGCCTTCGAGCTGCTGCAGATTGGACCAGCAGGGAAGGCTGA |
| | | CCTGCCCCCCATCAATGGCCCGGTGGACGTGGACCGAGGTCCC |
| | | ATCATGCCCGTGCCCATGGGCATCCGGCCCGTGCTCAGCAAGT |
| | | ACCGAGTGGAGGTGCTGTTCTGGGGCCTACGGGACCTAAAGCG |
| | | GGTGAACCTGGCCCAGGTGGACCGGCCACGGGTGGACATCGA |
| | | GTGTGCAGGGAAGGGGGTGCAGTCGTCCCTGATCCACAATTAT |
| | | AAGAAGAACCCCAACTTCAACACCCTCGTCAAGTGGTTTGAAGT |
| | | GGACCTCCCAGAGAACGAGCTGCTGCACCCGCCCTTGAACATC |
| | | CGTGTGGTGGACTGCCGGGCCTTCGGTCGCTACACACTGGTGG |
| | | GCTCCCATGCCGTCAGCTCCCTGCGACGCTTCATCTACCGGCC |
| | | CCCAGACCGCTCGGCCCCCAGCTGGAACACCACGGGGGAGGT |
| | | TGTGGTGACTATGGAGCCAGAGGTACCCATCAAGAAACTGGAG |
| | | ACCATGGTGAAGCTGGACGCGACTTCTGAAGCTGTTGTCAAGGT |
| | | GGATGTGGCTGAGGAGGAGAAGGAGAAGAAGAAGAAGAAGAAG |
| | | GGCACTGCGGAGGAGCCAGAGGAGGAGGAGCCAGACGAGAGC |
| | | ATGCTGGACTGGTGGTCCAAGTACTTTGCCTCCATTGACACCAT |
| | | GAAGGAGCAACTTCGACAACAAGAGCCCTCTGGAATTGACTTGG |
| | | AGGAGAAGGAGGAAGTGGACAATACCGAGGGCCTGAAGGGGT |
| | | CAATGAAGGGCAAGGAGAAGGCAAGGGCTGCCAAAGAGGAGAA |
| | | GAAGAAGAAAACTCAGAGCTCTGGCTCTGGCCAGGGGTCCGAG |
| | | GCCCCCGAGAAGAAGAAACCCAAGATTGATGAGCTTAAGGTATA |
| | | CCCCAAAGAGCTGGAGTCCGAGTTTGATAACTTTGAGGACTGGC |
| | | TGCACACTTTCAACTTGCTTCGGGGCAAGACCGGGGATGATGA |
| | | GGATGGCTCCACCGAGGAGGAGCGCATTGTGGGACGCTTCAAG |
| | | GGCTCCCTCTGCGTGTACAAAGTGCCACTCCCAGAGGACGTGT |
| | | CCCGGGAAGCCGGCTACGACTCCACCTACGGCATGTTCCAGGG |
| | | CATCCCGAGCAATGACCCCATCAATGTGCTGGTCCGAGTCTATG |
| | | TGGTCCGGGCCACGGACCTGCACCCTGCTGACATCAACGGCAA |
| | | AGCTGACCCCTACATCGCCATCCGGCTAGGCAAGACTGACATC |
| | | CGCGACAAGGAGAACTACATCTCCAAGCAGCTCAACCCTGTCTT |
| | | TGGGAAGTCCTTTGACATCGAGGCCTCCTTCCCCATGGAATCCA |
| | | TGCTGACGGTGGCTGTGTATGACTGGGACCTGGTGGGCACTGA |
| | | TGACCTCATTGGGGAAACCAAGATCGACCTGGAGAACCGCTTCT |
| | | ACAGCAAGCACCGCGCCACCTGCGGCATCGCCCAGACCTACTC |
| | | CACACATGGCTACAATATCTGGCGGGACCCCATGAAGCCCAGC |
| | | CAGATCCTGACCCGCCTCTGCAAAGACGGCAAAGTGGACGGCC |
| | | CCCACTTTGGGCCCCCTGGGAGAGTGAAGGTGGCCAACCGCGT |
| | | CTTCACTGGGCCCTCTGAGATTGAGGACGAGAACGGTCAGAGG |
| | | AAGCCCACAGACGAGCATGTGGCGCTGTTGGCCCTGAGGCACT |
| | | GGGAGGACATCCCCCGCGCAGGCTGCCGCCTGGTGCCAGAGC |
| | | ATGTGGAGACGAGGCCGCTGCTCAACCCCGACAAGCCGGGCAT |
| | | CGAGCAGGGCCGCCTGGAGCTGTGGGTGGACATGTTCCCCATG |
| | | GACATGCCAGCCCCTGGGACGCCTCTGGACATCTCACCTCGGA |
| | | AGCCCAAGAAGTACGAGCTGCGGGTCATCATCTGGAACACAGA |
| | | TGAGGTGGTCTTGGAGGACGACGACTTCTTCACAGGGGAGAAG |
| | | TCCAGTGACATCTTCGTGAGGGGGTGGCTGAAGGGCCAGCAGG |
| | | AGGACAAGCAGGACACAGACGTCCACTACCACTCCCTCACTGG |
| | | CGAGGGCAACTTCAACTGGCGCTACCTGTTCCCCTTCGACTACC |
| | | TGGCGGCGGAGGAGAAGATCGTCATCTCCAAGAAGGAGTCCAT |
| | | GTTCTCCTGGGACGAGACCGAGTACAAGATCCCCGCGCGGCTC |
| | | ACCCTGCAGATCTGGGATGCGGACCACTTCTCCGCTGACGACTT |
| | | CCTGGGGGCCATCGAGCTGGACCTGAACCGGTTCCCGCGGGG |
| | | CGCAAAGACAGCCAAGCAGTGCACCATGGAGATGGCCACCGGG |
| | | GAGGTGGACGTGCCCCTCGTGTCCATCTTCAAGCAAAAGCGCG |
| | | TCAAAGGCTGGTGGCCCCTCCTGGCCCGCAATGAGAACGATGA |
| | | GTTTGAGCTCACGGGCAAGGTGGAGGCTGAGCTGCATTTACTG |
| | | ACAGCAGAGGAGGCAGAGAAGAACCCAGTGGGCCTGGCCCGC |
| | | AATGAACCTGACCCCCTAGAGAAACCCAACCGGCCCGACACGG |
| | | CCTTCGTCTGGTTCCTCAACCCTCTCAAGTCCATCAAGTACCTCA |
| | | TCTGCACCCGGTACAAGTGGCTCATCATCAAGATCGTGCTGGCG |
| | | CTGTTGGGGCTGCTCATGTTGGGGCTCTTCCTCTACAGCCTCCC |
| | | TGGCTACATGGTCAAAAAGCTCCTTGGGGCATGAAGGCCGCCA |
| | | GCTCCCGCCAGCCGCTCCCCAGCCCTGCCGCATTTCCTTTCAG |
| | | TGGCTTGGACTCTTTCCCATCTCCCCTGGGGAGCCTGAGGAGC |
| | | CCAGCGTCCACTCTTCATGCCTTGGGCCGAGCCTGCCTCCTGC |
| | | TTGCGGGGGCCGCCTGTCCTCACTGCCCCAGGCTGCGGCTTGC |
| | | CCAGTCCCGCCCCTCTGACCCCTGCCTGTGGGCTGGGGAGCCT |
| | | TGGATGGGGTGGGGACCTGGAATGGGTCTCTCTTGCCCCACCT |
| | | GGCTGAGGCGCCACCCTTCTTCAGGCCCAGGCTCCAGAGGAAG |
| | | ACTCCTGAAACCCTCCCCAGGTCTTCCAAGTACAGGATTGAAGC |
| | | TTTAGTGAAATTAACCAAGGACCATGGGTCAGTGCCCAGGGCTT |
| | | TAAAAAGAATGAACGAGCAAAAGGTATCCCCGCCGTGACCCCTG |
| | | CAGATAGCACCGGTCTTTGATCCGCAGCAGGGGCCAGACCCTG |
| | | CCCACAAGTCCCAGCGCGGCTGCTTCTGCCACTGCTGGGCTCC |
| | | ACTTGGCTCCTCTCACTTCCCAGGGGGTCGCCTGTCCTGCCTGT |
| | | GGGTTTCCATGGCTTCCCAGAGCTCCCTCTGCCCCAGCCAGCG |
| | | CCTCCAGGCCCAGCTGAGGAGCTGTGAGAAGCAGCAGAGGGG |
| | | ACTCCCCATCCCGGGCACACCCTGTCCTCCCACCCCTGCCCCC |
| | | TTGCCCTTCCAGCCCTTTCAGCTGCAGCTGGGAGCTGGCCCGT |
| | | CAAGTGCTGCCCCTGCCTGTGTCTGGGTTTCTGTTGGCTGTTTT |
| | | TCTTTTCTTGAGTGGTGATTTTTCTCTAAATAAAAGAAGTCAAGC |
| | | ACTGAAAAAAAAAAAAAAAA |
| 13 | OTOF-208 transcript | CCGTGAGTTCTGCCCAGGCCCTGTGAGCTCACCAGAGCCACAG |
| | (NM_194322.2), | ACTCACAGCCCAGAGGTGGCTTCTTCCTTCAGGAACTGAAGAAC |
| | human otoferlin | CCCCATGAACACCAACATCTCCAGGTTCTGAGAACAGAACCTGG |
| | transcript variant 3, | GAAATTGATGACTTCCTCATGATGACCGATACTCAGGATGGCCC |
| | 3924 bp, encodes | TAGCGAGAGCTCCCAGATCATGAGGTCCCTCACTCCCCTGATCA |
| | the protein of SEQ | ACAGGGAGGAGGCATTTGGGGAGGCTGGGGAGGCGGGGCTGT |
| | ID NO: 4 | GGCCCAGCATCACCCACACTCCTGATTCACAGGAAGAAGGCCT |
| | | GAACGACATACAGGAGATGATCAAAACGGAGAAGTCCTACCCTG |
| | | AGCGTCGCCTGCGGGGCGTCCTGGAGGAGCTGAGCTGTGGCT |
| | | GCTGCCGCTTCCTCTCCCTCGCTGACAAGGACCAGGGCCACTC |
| | | ATCCCGCACCAGGCTTGACCGGGAGCGCCTCAAGTCCTGCATG |
| | | AGGGAGCTGGAAAACATGGGGCAGCAGGCCAGGATGCTGCGG |
| | | GCCCAGGTGAAGCGGCACACGGTGCGGGACAAGCTGAGGCTG |
| | | TGCCAGAACTTCCTGCAGAAGCTGCGCTTCCTGGCGGACGAGC |
| | | CCCAGCACAGCATTCCCGACATCTTCATCTGGATGATGAGCAAC |
| | | AACAAGCGTGTCGCCTATGCCCGTGTGCCCTCCAAGGACCTGC |
| | | TCTTCTCCATCGTGGAGGAGGAGACTGGCAAGGACTGCGCCAA |
| | | GGTCAAGACGCTCTTCCTTAAGCTGCCAGGGAAGCGGGGCTTC |
| | | GGCTCGGCAGGCTGGACAGTGCAGGCCAAGGTGGAGCTGTAC |
| | | CTGTGGCTGGGCCTCAGCAAACAGCGCAAGGAGTTCCTGTGCG |
| | | GCCTGCCCTGTGGCTTCCAGGAGGTCAAGGCAGCCCAGGGCCT |
| | | GGGCCTGCATGCCTTCCCACCCGTCAGCCTGGTCTACACCAAG |
| | | AAGCAGGCGTTCCAGCTCCGAGCGCACATGTACCAGGCCCGCA |
| | | GCCTCTTTGCCGCCGACAGCAGCGGACTCTCAGACCCCTTTGC |
| | | CCGCGTCTTCTTCATCAATCAGAGTCAGTGCACAGAGGTGCTGA |
| | | ATGAGACCCTGTGTCCCACCTGGGACCAGATGCTGGTGTTCGA |
| | | CAACCTGGAGCTCTATGGTGAAGCTCATGAGCTGAGGGACGAT |
| | | CCGCCCATCATTGTCATTGAAATCTATGACCAGGATTCCATGGG |
| | | CAAAGCTGACTTCATGGGCCGGACCTTCGCCAAACCCCTGGTG |
| | | AAGATGGCAGACGAGGCGTACTGCCCACCCCGCTTCCCACCTC |
| | | AGCTCGAGTACTACCAGATCTACCGTGGCAACGCCACAGCTGG |
| | | AGACCTGCTGGCGGCCTTCGAGCTGCTGCAGATTGGACCAGCA |
| | | GGGAAGGCTGACCTGCCCCCCATCAATGGCCCGGTGGACGTG |
| | | GACCGAGGTCCCATCATGCCCGTGCCCATGGGCATCCGGCCCG |
| | | TGCTCAGCAAGTACCGAGTGGAGGTGCTGTTCTGGGGCCTACG |
| | | GGACCTAAAGCGGGTGAACCTGGCCCAGGTGGACCGGCCACG |
| | | GGTGGACATCGAGTGTGCAGGGAAGGGGGTGCAGTCGTCCCT |
| | | GATCCACAATTATAAGAAGAACCCCAACTTCAACACCCTCGTCAA |
| | | GTGGTTTGAAGTGGACCTCCCAGAGAACGAGCTGCTGCACCCG |
| | | CCCTTGAACATCCGTGTGGTGGACTGCCGGGCCTTCGGTCGCT |
| | | ACACACTGGTGGGCTCCCATGCCGTCAGCTCCCTGCGACGCTT |
| | | CATCTACCGGCCCCCAGACCGCTCGGCCCCCAGCTGGAACACC |
| | | ACGGTCAGGCTTCTCCGGCGCTGCCGTGTGCTGTGCAATGGGG |
| | | GCTCCTCCTCTCACTCCACAGGGGAGGTTGTGGTGACTATGGA |
| | | GCCAGAGGTACCCATCAAGAAACTGGAGACCATGGTGAAGCTG |
| | | GACGCGACTTCTGAAGCTGTTGTCAAGGTGGATGTGGCTGAGG |
| | | AGGAGAAGGAGAAGAAGAAGAAGAAGAAGGGCACTGCGGAGG |
| | | AGCCAGAGGAGGAGGAGCCAGACGAGAGCATGCTGGACTGGT |
| | | GGTCCAAGTACTTTGCCTCCATTGACACCATGAAGGAGCAACTT |
| | | CGACAACAAGAGCCCTCTGGAATTGACTTGGAGGAGAAGGAGG |
| | | AAGTGGACAATACCGAGGGCCTGAAGGGGTCAATGAAGGGCAA |
| | | GGAGAAGGCAAGGGCTGCCAAAGAGGAGAAGAAGAAGAAAACT |
| | | CAGAGCTCTGGCTCTGGCCAGGGGTCCGAGGCCCCCGAGAAG |
| | | AAGAAACCCAAGATTGATGAGCTTAAGGTATACCCCAAAGAGCT |
| | | GGAGTCCGAGTTTGATAACTTTGAGGACTGGCTGCACACTTTCA |
| | | ACTTGCTTCGGGGCAAGACCGGGGATGATGAGGATGGCTCCAC |
| | | CGAGGAGGAGCGCATTGTGGGACGCTTCAAGGGCTCCCTCTGC |
| | | GTGTACAAAGTGCCACTCCCAGAGGACGTGTCCCGGGAAGCCG |
| | | GCTACGACTCCACCTACGGCATGTTCCAGGGCATCCCGAGCAA |
| | | TGACCCCATCAATGTGCTGGTCCGAGTCTATGTGGTCCGGGCC |
| | | ACGGACCTGCACCCTGCTGACATCAACGGCAAAGCTGACCCCT |
| | | ACATCGCCATCCGGCTAGGCAAGACTGACATCCGCGACAAGGA |
| | | GAACTACATCTCCAAGCAGCTCAACCCTGTCTTTGGGAAGTCCT |
| | | TTGACATCGAGGCCTCCTTCCCCATGGAATCCATGCTGACGGTG |
| | | GCTGTGTATGACTGGGACCTGGTGGGCACTGATGACCTCATTG |
| | | GGGAAACCAAGATCGACCTGGAGAACCGCTTCTACAGCAAGCA |
| | | CCGCGCCACCTGCGGCATCGCCCAGACCTACTCCACACATGGC |
| | | TACAATATCTGGCGGGACCCCATGAAGCCCAGCCAGATCCTGA |
| | | CCCGCCTCTGCAAAGACGGCAAAGTGGACGGCCCCCACTTTGG |
| | | GCCCCCTGGGAGAGTGAAGGTGGCCAACCGCGTCTTCACTGGG |
| | | CCCTCTGAGATTGAGGACGAGAACGGTCAGAGGAAGCCCACAG |
| | | ACGAGCATGTGGCGCTGTTGGCCCTGAGGCACTGGGAGGACAT |
| | | CCCCCGCGCAGGCTGCCGCCTGGTGCCAGAGCATGTGGAGAC |
| | | GAGGCCGCTGCTCAACCCCGACAAGCCGGGCATCGAGCAGGG |
| | | CCGCCTGGAGCTGTGGGTGGACATGTTCCCCATGGACATGCCA |
| | | GCCCCTGGGACGCCTCTGGACATCTCACCTCGGAAGCCCAAGA |
| | | AGTACGAGCTGCGGGTCATCATCTGGAACACAGATGAGGTGGT |
| | | CTTGGAGGACGACGACTTCTTCACAGGGGAGAAGTCCAGTGAC |
| | | ATCTTCGTGAGGGGGTGGCTGAAGGGCCAGCAGGAGGACAAG |
| | | CAGGACACAGACGTCCACTACCACTCCCTCACTGGCGAGGGCA |
| | | ACTTCAACTGGCGCTACCTGTTCCCCTTCGACTACCTGGCGGCG |
| | | GAGGAGAAGATCGTCATCTCCAAGAAGGAGTCCATGTTCTCCTG |
| | | GGACGAGACCGAGTACAAGATCCCCGCGCGGCTCACCCTGCAG |
| | | ATCTGGGATGCGGACCACTTCTCCGCTGACGACTTCCTGGGGG |
| | | CCATCGAGCTGGACCTGAACCGGTTCCCGCGGGGCGCAAAGAC |
| | | AGCCAAGCAGTGCACCATGGAGATGGCCACCGGGGAGGTGGA |
| | | CGTGCCCCTCGTGTCCATCTTCAAGCAAAAGCGCGTCAAAGGCT |
| | | GGTGGCCCCTCCTGGCCCGCAATGAGAACGATGAGTTTGAGCT |
| | | CACGGGCAAGGTGGAGGCTGAGCTGCATTTACTGACAGCAGAG |
| | | GAGGCAGAGAAGAACCCAGTGGGCCTGGCCCGCAATGAACCTG |
| | | ACCCCCTAGAGAAACCCAACCGGCCCGACACGAGCTTCATCTG |
| | | GTTCCTGAACCCTCTCAAGTCGGCTCGCTACTTCTTGTGGCACA |
| | | CGTATCGCTGGCTGCTCCTCAAACTGTTGCTGCTCCTGCTGCTG |
| | | CTCCTCCTCCTCGCCCTGTTCCTCTACTCTGTGCCTGGCTACCT |
| | | GGTCAAGAAAATCCTCGGGGCCTGAGCCCAGTGGCCTCCTGGC |
| | | CGGCCCGACACGGCCTTCGTCTGGTTCCTCAACCCTCTCAAGTC |
| | | CATCAAGTACCTCATCTGCACCCGGTACAAGTGGCTCATCATCA |
| | | AGATCGTGCTGGCGCTGTTGGGGCTGCTCATGTTGGGGCTCTT |
| | | CCTCTACAGCCTCCCTGGCTACATGGTCAAAAAGCTCCTTGGGG |
| | | CATGAAGGCCGCCAGCTCCCGCCAGCCGCTCCCCAGCCCTGCC |
| | | GCATTTCCTTTCAGTGGCTTGGACTCTTTCCCATCTCCCCTGGG |
| | | GAGCCTGAGGAGCCCAGCGTCCACTCTTCATGCCTTGGGCCGA |
| | | GCCTGCCTCCTGCTTGCGGGGGCCGCCTGTCCTCACTGCCCCA |
| | | GGCTGCGGCTTGCCCAGTCCCGCCCCTCTGACCCCTGCCTGTG |
| | | GGCTGGGGAGCCTTGGATGGGGTGGGGACCTGGAATGGGTCT |
| | | CTCTTGCCCCACCTGGCTGAGGCGCCACCCTTCTTCAGGCCCA |
| | | GGCTCCAGAGGAAGACTCCTGAAACCCTCCCCAGGTCTTCCAA |
| | | GTACAGGATTGAAGCTTTAGTGAAATTAACCAAGGACCATGGGT |
| | | CAGTGCCCAGGGCTTTAAAAAGAATGAACGAGCAAAAGGTATCC |
| | | CCGCCGTGACCCCTGCAGATAGCACCGGTCTTTGATCCGCAGC |
| | | AGGGGCCAGACCCTGCCCACAAGTCCCAGCGCGGCTGCTTCTG |
| | | CCACTGCTGGGCTCCACTTGGCTCCTCTCACTTCCCAGGGGGT |
| | | CGCCTGTCCTGCCTGTGGGTTTCCATGGCTTCCCAGAGCTCCCT |
| | | CTGCCCCAGCCAGCGCCTCCAGGCCCAGCTGAGGAGCTGTGA |
| | | GAAGCAGCAGAGGGGACTCCCCATCCCGGGCACACCCTGTCCT |
| | | CCCACCCCTGCCCCCTTGCCCTTCCAGCCCTTTCAGCTGCAGCT |
| | | GGGAGCTGGCCCGTCAAGTGCTGCCCCTGCCTGTGTCTGGGTT |
| | | TCTGTTGGCTGTTTTTCTTTTCTTGAGTGGTGATTTTTCTCTAAAT |
| | | AAAAGAAGTCAAGCACTGAAAAAAAAAAAAAAAA |
| 14 | OTOF-205 transcript | ATCGGAGGGGGGTCGGGAGGAGGAGGAGGAGGCAGCGGCAG |
| | (NM_001287489.1), | AGAAGAGAGAGGCGTGTGAGCCGTGCTCCACCGGCTAGCTCCT |
| | human otoferlin | TCCCGCTGCTCCTGCCTGGCAGTGCCAGGCAGCCCACACCAGC |
| | transcript variant 5, | ATGGCCTTGCTCATCCACCTCAAGACAGTCTCGGAGCTGCGGG |
| | 6937 bp, encodes | GCAGGGGCGACCGGATCGCCAAAGTGACTTTCCGAGGGCAATC |
| | the protein of SEQ | CTTCTACTCTCGGGTCCTGGAGAACTGTGAGGATGTGGCTGACT |
| | ID NO: 5 | TTGATGAGACATTTCGGTGGCCGGTGGCCAGCAGCATCGACAG |
| | | AAATGAGATGCTGGAGATTCAGGTTTTCAACTACAGCAAAGTCTT |
| | | CAGCAACAAGCTCATCGGGACCTTCCGCATGGTGCTGCAGAAG |
| | | GTGGTAGAGGAGAGCCATGTGGAGGTGACTGACACGCTGATTG |
| | | ATGACAACAATGCTATCATCAAGACCAGCCTGTGCGTGGAGGTC |
| | | CGGTATCAGGCCACTGACGGCACAGTGGGCTCCTGGGACGATG |
| | | GGGACTTCCTGGGAGATGAGTCTCTTCAAGAGGAAGAGAAGGA |
| | | CAGCCAAGAGACGGATGGACTGCTCCCAGGCTCCCGGCCCAGC |
| | | TCCCGGCCCCCAGGAGAGAAGAGCTTCCGGAGAGCCGGGAGG |
| | | AGCGTGTTCTCCGCCATGAAGCTCGGCAAAAACCGGTCTCACAA |
| | | GGAGGAGCCCCAAAGACCAGATGAACCGGCGGTGCTGGAGAT |
| | | GGAAGACCTTGACCATCTGGCCATTCGGCTAGGAGATGGACTG |
| | | GATCCCGACTCGGTGTCTCTAGCCTCAGTCACAGCTCTCACCAC |
| | | TAATGTCTCCAACAAGCGATCTAAGCCAGACATTAAGATGGAGC |
| | | CAAGTGCTGGGCGGCCCATGGATTACCAGGTCAGCATCACGGT |
| | | GATCGAGGCCCGGCAGCTGGTGGGCTTGAACATGGACCCTGTG |
| | | GTGTGCGTGGAGGTGGGTGACGACAAGAAGTACACATCCATGA |
| | | AGGAGTCCACTAACTGCCCCTATTACAACGAGTACTTCGTCTTC |
| | | GACTTCCATGTCTCTCCGGATGTCATGTTTGACAAGATCATCAAG |
| | | ATTTCGGTGATTCACTCCAAGAACCTGCTGCGCAGTGGCACCCT |
| | | GGTGGGCTCCTTCAAAATGGACGTGGGAACCGTGTACTCGCAG |
| | | CCAGAGCACCAGTTCCATCACAAGTGGGCCATCCTGTCTGACCC |
| | | CGATGACATCTCCTCGGGGCTGAAGGGCTACGTGAAGTGTGAC |
| | | GTTGCCGTGGTGGGCAAAGGGGACAACATCAAGACGCCCCACA |
| | | AGGCCAATGAGACCGACGAAGATGACATTGAGGGGAACTTGCT |
| | | GCTCCCCGAGGGGGTGCCCCCCGAACGCCAGTGGGCCCGGTT |
| | | CTATGTGAAAATTTACCGAGCAGAGGGGCTGCCCCGTATGAACA |
| | | CAAGCCTCATGGCCAATGTAAAGAAGGCTTTCATCGGTGAAAAC |
| | | AAGGACCTCGTGGACCCCTACGTGCAAGTCTTCTTTGCTGGCCA |
| | | GAAGGGCAAGACTTCAGTGCAGAAGAGCAGCTATGAGCCCCTG |
| | | TGGAATGAGCAGGTCGTCTTTACAGACCTCTTCCCCCCACTCTG |
| | | CAAACGCATGAAGGTGCAGATCCGAGACTCGGACAAGGTCAAC |
| | | GACGTGGCCATCGGCACCCACTTCATTGACCTGCGCAAGATTTC |
| | | TAATGACGGAGACAAAGGCTTCCTGCCCACACTGGGCCCAGCC |
| | | TGGGTGAACATGTACGGCTCCACACGTAACTACACGCTGCTGGA |
| | | TGAGCATCAGGACCTGAACGAGGGCCTGGGGGAGGGTGTGTC |
| | | CTTCCGGGCCCGGCTCCTGCTGGGCCTGGCTGTGGAGATCGTA |
| | | GACACCTCCAACCCTGAGCTCACCAGCTCCACAGAGGTGCAGG |
| | | TGGAGCAGGCCACGCCCATCTCGGAGAGCTGTGCAGGTAAAAT |
| | | GGAAGAATTCTTTCTCTTTGGAGCCTTCCTGGAGGCCTCAATGA |
| | | TCGACCGGAGAAACGGAGACAAGCCCATCACCTTTGAGGTCAC |
| | | CATAGGCAACTATGGGAACGAAGTTGATGGCCTGTCCCGGCCC |
| | | CAGCGGCCTCGGCCCCGGAAGGAGCCGGGGGATGAGGAAGAA |
| | | GTAGACCTGATTCAGAACGCAAGTGATGACGAGGCCGGTGATG |
| | | CCGGGGACCTGGCCTCAGTCTCCTCCACTCCACCAATGCGGCC |
| | | CCAGGTCACCGACAGGAACTACTTCCATCTGCCCTACCTGGAGC |
| | | GAAAGCCCTGCATCTACATCAAGAGCTGGTGGCCGGACCAGCG |
| | | CCGCCGCCTCTACAATGCCAACATCATGGACCACATTGCCGACA |
| | | AGCTGGAAGAAGGCCTGAACGACATACAGGAGATGATCAAAAC |
| | | GGAGAAGTCCTACCCTGAGCGTCGCCTGCGGGGCGTCCTGGA |
| | | GGAGCTGAGCTGTGGCTGCTGCCGCTTCCTCTCCCTCGCTGAC |
| | | AAGGACCAGGGCCACTCATCCCGCACCAGGCTTGACCGGGAGC |
| | | GCCTCAAGTCCTGCATGAGGGAGCTGGAAAACATGGGGCAGCA |
| | | GGCCAGGATGCTGCGGGCCCAGGTGAAGCGGCACACGGTGCG |
| | | GGACAAGCTGAGGCTGTGCCAGAACTTCCTGCAGAAGCTGCGC |
| | | TTCCTGGCGGACGAGCCCCAGCACAGCATTCCCGACATCTTCAT |
| | | CTGGATGATGAGCAACAACAAGCGTGTCGCCTATGCCCGTGTG |
| | | CCCTCCAAGGACCTGCTCTTCTCCATCGTGGAGGAGGAGACTG |
| | | GCAAGGACTGCGCCAAGGTCAAGACGCTCTTCCTTAAGCTGCC |
| | | AGGGAAGCGGGGCTTCGGCTCGGCAGGCTGGACAGTGCAGGC |
| | | CAAGGTGGAGCTGTACCTGTGGCTGGGCCTCAGCAAACAGCGC |
| | | AAGGAGTTCCTGTGCGGCCTGCCCTGTGGCTTCCAGGAGGTCA |
| | | AGGCAGCCCAGGGCCTGGGCCTGCATGCCTTCCCACCCGTCAG |
| | | CCTGGTCTACACCAAGAAGCAGGCGTTCCAGCTCCGAGCGCAC |
| | | ATGTACCAGGCCCGCAGCCTCTTTGCCGCCGACAGCAGCGGAC |
| | | TCTCAGACCCCTTTGCCCGCGTCTTCTTCATCAATCAGAGTCAG |
| | | TGCACAGAGGTGCTGAATGAGACCCTGTGTCCCACCTGGGACC |
| | | AGATGCTGGTGTTCGACAACCTGGAGCTCTATGGTGAAGCTCAT |
| | | GAGCTGAGGGACGATCCGCCCATCATTGTCATTGAAATCTATGA |
| | | CCAGGATTCCATGGGCAAAGCTGACTTCATGGGCCGGACCTTC |
| | | GCCAAACCCCTGGTGAAGATGGCAGACGAGGCGTACTGCCCAC |
| | | CCCGCTTCCCACCTCAGCTCGAGTACTACCAGATCTACCGTGGC |
| | | AACGCCACAGCTGGAGACCTGCTGGCGGCCTTCGAGCTGCTGC |
| | | AGATTGGACCAGCAGGGAAGGCTGACCTGCCCCCCATCAATGG |
| | | CCCGGTGGACGTGGACCGAGGTCCCATCATGCCCGTGCCCATG |
| | | GGCATCCGGCCCGTGCTCAGCAAGTACCGAGTGGAGGTGCTGT |
| | | TCTGGGGCCTACGGGACCTAAAGCGGGTGAACCTGGCCCAGGT |
| | | GGACCGGCCACGGGTGGACATCGAGTGTGCAGGGAAGGGGGT |
| | | GCAGTCGTCCCTGATCCACAATTATAAGAAGAACCCCAACTTCA |
| | | ACACCCTCGTCAAGTGGTTTGAAGTGGACCTCCCAGAGAACGA |
| | | GCTGCTGCACCCGCCCTTGAACATCCGTGTGGTGGACTGCCGG |
| | | GCCTTCGGTCGCTACACACTGGTGGGCTCCCATGCCGTCAGCT |
| | | CCCTGCGACGCTTCATCTACCGGCCCCCAGACCGCTCGGCCCC |
| | | CAGCTGGAACACCACGGTCAGGCTTCTCCGGCGCTGCCGTGTG |
| | | CTGTGCAATGGGGGCTCCTCCTCTCACTCCACAGGGGAGGTTG |
| | | TGGTGACTATGGAGCCAGAGGTACCCATCAAGAAACTGGAGAC |
| | | CATGGTGAAGCTGGACGCGACTTCTGAAGCTGTTGTCAAGGTG |
| | | GATGTGGCTGAGGAGGAGAAGGAGAAGAAGAAGAAGAAGAAGG |
| | | GCACTGCGGAGGAGCCAGAGGAGGAGGAGCCAGACGAGAGCA |
| | | TGCTGGACTGGTGGTCCAAGTACTTTGCCTCCATTGACACCATG |
| | | AAGGAGCAACTTCGACAACAAGAGCCCTCTGGAATTGACTTGGA |
| | | GGAGAAGGAGGAAGTGGACAATACCGAGGGCCTGAAGGGGTC |
| | | AATGAAGGGCAAGGAGAAGGCAAGGGCTGCCAAAGAGGAGAAG |
| | | AAGAAGAAAACTCAGAGCTCTGGCTCTGGCCAGGGGTCCGAGG |
| | | CCCCCGAGAAGAAGAAACCCAAGATTGATGAGCTTAAGGTATAC |
| | | CCCAAAGAGCTGGAGTCCGAGTTTGATAACTTTGAGGACTGGCT |
| | | GCACACTTTCAACTTGCTTCGGGGCAAGACCGGGGATGATGAG |
| | | GATGGCTCCACCGAGGAGGAGCGCATTGTGGGACGCTTCAAGG |
| | | GCTCCCTCTGCGTGTACAAAGTGCCACTCCCAGAGGACGTGTC |
| | | CCGGGAAGCCGGCTACGACTCCACCTACGGCATGTTCCAGGGC |
| | | ATCCCGAGCAATGACCCCATCAATGTGCTGGTCCGAGTCTATGT |
| | | GGTCCGGGCCACGGACCTGCACCCTGCTGACATCAACGGCAAA |
| | | GCTGACCCCTACATCGCCATCCGGCTAGGCAAGACTGACATCC |
| | | GCGACAAGGAGAACTACATCTCCAAGCAGCTCAACCCTGTCTTT |
| | | GGGAAGTCCTTTGACATCGAGGCCTCCTTCCCCATGGAATCCAT |
| | | GCTGACGGTGGCTGTGTATGACTGGGACCTGGTGGGCACTGAT |
| | | GACCTCATTGGGGAAACCAAGATCGACCTGGAGAACCGCTTCTA |
| | | CAGCAAGCACCGCGCCACCTGCGGCATCGCCCAGACCTACTCC |
| | | ACACATGGCTACAATATCTGGCGGGACCCCATGAAGCCCAGCC |
| | | AGATCCTGACCCGCCTCTGCAAAGACGGCAAAGTGGACGGCCC |
| | | CCACTTTGGGCCCCCTGGGAGAGTGAAGGTGGCCAACCGCGTC |
| | | TTCACTGGGCCCTCTGAGATTGAGGACGAGAACGGTCAGAGGA |
| | | AGCCCACAGACGAGCATGTGGCGCTGTTGGCCCTGAGGCACTG |
| | | GGAGGACATCCCCCGCGCAGGCTGCCGCCTGGTGCCAGAGCA |
| | | TGTGGAGACGAGGCCGCTGCTCAACCCCGACAAGCCGGGCATC |
| | | GAGCAGGGCCGCCTGGAGCTGTGGGTGGACATGTTCCCCATGG |
| | | ACATGCCAGCCCCTGGGACGCCTCTGGACATCTCACCTCGGAA |
| | | GCCCAAGAAGTACGAGCTGCGGGTCATCATCTGGAACACAGAT |
| | | GAGGTGGTCTTGGAGGACGACGACTTCTTCACAGGGGAGAAGT |
| | | CCAGTGACATCTTCGTGAGGGGGTGGCTGAAGGGCCAGCAGGA |
| | | GGACAAGCAGGACACAGACGTCCACTACCACTCCCTCACTGGC |
| | | GAGGGCAACTTCAACTGGCGCTACCTGTTCCCCTTCGACTACCT |
| | | GGCGGCGGAGGAGAAGATCGTCATCTCCAAGAAGGAGTCCATG |
| | | TTCTCCTGGGACGAGACCGAGTACAAGATCCCCGCGCGGCTCA |
| | | CCCTGCAGATCTGGGATGCGGACCACTTCTCCGCTGACGACTT |
| | | CCTGGGGGCCATCGAGCTGGACCTGAACCGGTTCCCGCGGGG |
| | | CGCAAAGACAGCCAAGCAGTGCACCATGGAGATGGCCACCGGG |
| | | GAGGTGGACGTGCCCCTCGTGTCCATCTTCAAGCAAAAGCGCG |
| | | TCAAAGGCTGGTGGCCCCTCCTGGCCCGCAATGAGAACGATGA |
| | | GTTTGAGCTCACGGGCAAGGTGGAGGCTGAGCTGCATTTACTG |
| | | ACAGCAGAGGAGGCAGAGAAGAACCCAGTGGGCCTGGCCCGC |
| | | AATGAACCTGACCCCCTAGAGAAACCCAACCGGCCCGACACGG |
| | | CCTTCGTCTGGTTCCTCAACCCTCTCAAGTCCATCAAGTACCTCA |
| | | TCTGCACCCGGTACAAGTGGCTCATCATCAAGATCGTGCTGGCG |
| | | CTGTTGGGGCTGCTCATGTTGGGGCTCTTCCTCTACAGCCTCCC |
| | | TGGCTACATGGTCAAAAAGCTCCTTGGGGCATGAAGGCCGCCA |
| | | GCTCCCGCCAGCCGCTCCCCAGCCCTGCCGCATTTCCTTTCAG |
| | | TGGCTTGGACTCTTTCCCATCTCCCCTGGGGAGCCTGAGGAGC |
| | | CCAGCGTCCACTCTTCATGCCTTGGGCCGAGCCTGCCTCCTGC |
| | | TTGCGGGGGCCGCCTGTCCTCACTGCCCCAGGCTGCGGCTTGC |
| | | CCAGTCCCGCCCCTCTGACCCCTGCCTGTGGGCTGGGGAGCCT |
| | | TGGATGGGGTGGGGACCTGGAATGGGTCTCTCTTGCCCCACCT |
| | | GGCTGAGGCGCCACCCTTCTTCAGGCCCAGGCTCCAGAGGAAG |
| | | ACTCCTGAAACCCTCCCCAGGTCTTCCAAGTACAGGATTGAAGC |
| | | TTTAGTGAAATTAACCAAGGACCATGGGTCAGTGCCCAGGGCTT |
| | | TAAAAAGAATGAACGAGCAAAAGGTATCCCCGCCGTGACCCCTG |
| | | CAGATAGCACCGGTCTTTGATCCGCAGCAGGGGCCAGACCCTG |
| | | CCCACAAGTCCCAGCGCGGCTGCTTCTGCCACTGCTGGGCTCC |
| | | ACTTGGCTCCTCTCACTTCCCAGGGGGTCGCCTGTCCTGCCTGT |
| | | GGGTTTCCATGGCTTCCCAGAGCTCCCTCTGCCCCAGCCAGCG |
| | | CCTCCAGGCCCAGCTGAGGAGCTGTGAGAAGCAGCAGAGGGG |
| | | ACTCCCCATCCCGGGCACACCCTGTCCTCCCACCCCTGCCCCC |
| | | TTGCCCTTCCAGCCCTTTCAGCTGCAGCTGGGAGCTGGCCCGT |
| | | CAAGTGCTGCCCCTGCCTGTGTCTGGGTTTCTGTTGGCTGTTTT |
| | | TCTTTTCTTGAGTGGTGATTTTTCTCTAAATAAAAGAAGTCAAGC |
| | | ACTGAAAAAAAAAAAAAAAA |
| 15 | mOTOF-201_1 | TTGGTTGCCTTGGTCTCTGTGGGCAGCAGCAGGAGGAGGCGGC |
| | transcript | AGCAGCCAGAGAAGAGGGAGGCGTGTGAGCCACACTCCACCAG |
| | (NM_031875.2), | CGAGCTTCTTCCCGCTGCTCTGGAACTGCCCAGGCTCTCCCCA |
| | mouse otoferlin | CCAGCATGGCCCTGATTGTTCACCTCAAGACTGTCTCAGAGCTC |
| | transcript variant 2, | CGAGGCAAAGGTGACCGGATTGCCAAAGTCACTTTCCGAGGGC |
| | 7129 bp, encodes | AGTCTTTCTACTCCCGGGTCCTGGAGAACTGCGAGGGTGTGGC |
| | the protein of SEQ | TGACTTTGATGAGACGTTCCGGTGGCCAGTGGCCAGCAGCATC |
| | ID NO: 6 | GACCGGAATGAAGTGTTGGAGATTCAGATTTTCAACTACAGCAA |
| | | AGTCTTCAGCAACAAGCTGATAGGGACCTTCTGCATGGTGCTGC |
| | | AGAAAGTGGTGGAGGAGAATCGGGTAGAGGTGACCGACACGCT |
| | | GATGGATGACAGCAATGCTATCATCAAGACCAGCCTGAGCATGG |
| | | AGGTCCGGTATCAGGCCACAGATGGCACTGTGGGCCCCTGGGA |
| | | TGATGGAGACTTCCTGGGAGATGAATCCCTCCAGGAGGAGAAG |
| | | GACAGCCAGGAGACAGATGGGCTGCTACCTGGTTCCCGACCCA |
| | | GCACCCGGATATCTGGCGAGAAGAGCTTTCGCAGAGCGGGAAG |
| | | GAGTGTGTTCTCGGCCATGAAACTCGGCAAAACTCGGTCCCACA |
| | | AAGAGGAGCCCCAAAGACAAGATGAGCCAGCAGTGCTGGAGAT |
| | | GGAGGACCTGGACCACCTAGCCATTCAGCTGGGGGATGGGCTG |
| | | GATCCTGACTCCGTGTCTCTAGCCTCGGTCACCGCTCTCACCAG |
| | | CAATGTCTCCAACAAACGGTCTAAGCCAGATATTAAGATGGAGC |
| | | CCAGTGCTGGAAGGCCCATGGATTACCAGGTCAGCATCACAGT |
| | | GATTGAGGCTCGGCAGCTGGTGGGCTTGAACATGGACCCTGTG |
| | | GTGTGTGTGGAGGTGGGTGATGACAAGAAATACACGTCAATGAA |
| | | GGAGTCCACAAACTGCCCTTACTACAACGAGTACTTTGTCTTCG |
| | | ACTTCCATGTCTCTCCTGATGTCATGTTTGACAAGATCATCAAGA |
| | | TCTCGGTTATCCATTCTAAGAACCTGCTTCGGAGCGGCACCCTG |
| | | GTGGGTTCCTTCAAAATGGATGTGGGGACTGTGTATTCCCAGCC |
| | | TGAACACCAGTTCCATCACAAATGGGCCATCCTGTCAGACCCCG |
| | | ATGACATCTCTGCTGGGTTGAAGGGTTATGTAAAGTGTGATGTC |
| | | GCTGTGGTGGGCAAGGGAGACAACATCAAGACACCCCACAAGG |
| | | CCAACGAGACGGATGAGGACGACATTGAAGGGAACTTGCTGCT |
| | | CCCCGAGGGCGTGCCCCCCGAACGGCAGTGGGCACGGTTCTA |
| | | TGTGAAAATTTACCGAGCAGAGGGACTGCCCCGGATGAACACAA |
| | | GCCTCATGGCCAACGTGAAGAAGGCGTTCATCGGTGAGAACAA |
| | | GGACCTCGTCGACCCCTATGTGCAAGTCTTCTTTGCTGGACAAA |
| | | AGGGCAAAACATCAGTGCAGAAGAGCAGCTATGAGCCGCTATG |
| | | GAATGAGCAGGTCGTCTTCACAGACTTGTTCCCCCCACTCTGCA |
| | | AACGCATGAAGGTGCAGATCCGGGACTCTGACAAGGTCAATGAT |
| | | GTGGCCATCGGCACCCACTTCATCGACCTGCGCAAGATTTCCAA |
| | | CGATGGAGACAAAGGCTTCCTGCCTACCCTCGGTCCAGCCTGG |
| | | GTGAACATGTACGGCTCCACGCGCAACTACACACTGCTGGACG |
| | | AGCACCAGGACTTGAATGAAGGCCTGGGGGAGGGTGTGTCCTT |
| | | CCGGGCCCGCCTCATGTTGGGACTAGCTGTGGAGATCCTGGAC |
| | | ACCTCCAACCCAGAGCTCACCAGCTCCACGGAGGTGCAGGTGG |
| | | AGCAGGCCACGCCTGTCTCGGAGAGCTGCACAGGGAGAATGGA |
| | | AGAATTTTTTCTATTTGGAGCCTTCTTGGAAGCCTCAATGATTGA |
| | | CCGGAAAAATGGGGACAAGCCAATTACCTTTGAGGTGACCATAG |
| | | GAAACTACGGCAATGAAGTCGATGGTATGTCCCGGCCCCTGAG |
| | | GCCTCGGCCCCGGAAAGAGCCTGGGGATGAAGAAGAGGTAGA |
| | | CCTGATTCAGAACTCCAGTGACGATGAAGGTGACGAAGCCGGG |
| | | GACCTGGCCTCGGTGTCCTCCACCCCACCTATGCGGCCCCAGA |
| | | TCACGGACAGGAACTATTTCCACCTGCCCTACCTGGAGCGCAAG |
| | | CCCTGCATCTATATCAAGAGCTGGTGGCCTGACCAGAGGCGGC |
| | | GCCTCTACAATGCCAACATCATGGATCACATTGCTGACAAGCTG |
| | | GAAGAAGGCCTGAATGATGTACAGGAGATGATCAAAACGGAGAA |
| | | GTCCTACCCGGAGCGCCGCCTGCGGGGTGTGCTAGAGGAACTC |
| | | AGCTGTGGCTGCCACCGCTTCCTCTCCCTCTCGGACAAGGACC |
| | | AGGGCCGCTCGTCCCGCACCAGGCTGGATCGAGAGCGTCTTAA |
| | | GTCCTGTATGAGGGAGTTGGAGAGCATGGGACAGCAGGCCAAG |
| | | AGCCTGAGGGCTCAGGTGAAGCGGCACACTGTTCGGGACAAGC |
| | | TGAGGTCATGCCAGAACTTTCTGCAGAAGCTACGCTTCCTGGCG |
| | | GATGAGCCCCAGCACAGCATTCCTGATGTGTTCATTTGGATGAT |
| | | GAGCAACAACAAACGTATCGCCTATGCCCGCGTGCCTTCCAAAG |
| | | ACCTGCTCTTCTCCATCGTGGAGGAGGAACTGGGCAAGGACTG |
| | | CGCCAAAGTCAAGACCCTCTTCCTGAAGCTGCCAGGGAAGAGG |
| | | GGCTTCGGCTCGGCAGGCTGGACAGTACAGGCCAAGCTGGAG |
| | | CTCTACCTGTGGCTGGGCCTCAGCAAGCAGCGAAAGGACTTCC |
| | | TGTGTGGTCTGCCCTGTGGCTTCGAGGAGGTCAAGGCAGCCCA |
| | | AGGCCTGGGCCTGCATTCCTTTCCGCCCATCAGCCTAGTCTACA |
| | | CCAAGAAGCAAGCCTTCCAGCTCCGAGCACACATGTATCAGGC |
| | | CCGAAGCCTCTTTGCTGCTGACAGCAGTGGGCTCTCTGATCCCT |
| | | TTGCCCGTGTCTTCTTCATCAACCAGAGCCAATGCACTGAGGTT |
| | | CTAAACGAGACACTGTGTCCCACCTGGGACCAGATGCTGGTATT |
| | | TGACAACCTGGAGCTGTACGGTGAAGCTCACGAGTTACGAGAT |
| | | GATCCCCCCATCATTGTCATTGAAATCTACGACCAGGACAGCAT |
| | | GGGCAAAGCCGACTTCATGGGCCGGACCTTCGCCAAGCCCCTG |
| | | GTGAAGATGGCAGATGAAGCATACTGCCCACCTCGCTTCCCGC |
| | | CGCAGCTTGAGTACTACCAGATCTACCGAGGCAGTGCCACTGC |
| | | CGGAGACCTACTGGCTGCCTTCGAGCTGCTGCAGATTGGGCCA |
| | | TCAGGGAAGGCTGACCTGCCACCCATCAATGGCCCAGTGGACA |
| | | TGGACAGAGGGCCCATCATGCCTGTGCCCGTGGGAATCCGGCC |
| | | AGTGCTCAGCAAGTACCGAGTGGAGGTGCTGTTCTGGGGCCTG |
| | | AGGGACCTAAAGAGGGTGAACCTGGCCCAGGTGGACCGACCAC |
| | | GGGTGGACATCGAGTGTGCAGGAAAGGGGGTACAATCCTCCCT |
| | | GATTCACAATTATAAGAAGAACCCCAACTTCAACACGCTGGTCAA |
| | | GTGGTTTGAAGTGGACCTCCCGGAGAATGAGCTCCTGCACCCA |
| | | CCCTTGAACATCCGAGTGGTAGATTGCCGGGCCTTTGGACGATA |
| | | CACCCTGGTGGGTTCCCACGCAGTCAGCTCACTGAGGCGCTTC |
| | | ATCTACCGACCTCCAGACCGCTCAGCCCCCAACTGGAACACCA |
| | | CAGTCAGGCTGCTCCGGGGCTGCCACAGGCTGCGCAATGGGG |
| | | GCCCCTCTTCTCGCCCCACAGGGGAGGTTGTAGTAAGCATGGA |
| | | GCCTGAGGAGCCAGTTAAGAAGCTGGAGACCATGGTGAAACTG |
| | | GATGCGACTTCTGATGCTGTGGTCAAGGTGGATGTGGCTGAAG |
| | | ATGAGAAGGAAAGGAAGAAGAAGAAAAAGAAAGGCCCGTCAGA |
| | | GGAGCCAGAGGAGGAAGAGCCCGATGAGAGCATGCTGGATTG |
| | | GTGGTCCAAGTACTTCGCCTCCATCGACACAATGAAGGAGCAAC |
| | | TTCGACAACATGAGACCTCTGGAACTGACTTGGAAGAGAAG GAA |
| | | GAGATGGAAAGCGCTGAGGGCCTGAAGGGACCAATGAAGAGCA |
| | | AGGAGAAGTCCAGAGCTGCAAAGGAGGAGAAAAAGAAGAAAAA |
| | | CCAGAGCCCTGGCCCTGGCCAGGGATCGGAGGCTCCTGAGAA |
| | | GAAGAAAGCCAAGATCGATGAGCTTAAGGTGTACCCCAAGGAG |
| | | CTGGAATCGGAGTTTGACAGCTTTGAGGACTGGCTGCACACCTT |
| | | CAACCTGTTGAGGGGCAAGACGGGAGATGATGAGGATGGCTCC |
| | | ACAGAGGAGGAGCGCATAGTAGGCCGATTCAAGGGCTCCCTCT |
| | | GTGTGTACAAAGTGCCACTCCCAGAAGATGTATCTCGAGAAGCT |
| | | GGCTATGATCCCACCTATGGAATGTTCCAGGGCATCCCAAGCAA |
| | | TGACCCCATCAATGTGCTGGTCCGAATCTATGTGGTCCGGGCCA |
| | | CAGACCTGCACCCGGCCGACATCAATGGCAAAGCTGACCCCTA |
| | | TATTGCCATCAAGTTAGGCAAGACCGACATCCGAGACAAGGAGA |
| | | ACTACATCTCCAAGCAGCTCAACCCTGTGTTTGGGAAGTCCTTT |
| | | GACATTGAGGCCTCCTTCCCCATGGAGTCCATGTTGACAGTGGC |
| | | CGTGTACGACTGGGATCTGGTGGGCACTGATGACCTCATCGGA |
| | | GAAACCAAGATTGACCTGGAAAACCGCTTCTACAGCAAGCATCG |
| | | CGCCACCTGCGGCATCGCACAGACCTATTCCATACATGGCTACA |
| | | ATATCTGGAGGGACCCCATGAAGCCCAGCCAGATCCTGACACG |
| | | CCTCTGTAAAGAGGGCAAAGTGGACGGCCCCCACTTTGGTCCC |
| | | CATGGGAGAGTGAGGGTTGCCAACCGTGTCTTCACGGGGCCTT |
| | | CAGAAATAGAGGATGAGAATGGTCAGAGGAAGCCCACAGATGA |
| | | GCACGTGGCACTGTCTGCTCTGAGACACTGGGAGGACATCCCC |
| | | CGGGTGGGCTGCCGCCTTGTGCCGGAACACGTGGAGACCAGG |
| | | CCGCTGCTCAACCCTGACAAGCCAGGCATTGAGCAGGGCCGCC |
| | | TGGAGCTGTGGGTGGACATGTTCCCCATGGACATGCCAGCCCC |
| | | TGGGACACCTCTGGATATATCCCCCAGGAAACCCAAGAAGTACG |
| | | AGCTGCGGGTCATCGTGTGGAACACAGACGAGGTGGTCCTGGA |
| | | AGACGATGATTTCTTCACGGGAGAGAAGTCCAGTGACATTTTTG |
| | | TGAGGGGGTGGCTGAAGGGCCAGCAGGAGGACAAACAGGACA |
| | | CAGATGTCCACTATCACTCCCTCACGGGGGAGGGCAACTTCAAC |
| | | TGGAGATACCTCTTCCCCTTCGACTACCTAGCGGCCGAAGAGAA |
| | | GATCGTTATGTCCAAAAAGGAGTCTATGTTCTCCTGGGATGAGA |
| | | CGGAGTACAAGATCCCTGCGCGGCTCACCCTGCAGATCTGGGA |
| | | CGCTGACCACTTCTCGGCTGACGACTTCCTGGGGGCTATCGAG |
| | | CTGGACCTGAACCGGTTCCCGAGGGGCGCTAAGACAGCCAAGC |
| | | AGTGCACCATGGAGATGGCCACCGGGGAGGTGGACGTACCCCT |
| | | GGTTTCCATCTTTAAACAGAAACGTGTCAAAGGCTGGTGGCCCC |
| | | TCCTGGCCCGCAATGAGAATGATGAGTTTGAGCTCACAGGCAAA |
| | | GTGGAGGCGGAGCTACACCTACTCACGGCAGAGGAGGCAGAG |
| | | AAGAACCCTGTGGGCCTGGCTCGCAATGAACCTGATCCCCTAG |
| | | AAAAACCCAATCGGCCGGACACAAGCTTCATCTGGTTCTTGAAC |
| | | CCTCTCAAGTCTGCCCGCTACTTCCTGTGGCATACCTACCGCTG |
| | | GCTACTCCTCAAATTCCTGCTGCTCTTCCTCCTGCTGCTGCTCTT |
| | | CGCCCTGTTTCTCTACTCTCTGCCTGGCTACCTGGCCAAGAAGA |
| | | TCCTTGGGGCCTGAGCCCTGCAGTCGCCTAGGCCTGCCGGCCT |
| | | GACACGGCATTCGTCTGGTTCCTGAACCCACTCAAATCTATCAA |
| | | GTACCTCATCTGCACCCGGTACAAGTGGCTGATCATCAAGATCG |
| | | TGCTGGCGCTGCTGGGGCTGCTCATGCTGGCCCTCTTCCTTTAC |
| | | AGCCTCCCAGGCTACATGGTCAAGAAGCTCCTAGGGGCCTGAA |
| | | GTGTGCCCCACCCCAGCCCGCTCCAGCATCCCTCCAGGGGCTG |
| | | CTGCGTATTTTGCCTTCCCTCACCTGGACTCTCTCCCAACTCCCT |
| | | GAGGAGCCCTCCCACGCCTGCCAGCCTTGAGCAAGACACCTGC |
| | | TTGCTGGACTTCATCCCCACCCCACACCCAAACTGTTGCTTGCC |
| | | TGATCTTGTCCCAGGCCTGCCTGGGGTTTGGGGCACAGTTGGC |
| | | CTCCAAAACCAGATACCCTCTTGTCTAAAGTACCAGGTTCCTCTG |
| | | CCCAACCCCAAGAGTGGTAGTGGCCCAACCCTCCCTGTGCTTTC |
| | | CAAATCTTGTCTTAAGGCACCAGTGAAATTAACCAAGAAACGCG |
| | | GAGCAATGCCCAAGGCTCTGATGAGTAGGAACACGTGGAAAGC |
| | | ACCAGGAATGCCAGCAGAGGCGAGGCGGCACACCTCTCTGCAG |
| | | AGCATCCAGGCCGAGCGGCGGGCAGCGGCCAGCTGCTTCTGC |
| | | GCATGCTCTCCTCTTGGCTCTGCTTCTTTCTCACAGTCACAGTCA |
| | | CTTCACAGCTTAGCCTTGGGCTTCCCATCACTTCCAGGGGTGCC |
| | | TCTGCCTTGGCCAGTGTGTGTCAGCTAGTACACAAGCTCCAAGT |
| | | GTGAATCAGGTGTACTGGCCGTCCTGAAGACTGACTGCCCTGTC |
| | | CTTCCTGCCGACAGCCACACCCGAGTGTACACTTAAAGCGGTG |
| | | CCCTTCTGCCTCTGTGGGCCTGCTGGCTGCTGTTCCTTTCTTGA |
| | | GTGTGATTTTTTTTTTCTCTCCCTCAATAAAATAAATCAAACTCTG |
| | | AGAC |
| 16 | mOTOF-201 2 | TTGGTTGCCTTGGTCTCTGTGGGCAGCAGCAGGAGGAGGCGGC |
| | transcript | AGCAGCCAGAGAAGAGGGAGGCGTGTGAGCCACACTCCACCAG |
| | (NM_001286421.1), | CGAGCTTCTTCCCGCTGCTCTGGAACTGCCCAGGCTCTCCCCA |
| | mouse otoferlin | CCAGCATGGCCCTGATTGTTCACCTCAAGACTGTCTCAGAGCTC |
| | transcript variant 3, | CGAGGCAAAGGTGACCGGATTGCCAAAGTCACTTTCCGAGGGC |
| | 7129 bp, encodes | AGTCTTTCTACTCCCGGGTCCTGGAGAACTGCGAGGGTGTGGC |
| | the protein of SEQ | TGACTTTGATGAGACGTTCCGGTGGCCAGTGGCCAGCAGCATC |
| | ID NO: 7 | GACCGGAATGAAGTGTTGGAGATTCAGATTTTCAACTACAGCAA |
| | | AGTCTTCAGCAACAAGCTGATAGGGACCTTCTGCATGGTGCTGC |
| | | AGAAAGTGGTGGAGGAGAATCGGGTAGAGGTGACCGACACGCT |
| | | GATGGATGACAGCAATGCTATCATCAAGACCAGCCTGAGCATGG |
| | | AGGTCCGGTATCAGGCCACAGATGGCACTGTGGGCCCCTGGGA |
| | | TGATGGAGACTTCCTGGGAGATGAATCCCTCCAGGAGGAGAAG |
| | | GACAGCCAGGAGACAGATGGGCTGCTACCTGGTTCCCGACCCA |
| | | GCACCCGGATATCTGGCGAGAAGAGCTTTCGCAGAGCGGGAAG |
| | | GAGTGTGTTCTCGGCCATGAAACTCGGCAAAACTCGGTCCCACA |
| | | AAGAGGAGCCCCAAAGACAAGATGAGCCAGCAGTGCTGGAGAT |
| | | GGAGGACCTGGACCACCTAGCCATTCAGCTGGGGGATGGGCTG |
| | | GATCCTGACTCCGTGTCTCTAGCCTCGGTCACCGCTCTCACCAG |
| | | CAATGTCTCCAACAAACGGTCTAAGCCAGATATTAAGATGGAGC |
| | | CCAGTGCTGGAAGGCCCATGGATTACCAGGTCAGCATCACAGT |
| | | GATTGAGGCTCGGCAGCTGGTGGGCTTGAACATGGACCCTGTG |
| | | GTGTGTGTGGAGGTGGGTGATGACAAGAAATACACGTCAATGAA |
| | | GGAGTCCACAAACTGCCCTTACTACAACGAGTACTTTGTCTTCG |
| | | ACTTCCATGTCTCTCCTGATGTCATGTTTGACAAGATCATCAAGA |
| | | TCTCGGTTATCCATTCTAAGAACCTGCTTCGGAGCGGCACCCTG |
| | | GTGGGTTCCTTCAAAATGGATGTGGGGACTGTGTATTCCCAGCC |
| | | TGAACACCAGTTCCATCACAAATGGGCCATCCTGTCAGACCCCG |
| | | ATGACATCTCTGCTGGGTTGAAGGGTTATGTAAAGTGTGATGTC |
| | | GCTGTGGTGGGCAAGGGAGACAACATCAAGACACCCCACAAGG |
| | | CCAACGAGACGGATGAGGACGACATTGAAGGGAACTTGCTGCT |
| | | CCCCGAGGGCGTGCCCCCCGAACGGCAGTGGGCACGGTTCTA |
| | | TGTGAAAATTTACCGAGCAGAGGGACTGCCCCGGATGAACACAA |
| | | GCCTCATGGCCAACGTGAAGAAGGCGTTCATCGGTGAGAACAA |
| | | GGACCTCGTCGACCCCTATGTGCAAGTCTTCTTTGCTGGACAAA |
| | | AGGGCAAAACATCAGTGCAGAAGAGCAGCTATGAGCCGCTATG |
| | | GAATGAGCAGGTCGTCTTCACAGACTTGTTCCCCCCACTCTGCA |
| | | AACGCATGAAGGTGCAGATCCGGGACTCTGACAAGGTCAATGAT |
| | | GTGGCCATCGGCACCCACTTCATCGACCTGCGCAAGATTTCCAA |
| | | CGATGGAGACAAAGGCTTCCTGCCTACCCTCGGTCCAGCCTGG |
| | | GTGAACATGTACGGCTCCACGCGCAACTACACACTGCTGGACG |
| | | AGCACCAGGACTTGAATGAAGGCCTGGGGGAGGGTGTGTCCTT |
| | | CCGGGCCCGCCTCATGTTGGGACTAGCTGTGGAGATCCTGGAC |
| | | ACCTCCAACCCAGAGCTCACCAGCTCCACGGAGGTGCAGGTGG |
| | | AGCAGGCCACGCCTGTCTCGGAGAGCTGCACAGGGAGAATGGA |
| | | AGAATTTTTTCTATTTGGAGCCTTCTTGGAAGCCTCAATGATTGA |
| | | CCGGAAAAATGGGGACAAGCCAATTACCTTTGAGGTGACCATAG |
| | | GAAACTACGGCAATGAAGTCGATGGTATGTCCCGGCCCCTGAG |
| | | GCCTCGGCCCCGGAAAGAGCCTGGGGATGAAGAAGAGGTAGA |
| | | CCTGATTCAGAACTCCAGTGACGATGAAGGTGACGAAGCCGGG |
| | | GACCTGGCCTCGGTGTCCTCCACCCCACCTATGCGGCCCCAGA |
| | | TCACGGACAGGAACTATTTCCACCTGCCCTACCTGGAGCGCAAG |
| | | CCCTGCATCTATATCAAGAGCTGGTGGCCTGACCAGAGGCGGC |
| | | GCCTCTACAATGCCAACATCATGGATCACATTGCTGACAAGCTG |
| | | GAAGAAGGCCTGAATGATGTACAGGAGATGATCAAAACGGAGAA |
| | | GTCCTACCCGGAGCGCCGCCTGCGGGGTGTGCTAGAGGAACTC |
| | | AGCTGTGGCTGCCACCGCTTCCTCTCCCTCTCGGACAAGGACC |
| | | AGGGCCGCTCGTCCCGCACCAGGCTGGATCGAGAGCGTCTTAA |
| | | GTCCTGTATGAGGGAGTTGGAGAGCATGGGACAGCAGGCCAAG |
| | | AGCCTGAGGGCTCAGGTGAAGCGGCACACTGTTCGGGACAAGC |
| | | TGAGGTCATGCCAGAACTTTCTGCAGAAGCTACGCTTCCTGGCG |
| | | GATGAGCCCCAGCACAGCATTCCTGATGTGTTCATTTGGATGAT |
| | | GAGCAACAACAAACGTATCGCCTATGCCCGCGTGCCTTCCAAAG |
| | | ACCTGCTCTTCTCCATCGTGGAGGAGGAACTGGGCAAGGACTG |
| | | CGCCAAAGTCAAGACCCTCTTCCTGAAGCTGCCAGGGAAGAGG |
| | | GGCTTCGGCTCGGCAGGCTGGACAGTACAGGCCAAGCTGGAG |
| | | CTCTACCTGTGGCTGGGCCTCAGCAAGCAGCGAAAGGACTTCC |
| | | TGTGTGGTCTGCCCTGTGGCTTCGAGGAGGTCAAGGCAGCCCA |
| | | AGGCCTGGGCCTGCATTCCTTTCCGCCCATCAGCCTAGTCTACA |
| | | CCAAGAAGCAAGCCTTCCAGCTCCGAGCACACATGTATCAGGC |
| | | CCGAAGCCTCTTTGCTGCTGACAGCAGTGGGCTCTCTGATCCCT |
| | | TTGCCCGTGTCTTCTTCATCAACCAGAGCCAATGCACTGAGGTT |
| | | CTAAACGAGACACTGTGTCCCACCTGGGACCAGATGCTGGTATT |
| | | TGACAACCTGGAGCTGTACGGTGAAGCTCACGAGTTACGAGAT |
| | | GATCCCCCCATCATTGTCATTGAAATCTACGACCAGGACAGCAT |
| | | GGGCAAAGCCGACTTCATGGGCCGGACCTTCGCCAAGCCCCTG |
| | | GTGAAGATGGCAGATGAAGCATACTGCCCACCTCGCTTCCCGC |
| | | CGCAGCTTGAGTACTACCAGATCTACCGAGGCAGTGCCACTGC |
| | | CGGAGACCTACTGGCTGCCTTCGAGCTGCTGCAGATTGGGCCA |
| | | TCAGGGAAGGCTGACCTGCCACCCATCAATGGCCCAGTGGACA |
| | | TGGACAGAGGGCCCATCATGCCTGTGCCCGTGGGAATCCGGCC |
| | | AGTGCTCAGCAAGTACCGAGTGGAGGTGCTGTTCTGGGGCCTG |
| | | AGGGACCTAAAGAGGGTGAACCTGGCCCAGGTGGACCGACCAC |
| | | GGGTGGACATCGAGTGTGCAGGAAAGGGGGTACAATCCTCCCT |
| | | GATTCACAATTATAAGAAGAACCCCAACTTCAACACGCTGGTCAA |
| | | GTGGTTTGAAGTGGACCTCCCGGAGAATGAGCTCCTGCACCCA |
| | | CCCTTGAACATCCGAGTGGTAGATTGCCGGGCCTTTGGACGATA |
| | | CACCCTGGTGGGTTCCCACGCAGTCAGCTCACTGAGGCGCTTC |
| | | ATCTACCGACCTCCAGACCGCTCAGCCCCCAACTGGAACACCA |
| | | CAGGGGAGGTTGTAGTAAGCATGGAGCCTGAGGAGCCAGTTAA |
| | | GAAGCTGGAGACCATGGTGAAACTGGATGCGACTTCTGATGCT |
| | | GTGGTCAAGGTGGATGTGGCTGAAGATGAGAAGGAAAGGAAGA |
| | | AGAAGAAAAAGAAAGGCCCGTCAGAGGAGCCAGAGGAGGAAGA |
| | | GCCCGATGAGAGCATGCTGGATTGGTGGTCCAAGTACTTCGCC |
| | | TCCATCGACACAATGAAGGAGCAACTTCGACAACATGAGACCTC |
| | | TGGAACTGACTTGGAAGAGAAGGAAGAGATGGAAAGCGCTGAG |
| | | GGCCTGAAGGGACCAATGAAGAGCAAGGAGAAGTCCAGAGCTG |
| | | CAAAGGAGGAGAAAAAGAAGAAAAACCAGAGCCCTGGCCCTGG |
| | | CCAGGGATCGGAGGCTCCTGAGAAGAAGAAAGCCAAGATCGAT |
| | | GAGCTTAAGGTGTACCCCAAGGAGCTGGAATCGGAGTTTGACA |
| | | GCTTTGAGGACTGGCTGCACACCTTCAACCTGTTGAGGGGCAA |
| | | GACGGGAGATGATGAGGATGGCTCCACAGAGGAGGAGCGCATA |
| | | GTAGGCCGATTCAAGGGCTCCCTCTGTGTGTACAAAGTGCCACT |
| | | CCCAGAAGATGTATCTCGAGAAGCTGGCTATGATCCCACCTATG |
| | | GAATGTTCCAGGGCATCCCAAGCAATGACCCCATCAATGTGCTG |
| | | GTCCGAATCTATGTGGTCCGGGCCACAGACCTGCACCCGGCCG |
| | | ACATCAATGGCAAAGCTGACCCCTATATTGCCATCAAGTTAGGC |
| | | AAGACCGACATCCGAGACAAGGAGAACTACATCTCCAAGCAGCT |
| | | CAACCCTGTGTTTGGGAAGTCCTTTGACATTGAGGCCTCCTTCC |
| | | CCATGGAGTCCATGTTGACAGTGGCCGTGTACGACTGGGATCT |
| | | GGTGGGCACTGATGACCTCATCGGAGAAACCAAGATTGACCTG |
| | | GAAAACCGCTTCTACAGCAAGCATCGCGCCACCTGCGGCATCG |
| | | CACAGACCTATTCCATACATGGCTACAATATCTGGAGGGACCCC |
| | | ATGAAGCCCAGCCAGATCCTGACACGCCTCTGTAAAGAGGGCA |
| | | AAGTGGACGGCCCCCACTTTGGTCCCCATGGGAGAGTGAGGGT |
| | | TGCCAACCGTGTCTTCACGGGGCCTTCAGAAATAGAGGATGAGA |
| | | ATGGTCAGAGGAAGCCCACAGATGAGCACGTGGCACTGTCTGC |
| | | TCTGAGACACTGGGAGGACATCCCCCGGGTGGGCTGCCGCCTT |
| | | GTGCCGGAACACGTGGAGACCAGGCCGCTGCTCAACCCTGACA |
| | | AGCCAGGCATTGAGCAGGGCCGCCTGGAGCTGTGGGTGGACAT |
| | | GTTCCCCATGGACATGCCAGCCCCTGGGACACCTCTGGATATAT |
| | | CCCCCAGGAAACCCAAGAAGTACGAGCTGCGGGTCATCGTGTG |
| | | GAACACAGACGAGGTGGTCCTGGAAGACGATGATTTCTTCACG |
| | | GGAGAGAAGTCCAGTGACATTTTTGTGAGGGGGTGGCTGAAGG |
| | | GCCAGCAGGAGGACAAACAGGACACAGATGTCCACTATCACTC |
| | | CCTCACGGGGGAGGGCAACTTCAACTGGAGATACCTCTTCCCC |
| | | TTCGACTACCTAGCGGCCGAAGAGAAGATCGTTATGTCCAAAAA |
| | | GGAGTCTATGTTCTCCTGGGATGAGACGGAGTACAAGATCCCTG |
| | | CGCGGCTCACCCTGCAGATCTGGGACGCTGACCACTTCTCGGC |
| | | TGACGACTTCCTGGGGGCTATCGAGCTGGACCTGAACCGGTTC |
| | | CCGAGGGGCGCTAAGACAGCCAAGCAGTGCACCATGGAGATGG |
| | | CCACCGGGGAGGTGGACGTACCCCTGGTTTCCATCTTTAAACAG |
| | | AAACGTGTCAAAGGCTGGTGGCCCCTCCTGGCCCGCAATGAGA |
| | | ATGATGAGTTTGAGCTCACAGGCAAAGTGGAGGCGGAGCTACA |
| | | CCTACTCACGGCAGAGGAGGCAGAGAAGAACCCTGTGGGCCTG |
| | | GCTCGCAATGAACCTGATCCCCTAGAAAAACCCAATCGGCCGGA |
| | | CACAAGCTTCATCTGGTTCTTGAACCCTCTCAAGTCTGCCCGCT |
| | | ACTTCCTGTGGCATACCTACCGCTGGCTACTCCTCAAATTCCTG |
| | | CTGCTCTTCCTCCTGCTGCTGCTCTTCGCCCTGTTTCTCTACTCT |
| | | CTGCCTGGCTACCTGGCCAAGAAGATCCTTGGGGCCTGAGCCC |
| | | TGCAGTCGCCTAGGCCTGCCGGCCTGACACGGCATTCGTCTGG |
| | | TTCCTGAACCCACTCAAATCTATCAAGTACCTCATCTGCACCCG |
| | | GTACAAGTGGCTGATCATCAAGATCGTGCTGGCGCTGCTGGGG |
| | | CTGCTCATGCTGGCCCTCTTCCTTTACAGCCTCCCAGGCTACAT |
| | | GGTCAAGAAGCTCCTAGGGGCCTGAAGTGTGCCCCACCCCAGC |
| | | CCGCTCCAGCATCCCTCCAGGGGCTGCTGCGTATTTTGCCTTCC |
| | | CTCACCTGGACTCTCTCCCAACTCCCTGAGGAGCCCTCCCACG |
| | | CCTGCCAGCCTTGAGCAAGACACCTGCTTGCTGGACTTCATCCC |
| | | CACCCCACACCCAAACTGTTGCTTGCCTGATCTTGTCCCAGGCC |
| | | TGCCTGGGGTTTGGGGCACAGTTGGCCTCCAAAACCAGATACC |
| | | CTCTTGTCTAAAGTACCAGGTTCCTCTGCCCAACCCCAAGAGTG |
| | | GTAGTGGCCCAACCCTCCCTGTGCTTTCCAAATCTTGTCTTAAG |
| | | GCACCAGTGAAATTAACCAAGAAACGCGGAGCAATGCCCAAGG |
| | | CTCTGATGAGTAGGAACACGTGGAAAGCACCAGGAATGCCAGC |
| | | AGAGGCGAGGCGGCACACCTCTCTGCAGAGCATCCAGGCCGA |
| | | GCGGCGGGCAGCGGCCAGCTGCTTCTGCGCATGCTCTCCTCTT |
| | | GGCTCTGCTTCTTTCTCACAGTCACAGTCACTTCACAGCTTAGC |
| | | CTTGGGCTTCCCATCACTTCCAGGGGTGCCTCTGCCTTGGCCA |
| | | GTGTGTGTCAGCTAGTACACAAGCTCCAAGTGTGAATCAGGTGT |
| | | ACTGGCCGTCCTGAAGACTGACTGCCCTGTCCTTCCTGCCGACA |
| | | GCCACACCCGAGTGTACACTTAAAGCGGTGCCCTTCTGCCTCTG |
| | | TGGGCCTGCTGGCTGCTGTTCCTTTCTTGAGTGTGATTTTTTTTT |
| | | TCTCTCCCTCAATAAAATAAATCAAACTCTGAGAC |
| 17 | mOTOF-202_1 | TTGGTTGCCTTGGTCTCTGTGGGCAGCAGCAGGAGGAGGCGGC |
| | transcript | AGCAGCCAGAGAAGAGGGAGGCGTGTGAGCCACACTCCACCAG |
| | (NM_001100395.1), | CGAGCTTCTTCCCGCTGCTCTGGAACTGCCCAGGCTCTCCCCA |
| | mouse otoferlin | CCAGCATGGCCCTGATTGTTCACCTCAAGACTGTCTCAGAGCTC |
| | transcript variant 1, | CGAGGCAAAGGTGACCGGATTGCCAAAGTCACTTTCCGAGGGC |
| | 6881 bp, encodes | AGTCTTTCTACTCCCGGGTCCTGGAGAACTGCGAGGGTGTGGC |
| | the protein of SEQ | TGACTTTGATGAGACGTTCCGGTGGCCAGTGGCCAGCAGCATC |
| | ID NO: 8 | GACCGGAATGAAGTGTTGGAGATTCAGATTTTCAACTACAGCAA |
| | | AGTCTTCAGCAACAAGCTGATAGGGACCTTCTGCATGGTGCTGC |
| | | AGAAAGTGGTGGAGGAGAATCGGGTAGAGGTGACCGACACGCT |
| | | GATGGATGACAGCAATGCTATCATCAAGACCAGCCTGAGCATGG |
| | | AGGTCCGGTATCAGGCCACAGATGGCACTGTGGGCCCCTGGGA |
| | | TGATGGAGACTTCCTGGGAGATGAATCCCTCCAGGAGGAGAAG |
| | | GACAGCCAGGAGACAGATGGGCTGCTACCTGGTTCCCGACCCA |
| | | GCACCCGGATATCTGGCGAGAAGAGCTTTCGCAGCAAAGGCAG |
| | | AGAGAAGACCAAGGGAGGCAGAGATGGCGAGCACAAAGCGGG |
| | | AAGGAGTGTGTTCTCGGCCATGAAACTCGGCAAAACTCGGTCCC |
| | | ACAAAGAGGAGCCCCAAAGACAAGATGAGCCAGCAGTGCTGGA |
| | | GATGGAGGACCTGGACCACCTAGCCATTCAGCTGGGGGATGGG |
| | | CTGGATCCTGACTCCGTGTCTCTAGCCTCGGTCACCGCTCTCAC |
| | | CAGCAATGTCTCCAACAAACGGTCTAAGCCAGATATTAAGATGG |
| | | AGCCCAGTGCTGGAAGGCCCATGGATTACCAGGTCAGCATCAC |
| | | AGTGATTGAGGCTCGGCAGCTGGTGGGCTTGAACATGGACCCT |
| | | GTGGTGTGTGTGGAGGTGGGTGATGACAAGAAATACACGTCAAT |
| | | GAAGGAGTCCACAAACTGCCCTTACTACAACGAGTACTTTGTCT |
| | | TCGACTTCCATGTCTCTCCTGATGTCATGTTTGACAAGATCATCA |
| | | AGATCTCGGTTATCCATTCTAAGAACCTGCTTCGGAGCGGCACC |
| | | CTGGTGGGTTCCTTCAAAATGGATGTGGGGACTGTGTATTCCCA |
| | | GCCTGAACACCAGTTCCATCACAAATGGGCCATCCTGTCAGACC |
| | | CCGATGACATCTCTGCTGGGTTGAAGGGTTATGTAAAGTGTGAT |
| | | GTCGCTGTGGTGGGCAAGGGAGACAACATCAAGACACCCCACA |
| | | AGGCCAACGAGACGGATGAGGACGACATTGAAGGGAACTTGCT |
| | | GCTCCCCGAGGGCGTGCCCCCCGAACGGCAGTGGGCACGGTT |
| | | CTATGTGAAAATTTACCGAGCAGAGGGACTGCCCCGGATGAACA |
| | | CAAGCCTCATGGCCAACGTGAAGAAGGCGTTCATCGGTGAGAA |
| | | CAAGGACCTCGTCGACCCCTATGTGCAAGTCTTCTTTGCTGGAC |
| | | AAAAGGGCAAAACATCAGTGCAGAAGAGCAGCTATGAGCCGCT |
| | | ATGGAATGAGCAGGTCGTCTTCACAGACTTGTTCCCCCCACTCT |
| | | GCAAACGCATGAAGGTGCAGATCCGGGACTCTGACAAGGTCAA |
| | | TGATGTGGCCATCGGCACCCACTTCATCGACCTGCGCAAGATTT |
| | | CCAACGATGGAGACAAAGGCTTCCTGCCTACCCTCGGTCCAGC |
| | | CTGGGTGAACATGTACGGCTCCACGCGCAACTACACACTGCTG |
| | | GACGAGCACCAGGACTTGAATGAAGGCCTGGGGGAGGGTGTGT |
| | | CCTTCCGGGCCCGCCTCATGTTGGGACTAGCTGTGGAGATCCT |
| | | GGACACCTCCAACCCAGAGCTCACCAGCTCCACGGAGGTGCAG |
| | | GTGGAGCAGGCCACGCCTGTCTCGGAGAGCTGCACAGGGAGA |
| | | ATGGAAGAATTTTTTCTATTTGGAGCCTTCTTGGAAGCCTCAATG |
| | | ATTGACCGGAAAAATGGGGACAAGCCAATTACCTTTGAGGTGAC |
| | | CATAGGAAACTACGGCAATGAAGTCGATGGTATGTCCCGGCCC |
| | | CTGAGGCCTCGGCCCCGGAAAGAGCCTGGGGATGAAGAAGAG |
| | | GTAGACCTGATTCAGAACTCCAGTGACGATGAAGGTGACGAAGC |
| | | CGGGGACCTGGCCTCGGTGTCCTCCACCCCACCTATGCGGCCC |
| | | CAGATCACGGACAGGAACTATTTCCACCTGCCCTACCTGGAGCG |
| | | CAAGCCCTGCATCTATATCAAGAGCTGGTGGCCTGACCAGAGG |
| | | CGGCGCCTCTACAATGCCAACATCATGGATCACATTGCTGACAA |
| | | GCTGGAAGAAGGCCTGAATGATGTACAGGAGATGATCAAAACG |
| | | GAGAAGTCCTACCCGGAGCGCCGCCTGCGGGGTGTGCTAGAG |
| | | GAACTCAGCTGTGGCTGCCACCGCTTCCTCTCCCTCTCGGACAA |
| | | GGACCAGGGCCGCTCGTCCCGCACCAGGCTGGATCGAGAGCG |
| | | TCTTAAGTCCTGTATGAGGGAGTTGGAGAGCATGGGACAGCAG |
| | | GCCAAGAGCCTGAGGGCTCAGGTGAAGCGGCACACTGTTCGGG |
| | | ACAAGCTGAGGTCATGCCAGAACTTTCTGCAGAAGCTACGCTTC |
| | | CTGGCGGATGAGCCCCAGCACAGCATTCCTGATGTGTTCATTTG |
| | | GATGATGAGCAACAACAAACGTATCGCCTATGCCCGCGTGCCTT |
| | | CCAAAGACCTGCTCTTCTCCATCGTGGAGGAGGAACTGGGCAA |
| | | GGACTGCGCCAAAGTCAAGACCCTCTTCCTGAAGCTGCCAGGG |
| | | AAGAGGGGCTTCGGCTCGGCAGGCTGGACAGTACAGGCCAAG |
| | | CTGGAGCTCTACCTGTGGCTGGGCCTCAGCAAGCAGCGAAAGG |
| | | ACTTCCTGTGTGGTCTGCCCTGTGGCTTCGAGGAGGTCAAGGC |
| | | AGCCCAAGGCCTGGGCCTGCATTCCTTTCCGCCCATCAGCCTA |
| | | GTCTACACCAAGAAGCAAGCCTTCCAGCTCCGAGCACACATGTA |
| | | TCAGGCCCGAAGCCTCTTTGCTGCTGACAGCAGTGGGCTCTCT |
| | | GATCCCTTTGCCCGTGTCTTCTTCATCAACCAGAGCCAATGCAC |
| | | TGAGGTTCTAAACGAGACACTGTGTCCCACCTGGGACCAGATGC |
| | | TGGTATTTGACAACCTGGAGCTGTACGGTGAAGCTCACGAGTTA |
| | | CGAGATGATCCCCCCATCATTGTCATTGAAATCTACGACCAGGA |
| | | CAGCATGGGCAAAGCCGACTTCATGGGCCGGACCTTCGCCAAG |
| | | CCCCTGGTGAAGATGGCAGATGAAGCATACTGCCCACCTCGCTT |
| | | CCCGCCGCAGCTTGAGTACTACCAGATCTACCGAGGCAGTGCC |
| | | ACTGCCGGAGACCTACTGGCTGCCTTCGAGCTGCTGCAGATTG |
| | | GGCCATCAGGGAAGGCTGACCTGCCACCCATCAATGGCCCAGT |
| | | GGACATGGACAGAGGGCCCATCATGCCTGTGCCCGTGGGAATC |
| | | CGGCCAGTGCTCAGCAAGTACCGAGTGGAGGTGCTGTTCTGGG |
| | | GCCTGAGGGACCTAAAGAGGGTGAACCTGGCCCAGGTGGACC |
| | | GACCACGGGTGGACATCGAGTGTGCAGGAAAGGGGGTACAATC |
| | | CTCCCTGATTCACAATTATAAGAAGAACCCCAACTTCAACACGCT |
| | | GGTCAAGTGGTTTGAAGTGGACCTCCCGGAGAATGAGCTCCTG |
| | | CACCCACCCTTGAACATCCGAGTGGTAGATTGCCGGGCCTTTG |
| | | GACGATACACCCTGGTGGGTTCCCACGCAGTCAGCTCACTGAG |
| | | GCGCTTCATCTACCGACCTCCAGACCGCTCAGCCCCCAACTGG |
| | | AACACCACAGGGGAGGTTGTAGTAAGCATGGAGCCTGAGGAGC |
| | | CAGTTAAGAAGCTGGAGACCATGGTGAAACTGGATGCGACTTCT |
| | | GATGCTGTGGTCAAGGTGGATGTGGCTGAAGATGAGAAGGAAA |
| | | GGAAGAAGAAGAAAAAGAAAGGCCCGTCAGAGGAGCCAGAGGA |
| | | GGAAGAGCCCGATGAGAGCATGCTGGATTGGTGGTCCAAGTAC |
| | | TTCGCCTCCATCGACACAATGAAGGAGCAACTTCGACAACATGA |
| | | GACCTCTGGAACTGACTTGGAAGAGAAGGAAGAGATGGAAAGC |
| | | GCTGAGGGCCTGAAGGGACCAATGAAGAGCAAGGAGAAGTCCA |
| | | GAGCTGCAAAGGAGGAGAAAAAGAAGAAAAACCAGAGCCCTGG |
| | | CCCTGGCCAGGGATCGGAGGCTCCTGAGAAGAAGAAAGCCAAG |
| | | ATCGATGAGCTTAAGGTGTACCCCAAGGAGCTGGAATCGGAGTT |
| | | TGACAGCTTTGAGGACTGGCTGCACACCTTCAACCTGTTGAGGG |
| | | GCAAGACGGGAGATGATGAGGATGGCTCCACAGAGGAGGAGC |
| | | GCATAGTAGGCCGATTCAAGGGCTCCCTCTGTGTGTACAAAGTG |
| | | CCACTCCCAGAAGATGTATCTCGAGAAGCTGGCTATGATCCCAC |
| | | CTATGGAATGTTCCAGGGCATCCCAAGCAATGACCCCATCAATG |
| | | TGCTGGTCCGAATCTATGTGGTCCGGGCCACAGACCTGCACCC |
| | | GGCCGACATCAATGGCAAAGCTGACCCCTATATTGCCATCAAGT |
| | | TAGGCAAGACCGACATCCGAGACAAGGAGAACTACATCTCCAAG |
| | | CAGCTCAACCCTGTGTTTGGGAAGTCCTTTGACATTGAGGCCTC |
| | | CTTCCCCATGGAGTCCATGTTGACAGTGGCCGTGTACGACTGG |
| | | GATCTGGTGGGCACTGATGACCTCATCGGAGAAACCAAGATTGA |
| | | CCTGGAAAACCGCTTCTACAGCAAGCATCGCGCCACCTGCGGC |
| | | ATCGCACAGACCTATTCCATACATGGCTACAATATCTGGAGGGA |
| | | CCCCATGAAGCCCAGCCAGATCCTGACACGCCTCTGTAAAGAG |
| | | GGCAAAGTGGACGGCCCCCACTTTGGTCCCCATGGGAGAGTGA |
| | | GGGTTGCCAACCGTGTCTTCACGGGGCCTTCAGAAATAGAGGA |
| | | TGAGAATGGTCAGAGGAAGCCCACAGATGAGCACGTGGCACTG |
| | | TCTGCTCTGAGACACTGGGAGGACATCCCCCGGGTGGGCTGCC |
| | | GCCTTGTGCCGGAACACGTGGAGACCAGGCCGCTGCTCAACCC |
| | | TGACAAGCCAGGCATTGAGCAGGGCCGCCTGGAGCTGTGGGTG |
| | | GACATGTTCCCCATGGACATGCCAGCCCCTGGGACACCTCTGG |
| | | ATATATCCCCCAGGAAACCCAAGAAGTACGAGCTGCGGGTCATC |
| | | GTGTGGAACACAGACGAGGTGGTCCTGGAAGACGATGATTTCTT |
| | | CACGGGAGAGAAGTCCAGTGACATTTTTGTGAGGGGGTGGCTG |
| | | AAGGGCCAGCAGGAGGACAAACAGGACACAGATGTCCACTATC |
| | | ACTCCCTCACGGGGGAGGGCAACTTCAACTGGAGATACCTCTTC |
| | | CCCTTCGACTACCTAGCGGCCGAAGAGAAGATCGTTATGTCCAA |
| | | AAAGGAGTCTATGTTCTCCTGGGATGAGACGGAGTACAAGATCC |
| | | CTGCGCGGCTCACCCTGCAGATCTGGGACGCTGACCACTTCTC |
| | | GGCTGACGACTTCCTGGGGGCTATCGAGCTGGACCTGAACCGG |
| | | TTCCCGAGGGGCGCTAAGACAGCCAAGCAGTGCACCATGGAGA |
| | | TGGCCACCGGGGAGGTGGACGTACCCCTGGTTTCCATCTTTAAA |
| | | CAGAAACGTGTCAAAGGCTGGTGGCCCCTCCTGGCCCGCAATG |
| | | AGAATGATGAGTTTGAGCTCACAGGCAAAGTGGAGGCGGAGCT |
| | | ACACCTACTCACGGCAGAGGAGGCAGAGAAGAACCCTGTGGGC |
| | | CTGGCTCGCAATGAACCTGATCCCCTAGAAAAACCCAACCGGCC |
| | | TGACACGGCATTCGTCTGGTTCCTGAACCCACTCAAATCTATCA |
| | | AGTACCTCATCTGCACCCGGTACAAGTGGCTGATCATCAAGATC |
| | | GTGCTGGCGCTGCTGGGGCTGCTCATGCTGGCCCTCTTCCTTT |
| | | ACAGCCTCCCAGGCTACATGGTCAAGAAGCTCCTAGGGGCCTG |
| | | AAGTGTGCCCCACCCCAGCCCGCTCCAGCATCCCTCCAGGGGC |
| | | TGCTGCGTATTTTGCCTTCCCTCACCTGGACTCTCTCCCAACTC |
| | | CCTGAGGAGCCCTCCCACGCCTGCCAGCCTTGAGCAAGACACC |
| | | TGCTTGCTGGACTTCATCCCCACCCCACACCCAAACTGTTGCTT |
| | | GCCTGATCTTGTCCCAGGCCTGCCTGGGGTTTGGGGCACAGTT |
| | | GGCCTCCAAAACCAGATACCCTCTTGTCTAAAGTACCAGGTTCC |
| | | TCTGCCCAACCCCAAGAGTGGTAGTGGCCCAACCCTCCCTGTG |
| | | CTTTCCAAATCTTGTCTTAAGGCACCAGTGAAATTAACCAAGAAA |
| | | CGCGGAGCAATGCCCAAGGCTCTGATGAGTAGGAACACGTGGA |
| | | AAGCACCAGGAATGCCAGCAGAGGCGAGGCGGCACACCTCTCT |
| | | GCAGAGCATCCAGGCCGAGCGGCGGGCAGCGGCCAGCTGCTT |
| | | CTGCGCATGCTCTCCTCTTGGCTCTGCTTCTTTCTCACAGTCACA |
| | | GTCACTTCACAGCTTAGCCTTGGGCTTCCCATCACTTCCAGGGG |
| | | TGCCTCTGCCTTGGCCAGTGTGTGTCAGCTAGTACACAAGCT |
| | | CCAAGTGTGAATCAGGTGTACTGGCCGTCCTGAAGACTGACTGC |
| | | CCTGTCCTTCCTGCCGACAGCCACACCCGAGTGTACACTTAAAG |
| | | CGGTGCCCTTCTGCCTCTGTGGGCCTGCTGGCTGCTGTTCCTTT |
| | | CTTGAGTGTGATTTTTTTTTTCTCTCCCTCAATAAAATAAATCAAA |
| | | CTCTGAGAC |
| 18 | mOTOF-202_2 | TTGGTTGCCTTGGTCTCTGTGGGCAGCAGCAGGAGGAGGCGGC |
| | transcript | AGCAGCCAGAGAAGAGGGAGGCGTGTGAGCCACACTCCACCAG |
| | (NM_001313767.1), | CGAGCTTCTTCCCGCTGCTCTGGAACTGCCCAGGCTCTCCCCA |
| | mouse otoferlin | CCAGCATGGCCCTGATTGTTCACCTCAAGACTGTCTCAGAGCTC |
| | transcript variant 4, | CGAGGCAAAGGTGACCGGATTGCCAAAGTCACTTTCCGAGGGC |
| | 6881 bp, encodes | AGTCTTTCTACTCCCGGGTCCTGGAGAACTGCGAGGGTGTGGC |
| | the protein of SEQ | TGACTTTGATGAGACGTTCCGGTGGCCAGTGGCCAGCAGCATC |
| | ID NO: 9 | GACCGGAATGAAGTGTTGGAGATTCAGATTTTCAACTACAGCAA |
| | | AGTCTTCAGCAACAAGCTGATAGGGACCTTCTGCATGGTGCTGC |
| | | AGAAAGTGGTGGAGGAGAATCGGGTAGAGGTGACCGACACGCT |
| | | GATGGATGACAGCAATGCTATCATCAAGACCAGCCTGAGCATGG |
| | | AGGTCCGGTATCAGGCCACAGATGGCACTGTGGGCCCCTGGGA |
| | | TGATGGAGACTTCCTGGGAGATGAATCCCTCCAGGAGGAGAAG |
| | | GACAGCCAGGAGACAGATGGGCTGCTACCTGGTTCCCGACCCA |
| | | GCACCCGGATATCTGGCGAGAAGAGCTTTCGCAGAGCGGGAAG |
| | | GAGTGTGTTCTCGGCCATGAAACTCGGCAAAACTCGGTCCCACA |
| | | AAGAGGAGCCCCAAAGACAAGATGAGCCAGCAGTGCTGGAGAT |
| | | GGAGGACCTGGACCACCTAGCCATTCAGCTGGGGGATGGGCTG |
| | | GATCCTGACTCCGTGTCTCTAGCCTCGGTCACCGCTCTCACCAG |
| | | CAATGTCTCCAACAAACGGTCTAAGCCAGATATTAAGATGGAGC |
| | | CCAGTGCTGGAAGGCCCATGGATTACCAGGTCAGCATCACAGT |
| | | GATTGAGGCTCGGCAGCTGGTGGGCTTGAACATGGACCCTGTG |
| | | GTGTGTGTGGAGGTGGGTGATGACAAGAAATACACGTCAATGAA |
| | | GGAGTCCACAAACTGCCCTTACTACAACGAGTACTTTGTCTTCG |
| | | ACTTCCATGTCTCTCCTGATGTCATGTTTGACAAGATCATCAAGA |
| | | TCTCGGTTATCCATTCTAAGAACCTGCTTCGGAGCGGCACCCTG |
| | | GTGGGTTCCTTCAAAATGGATGTGGGGACTGTGTATTCCCAGCC |
| | | TGAACACCAGTTCCATCACAAATGGGCCATCCTGTCAGACCCCG |
| | | ATGACATCTCTGCTGGGTTGAAGGGTTATGTAAAGTGTGATGTC |
| | | GCTGTGGTGGGCAAGGGAGACAACATCAAGACACCCCACAAGG |
| | | CCAACGAGACGGATGAGGACGACATTGAAGGGAACTTGCTGCT |
| | | CCCCGAGGGCGTGCCCCCCGAACGGCAGTGGGCACGGTTCTA |
| | | TGTGAAAATTTACCGAGCAGAGGGACTGCCCCGGATGAACACAA |
| | | GCCTCATGGCCAACGTGAAGAAGGCGTTCATCGGTGAGAACAA |
| | | GGACCTCGTCGACCCCTATGTGCAAGTCTTCTTTGCTGGACAAA |
| | | AGGGCAAAACATCAGTGCAGAAGAGCAGCTATGAGCCGCTATG |
| | | GAATGAGCAGGTCGTCTTCACAGACTTGTTCCCCCCACTCTGCA |
| | | AACGCATGAAGGTGCAGATCCGGGACTCTGACAAGGTCAATGAT |
| | | GTGGCCATCGGCACCCACTTCATCGACCTGCGCAAGATTTCCAA |
| | | CGATGGAGACAAAGGCTTCCTGCCTACCCTCGGTCCAGCCTGG |
| | | GTGAACATGTACGGCTCCACGCGCAACTACACACTGCTGGACG |
| | | AGCACCAGGACTTGAATGAAGGCCTGGGGGAGGGTGTGTCCTT |
| | | CCGGGCCCGCCTCATGTTGGGACTAGCTGTGGAGATCCTGGAC |
| | | ACCTCCAACCCAGAGCTCACCAGCTCCACGGAGGTGCAGGTGG |
| | | AGCAGGCCACGCCTGTCTCGGAGAGCTGCACAGGGAGAATGGA |
| | | AGAATTTTTTCTATTTGGAGCCTTCTTGGAAGCCTCAATGATTGA |
| | | CCGGAAAAATGGGGACAAGCCAATTACCTTTGAGGTGACCATAG |
| | | GAAACTACGGCAATGAAGTCGATGGTATGTCCCGGCCCCTGAG |
| | | GCCTCGGCCCCGGAAAGAGCCTGGGGATGAAGAAGAGGTAGA |
| | | CCTGATTCAGAACTCCAGTGACGATGAAGGTGACGAAGCCGGG |
| | | GACCTGGCCTCGGTGTCCTCCACCCCACCTATGCGGCCCCAGA |
| | | TCACGGACAGGAACTATTTCCACCTGCCCTACCTGGAGCGCAAG |
| | | CCCTGCATCTATATCAAGAGCTGGTGGCCTGACCAGAGGCGGC |
| | | GCCTCTACAATGCCAACATCATGGATCACATTGCTGACAAGCTG |
| | | GAAGAAGGCCTGAATGATGTACAGGAGATGATCAAAACGGAGAA |
| | | GTCCTACCCGGAGCGCCGCCTGCGGGGTGTGCTAGAGGAACTC |
| | | AGCTGTGGCTGCCACCGCTTCCTCTCCCTCTCGGACAAGGACC |
| | | AGGGCCGCTCGTCCCGCACCAGGCTGGATCGAGAGCGTCTTAA |
| | | GTCCTGTATGAGGGAGTTGGAGAGCATGGGACAGCAGGCCAAG |
| | | AGCCTGAGGGCTCAGGTGAAGCGGCACACTGTTCGGGACAAGC |
| | | TGAGGTCATGCCAGAACTTTCTGCAGAAGCTACGCTTCCTGGCG |
| | | GATGAGCCCCAGCACAGCATTCCTGATGTGTTCATTTGGATGAT |
| | | GAGCAACAACAAACGTATCGCCTATGCCCGCGTGCCTTCCAAAG |
| | | ACCTGCTCTTCTCCATCGTGGAGGAGGAACTGGGCAAGGACTG |
| | | CGCCAAAGTCAAGACCCTCTTCCTGAAGCTGCCAGGGAAGAGG |
| | | GGCTTCGGCTCGGCAGGCTGGACAGTACAGGCCAAGCTGGAG |
| | | CTCTACCTGTGGCTGGGCCTCAGCAAGCAGCGAAAGGACTTCC |
| | | TGTGTGGTCTGCCCTGTGGCTTCGAGGAGGTCAAGGCAGCCCA |
| | | AGGCCTGGGCCTGCATTCCTTTCCGCCCATCAGCCTAGTCTACA |
| | | CCAAGAAGCAAGCCTTCCAGCTCCGAGCACACATGTATCAGGC |
| | | CCGAAGCCTCTTTGCTGCTGACAGCAGTGGGCTCTCTGATCCCT |
| | | TTGCCCGTGTCTTCTTCATCAACCAGAGCCAATGCACTGAGGTT |
| | | CTAAACGAGACACTGTGTCCCACCTGGGACCAGATGCTGGTATT |
| | | TGACAACCTGGAGCTGTACGGTGAAGCTCACGAGTTACGAGAT |
| | | GATCCCCCCATCATTGTCATTGAAATCTACGACCAGGACAGCAT |
| | | GGGCAAAGCCGACTTCATGGGCCGGACCTTCGCCAAGCCCCTG |
| | | GTGAAGATGGCAGATGAAGCATACTGCCCACCTCGCTTCCCGC |
| | | CGCAGCTTGAGTACTACCAGATCTACCGAGGCAGTGCCACTGC |
| | | CGGAGACCTACTGGCTGCCTTCGAGCTGCTGCAGATTGGGCCA |
| | | TCAGGGAAGGCTGACCTGCCACCCATCAATGGCCCAGTGGACA |
| | | TGGACAGAGGGCCCATCATGCCTGTGCCCGTGGGAATCCGGCC |
| | | AGTGCTCAGCAAGTACCGAGTGGAGGTGCTGTTCTGGGGCCTG |
| | | AGGGACCTAAAGAGGGTGAACCTGGCCCAGGTGGACCGACCAC |
| | | GGGTGGACATCGAGTGTGCAGGAAAGGGGGTACAATCCTCCCT |
| | | GATTCACAATTATAAGAAGAACCCCAACTTCAACACGCTGGTCAA |
| | | GTGGTTTGAAGTGGACCTCCCGGAGAATGAGCTCCTGCACCCA |
| | | CCCTTGAACATCCGAGTGGTAGATTGCCGGGCCTTTGGACGATA |
| | | CACCCTGGTGGGTTCCCACGCAGTCAGCTCACTGAGGCGCTTC |
| | | ATCTACCGACCTCCAGACCGCTCAGCCCCCAACTGGAACACCA |
| | | CAGGGGAGGTTGTAGTAAGCATGGAGCCTGAGGAGCCAGTTAA |
| | | GAAGCTGGAGACCATGGTGAAACTGGATGCGACTTCTGATGCT |
| | | GTGGTCAAGGTGGATGTGGCTGAAGATGAGAAGGAAAGGAAGA |
| | | AGAAGAAAAAGAAAGGCCCGTCAGAGGAGCCAGAGGAGGAAGA |
| | | GCCCGATGAGAGCATGCTGGATTGGTGGTCCAAGTACTTCGCC |
| | | TCCATCGACACAATGAAGGAGCAACTTCGACAACATGAGACCTC |
| | | TGGAACTGACTTGGAAGAGAAGGAAGAGATGGAAAGCGCTGAG |
| | | GGCCTGAAGGGACCAATGAAGAGCAAGGAGAAGTCCAGAGCTG |
| | | CAAAGGAGGAGAAAAAGAAGAAAAACCAGAGCCCTGGCCCTGG |
| | | CCAGGGATCGGAGGCTCCTGAGAAGAAGAAAGCCAAGATCGAT |
| | | GAGCTTAAGGTGTACCCCAAGGAGCTGGAATCGGAGTTTGACA |
| | | GCTTTGAGGACTGGCTGCACACCTTCAACCTGTTGAGGGGCAA |
| | | GACGGGAGATGATGAGGATGGCTCCACAGAGGAGGAGCGCATA |
| | | GTAGGCCGATTCAAGGGCTCCCTCTGTGTGTACAAAGTGCCACT |
| | | CCCAGAAGATGTATCTCGAGAAGCTGGCTATGATCCCACCTATG |
| | | GAATGTTCCAGGGCATCCCAAGCAATGACCCCATCAATGTGCTG |
| | | GTCCGAATCTATGTGGTCCGGGCCACAGACCTGCACCCGGCCG |
| | | ACATCAATGGCAAAGCTGACCCCTATATTGCCATCAAGTTAGGC |
| | | AAGACCGACATCCGAGACAAGGAGAACTACATCTCCAAGCAGCT |
| | | CAACCCTGTGTTTGGGAAGTCCTTTGACATTGAGGCCTCCTTCC |
| | | CCATGGAGTCCATGTTGACAGTGGCCGTGTACGACTGGGATCT |
| | | GGTGGGCACTGATGACCTCATCGGAGAAACCAAGATTGACCTG |
| | | GAAAACCGCTTCTACAGCAAGCATCGCGCCACCTGCGGCATCG |
| | | CACAGACCTATTCCATACATGGCTACAATATCTGGAGGGACCCC |
| | | ATGAAGCCCAGCCAGATCCTGACACGCCTCTGTAAAGAGGGCA |
| | | AAGTGGACGGCCCCCACTTTGGTCCCCATGGGAGAGTGAGGGT |
| | | TGCCAACCGTGTCTTCACGGGGCCTTCAGAAATAGAGGATGAGA |
| | | ATGGTCAGAGGAAGCCCACAGATGAGCACGTGGCACTGTCTGC |
| | | TCTGAGACACTGGGAGGACATCCCCCGGGTGGGCTGCCGCCTT |
| | | GTGCCGGAACACGTGGAGACCAGGCCGCTGCTCAACCCTGACA |
| | | AGCCAGGCATTGAGCAGGGCCGCCTGGAGCTGTGGGTGGACAT |
| | | GTTCCCCATGGACATGCCAGCCCCTGGGACACCTCTGGATATAT |
| | | CCCCCAGGAAACCCAAGAAGTACGAGCTGCGGGTCATCGTGTG |
| | | GAACACAGACGAGGTGGTCCTGGAAGACGATGATTTCTTCACG |
| | | GGAGAGAAGTCCAGTGACATTTTTGTGAGGGGGTGGCTGAAGG |
| | | GCCAGCAGGAGGACAAACAGGACACAGATGTCCACTATCACTC |
| | | CCTCACGGGGGAGGGCAACTTCAACTGGAGATACCTCTTCCCC |
| | | TTCGACTACCTAGCGGCCGAAGAGAAGATCGTTATGTCCAAAAA |
| | | GGAGTCTATGTTCTCCTGGGATGAGACGGAGTACAAGATCCCTG |
| | | CGCGGCTCACCCTGCAGATCTGGGACGCTGACCACTTCTCGGC |
| | | TGACGACTTCCTGGGGGCTATCGAGCTGGACCTGAACCGGTTC |
| | | CCGAGGGGCGCTAAGACAGCCAAGCAGTGCACCATGGAGATGG |
| | | CCACCGGGGAGGTGGACGTACCCCTGGTTTCCATCTTTAAACAG |
| | | AAACGTGTCAAAGGCTGGTGGCCCCTCCTGGCCCGCAATGAGA |
| | | ATGATGAGTTTGAGCTCACAGGCAAAGTGGAGGCGGAGCTACA |
| | | CCTACTCACGGCAGAGGAGGCAGAGAAGAACCCTGTGGGCCTG |
| | | GCTCGCAATGAACCTGATCCCCTAGAAAAACCCAACCGGCCTGA |
| | | CACGGCATTCGTCTGGTTCCTGAACCCACTCAAATCTATCAAGT |
| | | ACCTCATCTGCACCCGGTACAAGTGGCTGATCATCAAGATCGTG |
| | | CTGGCGCTGCTGGGGCTGCTCATGCTGGCCCTCTTCCTTTACA |
| | | GCCTCCCAGGCTACATGGTCAAGAAGCTCCTAGGGGCCTGAAG |
| | | TGTGCCCCACCCCAGCCCGCTCCAGCATCCCTCCAGGGGCTGC |
| | | TGCGTATTTTGCCTTCCCTCACCTGGACTCTCTCCCAACTCCCT |
| | | GAGGAGCCCTCCCACGCCTGCCAGCCTTGAGCAAGACACCTGC |
| | | TTGCTGGACTTCATCCCCACCCCACACCCAAACTGTTGCTTGCC |
| | | TGATCTTGTCCCAGGCCTGCCTGGGGTTTGGGGCACAGTTGGC |
| | | CTCCAAAACCAGATACCCTCTTGTCTAAAGTACCAGGTTCCTCTG |
| | | CCCAACCCCAAGAGTGGTAGTGGCCCAACCCTCCCTGTGCTTTC |
| | | CAAATCTTGTCTTAAGGCACCAGTGAAATTAACCAAGAAACGCG |
| | | GAGCAATGCCCAAGGCTCTGATGAGTAGGAACACGTGGAAAGC |
| | | ACCAGGAATGCCAGCAGAGGCGAGGCGGCACACCTCTCTGCAG |
| | | AGCATCCAGGCCGAGCGGCGGGCAGCGGCCAGCTGCTTCTGC |
| | | GCATGCTCTCCTCTTGGCTCTGCTTCTTTCTCACAGTCACAGTCA |
| | | CTTCACAGCTTAGCCTTGGGCTTCCCATCACTTCCAGGGGTGCC |
| | | TCTGCCTTGGCCAGTGTGTGTCAGCTAGTACACAAGCTCCAAGT |
| | | GTGAATCAGGTGTACTGGCCGTCCTGAAGACTGACTGCCCTGTC |
| | | CTTCCTGCCGACAGCCACACCCGAGTGTACACTTAAAGCGGTG |
| | | CCCTTCTGCCTCTGTGGGCCTGCTGGCTGCTGTTCCTTTCTTGA |
| | | GTGTGATTTTTTTTTTCTCTCCCTCAATAAAATAAATCAAACTCTG |
| | | AGAC |

### Expression of OTOF in Mammalian Cells

Mutations in OTOF have been linked to sensorineural hearing loss and auditory neuropathy. The compositions and methods described herein increase the expression of WT OTOF protein by administering a first nucleic acid vector that contains a polynucleotide encoding an N-terminal portion of an OTOF protein and a second nucleic acid vector that contains a polynucleotide encoding a C-terminal portion of an OTOF protein. In order to utilize nucleic acid vectors for therapeutic application in the treatment of sensorineural hearing loss and auditory neuropathy, they can be directed to the interior of the cell, and, in particular, to specific cell types. A wide array of methods has been established for the delivery of proteins to mammalian cells and for the stable expression of genes encoding proteins in mammalian cells.

### Polynucleotides Encoding OTOF

One platform that can be used to achieve therapeutically effective intracellular concentrations of OTOF in mammalian cells is via the stable expression of the gene encoding OTOF (e.g., by integration into the nuclear or mitochondrial genome of a mammalian cell, or by episomal concatemer formation in the nucleus of a mammalian cell). The gene is a polynucleotide that encodes the primary amino acid sequence of the corresponding protein. In order to introduce exogenous genes into a mammalian cell, genes can be incorporated into a vector. Vectors can be introduced into a cell by a variety of methods, including transformation, transfection, transduction, direct uptake, projectile bombardment, and by encapsulation of the vector in a liposome. Examples of suitable methods of transfecting or transforming cells include calcium phosphate precipitation, electroporation, microinjection, infection, lipofection and direct uptake. Such methods are described in more detail, for example, in Green, et al., Molecular Cloning: A Laboratory Manual, Fourth Edition (Cold Spring Harbor University Press, New York 2014); and Ausubel, et al., Current Protocols in Molecular Biology (John Wiley & Sons, New York 2015), the disclosures of each of which are incorporated herein by reference.

OTOF can also be introduced into a mammalian cell by targeting vectors containing portions of a gene encoding an OTOF protein to cell membrane phospholipids. For example, vectors can be targeted to the phospholipids on the extracellular surface of the cell membrane by linking the vector molecule to a VSV-G protein, a viral protein with affinity for all cell membrane phospholipids. Such a construct can be produced using methods well known to those of skill in the field.

Recognition and binding of the polynucleotide encoding an OTOF protein by mammalian RNA polymerase is important for gene expression. As such, one may include sequence elements within the polynucleotide that exhibit a high affinity for transcription factors that recruit RNA polymerase and promote the assembly of the transcription complex at the transcription initiation site. Such sequence elements include, e.g., a mammalian promoter, the sequence of which can be recognized and bound by specific transcription initiation factors and ultimately RNA polymerase.

Polynucleotides suitable for use in the compositions and methods described herein also include those that encode an OTOF protein downstream of a mammalian promoter (e.g., a polynucleotide that encodes an N-terminal portion of an OTOF protein downstream of a mammalian promoter). Promoters that are useful for the expression of an OTOF protein in mammalian cells include constitutive promoters, cochlear hair cell-specific promoters, and inner hair cell-specific promoters. Constitutive promoters include the CAG promoter, the cytomegalovirus (CMV) promoter, the smCBA promoter, the EF1a promoter, and the PGK promoter. Cochlear hair cell-specific promoters include the Myosin 15 (Myo15) promoter, the Myosin 7A (Myo7A) promoter, the Myosin 6 (Myo6) promoter, the POU4F3 promoter, the Atonal BHLH Transcription Factor 1 (ATOH1) promoter, the LIM Homeobox 3 (LHX3) promoter, the α9 acetylcholine receptor (α9AChR) promoter, and the α10 acetylcholine receptor (α10AChR) promoter. Inner hair cell-specific promoters include the FGF8 promoter, the VGLUT3 promoter, and the OTOF promoter. Alternatively, promoters derived from viral genomes can also be used for the stable expression of these agents in mammalian cells. Examples of functional viral promoters that can be used to promote mammalian expression of these agents include adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein barr virus (EBV) promoter, and the Rous sarcoma virus (RSV) promoter.

### Murine Myosin 15 Promoters

In some embodiments, the Myo15 promoter for use in the compositions and methods described herein includes nucleic acid sequences from regions of the murine Myo15 locus that are capable of expressing a transgene specifically in hair cells, or variants thereof, such as a nucleic acid sequences that have at least 85% sequence identity (e.g., 85%, 90%, 95%, 96%, 97%, 98%, 99%, or more, sequence identity) to regions of the murine Myo15 locus that are capable of expressing a transgene specifically in hair cells. These regions include nucleic acid sequences immediately preceding the murine Myo15 translation start site and an upstream regulatory element that is located over 5 kb from the murine Myo15 translation start site. The Myo15 promoter for use in the compositions and methods described herein can optionally include a linker operably linking the regions of the murine Myo15 locus that are capable of expressing a transgene specifically in hair cells, or the regions of the murine Myo15 locus can be joined directly without an intervening linker.

In some embodiments, the Myo15 promoter for use in the compositions and methods described herein contains a first region (an upstream regulatory element) having at least 85% sequence identity (e.g., 85%, 90%, 95%, 96%, 97%, 98%, 99%, or more, sequence identity) to a region containing the first non-coding exon of the murine Myo15 gene (nucleic acids from −6755 to −7209 with respect to the murine Myo15 translation start site, the sequence of which is set forth in SEQ ID NO: 24) or a functional portion or derivative thereof joined (e.g., operably linked) to a second region having at least 85% sequence identity (e.g., 85%, 90%, 95%, 96%, 97%, 98%, 99%, or more, sequence identity) to the nucleic acid sequence immediately preceding the murine Myo15 translation start site (nucleic acids from −1 to −1157 with respect to the murine Myo15 translation start site, the sequence of which is set forth in SEQ ID NO: 25) or a functional portion or derivative thereof. The functional portion of SEQ ID NO: 24 may have the sequence of nucleic acids from −7166 to −7091 with respect to the murine Myo15 translation start site (set forth in SEQ ID NO: 26) and/or the sequence of nucleic acids from −7077 to −6983 with respect to the murine Myo15 translation start site (set forth in SEQ ID NO: 27). The first region may contain the nucleic acid sequence of SEQ ID NO: 26 fused to the nucleic acid sequence of SEQ ID NO: 27 with no intervening nucleic acids, as set forth in SEQ ID NO: 28, or the first region may contain the nucleic acid sequence of SEQ ID NO: 27 fused to the nucleic acid sequence of SEQ ID NO: 26 with no intervening nucleic acids, as set forth in SEQ ID NO: 29. Alternatively, the first region may contain the sequences of SEQ ID NO: 26 and SEQ ID NO: 27 joined by the endogenous intervening nucleic acid sequence (e.g., the first region may have or include the sequence of nucleic acids from −7166 to −6983 with respect to the murine Myo15 translation start site, as set forth in SEQ ID NO: 30 and SEQ ID NO: 50) or a nucleic acid linker. In a murine Myo15 promoter in which the first region contains both SEQ ID NO: 26 and SEQ ID NO: 27, the two sequences can be included in any order (e.g., SEQ ID NO: 26 may be joined to (e.g., precede) SEQ ID NO: 27, or SEQ ID NO: 27 may be joined to (e.g., precede) SEQ ID NO: 26). The functional portion of SEQ ID NO: 25 may have the sequence of nucleic acids from −590 to −509 with respect to the murine Myo15 translation start site (set forth in SEQ ID NO: 31) and/or the sequence of nucleic acids from −266 to −161 with respect to the murine Myo15 translation start site (set forth in SEQ ID NO: 32). In some embodiments, the sequence containing SEQ ID NO: 31 has the sequence of SEQ ID NO: 51. In some embodiments, the sequence containing SEQ ID NO: 32 has the sequence of SEQ ID NO: 52. The second region may contain the nucleic acid sequence of SEQ ID NO: 31 fused to the nucleic acid sequence of SEQ ID NO: 32 with no intervening nucleic acids, as set forth in SEQ ID NO: 33, or the second region may contain the nucleic acid sequence of SEQ ID NO: 32 fused to the nucleic acid sequence of SEQ ID NO: 31 with no intervening nucleic acids, as set forth in SEQ ID NO: 34. The second region may contain the nucleic acid sequence of SEQ ID NO: 51 fused to the nucleic acid sequence of SEQ ID NO: 52 with no intervening nucleic acids, as set forth in SEQ ID NO: 55, or the second region may contain the nucleic acid sequence of SEQ ID NO: 52 fused to the nucleic acid sequence of SEQ ID NO: 51 with no intervening nucleic acids. Alternatively, the second region may contain the sequences of SEQ ID NO: 31 and SEQ ID NO: 32 joined by the endogenous intervening nucleic acid sequence (e.g., the second region may have the sequence of nucleic acids from −590 to −161 with respect to the murine Myo15 translation start site, as set forth in SEQ ID NO: 35) or a nucleic acid linker. In a murine Myo15 promoter in which the second region contains both SEQ ID NO: 31 and SEQ ID NO: 32, the two sequences can be included in any order (e.g., SEQ ID NO: 31 may be joined to (e.g., precede) SEQ ID NO: 32, or SEQ ID NO: 32 may be joined to (e.g., precede) SEQ ID NO: 31).

The first region and the second region of the murine Myo15 promoter can be joined directly or can be joined by a nucleic acid linker. For example, the murine Myo15 promoter can contain the sequence of SEQ ID NO: 24 or a functional portion or derivative thereof (e.g., any one or more of SEQ ID NOs: 26-30 and 50, e.g., SEQ ID NOs 26 and 27) fused to the sequence of SEQ ID NO: 25 or a functional portion or derivative thereof (e.g., any one or more of SEQ ID NOs: 31-35, 51, 52, and 55, e.g., SEQ ID NOs 31 and 32) with no intervening nucleic acids. For example, the nucleic acid sequence of the murine Myo15 promoter that results from direct fusion of SEQ ID NO: 24 to SEQ ID NO: 25 is set forth in SEQ ID NO: 36. Alternatively, a linker can be used to join the sequence of SEQ ID NO: 24 or a functional portion or derivative thereof (e.g., any one or more of SEQ ID NOs: 26-30 and 50, e.g., SEQ ID NOs 26 and 27) to the sequence of SEQ ID NO: 25 or a functional portion or derivative thereof (e.g., any one or more of SEQ ID NOs: 31-35, 51, 52, and 55, e.g., SEQ ID NOs 31 and 32). Exemplary Myo15 promoters containing functional portions of both SEQ ID NO: 24 and SEQ ID NO: 25 are provided in SEQ ID NOs: 38, 39, 53, 54, 59, and 60.

The length of a nucleic acid linker for use in a murine Myo15 promoter described herein can be about 5 kb or less (e.g., about 5 kb, 4.5, kb, 4, kb, 3.5 kb, 3 kb, 2.5 kb, 2 kb, 1.5 kb, 1 kb, 900 bp, 800 bp, 700 bp, 600 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15, bp, 10 bp, 5 bp, 4 bp, 3 bp, 2 bp, or less). Nucleic acid linkers that can be used in the murine Myo15 promoter described herein do not disrupt the ability of the murine Myo15 promoter of the invention to induce transgene expression in hair cells.

In some embodiments, the sequence of SEQ ID NO: 24 or a functional portion or derivative thereof (e.g., any one or more of SEQ ID NOs: 26-30 and 50, e.g., SEQ ID NOs 26 and 27) is joined (e.g., operably linked) to the sequence of SEQ ID NO: 25 or a functional portion or derivative thereof (e.g., any one or more of SEQ ID NOs: 31-35, 51, 52, and 55, e.g., SEQ ID NOs 31 and 32), and, in some embodiments, the order of the regions is reversed (e.g., the sequence of SEQ ID NO: 25 or a functional portion or derivative thereof (e.g., any one or more of SEQ ID NOs: 31-35, 51, 52, and 55, e.g., SEQ ID NOs 31 and 32) is joined (e.g., operably linked) to the sequence of SEQ ID NO: 24 or a functional portion or derivative thereof (e.g., any one or more of SEQ ID NOs: 26-30 and 50, e.g., SEQ ID NOs 26 and 27)). For example, the nucleic acid sequence of a murine Myo15 promoter that results from direct fusion of SEQ ID NO: 25 to SEQ ID NO: 24 is set forth in SEQ ID NO: 37. An example of a murine Myo15 promoter in which a functional portion or derivative of SEQ ID NO: 25 precedes a functional portion or derivative of SEQ ID NO: 24 is provided in SEQ ID NO: 58. Regardless of order, the sequence of SEQ ID NO: 24 or a functional portion or derivative thereof and the sequence of SEQ ID NO: 25 or a functional portion or derivative thereof can be joined by direct fusion or a nucleic acid linker, as described above.

In some embodiments, the murine Myo15 promoter for use in the compositions and methods described herein contains a region having at least 85% sequence identity (e.g., 85%, 90%, 95%, 96%, 97%, 98%, 99%, or more, sequence identity) to a region containing the first non-coding exon of the murine Myo15 gene (nucleic acids from −6755 to −7209 with respect to the murine Myo15 translation start site, the sequence of which is set forth in SEQ ID NO: 24) or a functional portion or derivative thereof. The functional portion of SEQ ID NO: 24 may have the sequence of nucleic acids from −7166 to −7091 with respect to the murine Myo15 translation start site (set forth in SEQ ID NO: 26) and/or the sequence of nucleic acids from −7077 to −6983 with respect to the murine Myo15 translation start site (set forth in SEQ ID NO: 27). The murine Myo15 promoter may contain the nucleic acid sequence of SEQ ID NO: 26 fused to the nucleic acid sequence of SEQ ID NO: 27 with no intervening nucleic acids, as set forth in SEQ ID NO: 28, or the murine Myo15 promoter may contain the nucleic acid sequence of SEQ ID NO: 27 fused to the nucleic acid sequence of SEQ ID NO: 26 with no intervening nucleic acids, as set forth in SEQ ID NO: 29. Alternatively, the murine Myo15 promoter may contain the sequences of SEQ ID NO: 26 and SEQ ID NO: 27 joined by the endogenous intervening nucleic acid sequence (e.g., the first region may have or include the sequence of nucleic acids from −7166 to −6983 with respect to the murine Myo15 translation start site, as set forth in SEQ ID NO: 30 and SEQ ID NO: 50) or a nucleic acid linker. In a murine Myo15 promoter that contains both SEQ ID NO: 26 and SEQ ID NO: 27, the two sequences can be included in any order (e.g., SEQ ID NO: 26 may be joined to (e.g., precede) SEQ ID NO: 27, or SEQ ID NO: 27 may be joined to (e.g., precede) SEQ ID NO: 26).

In some embodiments, the murine Myo15 promoter for use in the compositions and methods described herein contains a region having at least 85% sequence identity (e.g., 85%, 90%, 95%, 96%, 97%, 98%, 99%, or more, sequence identity) to the nucleic acid sequence immediately upstream of the murine Myo15 translation start site (nucleic acids from −1 to −1157 with respect to the murine Myo15 translation start site, the sequence of which is set forth in SEQ ID NO: 25) or a functional portion or derivative thereof. The functional portion of SEQ ID NO: 25 may have the sequence of nucleic acids from −590 to −509 with respect to the murine Myo15 translation start site (set forth in SEQ ID NO: 31) and/or the sequence of nucleic acids from −266 to −161 with respect to the murine Myo15 translation start site (set forth in SEQ ID NO: 32). In some embodiments, the sequence containing SEQ ID NO: 31 has the sequence of SEQ ID NO: 51. In some embodiments, the sequence containing SEQ ID NO: 32 has the sequence of SEQ ID NO: 52. The murine Myo15 promoter may contain the nucleic acid sequence of SEQ ID NO: 31 fused to the nucleic acid sequence of SEQ ID NO: 32 with no intervening nucleic acids, as set forth in SEQ ID NO: 33, or the murine Myo15 promoter may contain the nucleic acid sequence of SEQ ID NO: 32 fused to the nucleic acid sequence of SEQ ID NO: 31 with no intervening nucleic acids, as set forth in SEQ ID NO: 34. The murine Myo15 promoter may contain the nucleic acid sequence of SEQ ID NO: 51 fused to the nucleic acid sequence of SEQ ID NO: 52 with no intervening nucleic acids, as set forth in SEQ ID NO: 55, or the murine Myo15 promoter may contain the nucleic acid sequence of SEQ ID NO: 52 fused to the nucleic acid sequence of SEQ ID NO: 51 with no intervening nucleic acids. Alternatively, the murine Myo15 promoter may contain the sequences of SEQ ID NO: 31 and SEQ ID NO: 32 joined by the endogenous intervening nucleic acid sequence (e.g., the second region may have the sequence of nucleic acids from −590 to −161 with respect to the murine Myo15 translation start site, as set forth in SEQ ID NO: 35) or a nucleic acid linker. In a murine Myo15 promoter that contains both SEQ ID NO: 31 and SEQ ID NO: 32, the two sequences can be included in any order (e.g., SEQ ID NO: 31 may be joined to (e.g., precede) SEQ ID NO: 32, or SEQ ID NO: 32 may be joined to (e.g., precede) SEQ ID NO: 31).

In some embodiments, the murine Myo15 promoter for use in the compositions and methods described herein contains a functional portion or derivative of a region having at least 85% sequence identity (e.g., 85%, 90%, 95%, 96%, 97%, 98%, 99%, or more, sequence identity) to a region containing the first non-coding exon of the Myo15 gene (nucleic acids from −6755 to −7209 with respect to the murine Myo15 translation start site, the sequence of which is set forth in SEQ ID NO: 24) flanked on both sides by a functional portion or derivative of a region having at least 85% sequence identity (e.g., 85%, 90%, 95%, 96%, 97%, 98%, 99%, or more, sequence identity) to the nucleic acid sequence immediately upstream of the murine Myo15 translation start site (nucleic acids from −1 to −1157 with respect to the murine Myo15 translation start site, the sequence of which is set forth in SEQ ID NO: 25). For example, a functional portion or derivative of SEQ ID NO: 25, such as SEQ ID NO: 31 or 51 may be directly fused or joined by a nucleic acid linker to a portion of SEQ ID NO: 24, such as any one of SEQ ID NOs: 26-30 and 50, which is directly fused or joined by a nucleic acid linker to a different functional portion of SEQ ID NO: 25, such as SEQ ID NO: 32 or 52. In other embodiments, a functional portion or derivative of SEQ ID NO: 25, such as SEQ ID NO: 32 or 52 may be directly fused or joined by a nucleic acid linker to a portion of SEQ ID NO: 24, such as any one of SEQ ID NOs: 26-30 and 50, which is directly fused or joined by a nucleic acid linker to a different functional portion of SEQ ID NO: 25, such as SEQ ID NO: 31 or 51. For example, polynucleotides having at least 85% sequence identity (e.g., 85%, 90%, 95%, 96%, 97%, 98%, 99%, or more, sequence identity) to the nucleic acid sequence of SEQ ID NOs: 51, 50, and 52 can be fused to produce a polynucleotide having at least 85% sequence identity (e.g., 85%, 90%, 95%, 96%, 97%, 98%, 99%, or more, sequence identity) to the nucleic acid sequence of SEQ ID NO: 56. In some embodiments, polynucleotides having at least 85% sequence identity (e.g., 85%, 90%, 95%, 96%, 97%, 98%, 99%, or more, sequence identity) to the nucleic acid sequence of SEQ ID NOs: 52, 50, and 51 can be fused to produce a polynucleotide having at least 85% sequence identity (e.g., 85%, 90%, 95%, 96%, 97%, 98%, 99%, or more, sequence identity) to the nucleic acid sequence of SEQ ID NO: 57.

### Human Myosin 15 Promoters

In some embodiments, the Myo15 promoter for use in the compositions and methods described herein includes nucleic acid sequences from regions of the human Myo15 locus that are capable of expressing a transgene specifically in hair cells, or variants thereof, such as a nucleic acid sequences that have at least 85% sequence identity (e.g., 85%, 90%, 95%, 96%, 97%, 98%, 99%, or more, sequence identity) to regions of the human Myo15 locus that are capable of expressing a transgene specifically in hair cells. The Myo15 promoter for use in the compositions and methods described herein can optionally include a linker operably linking the regions of the human Myo15 locus that are capable of expressing a transgene specifically in hair cells, or the regions of the human Myo15 locus can be joined directly without an intervening linker.

In some embodiments, the Myo15 promoter for use in the compositions and methods described herein contains a first region having at least 85% sequence identity (e.g., 85%, 90%, 95%, 96%, 97%, 98%, 99%, or more, sequence identity) to the sequence set forth in SEQ ID NO: 40 or a functional portion or derivative thereof joined (e.g., operably linked) to a second region having at least 85% sequence identity (e.g., 85%, 90%, 95%, 96%, 97%, 98%, 99%, or more, sequence identity) to the sequence set forth in SEQ ID NO: 41 or a functional portion or derivative thereof. The functional portion of SEQ ID NO: 40 may have the sequence set forth in SEQ ID NO: 42. The functional portion of SEQ ID NO: 41 may have the sequence set forth in SEQ ID NO: 43 and/or the sequence set forth in SEQ ID NO: 44. The second region may contain the nucleic acid sequence of SEQ ID NO: 43 fused to the nucleic acid sequence of SEQ ID NO: 44 with no intervening nucleic acids, as set forth in SEQ ID NO: 45, or the second region may contain the nucleic acid sequence of SEQ ID NO: 44 fused to the nucleic acid sequence of SEQ ID NO: 43 with no intervening nucleic acids, as set forth in SEQ ID NO: 46. Alternatively, the second region may contain the sequences of SEQ ID NO: 43 and SEQ ID NO: 44 joined by the endogenous intervening nucleic acid sequence (as set forth in SEQ ID NO: 47) or a nucleic acid linker. In a human Myo15 promoter in which the second region contains both SEQ ID NO: 43 and SEQ ID NO: 44, the two sequences can be included in any order (e.g., SEQ ID NO: 43 may be joined to (e.g., precede) SEQ ID NO: 44, or SEQ ID NO: 44 may be joined to (e.g., precede) SEQ ID NO: 43).

The first region and the second region of the human Myo15 promoter can be joined directly or can be joined by a nucleic acid linker. For example, the human Myo15 promoter can contain the sequence of SEQ ID NO: 40 or a functional portion or derivative thereof (e.g., SEQ ID NO: 42) fused to the sequence of SEQ ID NO: 41 or a functional portion or derivative thereof (e.g., any one or more of SEQ ID NOs: 43-47, e.g., SEQ ID NOs: 43 and/or 44) with no intervening nucleic acids. Alternatively, a linker can be used to join the sequence of SEQ ID NO: 40 or a functional portion or derivative thereof (e.g., SEQ ID NO: 42) to the sequence of SEQ ID NO: 41 or a functional portion or derivative thereof (e.g., any one or more of SEQ ID NOs: 43-47, e.g., SEQ ID NOs: 43 and/or 44). Exemplary human Myo15 promoters containing functional portions of both SEQ ID NO: 40 and SEQ ID NO: 41 are provided in SEQ ID NOs: 48 and 49.

In some embodiments, the sequence of SEQ ID NO: 40 or a functional portion or derivative thereof (e.g., SEQ ID NO: 42) is joined (e.g., operably linked) to the sequence of SEQ ID NO: 41 or a functional portion or derivative thereof (e.g., any one or more of SEQ ID NOs: 43-47, e.g., SEQ ID NOs: 43 and 44), and, in some embodiments, the order of the regions is reversed (e.g., the sequence of SEQ ID NO: 41 or a functional portion or derivative thereof (e.g., any one or more of SEQ ID NOs: 43-47, e.g., SEQ ID NOs: 43 and/or 44) is joined (e.g., operably linked) to the sequence of SEQ ID NO: 40 or a functional portion or derivative thereof (e.g., SEQ ID NO: 42). Regardless of order, the sequence of SEQ ID NO: 40 or a functional portion or derivative thereof and the sequence of SEQ ID NO: 41 or a functional portion or derivative thereof can be joined by direct fusion or a nucleic acid linker, as described above.

In some embodiments, the human Myo15 promoter for use in the compositions and methods described herein contains a region having at least 85% sequence identity (e.g., 85%, 90%, 95%, 96%, 97%, 98%, 99%, or more, sequence identity) to a region containing the sequence set forth in SEQ ID NO: 40 or a functional portion or derivative thereof. The functional portion of SEQ ID NO: 40 may have the sequence of nucleic acids set forth in SEQ ID NO: 42.

In some embodiments, the human Myo15 promoter for use in the compositions and methods described herein contains a region having at least 85% sequence identity (e.g., 85%, 90%, 95%, 96%, 97%, 98%, 99%, or more, sequence identity) to the sequence set forth in SEQ ID NO: 41 or a functional portion or derivative thereof. The functional portion of SEQ ID NO: 41 may have the sequence set forth in SEQ ID NO: 43 and/or the sequence set forth in SEQ ID NO: 44. The human Myo15 promoter may contain the nucleic acid sequence of SEQ ID NO: 43 fused to the nucleic acid sequence of SEQ ID NO: 44 with no intervening nucleic acids, as set forth in SEQ ID NO: 45, or the human Myo15 promoter may contain the nucleic acid sequence of SEQ ID NO: 44 fused to the nucleic acid sequence of SEQ ID NO: 43 with no intervening nucleic acids, as set forth in SEQ ID NO: 46. Alternatively, the human Myo15 promoter may contain the sequences of SEQ ID NO: 43 and SEQ ID NO: 44 joined by the endogenous intervening nucleic acid sequence (e.g., as set forth in SEQ ID NO: 47) or a nucleic acid linker. In a human Myo15 promoter that contains both SEQ ID NO: 43 and SEQ ID NO: 44, the two sequences can be included in any order (e.g., SEQ ID NO: 43 may be joined to (e.g., precede) SEQ ID NO: 44, or SEQ ID NO: 44 may be joined to (e.g., precede) SEQ ID NO: 43).

The length of a nucleic acid linker for use in a human Myo15 promoter described herein can be about 5 kb or less (e.g., about 5 kb, 4.5, kb, 4, kb, 3.5 kb, 3 kb, 2.5 kb, 2 kb, 1.5 kb, 1 kb, 900 bp, 800 bp, 700 bp, 600 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15, bp, 10 bp, 5 bp, 4 bp, 3 bp, 2 bp, or less). Nucleic acid linkers that can be used in the human Myo15 promoters described herein do not disrupt the ability of the human Myo15 promoter of the invention to induce transgene expression in hair cells.

The foregoing Myol 5 promoter sequences are summarized in Table 3, below.

TABLE 3
Exemplary nucleotide sequences for use in the Myo15 promoter described herein

| SEQ ID | Description of nucleic | |
|---|---|---|
| NO. | acid sequence | Nucleic Acid Sequence |
| 24 | Region containing non- | CTGCAGCTCAGCCTACTACTTGCTTTCCAGGCTGTTCCTAGT |
| | coding exon 1 of Myo15 | TCCCATGTCAGCTGCTTGTGCTTTCCAGAGACAAAACAGGAA |
| | (−6755 to −7209) | TAATAGATGTCATTAAATATACATTGGGCCCCAGGCGGTCAAT |
| | | GTGGCAGCCTGAGCCTCCTTTCCATCTCTGTGGAGGCAGAC |
| | | ATAGGACCCCCAACAAACAGCATGCAGGTTGGGAGCCAGCC |
| | | ACAGGACCCAGGTAAGGGGCCCTGGGTCCTTAAGCTTCTGC |
| | | CACTGGCTCCGGCATTGCAGAGAGAAGAGAAGGGGCGGCA |
| | | GAGCTGAACCTTAGCCTTGCCTTCCTGGGTACCCTTCTGAGC |
| | | CTCACTGTCTTCTGTGAGATGGGCAAAGTGCGGGTGTGACTC |
| | | CTTGGCAACGGTGTTACACCAGGGCAGGTAAAGTTGTAGTTA |
| | | TTTGTGGGGTACACCAGGACTGTTAAAGGTGTAACTAT |
| 25 | Region immediately | GGTCTCACCCAGCATTTTCACTTCTAATAAGTTCAAATGTGAT |
| | preceding the | ACGGCACCTTTCTAAAAATTAGTTTTCAGGGAAATAGGGTTCA |
| | translation start site | AAACTGGTAGTGGTAGGGTCCATTCTCACGACCCCCAGGCCT |
| | of Myo15 (−1 to −1157) | GCTAACCCTGACCAAGCTACCTATTACTTACCCTCCTCTTTCT |
| | | CCTCCTCCTCTTTCTCCTTCTCCTGCTTCCCCTCTTCCTTCTC |
| | | CCTCCCTTCCTCTCCCTCCTCCCCCTCCTTGGCTGTGATCAG |
| | | ATCCAGAGCCTGAATGAGCCTCCTGACCCCACACCCCCACTA |
| | | GCATGGGCCTGCAAGTGCCCAGAAGTCCCTCCTGCCTCCTA |
| | | AACTGCCCAGCCGATCCATTAGCTCTTCCTTCTTCCCAGTGA |
| | | AAGAAGCAGGCACAGCCTGTCCCTCCCGTTCTACAGAAAGG |
| | | AAGCTACAGCACAGGGAGGGCCAAAGGCCTTCCTGGGACTA |
| | | GACAGTTGATCAACAGCAGGACTGGAGAGCTGGGCTCCATTT |
| | | TTGTTCCTTGGTGCCCTGCCCCTCCCCATGACCTGCAGAGAC |
| | | ATTCAGCCTGCCAGGCTTTATGAGGTGGGAGCTGGGCTCTC |
| | | CCTGATGTATTATTCAGCTCCCTGGAGTTGGCCAGCTCCTGT |
| | | TACACTGGCCACAGCCCTGGGCATCCGCTTCTCACTTCTAGT |
| | | TTCCCCTCCAAGGTAATGTGGTGGGTCATGATCATTCTATCCT |
| | | GGCTTCAGGGACCTGACTCCACTTTGGGGCCATTCGAGGGG |
| | | TCTAGGGTAGATGATGTCCCCCTGTGGGGATTAATGTCCTGC |
| | | TCTGTAAAACTGAGCTAGCTGAGATCCAGGAGGGCTTGGCCA |
| | | GAGACAGCAAGTTGTTGCCATGGTGACTTTAAAGCCAGGTTG |
| | | CTGCCCCAGCACAGGCCTCCCAGTCTACCCTCACTAGAAAAC |
| | | AACACCCAGGCACTTTCCACCACCTCTCAAAGGTGAAACCCA |
| | | AGGCTGGTCTAGAGAATGAATTATGGATCCTCGCTGTCCGTG |
| | | CCACCCAGCTAGTCCCAGCGGCTCAGACACTGAGGAGAGAC |
| | | TGTAGGTTCAGCTACAAGCAAAAAGACCTAGCTGGTCTCCAA |
| | | GCAGTGTCTCCAAGTCCCTGAACCTGTGACACCTGCCCCAG |
| | | GCATCATCAGGCACAGAGGGCCACC |
| 26 | Portion of SEQ ID NO: | CCCATGTCAGCTGCTTGTGCTTTCCAGAGACAAAACAGGAAT |
| | 24 (−7166 to −7091) | AATAGATGTCATTAAATATACATTGGGCCCCAGG |
| 27 | Portion of SEQ ID NO: | AGCCTGAGCCTCCTTTCCATCTCTGTGGAGGCAGACATAGGA |
| | 24 (−7077 to −6983) | CCCCCAACAAACAGCATGCAGGTTGGGAGCCAGCCACAGGA |
| | | CCCAGGTAAGGG |
| 28 | Portion of SEQ ID NO: | CCCATGTCAGCTGCTTGTGCTTTCCAGAGACAAAACAGGAAT |
| | 24 (SEQ ID NO: 26 | AATAGATGTCATTAAATATACATTGGGCCCCAGGAGCCTGAG |
| | fused to SEQ ID NO: | CCTCCTTTCCATCTCTGTGGAGGCAGACATAGGACCCCCAAC |
| | 27) | AAACAGCATGCAGGTTGGGAGCCAGCCACAGGACCCAGGTA |
| | | AGGG |
| 29 | Portion of SEQ ID NO: | AGCCTGAGCCTCCTTTCCATCTCTGTGGAGGCAGACATAGGA |
| | 24 (SEQ ID NO: 27 | CCCCCAACAAACAGCATGCAGGTTGGGAGCCAGCCACAGGA |
| | fused to SEQ ID NO: | CCCAGGTAAGGGCCCATGTCAGCTGCTTGTGCTTTCCAGAGA |
| | 26) | CAAAACAGGAATAATAGATGTCATTAAATATACATTGGGCCCC |
| | | AGG |
| 30 | Portion of SEQ ID NO: | CCCATGTCAGCTGCTTGTGCTTTCCAGAGACAAAACAGGAAT |
| | 24 (−7166 to −6983) | AATAGATGTCATTAAATATACATTGGGCCCCAGGCGGTCAAT |
| | | GTGGCAGCCTGAGCCTCCTTTCCATCTCTGTGGAGGCAGAC |
| | | ATAGGACCCCCAACAAACAGCATGCAGGTTGGGAGCCAGCC |
| | | ACAGGACCCAGGTAAGGG |
| 31 | Portion of SEQ ID NO: | TGAGGTGGGAGCTGGGCTCTCCCTGATGTATTATTCAGCTCC |
| | 25 (−590 to −509) | CTGGAGTTGGCCAGCTCCTGTTACACTGGCCACAGCCCTG |
| 32 | Portion of SEQ ID NO: | CACAGGCCTCCCAGTCTACCCTCACTAGAAAACAACACCCAG |
| | 25 (−266 to −161) | GCACTTTCCACCACCTCTCAAAGGTGAAACCCAAGGCTGGTC |
| | | TAGAGAATGAATTATGGATCCT |
| 33 | Portion of SEQ ID NO: | TGAGGTGGGAGCTGGGCTCTCCCTGATGTATTATTCAGCTCC |
| | 25 | CTGGAGTTGGCCAGCTCCTGTTACACTGGCCACAGCCCTGC |
| | (SEQ ID NO: 31 fused | ACAGGCCTCCCAGTCTACCCTCACTAGAAAACAACACCCAGG |
| | to SEQ ID NO: 32) | CACTTTCCACCACCTCTCAAAGGTGAAACCCAAGGCTGGTCT |
| | | AGAGAATGAATTATGGATCCT |
| 34 | Portion of SEQ ID NO: | CACAGGCCTCCCAGTCTACCCTCACTAGAAAACAACACCCAG |
| | 25 | GCACTTTCCACCACCTCTCAAAGGTGAAACCCAAGGCTGGTC |
| | (SEQ ID NO: 32 fused | TAGAGAATGAATTATGGATCCTTGAGGTGGGAGCTGGGCTCT |
| | to SEQ ID NO: 31) | CCCTGATGTATTATTCAGCTCCCTGGAGTTGGCCAGCTCCTG |
| | | TTACACTGGCCACAGCCCTG |
| 35 | Portion of SEQ ID NO: | TGAGGTGGGAGCTGGGCTCTCCCTGATGTATTATTCAGCTCC |
| | 25 | CTGGAGTTGGCCAGCTCCTGTTACACTGGCCACAGCCCTGG |
| | (−590 to −161) | GCATCCGCTTCTCACTTCTAGTTTCCCCTCCAAGGTAATGTG |
| | | GTGGGTCATGATCATTCTATCCTGGCTTCAGGGACCTGACTC |
| | | CACTTTGGGGCCATTCGAGGGGTCTAGGGTAGATGATGTCC |
| | | CCCTGTGGGGATTAATGTCCTGCTCTGTAAAACTGAGCTAGC |
| | | TGAGATCCAGGAGGGCTTGGCCAGAGACAGCAAGTTGTTGC |
| | | CATGGTGACTTTAAAGCCAGGTTGCTGCCCCAGCACAGGCCT |
| | | CCCAGTCTACCCTCACTAGAAAACAACACCCAGGCACTTTCC |
| | | ACCACCTCTCAAAGGTGAAACCCAAGGCTGGTCTAGAGAATG |
| | | AATTATGGATCCT |
| 36 | SEQ ID NO: 24 fused | CTGCAGCTCAGCCTACTACTTGCTTTCCAGGCTGTTCCTAGT |
| | to SEQ ID NO: 25 | TCCCATGTCAGCTGCTTGTGCTTTCCAGAGACAAAACAGGAA |
| | | TAATAGATGTCATTAAATATACATTGGGCCCCAGGCGGTCAAT |
| | | GTGGCAGCCTGAGCCTCCTTTCCATCTCTGTGGAGGCAGAC |
| | | ATAGGACCCCCAACAAACAGCATGCAGGTTGGGAGCCAGCC |
| | | ACAGGACCCAGGTAAGGGGCCCTGGGTCCTTAAGCTTCTGC |
| | | CACTGGCTCCGGCATTGCAGAGAGAAGAGAAGGGGCGGCA |
| | | GAGCTGAACCTTAGCCTTGCCTTCCTGGGTACCCTTCTGAGC |
| | | CTCACTGTCTTCTGTGAGATGGGCAAAGTGCGGGTGTGACTC |
| | | CTTGGCAACGGTGTTACACCAGGGCAGGTAAAGTTGTAGTTA |
| | | TTTGTGGGGTACACCAGGACTGTTAAAGGTGTAACTATGGTC |
| | | TCACCCAGCATTTTCACTTCTAATAAGTTCAAATGTGATACGG |
| | | CACCTTTCTAAAAATTAGTTTTCAGGGAAATAGGGTTCAAAAC |
| | | TGGTAGTGGTAGGGTCCATTCTCACGACCCCCAGGCCTGCT |
| | | AACCCTGACCAAGCTACCTATTACTTACCCTCCTCTTTCTCCT |
| | | CCTCCTCTTTCTCCTTCTCCTGCTTCCCCTCTTCCTTCTCCCT |
| | | CCCTTCCTCTCCCTCCTCCCCCTCCTTGGCTGTGATCAGATC |
| | | CAGAGCCTGAATGAGCCTCCTGACCCCACACCCCCACTAGC |
| | | ATGGGCCTGCAAGTGCCCAGAAGTCCCTCCTGCCTCCTAAAC |
| | | TGCCCAGCCGATCCATTAGCTCTTCCTTCTTCCCAGTGAAAG |
| | | AAGCAGGCACAGCCTGTCCCTCCCGTTCTACAGAAAGGAAG |
| | | CTACAGCACAGGGAGGGCCAAAGGCCTTCCTGGGACTAGAC |
| | | AGTTGATCAACAGCAGGACTGGAGAGCTGGGCTCCATTTTTG |
| | | TTCCTTGGTGCCCTGCCCCTCCCCATGACCTGCAGAGACATT |
| | | CAGCCTGCCAGGCTTTATGAGGTGGGAGCTGGGCTCTCCCT |
| | | GATGTATTATTCAGCTCCCTGGAGTTGGCCAGCTCCTGTTAC |
| | | ACTGGCCACAGCCCTGGGCATCCGCTTCTCACTTCTAGTTTC |
| | | CCCTCCAAGGTAATGTGGTGGGTCATGATCATTCTATCCTGG |
| | | CTTCAGGGACCTGACTCCACTTTGGGGCCATTCGAGGGGTC |
| | | TAGGGTAGATGATGTCCCCCTGTGGGGATTAATGTCCTGCTC |
| | | TGTAAAACTGAGCTAGCTGAGATCCAGGAGGGCTTGGCCAG |
| | | AGACAGCAAGTTGTTGCCATGGTGACTTTAAAGCCAGGTTGC |
| | | TGCCCCAGCACAGGCCTCCCAGTCTACCCTCACTAGAAAACA |
| | | ACACCCAGGCACTTTCCACCACCTCTCAAAGGTGAAACCCAA |
| | | GGCTGGTCTAGAGAATGAATTATGGATCCTCGCTGTCCGTGC |
| | | CACCCAGCTAGTCCCAGCGGCTCAGACACTGAGGAGAGACT |
| | | GTAGGTTCAGCTACAAGCAAAAAGACCTAGCTGGTCTCCAAG |
| | | CAGTGTCTCCAAGTCCCTGAACCTGTGACACCTGCCCCAGG |
| | | CATCATCAGGCACAGAGGGCCACC |
| 37 | SEQ ID NO: 25 fused | GGTCTCACCCAGCATTTTCACTTCTAATAAGTTCAAATGTGAT |
| | to SEQ ID NO: 24 | ACGGCACCTTTCTAAAAATTAGTTTTCAGGGAAATAGGGTTCA |
| | | AAACTGGTAGTGGTAGGGTCCATTCTCACGACCCCCAGGCCT |
| | | GCTAACCCTGACCAAGCTACCTATTACTTACCCTCCTCTTTCT |
| | | CCTCCTCCTCTTTCTCCTTCTCCTGCTTCCCCTCTTCCTTCTC |
| | | CCTCCCTTCCTCTCCCTCCTCCCCCTCCTTGGCTGTGATCAG |
| | | ATCCAGAGCCTGAATGAGCCTCCTGACCCCACACCCCCACTA |
| | | GCATGGGCCTGCAAGTGCCCAGAAGTCCCTCCTGCCTCCTA |
| | | AACTGCCCAGCCGATCCATTAGCTCTTCCTTCTTCCCAGTGA |
| | | AAGAAGCAGGCACAGCCTGTCCCTCCCGTTCTACAGAAAGG |
| | | AAGCTACAGCACAGGGAGGGCCAAAGGCCTTCCTGGGACTA |
| | | GACAGTTGATCAACAGCAGGACTGGAGAGCTGGGCTCCATTT |
| | | TTGTTCCTTGGTGCCCTGCCCCTCCCCATGACCTGCAGAGAC |
| | | ATTCAGCCTGCCAGGCTTTATGAGGTGGGAGCTGGGCTCTC |
| | | CCTGATGTATTATTCAGCTCCCTGGAGTTGGCCAGCTCCTGT |
| | | TACACTGGCCACAGCCCTGGGCATCCGCTTCTCACTTCTAGT |
| | | TTCCCCTCCAAGGTAATGTGGTGGGTCATGATCATTCTATCCT |
| | | GGCTTCAGGGACCTGACTCCACTTTGGGGCCATTCGAGGGG |
| | | TCTAGGGTAGATGATGTCCCCCTGTGGGGATTAATGTCCTGC |
| | | TCTGTAAAACTGAGCTAGCTGAGATCCAGGAGGGCTTGGCCA |
| | | GAGACAGCAAGTTGTTGCCATGGTGACTTTAAAGCCAGGTTG |
| | | CTGCCCCAGCACAGGCCTCCCAGTCTACCCTCACTAGAAAAC |
| | | AACACCCAGGCACTTTCCACCACCTCTCAAAGGTGAAACCCA |
| | | AGGCTGGTCTAGAGAATGAATTATGGATCCTCGCTGTCCGTG |
| | | CCACCCAGCTAGTCCCAGCGGCTCAGACACTGAGGAGAGAC |
| | | TGTAGGTTCAGCTACAAGCAAAAAGACCTAGCTGGTCTCCAA |
| | | GCAGTGTCTCCAAGTCCCTGAACCTGTGACACCTGCCCCAG |
| | | GCATCATCAGGCACAGAGGGCCACCCTGCAGCTCAGCCTAC |
| | | TACTTGCTTTCCAGGCTGTTCCTAGTTCCCATGTCAGCTGCTT |
| | | GTGCTTTCCAGAGACAAAACAGGAATAATAGATGTCATTAAAT |
| | | ATACATTGGGCCCCAGGCGGTCAATGTGGCAGCCTGAGCCT |
| | | CCTTTCCATCTCTGTGGAGGCAGACATAGGACCCCCAACAAA |
| | | CAGCATGCAGGTTGGGAGCCAGCCACAGGACCCAGGTAAGG |
| | | GGCCCTGGGTCCTTAAGCTTCTGCCACTGGCTCCGGCATTG |
| | | CAGAGAGAAGAGAAGGGGCGGCAGAGCTGAACCTTAGCCTT |
| | | GCCTTCCTGGGTACCCTTCTGAGCCTCACTGTCTTCTGTGAG |
| | | ATGGGCAAAGTGCGGGTGTGACTCCTTGGCAACGGTGTTAC |
| | | ACCAGGGCAGGTAAAGTTGTAGTTATTTGTGGGGTACACCAG |
| | | GACTGTTAAAGGTGTAACTAT |
| 38 | Portion of SEQ ID NO: | CTGCAGCTCAGCCTACTACTTGCTTTCCAGGCTGTTCCTAGT |
| | 24 that contains SEQ | TCCCATGTCAGCTGCTTGTGCTTTCCAGAGACAAAACAGGAA |
| | ID NO: 26 and SEQ ID | TAATAGATGTCATTAAATATACATTGGGCCCCAGGCGGTCAAT |
| | NO: 27 fused to portion | GTGGCAGCCTGAGCCTCCTTTCCATCTCTGTGGAGGCAGAC |
| | of SEQ ID NO: 25 that | ATAGGACCCCCAACAAACAGCATGCAGGTTGGGAGCCAGCC |
| | contains SEQ ID NO: | ACAGGACCCAGGTAAGGGGCCCTGGGTCCTTAAGCTTCTGC |
| | 31 and SEQ ID NO: 32 | CACTGGCTCCGGCATTGCAGAGAGAAGAGAAGGGGCGGCA |
| | | GACTGGAGAGCTGGGCTCCATTTTTGTTCCTTGGTGCCCTGC |
| | | CCCTCCCCATGACCTGCAGAGACATTCAGCCTGCCAGGCTTT |
| | | ATGAGGTGGGAGCTGGGCTCTCCCTGATGTATTATTCAGCTC |
| | | CCTGGAGTTGGCCAGCTCCTGTTACACTGGCCACAGCCCTG |
| | | GGCATCCGCTTCTCACTTCTAGTTTCCCCTCCAAGGTAATGT |
| | | GGTGGGTCATGATCATTCTATCCTGGCTTCAGGGACCTGACT |
| | | CCACTTTGGGGCCATTCGAGGGGTCTAGGGTAGATGATGTC |
| | | CCCCTGTGGGGATTAATGTCCTGCTCTGTAAAACTGAGCTAG |
| | | CTGAGATCCAGGAGGGCTTGGCCAGAGACAGCAAGTTGTTG |
| | | CCATGGTGACTTTAAAGCCAGGTTGCTGCCCCAGCACAGGC |
| | | CTCCCAGTCTACCCTCACTAGAAAACAACACCCAGGCACTTT |
| | | CCACCACCTCTCAAAGGTGAAACCCAAGGCTGGTCTAGAGAA |
| | | TGAATTATGGATCCTCGCTGTCCGTGCCACCCAGCTAGTCCC |
| | | AGCGGCTCAGACACTGAGGAGAGACTGTAGGTTCAGCTACA |
| | | AGCAAAAAGACCTAGCTGGTCTCCAAGCAGTGTCTCCAAGTC |
| | | CCTGAACCTGTGACACCTGCCCCAGGCATCATCAGGCACAG |
| | | AGGGCCACC |
| 39 | Portion of SEQ ID NO: | CTGCAGCTCAGCCTACTACTTGCTTTCCAGGCTGTTCCTAGT |
| | 24 that contains SEQ | TCCCATGTCAGCTGCTTGTGCTTTCCAGAGACAAAACAGGAA |
| | ID NO: 26 and SEQ ID | TAATAGATGTCATTAAATATACATTGGGCCCCAGGCGGTCAAT |
| | NO: 27 fused to portion | GTGGCAGCCTGAGCCTCCTTTCCATCTCTGTGGAGGCAGAC |
| | of SEQ ID NO: 25 that | ATAGGACCCCCAACAAACAGCATGCAGGTTGGGAGCCAGCC |
| | contains SEQ ID NO: | ACAGGACCCAGGTAAGGGGCCCTGGGTCCTTTTTATGAGGT |
| | 31 and SEQ ID NO: 32 | GGGAGCTGGGCTCTCCCTGATGTATTATTCAGCTCCCTGGAG |
| | | TTGGCCAGCTCCTGTTACACTGGCCACAGCCCTGGGCATCC |
| | | GCTGCCATGGTGACTTTAAAGCCAGGTTGCTGCCCCAGCACA |
| | | GGCCTCCCAGTCTACCCTCACTAGAAAACAACACCCAGGCAC |
| | | TTTCCACCACCTCTCAAAGGTGAAACCCAAGGCTGGTCTAGA |
| | | GAATGAATTATGGATCCTCGCTGTCCGTGCCACCCAGCTAGT |
| | | CCCAGCGGCTCAGACACTG |
| 40 | Region 1 of the human | GTATGCCTTTTGAGATGGATGCAGCAGGTTCTGTGAGGCTGC |
| | Myo15 promoter | CAGGAGGGGTAGAGTTCCCGGGGGCCTCGGGCCCCGCTGG |
| | | AGTGTGGAGCAGGCCCATGCTCAGCTCTCCAGGCTGTTCGT |
| | | GGCTCCCCTGTCAGCTGCTCACTCCTTTCCAGAGACAAAACA |
| | | GGAATAATAGACATCATTAAATATACATAGGGCCCCAGGCGG |
| | | TCGGCGTGGTGGGCTGGGCCTCCCTTCC |
| 41 | Region 2 of human | TGCCCTGCCTTCTGAGCCGGCAGCCTGGCTCCCCACCCCAT |
| | Myo15 promoter | GTATTATTCAGCTCCTGAGAGCCAGCCAGCTCCTGTTACACT |
| | | GACCGCAGCCCAGCACCTGCTCTGCCCATTCCCCTCCTCCC |
| | | TTGCCTAGGACCTAGAGGGTTCAAAGTTCTCCTCCAAGATGA |
| | | CTTGGTGGGCTTTGGCCATCCCACCCTAGGCCCCACTTCTG |
| | | GCCCAGTGCAGGTGTGCTGGTGATTTAGGGCAGGTGGCATT |
| | | CCATCTCTGTGGCTCAATGTCTTCCTCTGTGAAGCCGAAGTG |
| | | ACCCAAGGGCTCCCTTCATGGGGTTGAGCCAGCTGTGGCCC |
| | | AGGGAGGGCCTAACCAGGATGAGCACTGATGTTGCCATGAC |
| | | GACTCCGAGGCCAGAATGTCTCCCCCAGCACAGGCCTCATA |
| | | GGCAGGCTTCCCCATCCTGGTAAACAACACCCACACACTTTC |
| | | TACTACTGCTCTAGGGTGAAACCCAAGGCGCTCTAGAGGAGA |
| | | TGAATTATGGATCCGCCCTCCCGGAATCCTGGCTCGGCCCTC |
| | | CCCACGCCACCCAGGGCCAGTCGGGTCTGCTCACAGCCCGA |
| | | GGAGGCCGCGTGTCCAGCCGCGGGCAAGAGACAGAGCAGG |
| | | TCCCTGTGTCTCCAAGTCCCTGAGCCCGTGACACCGGCCCC |
| | | AGGCCCTGTAGAGAGCAGGCAGCCACC |
| 42 | Portion of SEQ ID NO: | CCCCTGTCAGCTGCTCACTCCTTTCCAGAGACAAAACAGGAA |
| | 40 | TAATAGACATCATTAAATATACATAGGGCCCCAGG |
| 43 | Portion of SEQ ID NO: | TGAGCCGGCAGCCTGGCTCCCCACCCCATGTATTATTCAGCT |
| | 41 | CCTGAGAGCCAGCCAGCTCCTGTTACACTGACCGCAGCCC |
| 44 | Portion of SEQ ID NO: | CACAGGCCTCATAGGCAGGCTTCCCCATCCTGGTAAACAACA |
| | 41 | CCCACACACTTTCTACTACTGCTCTAGGGTGAAACCCAAGGC |
| | | GCTCTAGAGGAGATGAATTATGGATCC |
| 45 | Portion of SEQ ID NO: | TGAGCCGGCAGCCTGGCTCCCCACCCCATGTATTATTCAGCT |
| | 41 | CCTGAGAGCCAGCCAGCTCCTGTTACACTGACCGCAGCCCC |
| | (SEQ ID NO: 43 fused | ACAGGCCTCATAGGCAGGCTTCCCCATCCTGGTAAACAACAC |
| | to SEQ ID NO: 44) | CCACACACTTTCTACTACTGCTCTAGGGTGAAACCCAAGGCG |
| | | CTCTAGAGGAGATGAATTATGGATCC |
| 46 | Portion of SEQ ID NO: | CACAGGCCTCATAGGCAGGCTTCCCCATCCTGGTAAACAACA |
| | 41 | CCCACACACTTTCTACTACTGCTCTAGGGTGAAACCCAAGGC |
| | (SEQ ID NO: 44 fused | GCTCTAGAGGAGATGAATTATGGATCCTGAGCCGGCAGCCT |
| | to SEQ ID NO: 43) | GGCTCCCCACCCCATGTATTATTCAGCTCCTGAGAGCCAGCC |
| | | AGCTCCTGTTACACTGACCGCAGCCC |
| 47 | Portion of SEQ ID NO: | TGAGCCGGCAGCCTGGCTCCCCACCCCATGTATTATTCAGCT |
| | 41 (contiguous | CCTGAGAGCCAGCCAGCTCCTGTTACACTGACCGCAGCCCA |
| | sequence including | GCACCTGCTCTGCCCATTCCCCTCCTCCCTTGCCTAGGACCT |
| | SEQ ID NO: 43 and | AGAGGGTTCAAAGTTCTCCTCCAAGATGACTTGGTGGGCTTT |
| | SEQ ID NO: 44) | GGCCATCCCACCCTAGGCCCCACTTCTGGCCCAGTGCAGGT |
| | | GTGCTGGTGATTTAGGGCAGGTGGCATTCCATCTCTGTGGCT |
| | | CAATGTCTTCCTCTGTGAAGCCGAAGTGACCCAAGGGCTCCC |
| | | TTCATGGGGTTGAGCCAGCTGTGGCCCAGGGAGGGCCTAAC |
| | | CAGGATGAGCACTGATGTTGCCATGACGACTCCGAGGCCAG |
| | | AATGTCTCCCCCAGCACAGGCCTCATAGGCAGGCTTCCCCAT |
| | | CCTGGTAAACAACACCCACACACTTTCTACTACTGCTCTAGG |
| | | GTGAAACCCAAGGCGCTCTAGAGGAGATGAATTATGGATCC |
| 48 | Polynucleotide | GTATGCCTTTTGAGATGGATGCAGCAGGTTCTGTGAGGCTGC |
| | containing SEQ ID NO: | CAGGAGGGGTAGAGTTCCCGGGGGCCTCGGGCCCCGCTGG |
| | 40 and SEQ ID NO: 41 | AGTGTGGAGCAGGCCCATGCTCAGCTCTCCAGGCTGTTCGT |
| | | GGCTCCCCTGTCAGCTGCTCACTCCTTTCCAGAGACAAAACA |
| | | GGAATAATAGACATCATTAAATATACATAGGGCCCCAGGCGG |
| | | TCGGCGTGGTGGGCTGGGCCTCCCTTCCCCATAACACTGAG |
| | | CTGCTCTGCTGGGCCAACCGTGCTCCTGGGCCAGCCAGAGG |
| | | ACCCCCATGAGGCGGCATGCAGGCGGGGAGCAGGCCACAG |
| | | AACGCAGGTAAGGAGACCTTAGCCTAGAGTCCTTGGGGTCT |
| | | GTCACTGGCCACCCTCGCATCCCAGGCTGCAGGAAACTGAG |
| | | GCCCAGAGAGGACAAGGACTTTCCTGGACCCACACAGCCAG |
| | | TCAGTGACAGAGCCTAGGGTCTGAGCCAGGCCTGACCCAAC |
| | | CTCCATTTCTGCCTCTCTACCCCTGCCCCCGCCCCAACACAC |
| | | ACACACACACAAGTGGAGTTCCACTGAAACGCCCCTCCTTGC |
| | | CCTGCCTTCTGAGCCGGCAGCCTGGCTCCCCACCCCATGTA |
| | | TTATTCAGCTCCTGAGAGCCAGCCAGCTCCTGTTACACTGAC |
| | | CGCAGCCCAGCACCTGCTCTGCCCATTCCCCTCCTCCCTTGC |
| | | CTAGGACCTAGAGGGTTCAAAGTTCTCCTCCAAGATGACTTG |
| | | GTGGGCTTTGGCCATCCCACCCTAGGCCCCACTTCTGGCCC |
| | | AGTGCAGGTGTGCTGGTGATTTAGGGCAGGTGGCATTCCAT |
| | | CTCTGTGGCTCAATGTCTTCCTCTGTGAAGCCGAAGTGACCC |
| | | AAGGGCTCCCTTCATGGGGTTGAGCCAGCTGTGGCCCAGGG |
| | | AGGGCCTAACCAGGATGAGCACTGATGTTGCCATGACGACT |
| | | CCGAGGCCAGAATGTCTCCCCCAGCACAGGCCTCATAGGCA |
| | | GGCTTCCCCATCCTGGTAAACAACACCCACACACTTTCTACT |
| | | ACTGCTCTAGGGTGAAACCCAAGGCGCTCTAGAGGAGATGA |
| | | ATTATGGATCCGCCCTCCCGGAATCCTGGCTCGGCCCTCCC |
| | | CACGCCACCCAGGGCCAGTCGGGTCTGCTCACAGCCCGAG |
| | | GAGGCCGCGTGTCCAGCCGCGGGCAAGAGACAGAGCAGGT |
| | | CCCTGTGTCTCCAAGTCCCTGAGCCCGTGACACCGGCCCCA |
| | | GGCCCTGTAGAGAGCAGGCAGCCACC |
| 49 | Polynucleotide | GCAGGCCCATGCTCAGCTCTCCAGGCTGTTCGTGGCTCCCC |
| | containing SEQ ID NO: | TGTCAGCTGCTCACTCCTTTCCAGAGACAAAACAGGAATAAT |
| | 42, SEQ ID NO: 43, | AGACATCATTAAATATACATAGGGCCCCAGGCGGTCGGCGTG |
| | and SEQ ID NO: 44 | GTGGGCTGGGCCTCCCTTCCCCATAACACTGAGCTGCTCTG |
| | | CTGGGCCAACCGTGCTCCTGGGCCAGCCAGAGGACCCCCAT |
| | | GAGGCGGCATGCAGGCGGGGAGCAGGCCACAGAACGCAGG |
| | | TAAGGAGACCTTGCCTTCTGAGCCGGCAGCCTGGCTCCCCA |
| | | CCCCATGTATTATTCAGCTCCTGAGAGCCAGCCAGCTCCTGT |
| | | TACACTGACCGCAGCCCAGCACCTGCTCTGCCCATTCCCCTC |
| | | CTCCCTTGCCTAGGACCTAGAGGGTTCAAAGTTCTCCTCCAA |
| | | GATGACTTGGTGGGCTTTGGCCATCGGGCCTAACCAGGATG |
| | | AGCACTGATGTTGCCATGACGACTCCGAGGCCAGAATGTCTC |
| | | CCCCAGCACAGGCCTCATAGGCAGGCTTCCCCATCCTGGTA |
| | | AACAACACCCACACACTTTCTACTACTGCTCTAGGGTGAAAC |
| | | CCAAGGCGCTCTAGAGGAGATGAATTATGGATCCGCCCTCC |
| | | CGGAATCCTGGCTCGGCCCTCCCCACGC |
| 50 | Portion of SEQ ID NO: | CTGCAGCTCAGCCTACTACTTGCTTTCCAGGCTGTTCCTAGT |
| | 24 that contains SEQ | TCCCATGTCAGCTGCTTGTGCTTTCCAGAGACAAAACAGGAA |
| | ID NO: 26 and SEQ ID | TAATAGATGTCATTAAATATACATTGGGCCCCAGGCGGTCAAT |
| | NO: 27 | GTGGCAGCCTGAGCCTCCTTTCCATCTCTGTGGAGGCAGAC |
| | | ATAGGACCCCCAACAAACAGCATGCAGGTTGGGAGCCAGCC |
| | | ACAGGACCCAGGTAAGGGGCCCTGGGTCCTT |
| 51 | Portion of SEQ ID NO: | TTTATGAGGTGGGAGCTGGGCTCTCCCTGATGTATTATTCAG |
| | 25 that contains SEQ | CTCCCTGGAGTTGGCCAGCTCCTGTTACACTGGCCACAGCC |
| | ID NO: 31 | CTGGGCATCCGC |
| 52 | Portion of SEQ ID NO: | TGCCATGGTGACTTTAAAGCCAGGTTGCTGCCCCAGCACAG |
| | 25 that contains SEQ | GCCTCCCAGTCTACCCTCACTAGAAAACAACACCCAGGCACT |
| | ID NO: 32 | TTCCACCACCTCTCAAAGGTGAAACCCAAGGCTGGTCTAGAG |
| | | AATGAATTATGGATCCTCGCTGTCCGTGCCACCCAGCTAGTC |
| | | CCAGCGGCTCAGACACTG |
| 53 | SEQ ID NO: 50 fused | CTGCAGCTCAGCCTACTACTTGCTTTCCAGGCTGTTCCTAGT |
| | to SEQ ID NO: 51 | TCCCATGTCAGCTGCTTGTGCTTTCCAGAGACAAAACAGGAA |
| | | TAATAGATGTCATTAAATATACATTGGGCCCCAGGCGGTCAAT |
| | | GTGGCAGCCTGAGCCTCCTTTCCATCTCTGTGGAGGCAGAC |
| | | ATAGGACCCCCAACAAACAGCATGCAGGTTGGGAGCCAGCC |
| | | ACAGGACCCAGGTAAGGGGCCCTGGGTCCTTTTTATGAGGT |
| | | GGGAGCTGGGCTCTCCCTGATGTATTATTCAGCTCCCTGGAG |
| | | TTGGCCAGCTCCTGTTACACTGGCCACAGCCCTGGGCATCC |
| | | GC |
| 54 | SEQ ID NO: 50 fused | CTGCAGCTCAGCCTACTACTTGCTTTCCAGGCTGTTCCTAGT |
| | to SEQ ID NO: 52 | TCCCATGTCAGCTGCTTGTGCTTTCCAGAGACAAAACAGGAA |
| | | TAATAGATGTCATTAAATATACATTGGGCCCCAGGCGGTCAAT |
| | | GTGGCAGCCTGAGCCTCCTTTCCATCTCTGTGGAGGCAGAC |
| | | ATAGGACCCCCAACAAACAGCATGCAGGTTGGGAGCCAGCC |
| | | ACAGGACCCAGGTAAGGGGCCCTGGGTCCTTTGCCATGGTG |
| | | ACTTTAAAGCCAGGTTGCTGCCCCAGCACAGGCCTCCCAGTC |
| | | TACCCTCACTAGAAAACAACACCCAGGCACTTTCCACCACCT |
| | | CTCAAAGGTGAAACCCAAGGCTGGTCTAGAGAATGAATTATG |
| | | GATCCTCGCTGTCCGTGCCACCCAGCTAGTCCCAGCGGCTC |
| | | AGACACTG |
| 55 | SEQ ID NO: 51 fused | TTTATGAGGTGGGAGCTGGGCTCTCCCTGATGTATTATTCAG |
| | to SEQ ID NO: 52 | CTCCCTGGAGTTGGCCAGCTCCTGTTACACTGGCCACAGCC |
| | | CTGGGCATCCGCTGCCATGGTGACTTTAAAGCCAGGTTGCTG |
| | | CCCCAGCACAGGCCTCCCAGTCTACCCTCACTAGAAAACAAC |
| | | ACCCAGGCACTTTCCACCACCTCTCAAAGGTGAAACCCAAGG |
| | | CTGGTCTAGAGAATGAATTATGGATCCTCGCTGTCCGTGCCA |
| | | CCCAGCTAGTCCCAGCGGCTCAGACACTG |
| 56 | SEQ ID NO: 51 fused | TTTATGAGGTGGGAGCTGGGCTCTCCCTGATGTATTATTCAG |
| | to SEQ ID NO: 50, | CTCCCTGGAGTTGGCCAGCTCCTGTTACACTGGCCACAGCC |
| | which is fused to SEQ | CTGGGCATCCGCCTGCAGCTCAGCCTACTACTTGCTTTCCAG |
| | ID NO: 52 | GCTGTTCCTAGTTCCCATGTCAGCTGCTTGTGCTTTCCAGAG |
| | | ACAAAACAGGAATAATAGATGTCATTAAATATACATTGGGCCC |
| | | CAGGCGGTCAATGTGGCAGCCTGAGCCTCCTTTCCATCTCTG |
| | | TGGAGGCAGACATAGGACCCCCAACAAACAGCATGCAGGTT |
| | | GGGAGCCAGCCACAGGACCCAGGTAAGGGGCCCTGGGTCC |
| | | TTTGCCATGGTGACTTTAAAGCCAGGTTGCTGCCCCAGCACA |
| | | GGCCTCCCAGTCTACCCTCACTAGAAAACAACACCCAGGCAC |
| | | TTTCCACCACCTCTCAAAGGTGAAACCCAAGGCTGGTCTAGA |
| | | GAATGAATTATGGATCCTCGCTGTCCGTGCCACCCAGCTAGT |
| | | CCCAGCGGCTCAGACACTG |
| 57 | SEQ ID NO: 52 fused | TGCCATGGTGACTTTAAAGCCAGGTTGCTGCCCCAGCACAG |
| | to SEQ ID NO: 50, | GCCTCCCAGTCTACCCTCACTAGAAAACAACACCCAGGCACT |
| | which is fused to SEQ | TTCCACCACCTCTCAAAGGTGAAACCCAAGGCTGGTCTAGAG |
| | ID NO: 51 | AATGAATTATGGATCCTCGCTGTCCGTGCCACCCAGCTAGTC |
| | | CCAGCGGCTCAGACACTGCTGCAGCTCAGCCTACTACTTGCT |
| | | TTCCAGGCTGTTCCTAGTTCCCATGTCAGCTGCTTGTGCTTTC |
| | | CAGAGACAAAACAGGAATAATAGATGTCATTAAATATACATTG |
| | | GGCCCCAGGCGGTCAATGTGGCAGCCTGAGCCTCCTTTCCA |
| | | TCTCTGTGGAGGCAGACATAGGACCCCCAACAAACAGCATG |
| | | CAGGTTGGGAGCCAGCCACAGGACCCAGGTAAGGGGCCCT |
| | | GGGTCCTTTTTATGAGGTGGGAGCTGGGCTCTCCCTGATGTA |
| | | TTATTCAGCTCCCTGGAGTTGGCCAGCTCCTGTTACACTGGC |
| | | CACAGCCCTGGGCATCCGC |
| 58 | SEQ ID NO: 51 fused | TTTATGAGGTGGGAGCTGGGCTCTCCCTGATGTATTATTCAG |
| | to SEQ ID NO: 52, | CTCCCTGGAGTTGGCCAGCTCCTGTTACACTGGCCACAGCC |
| | which is fused to SEQ | CTGGGCATCCGCTGCCATGGTGACTTTAAAGCCAGGTTGCTG |
| | ID NO: 50 | CCCCAGCACAGGCCTCCCAGTCTACCCTCACTAGAAAACAAC |
| | | ACCCAGGCACTTTCCACCACCTCTCAAAGGTGAAACCCAAGG |
| | | CTGGTCTAGAGAATGAATTATGGATCCTCGCTGTCCGTGCCA |
| | | CCCAGCTAGTCCCAGCGGCTCAGACACTGCTGCAGCTCAGC |
| | | CTACTACTTGCTTTCCAGGCTGTTCCTAGTTCCCATGTCAGCT |
| | | GCTTGTGCTTTCCAGAGACAAAACAGGAATAATAGATGTCATT |
| | | AAATATACATTGGGCCCCAGGCGGTCAATGTGGCAGCCTGA |
| | | GCCTCCTTTCCATCTCTGTGGAGGCAGACATAGGACCCCCAA |
| | | CAAACAGCATGCAGGTTGGGAGCCAGCCACAGGACCCAGGT |
| | | AAGGGGCCCTGGGTCCTT |
| 59 | Portion of SEQ ID NO: | TGCAGCTCAGCCTACTACTTGCTTTCCAGGCTGTTCCTAGTT |
| | 24 that contains SEQ | CCCATGTCAGCTGCTTGTGCTTTCCAGAGACAAAACAGGAAT |
| | ID NO: 26 and SEQ ID | AATAGATGTCATTAAATATACATTGGGCCCCAGGCGGTCAAT |
| | NO: 27 fused to portion | GTGGCAGCCTGAGCCTCCTTTCCATCTCTGTGGAGGCAGAC |
| | of SEQ ID NO: 25 that | ATAGGACCCCCAACAAACAGCATGCAGGTTGGGAGCCAGCC |
| | contains SEQ ID NO: | ACAGGACCCAGGTAAGGGGCCCTGGGTCCTTAAGCTTCTGC |
| | 31 and SEQ ID NO: 32 | CACTGGCTCCGGCATTGCAGAGAGAAGAGAAGGGGCGGCA |
| | | GACTGGAGAGCTGGGCTCCATTTTTGTTCCTTGGTGCCCTGC |
| | | CCCTCCCCATGACCTGCAGAGACATTCAGCCTGCCAGGCTTT |
| | | ATGAGGTGGGAGCTGGGCTCTCCCTGATGTATTATTCAGCTC |
| | | CCTGGAGTTGGCCAGCTCCTGTTACACTGGCCACAGCCCTG |
| | | GGCATCCGCTTCTCACTTCTAGTTTCCCCTCCAAGGTAATGT |
| | | GGTGGGTCATGATCATTCTATCCTGGCTTCAGGGACCTGACT |
| | | CCACTTTGGGGCCATTCGAGGGGTCTAGGGTAGATGATGTC |
| | | CCCCTGTGGGGATTAATGTCCTGCTCTGTAAAACTGAGCTAG |
| | | CTGAGATCCAGGAGGGCTTGGCCAGAGACAGCAAGTTGTTG |
| | | CCATGGTGACTTTAAAGCCAGGTTGCTGCCCCAGCACAGGC |
| | | CTCCCAGTCTACCCTCACTAGAAAACAACACCCAGGCACTTT |
| | | CCACCACCTCTCAAAGGTGAAACCCAAGGCTGGTCTAGAGAA |
| | | TGAATTATGGATCCTCGCTGTCCGTGCCACCCAGCTAGTCCC |
| | | AGCGGCTCAGACACTGAGGAGAGACTGTAGGTTCAGCTACA |
| | | AGCAAAAAGACCTAGCTGGTCTCCAAGCAGTGTCTCCAAGTC |
| | | CCTGAACCTGTGACACCTGCCCCAGGCATCATCAGGCACAG |
| | | AGGGCCACC |
| 60 | Portion of SEQ ID NO: | TGCAGCTCAGCCTACTACTTGCTTTCCAGGCTGTTCCTAGTT |
| | 24 that contains SEQ | CCCATGTCAGCTGCTTGTGCTTTCCAGAGACAAAACAGGAAT |
| | ID NO: 26 and SEQ ID | AATAGATGTCATTAAATATACATTGGGCCCCAGGCGGTCAAT |
| | NO: 27 fused to portion | GTGGCAGCCTGAGCCTCCTTTCCATCTCTGTGGAGGCAGAC |
| | of SEQ ID NO: 25 that | ATAGGACCCCCAACAAACAGCATGCAGGTTGGGAGCCAGCC |
| | contains SEQ ID NO: | ACAGGACCCAGGTAAGGGGCCCTGGGTCCTTTTTATGAGGT |
| | 31 and SEQ ID NO: 32 | GGGAGCTGGGCTCTCCCTGATGTATTATTCAGCTCCCTGGAG |
| | | TTGGCCAGCTCCTGTTACACTGGCCACAGCCCTGGGCATCC |
| | | GCTGCCATGGTGACTTTAAAGCCAGGTTGCTGCCCCAGCACA |
| | | GGCCTCCCAGTCTACCCTCACTAGAAAACAACACCCAGGCAC |
| | | TTTCCACCACCTCTCAAAGGTGAAACCCAAGGCTGGTCTAGA |
| | | GAATGAATTATGGATCCTCGCTGTCCGTGCCACCCAGCTAGT |
| | | CCCAGCGGCTCAGACACTG |

Additional Myo15 promoters useful in conjunction with the compositions and methods described herein include nucleic acid molecules that have at least 85% sequence identity (e.g., 85%, 90%, 95%, 96%, 97%, 98%, 99%, or more, sequence identity) to the nucleic acid sequences set forth in Table 3, as well as functional portions or derivatives of the nucleic acid sequences set forth in Table 3. The Myo15 promoters listed in Table 3 are characterized in U.S. Provisional Application Nos. 62/663,679, 62/802,874, 62/928,311, and 62/965,773 and International Application No. PCT/US2019/029336, which are incorporated herein by reference.

Once a polynucleotide encoding OTOF has been incorporated into the nuclear DNA of a mammalian cell or stabilized in an episomal monomer or concatemer, the transcription of this polynucleotide can be induced by methods known in the art. For example, expression can be induced by exposing the mammalian cell to an external chemical reagent, such as an agent that modulates the binding of a transcription factor and/or RNA polymerase to the mammalian promoter and thus regulates gene expression. The chemical reagent can serve to facilitate the binding of RNA polymerase and/or transcription factors to the mammalian promoter, e.g., by removing a repressor protein that has bound the promoter. Alternatively, the chemical reagent can serve to enhance the affinity of the mammalian promoter for RNA polymerase and/or transcription factors such that the rate of transcription of the gene located downstream of the promoter is increased in the presence of the chemical reagent. Examples of chemical reagents that potentiate polynucleotide transcription by the above mechanisms include tetracycline and doxycycline. These reagents are commercially available (Life Technologies, Carlsbad, CA) and can be administered to a mammalian cell in order to promote gene expression according to established protocols.

Other DNA sequence elements that may be included in the nucleic acid vectors for use in the compositions and methods described herein include enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that encode an OTOF protein and additionally include a mammalian enhancer sequence. Many enhancer sequences are now known from mammalian genes, and examples include enhancers from the genes that encode mammalian globin, elastase, albumin, α-fetoprotein, and insulin. Enhancers for use in the compositions and methods described herein also include those that are derived from the genetic material of a virus capable of infecting a eukaryotic cell. Examples include the SV40 enhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription are disclosed in Yaniv, et al., Nature 297:17 (1982). An enhancer may be spliced into a vector containing a polynucleotide encoding an OTOF protein, for example, at a position 5′ or 3′ to this gene. In a preferred orientation, the enhancer is positioned at the 5′ side of the promoter, which in turn is located 5′ relative to the polynucleotide encoding an OTOF protein.

The nucleic acid vectors described herein may include a Woodchuck Posttranscriptional Regulatory Element (WPRE). The WPRE acts at the mRNA level, by promoting nuclear export of transcripts and/or by increasing the efficiency of polyadenylation of the nascent transcript, thus increasing the total amount of mRNA in the cell. The addition of the WPRE to a vector can result in a substantial improvement in the level of transgene expression from several different promoters, both in vitro and in vivo. The WPRE can be located in the second nucleic acid vector between the polynucleotide encoding a C-terminal portion of an OTOF protein and the poly(A) sequence. In the compositions and methods described herein, the WPRE can have the sequence:

(SEQ ID NO: 23)
GATCCAATCAACCTCTGGATTACAAAATTTGTGAAAGATTGACTGGTATTCTTAACTA
TGTTGCTCCTTTTACGCTATGTGGATACGCTGCTTTAATGCCTTTGTATCATGCTATT
GCTTCCCGTATGGCTTTCATTTTCTCCTCCTTGTATAAATCCTGGTTGCTGTCTCTTT
ATGAGGAGTTGTGGCCCGTTGTCAGGCAACGTGGCGTGGTGTGCACTGTGTTTGCT
GACGCAACCCCCACTGGTTGGGGCATTGCCACCACCTGTCAGCTCCTTTCCGGGAC
TTTCGCTTTCCCCCTCCCTATTGCCACGGCGGAACTCATCGCCGCCTGCCTTGCCC
GCTGCTGGACAGGGGCTCGGCTGTTGGGCACTGACAATTCCGTGGTGTTGTCGGG
GAAATCATCGTCCTTTCCTTGGCTGCTCGCCTGTGTTGCCACCTGGATTCTGCGCG
GGACGTCCTTCTGCTACGTCCCTTCGGCCCTCAATCCAGCGGACCTTCCTTCCCGC
GGCCTGCTGCCGGCTCTGCGGCCTCTTCCGCGTCTTCGA.
The WPRE can also have the sequence:
(SEQ ID NO: 61)
AATCAACCTCTGGATTACAAAATTTGTGAAAGATTGACTGGTATTCTTAACTATGTTG
CTCCTTTTACGCTATGTGGATACGCTGCTTTAATGCCTTTGTATCATGCTATTGCTTC
CCGTATGGCTTTCATTTTCTCCTCCTTGTATAAATCCTGGTTAGTTCTTGCCACGGCG
GAACTCATCGCCGCCTGCCTTGCCCGCTGCTGGACAGGGGCTCGGCTGTTGGGCA
CTGACAATTCCGTGGTGTTTATTTGTGAAATTTGTGATGCTATTGCTTTATTTGTAAC
CATCTAGCTTTATTTGTGAAATTTGTGATGCTATTGCTTTATTTGTAACCATTATAAGC
TGCAATAAACAAGTTAACAACAACAATTGCATTCATTTTATGTTTCAGGTTCAGGGGG
AGATGTGGGAGGTTTTTTAAA.

In some embodiments, the nucleic acid vectors for use in the compositions and methods described herein include a reporter sequence, which can be useful in verifying OTOF gene expression, for example, in specific cells and tissues (e.g., in cochlear hair cells). Reporter sequences that may be provided in a transgene include DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements which drive their expression, the reporter sequences provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA), and immunohistochemistry. For example, where the marker sequence is the LacZ gene, the presence of the vector carrying the signal is detected by assays for β-galactosidase activity. Where the transgene is green fluorescent protein or luciferase, the vector carrying the signal may be measured visually by color or light production in a luminometer.

### Overlapping Dual Vectors

One approach for expressing large proteins in mammalian cells involves the use of overlapping dual vectors. This approach is based on the use of two nucleic acid vectors, each of which contains a portion of a polynucleotide that encodes a protein of interest and has a defined region of sequence overlap with the other polynucleotide. Homologous recombination can occur at the region of overlap and lead to the formation of a single nucleic acid molecule that encodes the full-length protein of interest.

Overlapping dual vectors for use in the methods and compositions described herein contain at least one kilobase (kb) of overlapping sequence (e.g., 1 kb, 1.5 kb, 2 kb, 2.5 kb, 3 kb or more of overlapping sequence). The nucleic acid vectors are designed such that the overlapping region is centered at an OTOF exon boundary, with an equal amount of overlap on either side of the boundary. The boundaries are chosen based on the size of the promoter and the locations of the portions of the polynucleotide that encode OTOF C2 domains. Overlapping regions are centered on exon boundaries that occur outside of the portion of the polynucleotide that encodes the C2C domain (e.g., after the portion of the polynucleotide that encodes the C2C domain). Exon boundaries within the portion of the polynucleotide that encodes the C2D domain can be selected as the center of the overlapping region, or exon boundaries located after the portion of the polynucleotide that encodes the C2D domain and before the portion of the polynucleotide that encodes the C2E domain can serve as the center of an overlapping region. The nucleic acid vectors for use in the methods and compositions described herein are also designed such that approximately half of the OTOF gene is contained within each vector (e.g., each vector contains a polynucleotide that encodes approximately half of the OTOF protein).

One exemplary overlapping dual vector system includes a first nucleic acid vector containing a CAG promoter operably linked to exons 1-28 and the 500 kb immediately 3′ of the exon 28/29 boundary of a polynucleotide encoding an OTOF protein (e.g., human OTOF, e.g., SEQ ID NO: 1 or SEQ ID NO: 5, or mouse OTOF, e.g., SEQ ID NO: 6); and a second nucleic acid vector containing the 500 kb immediately 5′ of the exon 28/29 boundary and exons 29-48 of a polynucleotide encoding an OTOF protein (e.g., human OTOF, e.g., SEQ ID NO: 1 or SEQ ID NO: 5, or mouse OTOF, e.g., SEQ ID NO: 6) and a poly(A) sequence (e.g., a bovine growth hormone (bGH) poly(A) signal sequence). In this overlapping dual vector system, the overlapping sequence is centered at the exon 28/29 boundary, which is after the portion of the polynucleotide that encodes the C2D domain. Another exemplary overlapping dual vector system includes a first nucleic acid vector containing a CAG promoter operably linked to exons 1-24 and the 500 kb immediately 3′ of the exon 24/25 boundary of a polynucleotide encoding an OTOF protein (e.g., human OTOF, e.g., SEQ ID NO: 1 or SEQ ID NO: 5, or mouse OTOF, e.g., SEQ ID NO: 6); and a second nucleic acid vector containing the 500 kb immediately 5′ of the exon 24/25 boundary and exons 25-48 of a polynucleotide encoding an OTOF protein (e.g., human OTOF, e.g., SEQ ID NO: 1 or SEQ ID NO: 5, or mouse OTOF, e.g., SEQ ID NO: 6) and a poly(A) sequence (e.g., a bGH poly(A) signal sequence). In this overlapping dual vector system, the overlapping sequence is centered at the exon 24/25 boundary, which is within the portion of the polynucleotide that encodes the C2D domain. The two exon boundaries described above can be used with any promoter that is a similar size to the CAG promoter (e.g., the CMV promoter or smCBA promoter), such as promoters that are 1 kb or shorter (e.g., approximately 1 kb, 950 bp, 900 bp, 850 bp, 800 bp, 750 bp, 700 bp, 650 bp, 600 bp, 550 bp 500 bp, 450 bp, 400 bp, 350 bp, 300 bp or shorter). For example, in either of the foregoing dual vector systems, the CMV promoter or the smCBA promoter, can be used in the place of the CAG promoter. A Myo15 promoter having a sequence that is 1 kb or shorter (e.g., a Myo15 promoter described hereinabove, e.g., a Myo15 promoter having the sequence of any one of SEQ ID NOs: 38, 39, or 49-60) can also be used in place of the CAG promoter. Alternatively, a different exon boundary can be chosen that is within or after the portion of the polynucleotide that encodes the C2D domain and before the portion of the polynucleotide that encodes the C2E domain. The nucleic acid vectors containing promoters of this size can optionally contain OTOF UTRs. For example, in the foregoing overlapping dual vector system in which the overlapping region is centered at the exon 28/29 boundary of OTOF, the second nucleic acid vector can contain the full length OTOF 3′ UTR (e.g., the 1035 bp human OTOF 3′ UTR in dual vector systems encoding human OTOF, or the 1001 bp mouse OTOF 3′ UTR in dual vector systems encoding mouse OTOF). In the foregoing overlapping dual vector system in which the overlapping region is centered at the exon 24/25 boundary of OTOF, neither the first nor the second nucleic acid vector contains an OTOF UTR.

In some embodiments, the first nucleic acid vector in the overlapping dual vector system contains a long promoter (e.g., a promoter that is longer than 1 kb, e.g., 1.1 kb, 1.25 kb, 1.5 kb, 1.75 kb, 2 kb, 2.5 kb, 3 kb or longer). In such overlapping dual vector systems, the overlapping region is centered at an exon boundary that is located after the portion of the polynucleotide that encodes the C2C domain and before the portion of the polynucleotide that encodes the C2D domain. For example, an overlapping dual vector system for use in the methods and compositions described herein includes a first nucleic acid vector containing a Myo15 promoter that is longer than 1 kb (e.g., SEQ ID NO: 36) operably linked exons 1-21 and the 500 kb immediately 3′ of the exon 21/22 boundary of a polynucleotide encoding an OTOF protein (e.g., human OTOF, e.g., SEQ ID NO: 1 or SEQ ID NO: 5); and a second nucleic acid vector containing the 500 kb immediately 5′ of the exon 21/22 boundary and exons 22-48 of a polynucleotide encoding an OTOF protein (e.g., human OTOF, e.g., SEQ ID NO: 1 or SEQ ID NO: 5) and a poly(A) sequence (e.g., a bGH poly(A) signal sequence). In this overlapping dual vector system, neither the first nor the second nucleic acid vector includes an OTOF UTR. A short promoter (e.g., a CMV promoter, CAG promoter, smCBA promoter, or a Myo15 promoter having a sequence that is 1 kb or shorter, e.g., a Myo15 promoter having the sequence of any one of SEQ ID NOs: 38, 39, or 49-60) can also be used in this dual vector system (e.g., a dual vector system in which the overlapping region is centered at the exon 21/22 boundary). If a short promoter is used, additional elements, such as a 5′ OTOF UTR, can be included in the first vector (e.g., the vector containing exons 1-21 and the 500 kb immediately 3′ of the exon 21/22 boundary of a polynucleotide encoding an OTOF protein).

### Trans-Splicing Dual Vectors

A second approach for expressing large proteins in mammalian cells involves the use of trans-splicing dual vectors. In this approach, two nucleic acid vectors are used that contain distinct nucleic acid sequences, and the polynucleotide encoding the N-terminal portion of the protein of interest and the polynucleotide encoding the C-terminal portion of the protein of interest do not overlap. Instead, the first nucleic acid vector includes a splice donor sequence 3′ of the polynucleotide encoding the N-terminal portion of the protein of interest, and the second nucleic acid vector includes a splice acceptor sequence 5′ of the polynucleotide encoding the C-terminal portion of the protein of interest. When the first and second nucleic acids are present in the same cell, their ITRs can concatemerize, forming a single nucleic acid structure in which the concatemerized ITRs are positioned between the splice donor and splice acceptor. Trans-splicing then occurs during transcription, producing a nucleic acid molecule in which the polynucleotides encoding the N-terminal and C-terminal portions of the protein of interest are contiguous, thereby forming the full-length coding sequence.

Trans-splicing dual vectors for use in the methods and compositions described herein are designed such that approximately half of the OTOF gene is contained within each vector (e.g., each vector contains a polynucleotide that encodes approximately half of the OTOF protein). The determination of how to split the polynucleotide sequence between the two nucleic acid vectors is made based on the size of the promoter and the locations of the portions of the polynucleotide that encode the OTOF C2 domains. When a short promoter is used in the trans-splicing dual vector system (e.g., a promoter that is 1 kb or shorter, e.g., approximately 1 kb, 950 bp, 900 bp, 850 bp, 800 bp, 750 bp, 700 bp, 650 bp, 600 bp, 550 bp 500 bp, 450 bp, 400 bp, 350 bp, 300 bp or shorter), such as a CAG promoter, a CMV promoter, a smCBA promoter, or a Myo15 promoter having a sequence that is 1 kb or shorter (e.g., a Myo15 promoter described hereinabove, e.g., a Myo15 promoter having the sequence of any one of SEQ ID NOs: 38, 39, or 49-60) the OTOF polynucleotide sequence is divided between the two nucleic acid vectors at an exon boundary that occurs after the portion of the polynucleotide that encodes the C2D domain and before the portion of the polynucleotide that encodes the C2E domain, for example, the exon 26/27 boundary. The nucleic acid vectors containing promoters of this size can optionally contain OTOF UTRs (e.g., both the 5′ and 3′ OTOF UTRs, e.g., full-length UTRs). When a long promoter is used in the trans-splicing dual vector system (e.g., a promoter that is longer than 1 kb, e.g., 1.1 kb, 1.25 kb, 1.5 kb, 1.75 kb, 2 kb, 2.5 kb, 3 kb or longer), such as a Myo15 promoter that is longer than 1 kb (e.g., SEQ ID NO: 36), the OTOF polynucleotide sequence will be divided between the two nucleic acid vectors at an exon boundary that occurs after the portion of the polynucleotide that encodes the C2C domain, and either before the portion of the polynucleotide that encodes the C2D domain, such as the exon 19/20 boundary, or within the portion of the polynucleotide that encodes the C2D domain, such as the exon 25/26 boundary. A short promoter (e.g., a CMV promoter, smCBA promoter, CAG promoter, or a Myo15 promoter having a sequence that is 1 kb or shorter, e.g., a Myo15 promoter having the sequence of any one of SEQ ID NOs: 38, 39, or 49-60) can also be used in the dual vector systems designed for large promoters, in which case additional elements (e.g., OTOF UTR sequences) may be included in the first vector (e.g., the vector containing the portion of the polynucleotide the encodes the C2C domain).

One exemplary trans-splicing dual vector system that uses a short promoter includes a first nucleic acid vector containing a CAG promoter operably linked to exons 1-26 of a polynucleotide encoding an OTOF protein (e.g., human OTOF, e.g., SEQ ID NO: 1 or SEQ ID NO: 5) and a splice donor sequence 3′ of the polynucleotide sequence; and a second nucleic acid vector containing a splice acceptor sequence 5′ of exons 27-48 of a polynucleotide encoding an OTOF protein (e.g., human OTOF, e.g., SEQ ID NO: 1 or SEQ ID NO: 5) and a poly(A) sequence (e.g., a bGH poly(A) signal sequence). An alternative trans-splicing dual vector system includes a first nucleic acid vector containing a CAG promoter operably linked to exons 1-28 of a polynucleotide encoding an OTOF protein (e.g., mouse OTOF, e.g., SEQ ID NO: 6) and a splice donor sequence 3′ of the polynucleotide sequence; and a second nucleic acid vector containing a splice acceptor sequence 5′ of exons 29-48 of a polynucleotide encoding an OTOF protein (e.g., mouse OTOF, e.g., SEQ ID NO: 6) and a poly(A) sequence (e.g., a bGH poly(A) signal sequence). The CMV promoter, smCBA promoter, or a Myo15 promoter having a sequence that is 1 kb or shorter (e.g., a Myo15 promoter described hereinabove, e.g., a Myo15 promoter having the sequence of any one of SEQ ID NOs: 38, 39, or 49-60) can be used in place of the CAG promoter either of the foregoing dual vector systems. These nucleic acid vectors can also contain full length 5′ and 3′ OTOF UTRs in the first and second nucleic acid vectors, respectively (e.g., the first nucleic acid vector can contain the 5′ human OTOF UTR (127 bp) in dual vector systems encoding human OTOF, or the 5′ mouse UTR (134 bp) in dual vector systems encoding mouse OTOF; and the second nucleic acid vector can contain the 3′ human OTOF UTR (1035 bp) in dual vector systems encoding human OTOF, or the 3′ mouse OTOF UTR (1001 bp) in dual vector systems encoding mouse OTOF).

An exemplary trans-splicing dual vector system that uses a long promoter includes a first nucleic acid vector containing a Myo15 promoter that is longer than 1 kb (e.g., SEQ ID NO: 36) operably linked to exons 1-19 of a polynucleotide encoding an OTOF protein (e.g., human OTOF, e.g., SEQ ID NO: 1 or SEQ ID NO: 5) and a splice donor sequence 3′ of the polynucleotide sequence; and a second nucleic acid vector containing a splice acceptor sequence 5′ of exons 20-48 of a polynucleotide encoding an OTOF protein (e.g., human OTOF, e.g., SEQ ID NO: 1 or SEQ ID NO: 5) and a poly(A) sequence (e.g., a bGH poly(A) signal sequence). Alternatively, the trans-splicing dual vector system can include a first nucleic acid vector containing a Myo15 promoter that is longer than 1 kb (e.g., SEQ ID NO: 36) operably linked to exons 1-20 of a polynucleotide encoding an OTOF protein (e.g., mouse OTOF, e.g., SEQ ID NO: 6) and a splice donor sequence 3′ of the polynucleotide sequence; and a second nucleic acid vector containing a splice acceptor sequence 5′ of exons 21-48 of a polynucleotide encoding an OTOF protein (e.g., mouse OTOF, e.g., SEQ ID NO: 6) and a poly(A) sequence (e.g., a bGH poly(A) signal sequence). Neither the first nor the second nucleic acid vector in either of the foregoing Myo15 promoter trans-splicing dual vector systems contains an OTOF UTR. A short promoter (e.g., a CMV promoter, smCBA promoter, CAG promoter, or a Myo15 promoter having a sequence that is 1 kb or shorter, e.g., a Myo15 promoter having the sequence of any one of SEQ ID NOs: 38, 39, or 49-60) can also be used in the foregoing dual vector systems designed for large promoters. If these dual vector systems contain a short promoter, they may also include a 5′ OTOF UTR in the first vector.

To accommodate an OTOF UTR, the OTOF coding sequence can be divided in a different position. For example, in a trans-splicing dual vector system in which the first nucleic acid vector contains a Myo15 promoter that is longer than 1 kb (e.g., SEQ ID NO: 36) operably linked to exons 1-25 of a polynucleotide encoding an OTOF protein (e.g., human OTOF, e.g., SEQ ID NO: 1 or SEQ ID NO: 5) and a splice donor sequence 3′ of the polynucleotide sequence; and the second nucleic acid vector contains a splice acceptor sequence 5′ of exons 26-48 of a polynucleotide encoding an OTOF protein (e.g., human OTOF, e.g., SEQ ID NO: 1 or SEQ ID NO: 5) and a poly(A) sequence (e.g., a bGH poly(A) signal sequence), the second nucleic acid can also contain a full length OTOF 3′ UTR (e.g., the 1035 bp human OTOF 3′ UTR). For mouse OTOF, the trans-splicing dual vector system can contain a 3′ UTR if the first nucleic acid vector contains a Myo15 promoter that is longer than 1 kb (e.g., SEQ ID NO: 36) operably linked to exons 1-24 of a polynucleotide encoding an OTOF protein (e.g., mouse OTOF, e.g., SEQ ID NO: 6) and a splice donor sequence 3′ of the polynucleotide sequence; and the second nucleic acid vector contains a splice acceptor sequence 5′ of exons 25-48 of a polynucleotide encoding an OTOF protein (e.g., mouse OTOF, e.g., SEQ ID NO: 6) and a poly(A) sequence (e.g., a bGH poly(A) signal sequence). In this dual vector system, the second nucleic acid can also contain a full length OTOF 3′ UTR (e.g., the 1001 bp mouse OTOF 3′ UTR). A short promoter (e.g., a CMV promoter, smCBA promoter, CAG promoter, or a Myo15 promoter having a sequence that is 1 kb or shorter, e.g., a Myo15 promoter having the sequence of any one of SEQ ID NOs: 38, 39, or 49-60) can also be used in the foregoing dual vector systems designed for large promoters. If these dual vector systems contain a short promoter, they may also include a 5′ OTOF UTR in the first vector.

### Dual Hybrid Vectors

A third approach for expressing large proteins in mammalian cells involves the use of dual hybrid vectors. This approach combines elements of the overlapping dual vector strategy and the trans-splicing strategy in that it features both an overlapping region at which homologous recombination can occur and splice donor and splice acceptor sequences. In dual hybrid vector systems, the overlapping region is a recombinogenic region that is contained in both the first and second nucleic acid vectors, rather than a portion of the polynucleotide sequence encoding the protein of interest—the polynucleotide encoding the N-terminal portion of the protein of interest and the polynucleotide encoding the C-terminal portion of the protein of interest do not overlap in this approach. The recombinogenic region is 3′ of the splice donor sequence in the first nucleic acid vector and 5′ of the splice acceptor sequence in the second nucleic acid sequence. The first and second nucleic acid sequences can then join to form a single sequence based on one of two mechanisms: 1) recombination at the overlapping region, or 2) concatemerization of the ITRs. The remaining recombinogenic region(s) and/or the concatemerized ITRs can be removed by splicing, leading to the formation of a contiguous polynucleotide sequence that encodes the full-length protein of interest.

Recombinogenic regions that can be used in the compositions and methods described herein include the F1 phage AK gene having a sequence of: GGGATTTTGCCGATTTCGGCCTATTGGTTAA AAAATGAGCTGATTTAACAAAAATTTAACGCGAATTTTAACAAAAT (SEQ ID NO: 19) and alkaline phosphatase (AP) gene fragments as described in U.S. Pat. No. 8,236,557, which are incorporated herein by reference. In some embodiments, the AP gene fragment has the sequence of:

(SEQ ID NO: 62)
CCCCGGGTGCGCGGCGTCGGTGGTGCCGGGGGGGGCGCCAGGTCGCAGGCGG
TGTAGGGCTCCAGGCAGGCGGCGAAGGCCATGACGTGCGCTATGAAGGTCTGCTC
CTGCACGCCGTGAACCAGGTGCGCCTGCGGGCCGCGCGCGAACACCGCCACGTC
CTCGCCTGCGTGGGTCTCTTCGTCCAGGGGCACTGCTGACTGCTGCCGATACTCG
GGGCTCCCGCTCTCGCTCTCGGTAACATCCGGCCGGGCGCCGTCCTTGAGCACAT
AGCCTGGACCGTTTCCGTATAGGAGGACCGTGTAGGCCTTCCTGTCCCGGGCCTTG
CCAGCGGCCAGCCCGATGAAGGAGCTCCCTCGCAGGGGGTAGCCTCCGAAGGAG
AAGACGTGGGAGTGGTCGGCAGTGACGAGGCTCAGCGTGTCCTCCTCGCTGGTGA
GCTGGCCCGCCCTCTCAATGGCGTCGTCGAACATGATCGTCTCAGTCAGTGCCCGG
TAAGCCCTGCTTTCATGATGACCATGGTCGATGCGACCACCCTCCACGAAGAGGAA
GAAGCCGCGGGGGTGTCTGCTCAGCAGGCGCAGGGCAGCCTCTGTCATCTCCATC
AGGGAGGGGTCCAGTGTGGAGTCTCGGTGGATCTCGTATTTCATGTCTCCAGGCTC
AAAGAGACCCATGAGATGGGTCACAGACGGGTCCAGGGAAGCCTGCATGAGCTCA
GTGCGGTTCCACACGTACCGGGCACCCTGGCGTTCGCCGAGCCATTCCTGCACCA
GATTCTTCCCGTCCAGCCTGGTCCCACCTTGGCTGTAGTCATCTGGGTACTCAGGG
TCTGGGGTTCCCATGCGAAACATGTACTTTCGGCCTCCA.
In some embodiments, the AP gene fragment has the sequence of:
(SEQ ID NO: 63)
CCCCGGGTGCGCGGCGTCGGTGGTGCCGGGGGGGGCGCCAGGTCGCAGGCGG
TGTAGGGCTCCAGGCAGGCGGCGAAGGCCATGACGTGCGCTATGAAGGTCTGCTC
CTGCACGCCGTGAACCAGGTGCGCCTGCGGGCCGCGCGCGAACACCGCCACGTC
CTCGCCTGCGTGGGTCTCTTCGTCCAGGGGCACTGCTGACTGCTGCCGATACTCG
GGGCTCCCGCTCTCGCTCTCGGTAACATCCGGCCGGGCGCCGTCCTTGAGCACAT
AGCCTGGACCGTTTCCGTATAGGAGGACCGTGTAGGCCTTCCTGTCCCGGGCCTTG
CCAGCGGCCAGCCCGATGAAGGAGCTCCCTCGCAGGGGGTAGCCTCCGAAGGAG
AAGACGTGGGAGTGGTCGGCAGTGACGAGGCTCAGCGTGTCCTCCTCG CTGGTGA.
In some embodiments, the AP gene fragment has the sequence of:
(SEQ ID NO: 64)
GCTGGCCCGCCCTCTCAATGGCGTCGTCGAACATGATCGTCTCAGTCAGTGCCCGG
TAAGCCCTGCTTTCATGATGACCATGGTCGATGCGACCACCCTCCACGAAGAGGAA
GAAGCCGCGGGGGTGTCTGCTCAGCAGGCGCAGGGCAGCCTCTGTCATCTCCATC
AGGGAGGGGTCCAGTGTGGAGTCTCGGTGGATCTCGTATTTCATGTCTCCAGGCTC
AAAGAGACCCATGAGATGGGTCACAGACGGGTCCAGGGAAGCCTGCATGAGCTCA
GTGCGGTTCCACACGTACCGGGCACCCTGGCGTTCGCCGAGCCATTCCTGCACCA
GATTCTTCCCGTCCAGCCTGGTCCCACCTTGGCTGTAGTCATCTGGGTACTCAGGG
TCTGGGGTTCCCATGCGAAACATGTACTTTCGGCCTCCA.
In some embodiments, the AP gene fragment has the sequence of:
(SEQ ID NO: 65)
CCCCGGGTGCGCGGCGTCGGTGGTGCCGGGGGGGGCGCCAGGTCGCAGGCGG
TGTAGGGCTCCAGGCAGGCGGCGAAGGCCATGACGTGCGCTATGAAGGTCTGCTC
CTGCACGCCGTGAACCAGGTGCGCCTGCGGGCCGCGCGCGAACACCGCCACGTC
CTCGCCTGCGTGGGTCTCTTCGTCCAGGGGCACTGCTGACTGCTGCCGATACTCG
GGGCTCCCGCTCTCGCTCTCGGTAACATCCGGCCGGGCGCCGTCCTTGAGCACAT
AGCCTGGACCGTTTC
In some embodiments, the AP gene fragment has the sequence of:
(SEQ ID NO: 66)
CGTATAGGAGGACCGTGTAGGCCTTCCTGTCCCGGGCCTTGCCAGCGGCCAGCCC
GATGAAGGAGCTCCCTCGCAGGGGGTAGCCTCCGAAGGAGAAGACGTGGGAGTGG
TCGGCAGTGACGAGGCTCAGCGTGTCCTCCTCGCTGGTGAGCTGGCCCGCCCTCT
CAATGGCGTCGTCGAACATGATCGTCTCAGTCAGTGCCCGGTAAGCCCTGCTTTCA
TGATGACCATGGTCGATGCGACCACCCTCCACGAAGAGGAAGAAGCCGCGGGGGT
GTCTGCTCAGCAGG.
In some embodiments, the AP gene fragment has the sequence of:
(SEQ ID NO: 67)
CGCAGGGCAGCCTCTGTCATCTCCATCAGGGAGGGGTCCAGTGTGGAGTCTCGGT
GGATCTCGTATTTCATGTCTCCAGGCTCAAAGAGACCCATGAGATGGGTCACAGAC
GGGTCCAGGGAAGCCTGCATGAGCTCAGTGCGGTTCCACACGTACCGGGCACCCT
GGCGTTCGCCGAGCCATTCCTGCACCAGATTCTTCCCGTCCAGCCTGGTCCCACCT
TGGCTGTAGTCATCTGGGTACTCAGGGTCTGGGGTTCCCATGCGAAACATGTACTTT
CGGCCTCCA.

An exemplary splice donor sequence for use in the methods and compositions described herein (e.g., in trans-splicing and dual hybrid approaches) has the sequence: GTAAGTATCAAGGTTACAAGAC AGGTTTAAGGAGACCAATAGAAACTGGGCTTGTCGAGACAGAGAAGACTCTTGCGTTTCT (SEQ ID NO: 20). An exemplary splice acceptor sequence for use in the methods and compositions described herein (e.g., in trans-splicing and dual hybrid approaches) has the sequence: GATAGGCACCTATTGG TCTTACTGACATCCACTTTGCCTTTCTCTCCACAG (SEQ ID NO: 21). The splice donor sequence GTAAGTATCAAGGTTACAAGACAGGTTTAAGGAGACCAATAGAAACTGGGCTTGTCGAGACAGAGAA GACTCTTGCGTTTCTGA (SEQ ID NO: 68) and the splice acceptor sequence TAGGCACCTATTGGTCTTACTGACATCCACTTTGCCTTTCTCTCCACAG (SEQ ID NO: 69) can also be used in the methods and compositions described herein. Additional examples of splice donor and splice acceptor sequences are known in the art.

Dual hybrid vectors for use in the methods and compositions described herein are designed such that approximately half of the OTOF gene is contained within each vector (e.g., each vector contains a polynucleotide that encodes approximately half of the OTOF protein). The determination of how to split the polynucleotide sequence between the two nucleic acid vectors is made based on the size of the promoter and the locations of the portions of the polynucleotide that encode the OTOF C2 domains. When a short promoter is used in the dual hybrid vector system (e.g., a promoter that is 1 kb or shorter, e.g., approximately 1 kb, 950 bp, 900 bp, 850 bp, 800 bp, 750 bp, 700 bp, 650 bp, 600 bp, 550 bp 500 bp, 450 bp, 400 bp, 350 bp, 300 bp or shorter), such as CAG, CMV, smCBA, or a Myo15 promoter having a sequence that is 1 kb or shorter (e.g., a Myo15 promoter described hereinabove, e.g., a Myo15 promoter having the sequence of any one of SEQ ID NOs: 38, 39, or 49-60), the OTOF polynucleotide sequence is divided between the two nucleic acid vectors at an exon boundary that occurs after the portion of the polynucleotide that encodes the C2D domain and before the portion of the polynucleotide that encodes C2E domain, for example, the exon 26/27 boundary. The nucleic acid vectors containing promoters of this size can optionally contain OTOF UTRs (e.g., full-length 5′ and 3′ UTRs). When a long promoter is used in the dual hybrid vector system (e.g., a promoter that is longer than 1 kb, e.g., 1.1 kb, 1.25 kb, 1.5 kb, 1.75 kb, 2 kb, 2.5 kb, 3 kb or longer), such as a Myo15 promoter that is longer than 1 kb (e.g., SEQ ID NO: 36), the OTOF polynucleotide sequence will be divided between the two nucleic acid vectors at an exon boundary that occurs after the portion of the polynucleotide that encodes the C2C domain, and either before the portion of the polynucleotide that encodes the C2D domain, such as the exon 19/20 boundary, or within the portion of the polynucleotide that encodes the C2D domain, such as the exon 25/26 boundary. A short promoter (e.g., a CMV promoter, CAG promoter, smCBA promoter, or a Myo15 promoter having a sequence that is 1 kb or shorter, e.g., a Myo15 promoter having the sequence of any one of SEQ ID NOs: 38, 39, or 49-60) can also be used in the dual vector systems designed for large promoters, in which case additional elements (e.g., OTOF UTR sequences) may be included in the first vector (e.g., the vector containing the portion of the polynucleotide the encodes the C2C domain).

One exemplary dual hybrid vector system that uses a short promoter includes a first nucleic acid vector containing a CAG promoter operably linked to exons 1-26 of a polynucleotide encoding an OTOF protein (e.g., human OTOF, e.g., SEQ ID NO: 1 or SEQ ID NO: 5), a splice donor sequence 3′ of the polynucleotide sequence, and a recombinogenic region 3′ of the splice donor sequence; and a second nucleic acid vector containing a recombinogenic region, a splice acceptor sequence 3′ of the recombinogenic region, a polynucleotide 3′ of the splice acceptor sequence that contains exons 27-48 of a polynucleotide encoding an OTOF protein (e.g., human OTOF, e.g., SEQ ID NO: 1 or SEQ ID NO: 5), and a poly(A) sequence (e.g., a bGH poly(A) signal sequence). The first and second nucleic acid vectors can also contain the full length 5′ and 3′ OTOF UTRs, respectively (e.g., the 127 bp human OTOF 5′ UTR can be included in the first nucleic acid vector, and the 1035 bp human OTOF 3′ UTR can be included in the second nucleic acid vector). Another exemplary dual hybrid vector system that uses a short promoter includes a first nucleic acid vector containing a CAG promoter operably linked to exons 1-28 of a polynucleotide encoding an OTOF protein (e.g., mouse OTOF, e.g., SEQ ID NO: 6), a splice donor sequence 3′ of the polynucleotide sequence, and a recombinogenic region 3′ of the splice donor sequence; and a second nucleic acid vector containing a recombinogenic region, a splice acceptor sequence 3′ of the recombinogenic region, a polynucleotide 3′ of the splice acceptor sequence that contains exons 29-48 of a polynucleotide encoding an OTOF protein (e.g., mouse OTOF, e.g., SEQ ID NO: 6), and a poly(A) sequence (e.g., a bGH poly(A) signal sequence). The first and second nucleic acid vectors can also contain the full length 5′ and 3′ OTOF UTRs, respectively (e.g., the 134 bp mouse OTOF 5′ UTR can be included in the first nucleic acid vector, and the 1001 bp mouse OTOF 3′ UTR can be included in the second nucleic acid vector). The CMV promoter, smCBA promoter, or a Myo15 promoter having a sequence that is 1 kb or shorter (e.g., a Myo15 promoter described hereinabove, e.g., a Myo15 promoter having the sequence of any one of SEQ ID NOs: 38, 39, or 49-60) can be used in place of the CAG promoter either of the foregoing dual vector systems.

An exemplary dual hybrid vector system that uses a long promoter includes a first nucleic acid vector containing a Myo15 promoter that is longer than 1 kb (e.g., SEQ ID NO: 36) operably linked to exons 1-19 of a polynucleotide encoding an OTOF protein (e.g., human OTOF, e.g., SEQ ID NO: 1 or SEQ ID NO: 5), a splice donor sequence 3′ of the polynucleotide sequence, and a recombinogenic region 3′ of the splice donor sequence; and a second nucleic acid vector containing a recombinogenic region, a splice acceptor sequence 3′ of the recombinogenic region, a polynucleotide 3′ of the splice acceptor sequence that contains exons 20-48 of a polynucleotide encoding an OTOF protein (e.g., human OTOF, e.g., SEQ ID NO: 1 or SEQ ID NO: 5), and a poly(A) sequence (e.g., a bGH poly(A) signal sequence). Another exemplary dual hybrid vector system that uses a long promoter includes a first nucleic acid vector containing a Myo15 promoter that is longer than 1 kb (e.g., SEQ ID NO: 36) operably linked to exons 1-20 of a polynucleotide encoding an OTOF protein (e.g., mouse OTOF, e.g., SEQ ID NO: 6), a splice donor sequence 3′ of the polynucleotide sequence, and a recombinogenic region 3′ of the splice donor sequence; and a second nucleic acid vector containing a recombinogenic region, a splice acceptor sequence 3′ of the recombinogenic region, a polynucleotide 3′ of the splice acceptor sequence that contains exons 21-48 of a polynucleotide encoding an OTOF protein (e.g., mouse OTOF, e.g., SEQ ID NO: 6), and a poly(A) sequence (e.g., a bGH poly(A) signal sequence). Neither the first nor the second nucleic acid vector in either of the foregoing Myo15 promoter dual hybrid vector systems contains an OTOF UTR. A short promoter (e.g., a CMV promoter, smCBA promoter, CAG promoter, or a Myo15 promoter having a sequence that is 1 kb or shorter, e.g., a Myo15 promoter having the sequence of any one of SEQ ID NOs: 38, 39, or 49-60) can also be used in the foregoing dual vector systems designed for large promoters. If these dual vector systems contain a short promoter, they may also include a 5′ OTOF UTR in the first vector.

To accommodate an OTOF UTR, the OTOF coding sequence can be divided in a different position. For example, in a dual hybrid vector system in which the first nucleic acid vector contains a Myo15 promoter that is longer than 1 kb (e.g., SEQ ID NO: 36) operably linked to exons 1-25 of a polynucleotide encoding an OTOF protein (e.g., human OTOF, e.g., SEQ ID NO: 1 or SEQ ID NO: 5), a splice donor sequence 3′ of the polynucleotide sequence, and a recombinogenic region 3′ of the splice donor sequence; and the second nucleic acid vector contains a recombinogenic region, a splice acceptor sequence 3′ of the recombinogenic region, a polynucleotide 3′ of the splice acceptor sequence that contains exons 26-48 of a polynucleotide encoding an OTOF protein (e.g., human OTOF, e.g., SEQ ID NO: 1 or SEQ ID NO: 5), and a poly(A) sequence (e.g., a bGH poly(A) signal sequence), the second nucleic acid can also contain a full-length OTOF 3′ UTR (e.g., the 1035 bp human OTOF UTR). For mouse OTOF, the dual hybrid vector system can contain a 3′ UTR if the first nucleic acid vector contains a Myo15 promoter that is longer than 1 kb (e.g., SEQ ID NO: 36) operably linked to exons 1-24 of a polynucleotide encoding an OTOF protein (e.g., mouse OTOF, e.g., SEQ ID NO: 6), a splice donor sequence 3′ of the polynucleotide sequence, and a recombinogenic region 3′ of the splice donor sequence; and the second nucleic acid vector contains a recombinogenic region, a splice acceptor sequence 3′ of the recombinogenic region, a polynucleotide 3′ of the splice acceptor sequence that contains exons 25-48 of a polynucleotide encoding an OTOF protein (e.g., human OTOF, e.g., SEQ ID NO: 1 or SEQ ID NO: 5), and a poly(A) sequence (e.g., a bGH poly(A) signal sequence). In this dual hybrid vector system, the second nucleic acid can also contain a full-length OTOF 3′ UTR (e.g., the 1001 bp mouse OTOF UTR). A short promoter (e.g., a CMV promoter, smCBA promoter, CAG promoter, or a Myo15 promoter having a sequence that is 1 kb or shorter, e.g., a Myo15 promoter having the sequence of any one of SEQ ID NOs: 38, 39, or 49-60) can also be used in the foregoing dual vector systems designed for large promoters. If these dual vector systems contain a short promoter, they may also include a 5′ OTOF UTR in the first vector.

The dual hybrid vectors used in the methods and compositions described herein can optionally include a degradation signal sequence in both the first and second nucleic acid vectors. The degradation signal sequence can be included to prevent or reduce the expression of portions of the OTOF protein from polynucleotides that failed to recombine and/or undergo splicing. The degradation signal sequence is positioned 3′ of the recombinogenic region in the first nucleic acid vector, and is positioned between the recombinogenic region and the splice acceptor in the second nucleic acid vector. A degradation signal sequence that can be used in the compositions and methods described herein has the sequence of: GCCTGCAAGAACTGGTTCAGCAGCCTGAGCCACTTCGTGATCCACCTG (SEQ ID NO: 22).

Exemplary pairs of overlapping, trans-splicing, and dual hybrid vectors are described in Table 4 below.

Table 4: Exemplary pairs of overlapping, trans-splicing, and hybrid dual vectors for use in the methods and compositions described herein Vector Vector Type Vector Pair Pair Number 1 Overlapping First nucleic acid vector contains: CAG promoter operably linked to exons 1-24 and the 500 kb 3′ of the exon 24/25 boundary of a polynucleotide encoding a human OTOF protein

TABLE 4
Exemplary pairs of overlapping, trans-splicing, and hybrid dual vectors for use in the
methods and compositions described herein

| Vector | | |
|---|---|---|
| Pair | | |
| Number | Vector Type | Vector Pair |
| 1 | Overlapping | First nucleic acid vector contains: CAG promoter operably linked to exons |
| | | 1-24 and the 500 kb 3′ of the exon 24/25 boundary of a polynucleotide |
| | | encoding a human OTOF protein |
| | | Second nucleic acid vector contains: the 500 kb 5′ of the exon 24/25 |
| | | boundary and exons 25-48 of a polynucleotide encoding a human OTOF |
| | | protein and a bGH poly(A) sequence |
| 2 | Overlapping | First nucleic acid vector contains: CAG promoter operably linked to exons |
| | | 1-28 and the 500 kb 3′ of the exon 28/29 boundary of a polynucleotide |
| | | encoding a human OTOF protein |
| | | Second nucleic acid vector contains: the 500 kb 5′ of the exon 28/29 |
| | | boundary and exons 29-48 of a polynucleotide encoding a human OTOF |
| | | protein and a bGH poly(A) sequence |
| 3 | Overlapping | First nucleic acid vector contains: Myo15 promoter operably linked to |
| | | exons 1-21 and the 500 kb 3′ of the exon 21/22 boundary of a |
| | | polynucleotide encoding a human OTOF protein |
| | | Second nucleic acid vector contains: the 500 kb 5′ of the exon 21/22 |
| | | boundary and exons 22-48 of a polynucleotide encoding a human OTOF |
| | | protein and a bGH poly(A) sequence |
| 4 | Trans- | First nucleic acid vector contains: CAG promoter operably linked to exons |
| | splicing | 1-26 of a polynucleotide encoding a human OTOF protein and a splice |
| | | donor sequence 3′ of the polynucleotide |
| | | Second nucleic acid vector contains: a splice acceptor sequence 5′ of |
| | | exons 27-48 of a polynucleotide encoding a human OTOF protein and a |
| | | bGH poly(A) sequence |
| 5 | Trans- | First nucleic acid vector contains: Myo15 promoter operably linked to |
| | splicing | exons 1-19 of a polynucleotide encoding a human OTOF protein and a |
| | | splice donor sequence 3′ of the polynucleotide |
| | | Second nucleic acid vector contains: a splice acceptor sequence 5′ of |
| | | exons 20-48 of a polynucleotide encoding a human OTOF protein and a |
| | | bGH poly(A) sequence |
| 6 | Trans- | First nucleic acid vector contains: Myo15 promoter operably linked to |
| | splicing | exons 1-25 of a polynucleotide encoding a human OTOF protein and a |
| | | splice donor sequence 3′ of the polynucleotide |
| | | Second nucleic acid vector contains: a splice acceptor sequence 5′ of |
| | | exons 26-48 of a polynucleotide encoding a human OTOF protein and a |
| | | bGH poly(A) sequence |
| 7 | Hybrid | First nucleic acid vector contains: CAG promoter operably linked to exons |
| | | 1-26 of a polynucleotide encoding a human OTOF protein, a splice donor |
| | | sequence 3′ of the polynucleotide, and a recombinogenic region 3′ of the |
| | | splice donor sequence |
| | | Second nucleic acid vector contains: a recombinogenic region, a splice |
| | | acceptor sequence 3′ of the recombinogenic region, exons 27-48 of a |
| | | polynucleotide encoding a human OTOF protein 3′ of the splice acceptor |
| | | sequence, and a bGH poly(A) sequence |
| 8 | Hybrid | First nucleic acid vector contains: Myo15 promoter operably linked to |
| | | exons 1-19 of a polynucleotide encoding a human OTOF protein, a splice |
| | | donor sequence 3′ of the polynucleotide, and a recombinogenic region 3′ |
| | | of the splice donor sequence |
| | | Second nucleic acid vector contains: a recombinogenic region, a splice |
| | | acceptor sequence 3′ of the recombinogenic region, exons 20-48 of a |
| | | polynucleotide encoding a human OTOF protein 3′ of the splice acceptor |
| | | sequence, and a bGH poly(A) sequence |
| 9 | Hybrid | First nucleic acid vector contains: Myo15 promoter operably linked to |
| | | exons 1-25 of a polynucleotide encoding a human OTOF protein, a splice |
| | | donor sequence 3′ of the polynucleotide, and a recombinogenic region 3′ |
| | | of the splice donor sequence |
| | | Second nucleic acid vector contains: a recombinogenic region, a splice |
| | | acceptor sequence 3′ of the recombinogenic region, exons 26-48 of a |
| | | polynucleotide encoding a human OTOF protein 3′ of the splice acceptor |
| | | sequence, and a bGH poly(A) sequence |

### Vectors for the Expression of OTOF

In addition to achieving high rates of transcription and translation, stable expression of an exogenous gene in a mammalian cell can be achieved by integration of the polynucleotide containing the gene into the nuclear genome of the mammalian cell. A variety of vectors for the delivery and integration of polynucleotides encoding exogenous proteins into the nuclear DNA of a mammalian cell have been developed. Examples of expression vectors are disclosed in, e.g., WO 1994/011026 and are incorporated herein by reference. Expression vectors for use in the compositions and methods described herein contain a polynucleotide sequence that encodes a portion of OTOF, as well as, e.g., additional sequence elements used for the expression of these agents and/or the integration of these polynucleotide sequences into the genome of a mammalian cell. Certain vectors that can be used for the expression of OTOF include plasmids that contain regulatory sequences, such as promoter and enhancer regions, which direct gene transcription. Other useful vectors for expression of OTOF contain polynucleotide sequences that enhance the rate of translation of these genes or improve the stability or nuclear export of the mRNA that results from gene transcription. These sequence elements include, e.g., 5′ and 3′ untranslated regions and a polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

### AAV Vectors for Nucleic Acid Delivery

In some embodiments, nucleic acids of the compositions and methods described herein are incorporated into recombinant AAV (rAAV) vectors and/or virions in order to facilitate their introduction into a cell. rAAV vectors useful in the compositions and methods described herein are recombinant nucleic acid constructs that include (1) a heterologous sequence to be expressed (e.g., a polynucleotide encoding an N-terminal or C-terminal portion of an OTOF protein) and (2) viral sequences that facilitate stability and expression of the heterologous genes. The viral sequences may include those sequences of AAV that are required in cis for replication and packaging (e.g., functional ITRs) of the DNA into a virion. Such rAAV vectors may also contain marker or reporter genes. Useful rAAV vectors have one or more of the AAV WT genes deleted in whole or in part but retain functional flanking ITR sequences. The AAV ITRs may be of any serotype suitable for a particular application. For use in the methods and compositions described herein, the ITRs can be AAV2 ITRs. Methods for using rAAV vectors are described, for example, in Tal et al., J. Biomed. Sci. 7:279 (2000), and Monahan and Samulski, Gene Delivery 7:24 (2000), the disclosures of each of which are incorporated herein by reference as they pertain to AAV vectors for gene delivery.

The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described, for instance, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery. rAAV virions useful in conjunction with the compositions and methods described herein include those derived from a variety of AAV serotypes including AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, and PHP.S. For targeting cochlear hair cells, AAV1, AAV2, AAV6, AAV9, Anc80, Anc80L65, DJ/9, 7m8, and PHP.B may be particularly useful. Serotypes evolved for transduction of the retina may also be used in the methods and compositions described herein. The first and second nucleic acid vectors in the compositions and methods described herein may have the same serotype or different serotypes. Construction and use of AAV vectors and AAV proteins of different serotypes are described, for instance, in Chao et al., Mol. Ther. 2:619 (2000); Davidson et al., Proc. Natl. Acad. Sci. USA 97:3428 (2000); Xiao et al., J. Virol. 72:2224 (1998); Halbert et al., J. Virol. 74:1524 (2000); Halbert et al., J. Virol. 75:6615 (2001); and Auricchio et al., Hum. Molec. Genet. 10:3075 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype (e.g., AAV9) pseudotyped with a capsid gene derived from a serotype other than the given serotype (e.g., AAV1, AAV2, AAV3, AAV4, AAV5, AAV6, AAV7, AAV8, etc.). Techniques involving the construction and use of pseudotyped rAAV virions are known in the art and are described, for instance, in Duan et al., J. Virol. 75:7662 (2001); Halbert et al., J. Virol. 74:1524 (2000); Zolotukhin et al., Methods, 28:158 (2002); and Auricchio et al., Hum. Molec. Genet. 10:3075 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet., 25:436 (2000) and Kolman and Stemmer, Nat. Biotechnol. 19:423 (2001).

### Pharmaceutical Compositions

The nucleic acid vectors described herein may be incorporated into a vehicle for administration into a patient, such as a human patient suffering from sensorineural hearing loss or auditory neuropathy, as described herein. Pharmaceutical compositions containing vectors, such as viral vectors, that contain a polynucleotide encoding a portion of an OTOF protein can be prepared using methods known in the art. For example, such compositions can be prepared using, e.g., physiologically acceptable carriers, excipients or stabilizers (Remington's Pharmaceutical Sciences 16th edition, Osol, A. Ed. (1980); incorporated herein by reference), and in a desired form, e.g., in the form of lyophilized formulations or aqueous solutions.

Mixtures of the nucleic acid vectors (e.g., viral vectors) described herein may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated. For local administration to the inner ear, the composition may be formulated to contain a synthetic perilymph solution. An exemplary synthetic perilymph solution includes 20-200 mM NaCl, 1-5 mM KCl, 0.1-10 mM CaCl₂), 1-10 mM glucose, and 2-50 mM HEPEs, with a pH between about 6 and 9 and an osmolality of about 300 mOsm/kg. The person responsible for administration will, in any event, determine the appropriate dose for the individual subject. Moreover, for human administration, preparations may meet sterility, pyrogenicity, general safety, and purity standards as required by FDA Office of Biologics standards.

### Methods of Treatment

The compositions described herein may be administered to a subject with sensorineural hearing loss or auditory neuropathy by a variety of routes, such as local administration to the inner ear (e.g., administration into the perilymph or endolymph, e.g., through the oval window, round window, or horizontal canal, e.g., administration to a cochlear hair cell), intravenous, parenteral, intradermal, transdermal, intramuscular, intranasal, subcutaneous, percutaneous, intratracheal, intraperitoneal, intraarterial, intravascular, inhalation, perfusion, lavage, and oral administration. The most suitable route for administration in any given case will depend on the particular composition administered, the patient, pharmaceutical formulation methods, administration methods (e.g., administration time and administration route), the patient's age, body weight, sex, severity of the disease being treated, the patient's diet, and the patient's excretion rate. Compositions may be administered once, or more than once (e.g., once annually, twice annually, three times annually, bi-monthly, monthly, or bi-weekly). In some embodiments, the first and second nucleic acid vectors are administered simultaneously (e.g., in one composition). In some embodiments, the first and second nucleic acid vectors are administered sequentially (e.g., the second nucleic acid vector is administered immediately after the first nucleic acid vector, or 5 minutes, 10 minutes, 15 minutes, 20 minutes, 25 minutes, 30 minutes, 45 minutes, 1 hour, 2 hours, 8 hours, 12 hours, 1 day, 2 days, 7 days, two weeks, 1 month or more after the first nucleic acid vector). The first and second nucleic acid vector can have the same serotype or different serotypes (e.g., AAV serotypes).

Subjects that may be treated as described herein are subjects having or at risk of developing sensorineural hearing loss or auditory neuropathy. The compositions and methods described herein can be used to treat subjects having a mutation in OTOF (e.g., a mutation that reduces OTOF function or expression, or an OTOF mutation associated with sensorineural hearing loss), subjects having a family history of autosomal recessive sensorineural hearing loss or deafness (e.g., a family history of OTOF-related hearing loss), or subjects whose OTOF mutational status and/or OTOF activity level is unknown. The methods described herein may include a step of screening a subject for a mutation in OTOF prior to treatment with or administration of the compositions described herein. A subject can be screened for an OTOF mutation using standard methods known to those of skill in the art (e.g., genetic testing). The methods described herein may also include a step of assessing hearing in a subject prior to treatment with or administration of the compositions described herein. Hearing can be assessed using standard tests, such as audiometry, ABR, electrocochleography (ECOG), and otoacoustic emissions. The compositions and methods described herein may also be administered as a preventative treatment to patients at risk of developing hearing loss or auditory neuropathy, e.g., patients who have a family history of inherited hearing loss or patients carrying an OTOF mutation who do not yet exhibit hearing loss or impairment.

Treatment may include administration of a composition containing the nucleic acid vectors (e.g., AAV viral vectors) described herein in various unit doses. Each unit dose will ordinarily contain a predetermined-quantity of the therapeutic composition. The quantity to be administered, and the particular route of administration and formulation, are within the skill of those in the clinical arts. A unit dose need not be administered as a single injection but may include continuous infusion over a set period of time. Dosing may be performed using a syringe pump to control infusion rate in order to minimize damage to the cochlea. In cases in which the nucleic acid vectors are AAV vectors (e.g., AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, or PHP.S vectors), the viral vectors may be administered to the patient at a dose of, for example, from about 1×10¹⁰ vector genomes (VG) to 1×10¹⁵ VG (e.g., 1×10¹⁰ VG, 2×10¹⁰ VG, 3×10¹⁰ VG, 4×10¹⁰ VG, 5×10¹⁰ VG, 6×10¹⁰ VG, 7×10¹⁰ VG, 8×10¹⁰VG, 9×10¹⁰ VG, 1×10¹¹ VG, 2×10¹¹ VG, 3×10¹¹ VG, 4×10¹¹ VG, 5×10¹¹ VG, 6×10¹¹ VG, 7×10¹¹ VG, 8×10¹¹ VG, 9×10¹¹ VG, 1×10¹²VG, 2×10¹²VG, 3×10¹²VG, 4×10¹²VG, 5×10¹²VG, 6×10¹²VG, 7×10¹²VG, 8×10¹²VG, 9×10¹²VG, 1×10¹³VG, 2×10¹³VG, 3×10¹³VG, 4×10¹³VG, 5×10¹³ VG, 6×10¹³ VG, 7×10¹³ VG, 8×10¹³ VG, 9×10¹³ VG, 1×10¹⁴ VG, 2×10¹⁴ VG, 3×10¹⁴ VG, 4×10¹⁴ VG, 5×10¹⁴ VG, 6×10¹⁴ VG, 7×10¹⁴ VG, 8×10¹⁴ VG, 9×10¹⁴ VG, 1×10¹⁵ VG) in a volume of 1 μL to 200 μL (e.g., 1, 2, 3, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200 μL).

The compositions described herein are administered in an amount sufficient to improve hearing, increase WT OTOF expression (e.g., expression in a cochlear hair cell, e.g., an inner hair cell), or increase OTOF function. Hearing may be evaluated using standard hearing tests (e.g., audiometry, ABR, electrocochleography (ECOG), and otoacoustic emissions) and may be improved by 5% or more (e.g., 5%, 10%, 15%, 20%, 30%, 40%, 50%, 60%, 70%, 80%, 90%, 100% or more) compared to hearing measurements obtained prior to treatment. In some embodiments, the compositions are administered in an amount sufficient to improve the subject's ability to understand speech. The compositions described herein may also be administered in an amount sufficient to slow or prevent the development or progression of sensorineural hearing loss or auditory neuropathy (e.g., in subjects who carry a mutation in OTOF or have a family history of autosomal recessive hearing loss but do not exhibit hearing impairment, or in subjects exhibiting mild to moderate hearing loss). OTOF expression may be evaluated using immunohistochemistry, Western blot analysis, quantitative real-time PCR, or other methods known in the art for detection protein or mRNA, and may be increased by 5% or more (e.g., 5%, 10%, 15%, 20%, 30%, 40%, 50%, 60%, 70%, 80%, 90%, 100% or more) compared to OTOF expression prior to administration of the compositions described herein. OTOF function may be evaluated directly (e.g., using electrophysiological methods or imaging methods to assess exocytosis) or indirectly based on hearing tests, and may be increased by 5% or more (e.g., 5%, 10%, 15%, 20%, 30%, 40%, 50%, 60%, 70%, 80%, 90%, 100% or more) compared to OTOF function prior to administration of the compositions described herein. These effects may occur, for example, within 1 week, 2 weeks, 3 weeks, 4 weeks, 5 weeks, 6 weeks, 7 weeks, 8 weeks, 9 weeks, 10 weeks, 15 weeks, 20 weeks, 25 weeks, or more, following administration of the compositions described herein. The patient may be evaluated 1 month, 2 months, 3 months, 4 months, 5 months, 6 months or more following administration of the composition depending on the dose and route of administration used for treatment. Depending on the outcome of the evaluation, the patient may receive additional treatments.

### Kits

The compositions described herein can be provided in a kit for use in treating sensorineural hearing loss or auditory neuropathy (e.g., hearing loss associated with a mutation in OTOF). Compositions may include nucleic acid vectors described herein (e.g., a first nucleic acid vector containing a polynucleotide that encodes and N-terminal portion of an OTOF protein and a second nucleic acid vector containing a polynucleotide that encodes a C-terminal portion of an OTOF protein), optionally packaged in an AAV virus capsid (e.g., AAV1, AAV9, Anc80L65, DJ/9, or 7m8). The kit can further include a package insert that instructs a user of the kit, such as a physician, to perform the methods described herein. The kit may optionally include a syringe or other device for administering the composition.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1: Generation of Nucleic Acid Vectors that Recombine to Produce Full-Length OTOF

Gene fragments were synthesized and sub-cloned into an AAV2/cis-plasmid using restriction enzyme sites. Plasmids were maxi prepped to generate 1 mg of transfection grade plasmid. Inner ear-derived HEI-OC1 cells were seeded into a 12-well tissue culture dish 24 hours before plasmid transfection at a density of 200,000 cells/well. One microgram of each plasmid was transfected using Lipofectamine 3000 according to standard manufacturer's protocol. For wells that received both 5′ and 3′ plasmids, 1 μg of each was transfected for a total of 2 μg of DNA. As a positive control, full-length Otoferlin cDNA was also transfected. Cells were incubated with plasmid for 48 hours.

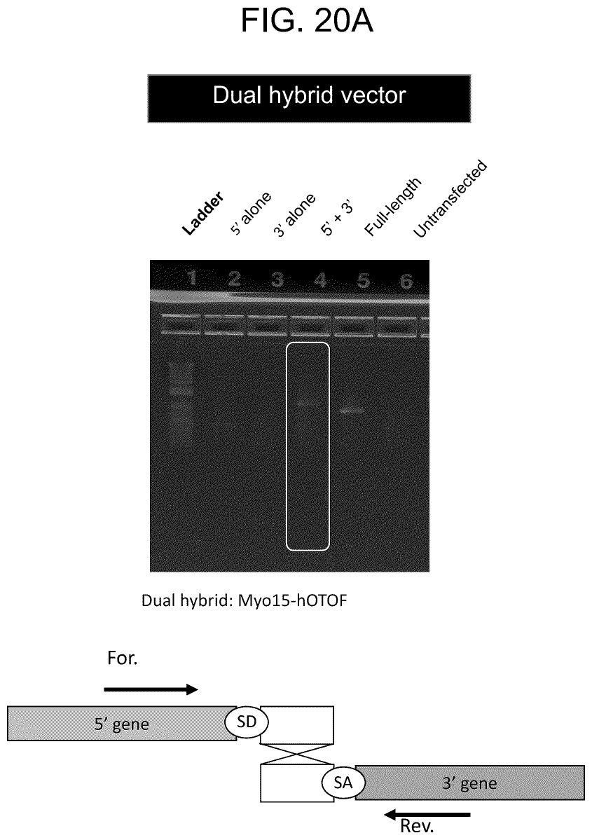

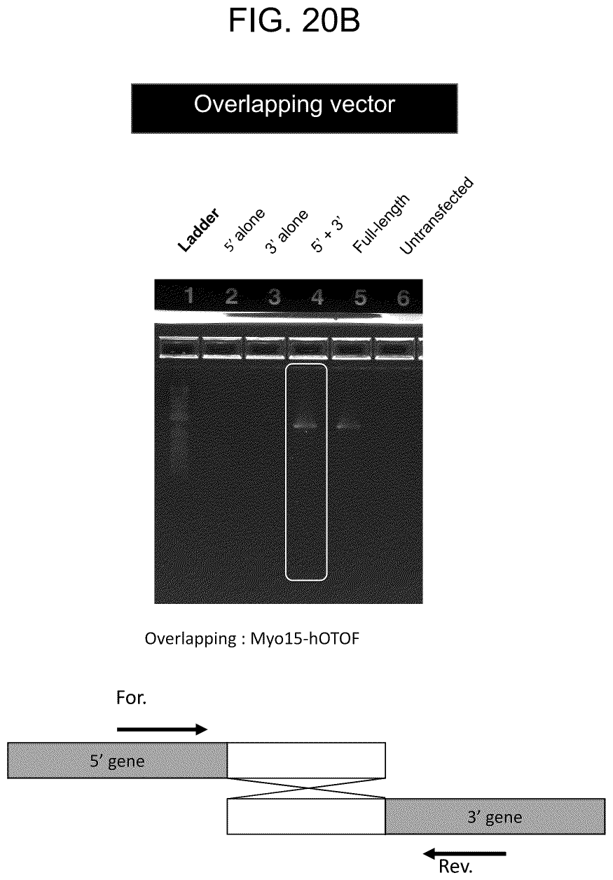

For PCR to check for recombination at the DNA level, genomic DNA was extracted from each well using Qiagen's DNeasy Blood and Tissue kit, according to standard manufacturer's protocol. PCR primers were designed to anneal to the plasmid outside of the region of overlap or splicing to generate an amplicon of ˜1200 bp. PCR was performed using My Taq 2× mastermix according to manufacturer's recommendations: annealing temperature of 58° C., elongation step of 30 seconds, and cycle number of 35×. Ten microliters of PCR product was run on a pre-cast 1.2% agarose E-gel and imaged on a BioRad gel doc imaging station. Both dual hybrid vectors (FIG. 20A) and overlapping vectors (FIG. 20B) showed evidence of recombination when the 5′ and 3′ plasmids were transfected together.

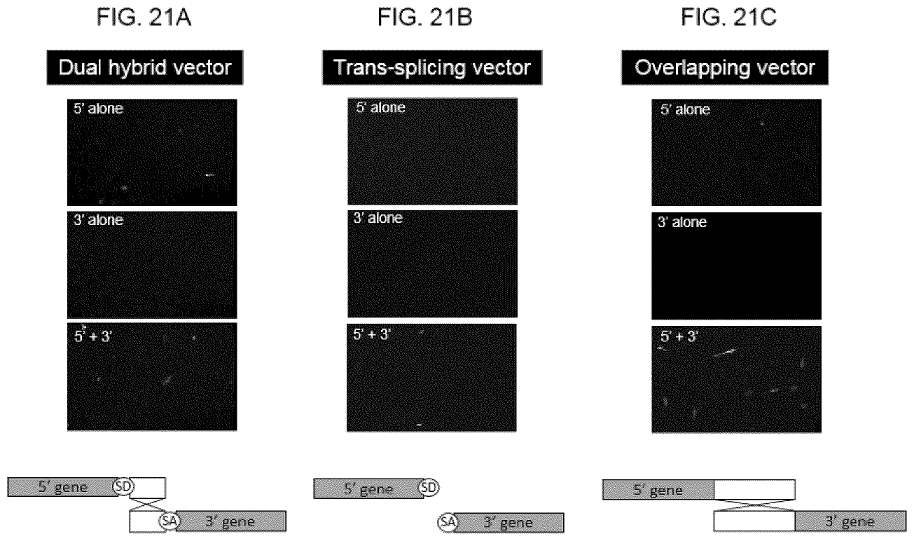

For immunofluorescence to check for recombination and generation of protein, cells were fixed with cold 4% PFA for 20 minutes at room temperature. Cells were washed three times with PBS and then permeabilized in a blocking solution of PBS with 10% normal donkey serum and 0.01% TritonX100. Cells were incubated in primary antibody overnight (mouse-anti-Otoferlin, Abcam ab53233) at a concentration of 1:1000 at 4° C. Cells were washed three times with PBS and incubated in secondary antibody for three hours at room temperature (donkey-anti-mouse Alexa Fluor 647, ThermoFisher A-31571). Cells were washed three times in PBS and stained with DAPI for 15 minutes at room temperature. Cells were imaged using a Zeiss inverted Apotome microscope. Increased staining was observed in cells transfected with both 5′ and 3′ plasmids compared to transfection of the 5′ or 3′ plasmid alone, indicating that the dual hybrid vector (FIG. 21A), trans-splicing vector (FIG. 21B), and overlapping vector (FIG. 21C) systems recombined in HEI-OC1 cells and generated OTOF protein.

### Example 2: Administration of a Composition Containing Dual Hybrid Vectors that Express OTOF to Mice Restores Electrophysiological Signatures of Hearing Function

Homozygous (HOM) OTOF-Q828X mice (a mouse model of human OTOF mutation p.Gln828Ter) were either left untreated or treated (by injection through the round window membrane) with 4E10 (4×10¹⁰) vector genomes (vg)/ear of an AAV1-Myo15 (SEQ ID NO: 38)-hOTOF (isoform 5, SEQ ID NO: 5) dual hybrid vector system in which exons 1-20 of the polynucleotide encoding the N-terminal portion of the OTOF protein and exons 21-46 and 48 of the polynucleotide encoding the C-terminal portion of the OTOF protein were delivered in separate vectors (FIG. 22A). An AP recombinogenic region (SEQ ID NO: 65) was included in both vectors of the dual hybrid vector system. Auditory brainstem response (ABR) thresholds were used to assess hearing function. Untreated animals (untreated Otof HOM) showed no detectable recovery in hearing function, whereas treated animals exhibited a robust recovery, which was consistent from four weeks post-treatment (Otof HOM at 4 weeks after treatment) to eight weeks post-treatment (Otof HOM at 8-11 weeks after treatment). ABR thresholds in heterozygous animals (Otof HET) were also tested.

In a separate set of experiments, homozygous OTOF-Q828X mice were either left untreated or treated (by injection through the round window membrane) with 4E10 (4×10¹⁰) vg/ear of an AAV1-truncated chimeric CMV-chicken β-actin (smCBA)-hOTOF (isoform 5, SEQ ID NO: 5) dual hybrid vector system as described above (FIG. 22B). The first vector contained exons 1-20 of the polynucleotide encoding the N-terminal portion of the OTOF protein and exons 21-46 and 48 of the polynucleotide encoding the C-terminal portion of the OTOF protein and both vectors contained an AP recombinogenic region (SEQ ID NO: 65). Untreated animals had no detectable recovery in hearing function, whereas treated animals exhibited a robust recovery at 4 weeks post-treatment (Otof HOM at 4 weeks after treatment). When these same animals were evaluated at 8 weeks post-treatment (Otof HOM at 8 weeks after treatment), ABR thresholds increased, suggesting less durable recovery with the smCBA promoter. ABR thresholds in heterozygous animals were also tested.

In yet another set of experiments, homozygous OTOF-Q828X mice were either left untreated or treated (by injection through the round window membrane) with an AAV1-smCBA-hOTOF (isoform 5, SEQ ID NO: 5) dual hybrid vector, as described above, at either 8E9 (8×10⁹) vg/ear (low dose), 1.6E10 (1.6×10¹⁰) vg vg/ear (mid dose), or 6.4E10 (6.4×10¹⁰) vg/ear (high dose). The first vector contained exons 1-20 of the polynucleotide encoding the N-terminal portion of the OTOF protein and exons 21-46 and 48 of the polynucleotide encoding the C-terminal portion of the OTOF protein and both vectors contained an AP recombinogenic region (SEQ ID NO: 65). ABR thresholds were used to assess hearing function at four weeks and eight weeks post-treatment (FIG. 22C). A dose-dependent recovery in ABR was observed at both timepoints. When comparing the eight weeks versus the four weeks timepoint, recovery of hearing function was steady for the low and mid doses but decreased for the high dose animals. ABR thresholds in heterozygous animals were also tested.

### Example 3: Administration of a Composition Containing Overlapping Dual Vectors that Express OTOF to a Subject with Sensorineural Hearing Loss

According to the methods disclosed herein, a physician of skill in the art can treat a patient, such as a human patient, with sensorineural hearing loss (e.g., sensorineural hearing loss associated with a mutation in OTOF) so as to improve or restore hearing. To this end, a physician of skill in the art can administer to the human patient a composition containing a first AAV vector (e.g., AAV1 or AAV9) containing a Myo15 promoter (e.g., SEQ ID NO: 36) operably linked to exons 1-21 of a polynucleotide encoding an OTOF protein (e.g., human OTOF, e.g., SEQ ID NO: 1 or SEQ ID NO: 5) and the 500 kb immediately 3′ of the exon 21/22 boundary, and a second AAV vector (e.g., AAV1 or AAV9) containing the 500 kb immediately 5′ of the exon 21/22 boundary and exons 22-48 of a polynucleotide encoding an OTOF protein (e.g., human OTOF, e.g., SEQ ID NO: 1 or SEQ ID NO: 5) and a bGH poly(A) sequence. The composition containing the overlapping dual AAV vectors may be administered to the patient, for example, by local administration to the inner ear (e.g., injection into the perilymph), to treat sensorineural hearing loss.

Following administration of the composition to a patient, a practitioner of skill in the art can monitor the expression of OTOF, and the patient's improvement in response to the therapy, by a variety of methods. For example, a physician can monitor the patient's hearing by performing standard tests, such as audiometry, ABR, electrocochleography (ECOG), and otoacoustic emissions following administration of the composition. A finding that the patient exhibits improved hearing in one or more of the tests following administration of the composition compared to hearing test results prior to administration of the composition indicates that the patient is responding favorably to the treatment. Subsequent doses can be determined and administered as needed.

### Example 4: Administration of a Composition Containing Trans-Splicing Dual Vectors that Express OTOF to a Subject with Sensorineural Hearing Loss

According to the methods disclosed herein, a physician of skill in the art can treat a patient, such as a human patient, with sensorineural hearing loss (e.g., sensorineural hearing loss associated with a mutation in OTOF) so as to improve or restore hearing. To this end, a physician of skill in the art can administer to the human patient a composition containing a first AAV vector (e.g., AAV1 or AAV9) containing a Myo15 promoter (e.g., SEQ ID NO: 36) operably linked to exons 1-19 of a polynucleotide encoding an OTOF protein (e.g., human OTOF, e.g., SEQ ID NO: 1 or SEQ ID NO: 5) and a splice donor sequence (e.g., SEQ ID NO: 20 or SEQ ID NO: 68) 3′ of the polynucleotide sequence, and a second AAV vector (e.g., AAV1 or AAV9) containing a splice acceptor sequence (e.g., SEQ ID NO: 21 or SEQ ID NO: 69) 5′ of exons 20-48 of a polynucleotide encoding an OTOF protein (e.g., human OTOF, e.g., SEQ ID NO: 1 or SEQ ID NO: 5) and a bGH poly(A) sequence. The composition containing the trans-splicing dual AAV vectors may be administered to the patient, for example, by local administration to the inner ear (e.g., injection into the perilymph), to treat sensorineural hearing loss.

Following administration of the composition to a patient, a practitioner of skill in the art can monitor the expression of OTOF, and the patient's improvement in response to the therapy, by a variety of methods. For example, a physician can monitor the patient's hearing by performing standard tests, such as audiometry, ABR, electrocochleography (ECOG), and otoacoustic emissions following administration of the composition. A finding that the patient exhibits improved hearing in one or more of the tests following administration of the composition compared to hearing test results prior to administration of the composition indicates that the patient is responding favorably to the treatment. Subsequent doses can be determined and administered as needed.

### Example 5: Administration of a Composition Containing Dual Hybrid Vectors that Express OTOF to a Subject with Sensorineural Hearing Loss

According to the methods disclosed herein, a physician of skill in the art can treat a patient, such as a human patient, with sensorineural hearing loss (e.g., sensorineural hearing loss associated with a mutation in OTOF) so as to improve or restore hearing. To this end, a physician of skill in the art can administer to the human patient a composition containing a first AAV vector (e.g., AAV1 or AAV9) containing a Myo15 promoter (e.g., SEQ ID NO: 36) operably linked to exons 1-19 of a polynucleotide encoding an OTOF protein (e.g., human OTOF, e.g., SEQ ID NO: 1 or SEQ ID NO: 5), a splice donor sequence (e.g., SEQ ID NO: 20 or SEQ ID NO: 68) 3′ of the polynucleotide sequence, and an F1 phage recombinogenic region (e.g., an F1 phage AK gene, SEQ ID NO: 19) 3′ of the splice donor sequence, and a second nucleic acid vector containing an F1 phage recombinogenic region (e.g., an F1 phage AK gene, SEQ ID NO: 19), a splice acceptor sequence (e.g., SEQ ID NO: 21 or SEQ ID NO: 69) 3′ of the recombinogenic region, a polynucleotide 3′ of the splice acceptor sequence that contains exons 20-48 of a polynucleotide encoding an OTOF protein (e.g., human OTOF, e.g., SEQ ID NO: 1 or SEQ ID NO: 5), and a bGH poly(A) sequence. The first and second dual hybrid AAV vectors can optionally include a degradation signal sequence (e.g., SEQ ID NO: 22) positioned 3′ of the recombinogenic region in the first nucleic acid vector, and positioned between the recombinogenic region and the splice acceptor sequence in the second nucleic acid vector. The composition containing the dual hybrid AAV vectors may be administered to the patient, for example, by local administration to the inner ear (e.g., injection into the perilymph), to treat sensorineural hearing loss.

Following administration of the composition to a patient, a practitioner of skill in the art can monitor the expression of OTOF, and the patient's improvement in response to the therapy, by a variety of methods. For example, a physician can monitor the patient's hearing by performing standard tests, such as audiometry, ABR, electrocochleography (ECOG), and otoacoustic emissions following administration of the composition. A finding that the patient exhibits improved hearing in one or more of the tests following administration of the composition compared to hearing test results prior to administration of the composition indicates that the patient is responding favorably to the treatment. Subsequent doses can be determined and administered as needed.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention. Other embodiments are in the claims.

1. A dual vector system comprising:
(a) a first adeno-associated virus serotype 1 (AAV1) vector comprising, in a 5′ to 3′ direction, a first AAV2 inverted terminal repeat (ITR) sequence, a Myosin 15 promoter having the sequence of SEQ ID NO: 38, a first coding polynucleotide that encodes an N-terminal portion of a human otoferlin (OTOF) protein isoform 5, a splice donor signal sequence, a first alkaline phosphatase (AP) gene fragment, and a second AAV2 ITR sequence; and
(b) a second AAV1 vector comprising, in a 5′ to 3′ direction, a first AAV2 ITR sequence, a second AP gene fragment, a splice acceptor signal sequence, a second coding polynucleotide that encodes a C-terminal of the human OTOF protein isoform 5, a polyadenylation (poly(A)) sequence, and a second AAV2 ITR sequence,
wherein
the first AP gene fragment and the second AP gene fragment have the same sequence, and
the first AAV1 vector and the second AAV1 vector undergo homologous recombination or concatemerization in a mammalian cell to form a nucleic acid that encodes a full-length human OTOF protein isoform 5.

2. The dual vector system of claim 1, wherein the first coding polynucleotide and the second coding polynucleotide do not overlap.

3. The dual vector system of claim 1, wherein neither the first AAV1 vector nor the second AAV1 vector encodes a full-length human OTOF protein isoform 5.

4. The dual vector system of claim 1, wherein the first coding polynucleotide and the second coding polynucleotide that encode the human OTOF protein isoform 5 do not comprise introns.

5. The dual vector system of claim 1, wherein the poly(A) sequence is a bovine growth hormone (bGH) poly(A) signal sequence.

6. The dual vector system of claim 1, wherein the sequence of the first AP gene fragment and the second AP gene fragment is SEQ ID NO: 65.
